(19) United States
(12) Patent Application Publication
(10) Pub. No.: US 2025/0281540 A1
(43) Pub. Date: Sep. 11, 2025

(54) Methods for Haematopoietic Stem Cell Transplantation

(71) Applicants: Ospedale San Raffaele S.r.l., Milan (IT); Fondazione Telethon ETS, Rome (IT)

(72) Inventors: Luigi Naldini, Milan (IT); Attya Omer Javed, Gorgonzola (IT); Gabriele Pedrazzani, Soresina (IT)

(21) Appl. No.: 18/859,880

(22) PCT Filed: Apr. 28, 2023
(86) PCT No.: PCT/EP2023/061382

§ 371 Date: Oct. 24, 2024

(30) Foreign Application Priority Data
Apr. 28, 2022 (IT) 102022000008531

(51) Int. Cl. A61K 35/28

(52) U.S. Cl. CPC A61K 35/28

(57) ABSTRACT

A method for haematopoietic stem and/or progenitor cell (HSPC) transplantation in a subject in need thereof, comprising the steps: (a) administering one or more HSPC mobiliser to the subject to mobilise the subject's endogenous HSPCs; and (b) administering a population of HSPCs to the subject.

### FIELD OF THE INVENTION

The present invention relates to methods for haematopoietic stem and/or progenitor cell (HSPC) transplantation. The invention also relates to the genetic modification of HSPCs for improving their engraftment during transplantation. The invention also relates to an RNA polynucleotide, and DNA polynucleotides and vectors encoding the same.

### BACKGROUND TO THE INVENTION

Hematopoietic stem cell transplantation (HSCT) is used to treat patients suffering from malignant and inherited diseases such as primary immunodeficiencies, Fanconi anemia, hemoglobinopathies and lysosomal storage disorders (see e.g. Granot, N., and Storb, R. (2020). Haematologica 105, 2716-2729).

However, the morbidity of HSCT remains of concern, in particular when considering application to non-malignant diseases. The main cause of HSCT morbidity, when using an allogeneic source of HSPCs, resides in the life-threatening and debilitating graft versus host disease (GvHD), and in the long-term irreversible complications (such as secondary malignancies) arising from the genotoxic side effects of conditioning regimens, which are required to deplete HSPCs residing in the bone marrow (BM) to make space for the donor HSPCs (see e.g. Copelan, E. A., et al. (2019). Blood Rev 34, 34-44).

The development of effective gene correction methods promoted the use of autologous HSPCs to treat inherited diseases (see e.g. Ferrari, G., et al. (2021). Nat Rev Genet 22, 216-234). While autologous HSPC gene therapy (HSPC-GT) eliminates the risk of GvHD, it maintains the requirement for partial or fully myeloablative conditioning. Current regimens involve non-specific, chemo- or radio-therapeutic treatments that have multiple short- and long-term adverse effects, and cause a prolonged immune suppression predisposing patients to severe and fatal infections (see e.g. Gyurkocza, B., and Sandmaier, B. M. (2014). Blood 124, 344-353). These treatments also damage the BM stroma and HSPC niche architecture and may in turn adversely affect the extent and kinetics of cells engraftment.

Strategies to bypass conditioning by increasing the input of donor cells or in vitro expansion prior to infusion have proved to be either inefficient or are currently not compatible with clinical use of human HSPCs. Strategies which use specific drugs that target HSPCs in the BM niche and spare non-hematopoietic cells (e.g. monoclonal antibodies coupled or not with toxin) are now reaching clinical testing, although profound cytopenias might result from the degree of ablation needed for sufficient engraftment.

Thus, there is a need for further strategies that reduce genotoxic conditioning regimens before HSPC transplantation

When the conditioning is milder, engraftment becomes a competitive process between endogenous and infused HSPCs (see e.g. Socie, G., et al. (1995). Leukemia Res 19, 497-504). Competition with residual cells in the recipient might be impaired when the infused cells undergo ex vivo genetic engineering. Culture conditions, exposure to viral vectors and electroporation of editing machinery can variably induce HSPC differentiation or apoptosis and modify expression of cell surface molecules relevant for BM homing and engraftment (see e.g. Hall, K. M., et al. (2006). Exp Hematol 34, 433-442). In addition, DNA double strand breaks induced by nuclease-based editors during HSPC gene editing may trigger a DNA damage response that limits hematopoietic repopulation (see e.g. Schiroli, G., et al. (2019). Cell Stem Cell 24, 551-565.e8).

Thus, there is a need for more efficient strategies to improve the ability of HSPCs to home and permanently repopulate the recipient BM.

### SUMMARY OF THE INVENTION

The inventors have developed HSCT strategies using HSPC mobilization as a conditioning regimen. HSPC mobilizers create an opportunity for seamless engraftment of exogenous HSPCs, which may effectively outcompete the mobilized endogenous HSPCs, to repopulate the depleted BM. The exogenous HSPCs may be administered at the peak of mobilization or after the peak of mobilization, before the depleted BM becomes repopulated by the endogenous HSPCs. These HSCT strategies may allow engraftment of ex vivo gene-modified HSPCs to therapeutically meaningful levels without raising serious toxicity concerns.

The inventors have shown that the exogenous HSPCs may have a competitive advantage as a result of their ex vivo culture and that this advantage can be enhanced by transient over-expression of engraftment enhancers such as CXCR4, CD47, ITGA4, and KIT. The inventors have shown that by using an optimized mRNA delivery platform, human HSPCs can be endowed with a transient engraftment advantage allowing them to outcompete the endogenous mobilized HSPCs for engraftment in depleted BM niches, thereby establishing stable long-term grafts.

The inventors have demonstrated the therapeutic efficacy of these strategies in a model of Hyper IgM Syndrome and in human hematochimeric mice, showing their applicability and versatility when coupled to gene transfer and editing strategies.

In one aspect, the present invention provides a population of haematopoietic stem and/or progenitor cell (HSPCs) for use in a method of therapy, the method comprising the steps of:
(a) administering one or more HSPC mobiliser to a subject to mobilise endogenous HSPCs from the subject's bone marrow; and
(b) administering the population of HSPCs to the subject.

In one aspect, the present invention provides a method for haematopoietic stem and/or progenitor cell (HSPC) transplantation in a subject in need thereof, comprising the steps:
(a) administering one or more HSPC mobiliser to the subject to mobilise the subject's endogenous HSPCs; and
(b) administering a population of HSPCs to the subject.

The population of HSPCs may be administered at or after the peak of mobilisation. In some embodiments, the population of HSPCs is administered at the peak of mobilisation. In some embodiments, the population of HSPCs is administered within about 9 hours, within about 6 hours, or within about 3 hours after step (a). In some embodiments, the population of HSPCs is administered about 2-4 hours or about 3 hours after step (a). In other embodiments, the population of HSPCs is administered concurrently with step (a).

Any suitable mobilisation regimen may be used. In some embodiments, the one or more HPSC mobiliser is selected from a granulocyte colony-stimulating factor (G-CSF), a CXCR4 antagonist and a VLA-4 antagonist, or any combination thereof. In some embodiments: (i) the subject is administered a G-CSF for at least about 5 days before the population of HSPCs is administered; (ii) the subject is administered a CXCR4 antagonist for at least about 1 day before the population of HSPCs is administered; and/or (iii) the subject is administered a VLA-4 antagonist for at least about 1 day before the population of HSPCs is administered. In some embodiments: (i) the subject is administered a G-CSF for about 7 days before the population of HSPCs is administered; (ii) the subject is administered a CXCR4 antagonist for about 2 days before the population of HSPCs is administered; and (iii) optionally, the subject is administered a VLA-4 antagonist for about 2 days before the population of HSPCs is administered.

In some embodiments, the method further comprises a step of harvesting the mobilized endogenous HSPCs prior to administration of the population of HSPCs.

Any suitable population of HSPCs may be administered. The population of HSPCs may be autologous HSPCs and/or allogenic HSPCs. In some embodiments, the population of HSPCs are autologous HSPCs. In some embodiments, the population of HSPCs is cultured ex vivo prior to administration. In some embodiments, the method further comprises a step of genetically engineering the population of HSPCs, prior to administering the population of HSPCs. In some embodiments, the population of HSPCs are genetically engineered to express a transgene, gene-edited, and/or gene-corrected.

The population of HSPCs may be genetically engineered to express one or more engraftment enhancer. In some embodiments, the one or more engraftment enhancer is expressed transiently. In some embodiments, the one or more engraftment enhancer are each expressed from an RNA polynucleotide comprising a protein-coding sequence encoding the engraftment enhancer. In some embodiments, the RNA is delivered to the population of HSPCs by electroporation or by lipid-mediated transfection. In some embodiments: (a) the protein-coding sequence is operably linked to a translation non-blocking eIF4G aptamer; (b) the protein-coding sequence is operably linked to a woodchuck hepatitis virus post-transcriptional regulatory element (WPRE); (c) the protein-coding sequence is operably linked to a polyA tail, wherein the polyA tail is at least about 100, at least about 110, at least about 120, at least about 130, at least about 140, or at least about 150 nucleotides in length; (d) the RNA polynucleotide is 5′ capped mRNA, wherein the 5′ cap is m7G(5′)ppp(5′)(2′OMeA)pG; and/or (e) the RNA polynucleotide comprises modified uridine, preferably pseudouridine.

Any suitable engraftment enhancer may be used. In some embodiments, the one or more engraftment enhancer is selected from C-X-C chemokine receptor type 4 (CXCR4) or a fragment or variant thereof, CD47 or a fragment or variant thereof, integrin alpha-4 (ITGA4) or a fragment or variant thereof, and tyrosine-protein kinase KIT (KIT) or a fragment or variant thereof, or any combination thereof. In some embodiments, the one or more engraftment enhancer comprises two or more, three or more, or four or more engraftment enhancers selected from: CXCR4 or a fragment or variant thereof, CD47 or a fragment or variant thereof, ITGA4 or a fragment or variant thereof, and KIT or a fragment or variant thereof.

In some embodiments, the population of HSPCs are genetically engineered to transiently express CXCR4 or a fragment or variant thereof. In some embodiments, the CXCR4 or a fragment or variant thereof comprises or consists of an amino acid sequence having at least 70% identity to any of SEQ ID NOs: 1-9. In some embodiments, the CXCR4 or a fragment or variant thereof comprises or consists of the amino acid sequence of SEQ ID NO: 2. In some embodiments, the CXCR4 or a fragment or variant thereof comprises or consists of the amino acid sequence of any of SEQ ID NOs: 3-9. In some embodiments, the CXCR4 or a fragment or variant thereof comprises or consists of the amino acid sequence of any of SEQ ID NOs: 6-9. In some embodiments, the CXCR4 variant has increased resistance to a CXCR4 antagonist and/or has maintained or increased response to SDF-1 compared to CXCR4.

In some embodiments, the population of HSPCs are genetically engineered to transiently express CD47 or a fragment or variant thereof. In some embodiments, the CD47 or a fragment or variant thereof comprises or consists of an amino acid sequence having at least 70% identity to any of SEQ ID NOs: 23-26. In some embodiments, the CD47 variant has maintained or increased response to its natural ligands compared to CD47.

In some embodiments, the population of HSPCs are genetically engineered to transiently express ITGA4 or a fragment or variant thereof. In some embodiments, the ITGA4 or a fragment or variant thereof comprises or consists of an amino acid sequence having at least 70% identity to SEQ ID NO: 29. In some embodiments, the ITGA4 variant has increased resistance to a VLA-4 antagonist and/or has maintained or increased response to its natural ligands compared to ITGA4.

In some embodiments, the population of HSPCs are genetically engineered to transiently express KIT or a fragment or variant thereof. In some embodiments, the KIT or a fragment or variant thereof comprises or consists of an amino acid sequence having at least 70% identity to SEQ ID NO: 31. In some embodiments, the KIT variant has increased resistance to a KIT-directed antibody or immunotoxin and/or has maintained or increased response to SCF compared to KIT.

The subject may be any subject in need thereof. In preferred embodiments, the subject does not undergo chemotherapy or radiotherapy conditioning prior to administration of the HSPCs. In some embodiments, the subject has a primary immunodeficiency, a lysosomal storage disorder, a haemoglobinopathy, or cancer. In some embodiments, the subject has a primary immunodeficiency, such as human primary combined immunodeficiency Hyper IgM Syndrome 1 (HIGM-1).

The chimerism level of the population of HSPCs in the subject's bone marrow may reach a level of at least 10%, at least 15%, at least 20%, at least 25%, at least 30%, at least 35%, or at least 40%. The chimerism level of the population of HSPCs in the subject's bone marrow may be stable for at least 24 weeks.

In one aspect, the present invention provides the use of a C-X-C chemokine receptor type 4 (CXCR4) variant, integrin alpha-4 (ITGA4), and/or tyrosine-protein kinase KIT (KIT), for increasing engraftment by haematopoietic stem and/or progenitor cells (HSPCs), wherein the CXCR4 variant comprises one or more amino acid substitution selected from: V160L, A175F, Q200A, D262N, and H281A. The HSPCs may be genetically engineered to express the CXCR4 variant, ITGA4, and/or KIT.

In one aspect, the present invention provides a method for increasing engraftment by haematopoietic stem and/or progenitor cells (HSPCs), wherein the method comprises the step of genetically engineering the HSPCs to express a CXCR4 variant, ITGA4, and/or KIT, wherein the CXCR4 variant comprises one or more amino acid substitution selected from: V160L, A175F, Q200A, D262N, and H281A.

In some embodiments, the CXCR4 variant, ITGA4, and/or KIT are expressed transiently or stably by the HSPCs. In some embodiments, the HSPCs are genetically engineered to transiently express the CXCR4 variant, ITGA4, and/or KIT. In some embodiments, the HSPCs are transduced or transfected with one or more vectors encoding the CXCR4 variant, ITGA4, and/or KIT, preferably wherein the one or more vectors are RNA vectors. In some embodiments, the HSPCs are genetically engineered to express two or more, three or more, or four or more of: the CXCR4 variant, ITGA4, KIT, CXCR4, and CD47.

In some embodiments, the CXCR4 variant comprises one or more amino acid substitution selected from: A175F, Q200A, D262N, and H281A. In some embodiments, the CXCR4 variant comprises one or more amino acid substitution selected from: A175F, D262N or H281A. In some embodiments, the CXCR4 variant comprises one or more amino acid substitution selected from: A175F or D262N. In some embodiments, the CXCR4 variant comprises the amino acid substitution A175F. In some embodiments, the CXCR4 variant comprises the amino acid substitution Q200A. In some embodiments, the CXCR4 variant comprises the amino acid substitution D262N. In some embodiments, the CXCR4 variant comprises the amino acid substitution H281A.

In some embodiments, the CXCR4 variant comprises or consists of the amino acid sequence of any of SEQ ID NOs: 3-9. In some embodiments, the CXCR4 variant comprises or consists of the amino acid sequence of any of SEQ ID NOs: 6-9.

In one aspect, the present invention provides population of genetically engineered haematopoietic stem and/or progenitor cells (HSPCs) obtainable by the method of the present invention.

In one aspect, the present invention provides a population of genetically engineered haematopoietic stem and/or progenitor cells (HSPCs), wherein the HSPCs are genetically engineered to express a CXCR4 variant, ITGA4, and/or KIT, preferably wherein the HSPCs are genetically engineered to transiently express a CXCR4 variant, ITGA4, and/or KIT, wherein the CXCR4 variant comprises one or more amino acid substitution selected from: V160L, A175F, Q200A, D262N, and H281A. In some embodiments, the CXCR4 variant comprises one or more amino acid substitution selected from: A175F, Q200A, D262N, and H281A.

In some embodiments, the HSPCs are genetically engineered to express two or more, three or more, or four or more of: the CXCR4 variant, ITGA4, KIT, CXCR4, and CD47, preferably wherein the HSPCs are genetically engineered to transiently express two or more, three or more, or four or more of: the CXCR4 variant, ITGA4, KIT, CXCR4, and CD47.

In one aspect, the present invention provides a pharmaceutical composition comprising the population of genetically engineered haematopoietic stem and/or progenitor cells (HSPCs) of the present invention and a pharmaceutically acceptable carrier, diluent or excipient.

In one aspect, the present invention provides a population of genetically engineered haematopoietic stem and/or progenitor cells (HSPCs) according to the present invention for use in therapy.

In one aspect, the present invention provides a population of genetically engineered haematopoietic stem and/or progenitor cells (HSPCs) according to the present invention for use in the treatment or prevention of cancer, an immune disorder, a lysosomal storage disorder, a bacterial or viral infection, a genetic disease, or a hemoglobinopathy.

In one aspect, the present invention provides a method for haematopoietic stem and/or progenitor cell (HSPC) transplantation, comprising the steps:
(a) providing a population of HSPCs which are genetically engineered to express a CXCR4 variant, ITGA4, and/or KIT, wherein the CXCR4 variant comprises one or more amino acid substitution selected from: V160L, A175F, Q200A, D262N, and H281A, preferably wherein the CXCR4 variant comprises one or more amino acid substitution selected from: A175F, Q200A, D262N, and H281A; and
(b) administering the HSPCs to a subject.

In one aspect, the present invention provides a method of treating or preventing cancer, an immune disorder, a lysosomal storage disorder, a bacterial or viral infection, a genetic disease, or a hemoglobinopathy, comprising the steps:
(a) providing a population of haematopoietic stem and/or progenitor cells (HSPCs) which are genetically engineered to express a CXCR4 variant, ITGA4, and/or KIT, wherein the CXCR4 variant comprises one or more amino acid substitution selected from: V160L, A175F, Q200A, D262N, and H281A, preferably wherein the CXCR4 variant comprises one or more amino acid substitution selected from: A175F, Q200A, D262N, and H281A; and
(b) administering the HSPCs to a subject.

The subject may be any subject in need thereof. In some embodiments, the subject is subjected to a mild myeloablative, reduced intensity or non-myeloablative conditioning regimen before administration of the HSPCs. In some embodiments the subject: (a) is subjected to a regimen for mobilisation of endogenous HSPCs; or (b) is subjected to conditioning with one or more HSPC-specific immunotoxins. In some embodiments, the subject does not undergo chemotherapy or radiotherapy conditioning before administration of the HSPCs.

In preferred embodiments, the subject is subjected to a regimen for mobilisation of endogenous HSPCs. The population of HSPCs may be administered at or after the peak of mobilisation. In some embodiments, the population of HSPCs is administered at the peak of mobilisation. In some embodiments, the population of HSPCs is administered within about 9 hours, within about 6 hours, or within about 3 hours after the regimen for mobilisation of endogenous HSPCs is completed. In some embodiments, the population of HSPCs is administered about 2-4 hours or about 3 hours after the regimen for mobilisation of endogenous HSPCs is completed. In some embodiments, the population of HSPCs is administered concurrently with the regimen for mobilisation of endogenous HSPCs.

Any suitable regimen may be used for mobilisation. In some embodiments, the regimen for mobilisation of endogenous HSPCs comprises administering one or more HPSC mobiliser selected from a granulocyte colony-stimulating factor (G-CSF), a CXCR4 antagonist and a VLA-4 antagonist, or any combination thereof. In some embodiments, the regimen for mobilisation of endogenous HSPCs comprises: (i) administering a G-CSF for at least about 5 days; (ii) administering a CXCR4 antagonist for at least about 1 day; and/or (iii) administering a VLA-4 antagonist for at least about 1 day. In some embodiments, the regimen for mobilisation of endogenous HSPCs comprises: (i) administering a G-CSF for about 7 days; (ii) administering a CXCR4 antagonist for about 2 days; and (iii) administering a VLA-4 antagonist for about 2 days.

In some embodiments, the regimen for mobilisation of endogenous HSPCs is followed by a step of harvesting the mobilized endogenous HSPCs prior to administration of the population of HSPCs.

In one aspect, the present invention provides an RNA polynucleotide comprising a protein-coding sequence. In some embodiments, the RNA polynucleotide comprises from 5′ to 3′: a m7G(5′)ppp(5′)(2′OMeA)pG cap; a translation non-blocking eIF4F aptamer; a Kozak sequence; a protein-coding sequence; a WPRE; and a polyA tail comprising at least about 100 nucleotides.

The protein-coding sequence may be operably linked to a Kozak sequence.

The protein-coding sequence may be operably linked to a translation non-blocking eIF4F aptamer. In some embodiments, the translation non-blocking eIF4F aptamer is a translation non-blocking eIF4G aptamer. In some embodiments, the translation non-blocking eIF4F aptamer comprises or consists of a nucleotide sequence having at least 90% identity to any of SEQ ID NOs: 33-36. In some embodiments, the translation non-blocking eIF4F aptamer comprises or consists of a nucleotide sequence having at least 90% identity to SEQ ID NO: 34.

The protein-coding sequence may be operably linked to a woodchuck hepatitis virus post-transcriptional regulatory element (WPRE). In some embodiments, the WPRE comprises or consists of a nucleotide sequence having at least 70% identity to SEQ ID NO: 37.

The protein-coding sequence may be operably linked to a polyA tail. In some embodiments, the polyA tail is at least about 100, at least about 110, at least about 120, at least about 130, at least about 140, or at least about 150 nucleotides in length.

The RNA polynucleotide may be a 5′ capped mRNA. In some embodiments, the 5′ cap is m7G(5′)ppp(5′)(2′OMeA)pG.

The RNA polynucleotide may comprise modified uridine. In some embodiments, the RNA polynucleotide comprises pseudouridine.

In preferred embodiments, the transgene is an engraftment enhancer. Any suitable engraftment enhancer may be used. In some embodiments, the engraftment enhancer is selected from CXCR4 or a fragment or variant thereof, CD47 or a fragment or variant thereof, ITGA4 or a fragment or variant thereof, and KIT or a fragment or variant thereof.

In some embodiments, the engraftment enhancer is CXCR4 or a fragment or variant thereof. In some embodiments, the CXCR4 or a fragment or variant thereof comprises or consists of an amino acid sequence having at least 70% identity to any of SEQ ID NOs: 1-9, preferably wherein the CXCR4 or a fragment or variant thereof comprises or consists of the amino acid sequence of any of SEQ ID NOs: 3-9, more preferably wherein the CXCR4 or a fragment or variant thereof comprises or consists of the amino acid sequence of any of SEQ ID NOs: 6-9. In some embodiments, the protein-coding sequence comprises or consists of a nucleotide sequence having at least 70% identity to any of SEQ ID NOs: 10-22, preferably wherein the protein-coding sequence comprises or consists of the nucleotide sequence of any of SEQ ID NOs: 19-22.

In some embodiments, the engraftment enhancer is CD47 or a fragment or variant thereof. In some embodiments, the CD47 or a fragment or variant thereof comprises or consists of an amino acid sequence having at least 70% identity to any of SEQ ID NOs: 23-26. In some embodiments, the protein-coding sequence comprises or consists of a nucleotide sequence having at least 70% identity to SEQ ID NO: 27 or 28.

In some embodiments, the engraftment enhancer is ITGA4 or a fragment or variant thereof. In some embodiments, the ITGA4 or a fragment or variant thereof comprises or consists of an amino acid sequence having at least 70% identity to SEQ ID NO: 29. In some embodiments, the protein-coding sequence comprises or consists of a nucleotide sequence having at least 70% identity to SEQ ID NO: 30.

In some embodiments, the engraftment enhancer is KIT or a fragment or variant thereof. In some embodiments, the KIT or a fragment or variant thereof comprises or consists of an amino acid sequence having at least 70% identity to SEQ ID NO: 31. In some embodiments, the protein-coding sequence comprises or consists of a nucleotide sequence having at least 70% identity to SEQ ID NO: 32.

In one aspect, the present invention provides a DNA polynucleotide encoding the RNA polynucleotide according to the present invention.

In one aspect, the present invention provides a vector comprising the DNA polynucleotide according to the present invention.

In one aspect, the present invention provides an isolated cell comprising the RNA polynucleotide according to the present invention, the DNA polynucleotide according to the present invention, or the vector according to the present invention.

In one aspect, the present invention provides a method for the production of the RNA polynucleotide according to the present invention, comprising the step of in vitro transcribing the DNA polynucleotide according to the present invention. In some embodiments, the method comprises transcribing the DNA with modified uridine, preferably pseudouridine. In some embodiments, the method comprises capping the RNA polynucleotide with m7G(5′)ppp(5′)(2′OMeA)pG. In some embodiments, the method comprise purifying the RNA polynucleotide.

In one aspect, the present invention provides a method for delivering the RNA polynucleotide according to the present invention, wherein the RNA polynucleotide is delivered to a population of HSPCs by electroporation or by lipid-mediated transfection.

In one aspect, the present invention provides the use of the RNA polynucleotide according to according to the present invention for increasing engraftment by haematopoietic stem and/or progenitor cells (HSPCs).

In one aspect, the present invention provides a method for increasing engraftment by haematopoietic stem and/or progenitor cells (HSPCs), wherein the method comprises the step of transfecting the RNA polynucleotide according to the present invention into the HSPCs.

In one aspect, the present invention provides an isolated haematopoietic stem and/or progenitor cell (HSPC) comprising the RNA polynucleotide according to the present invention.

In one aspect, the present invention provides a population of isolated haematopoietic stem and/or progenitor cells (HSPCs) according to the present invention.

In one aspect, the present invention provides a pharmaceutical composition comprising the isolated HSPC of the present invention, or a population of HSPCs of the present invention, and a pharmaceutically acceptable carrier, diluent or excipient.

In one aspect, the present invention provides an isolated HSPC according to the present invention, or a population of HSPCs according to the present invention, for use in therapy.

In one aspect, the present invention provides an isolated HSPC according to the present invention, or a population of HSPCs according to the present invention, for use in the treatment or prevention of cancer, an immune disorder, a lysosomal storage disorder, a bacterial or viral infection, a genetic disease, or a hemoglobinopathy.

In one aspect, the present invention provides a method for haematopoietic stem and/or progenitor cell (HSPC) transplantation, comprising the steps:
(a) providing a population of HSPCs comprising the RNA polynucleotide according to the present invention; and
(b) administering the HSPCs to a subject.

In one aspect, the present invention provides a method of treating or preventing cancer, an immune disorder, a lysosomal storage disorder, a bacterial or viral infection, a genetic disease, or a hemoglobinopathy, comprising the steps:
(a) providing a population of HSPCs comprising the RNA polynucleotide according to the present invention; and
(b) administering the HSPCs to a subject.

### DESCRIPTION OF DRAWINGS

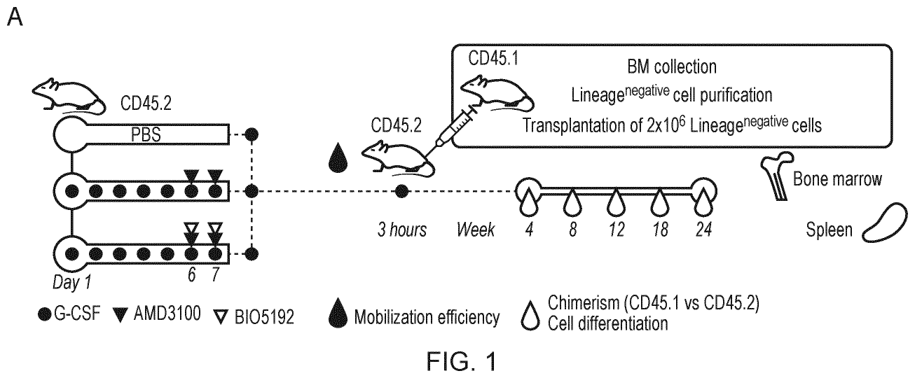

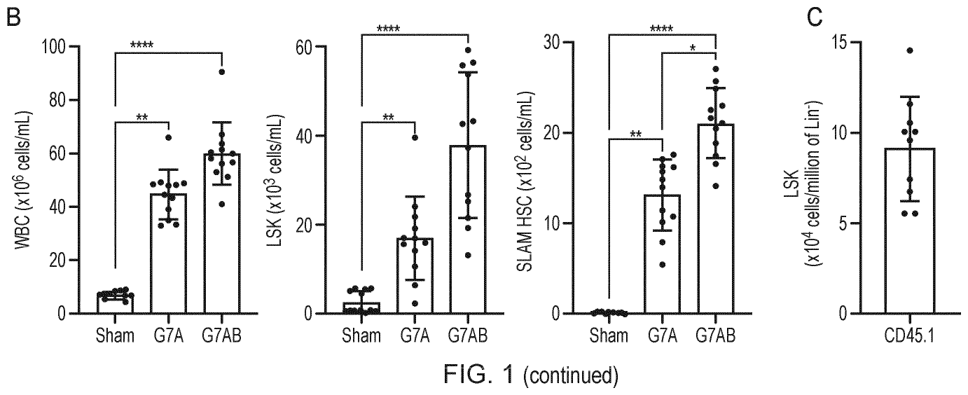

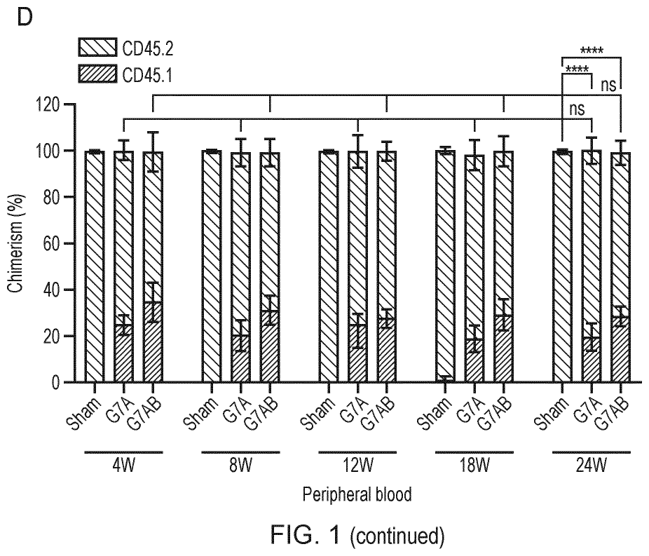

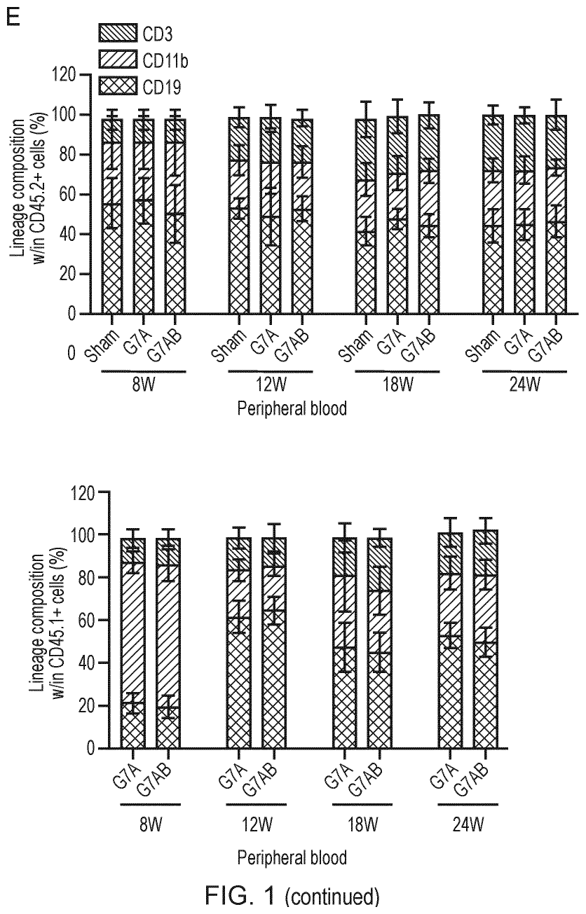

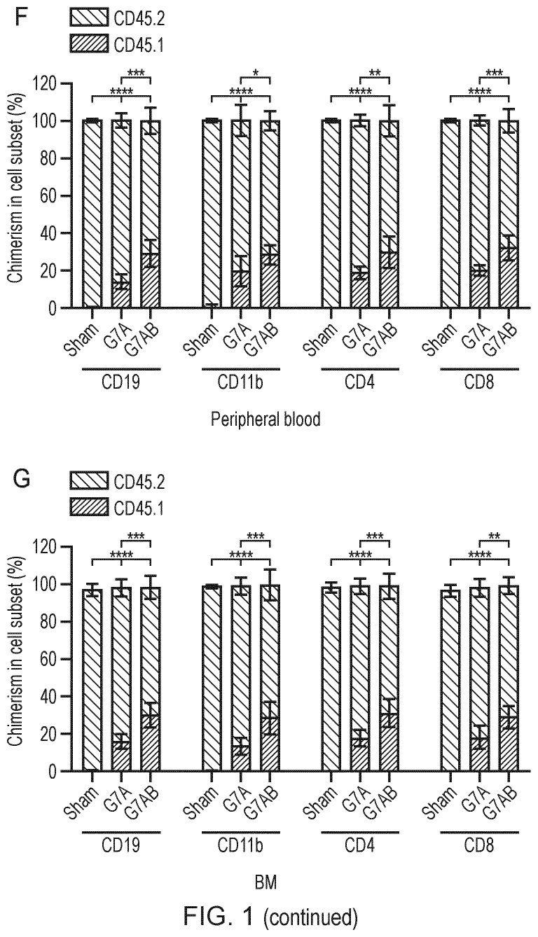

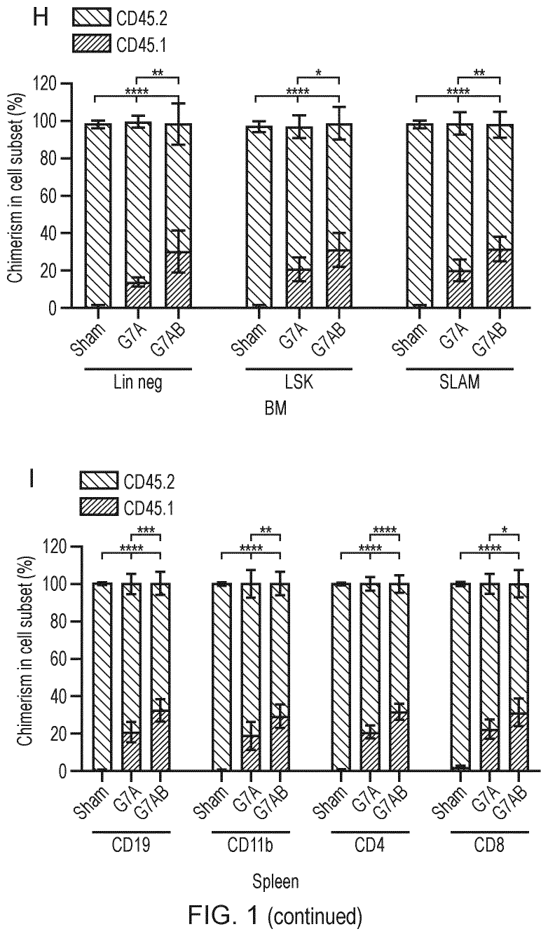

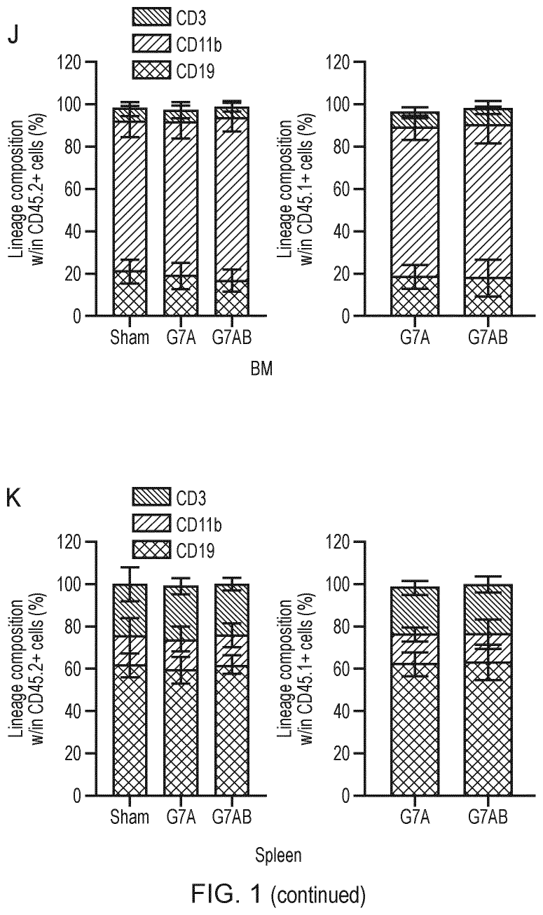

FIG. 1. Long-term donor chimerism is established by mobilization-based HSCT (M-HSCT)
(A) Schematic of the M-HSCT protocol. Recipient CD45.2 mice were mobilized with G-CSF (green dots) and AMD3100 (red triangle), with and without BIO5192 (blue triangle) (G7A; G7AB), and subsequently transplanted with CD45.1 2×10⁶ Lin⁻ cells, collected from the BM.
(B) Counts of mobilized WBC (left panel), LSK (middle panel) and SLAM HSC (right panel) per mL in the PB in non-mobilized (Sham) and G7A and G7AB mobilized mice. Kruskal-Wallis test, followed by post hoc analysis with Dunn's test.
(C) Counts of LSK cells per million of Lin⁻ CD45.1 donor cells, collected from the BM.
(D) Long-term follow-up of the donor CD45.1 (blue) and recipient CD45.2 (ochre) chimerism observed within total CD45+ cells in PB after transplanting 2×10⁶ Lin⁻ cells post-mobilization in recipient CD45.2 mice. Longitudinal comparisons, performed by mixed-effects model (REML), followed by post hoc analysis with Tukey's test (between groups) or by post hoc analysis with Dunnett's test (within groups).
(E) Reconstitution of myeloid and lymphoid lineages overtime of the recipient CD45.2+ cells (left panel) and CD45.1+ cells (right panel) in PB of recipient CD45.2 mice. Comparison of lineages between CD45.1 and CD45.2 cells performed at the last time point by mixed-effects model (REML), followed by post hoc analysis with Dunnett's.
(F-I) Chimerism of CD45.1 cells observed within CD19+ B cells, CD11b+ myeloid cells, CD4+ T helper cells and CD8+ T cytotoxic cells in PB (F), BM (G), within Lin⁻, LSK and SLAM HSC (H) and spleen (1). Mixed-effects model (REML), followed by post hoc analysis with Tukey's test (between groups) or by post hoc analysis with Dunnett's test (within groups).
(J-K) Myeloid and lymphoid lineage composition of CD45.2+ cells (left panel) and CD45.1+ cells (right panel) in the BM (J) and spleen (K) of CD45.2 mice. Comparison of lineages between CD45.1 and CD45.2 cells, performed by mixed-effects model (REML), followed by post hoc analysis with Dunnett's test.

Results are mean±SEM, with n≥10. P-values were defined as such (*P<0.05, **P<0.01, ***P<0.001, ****P<0.0001. “ns” means non-significance).

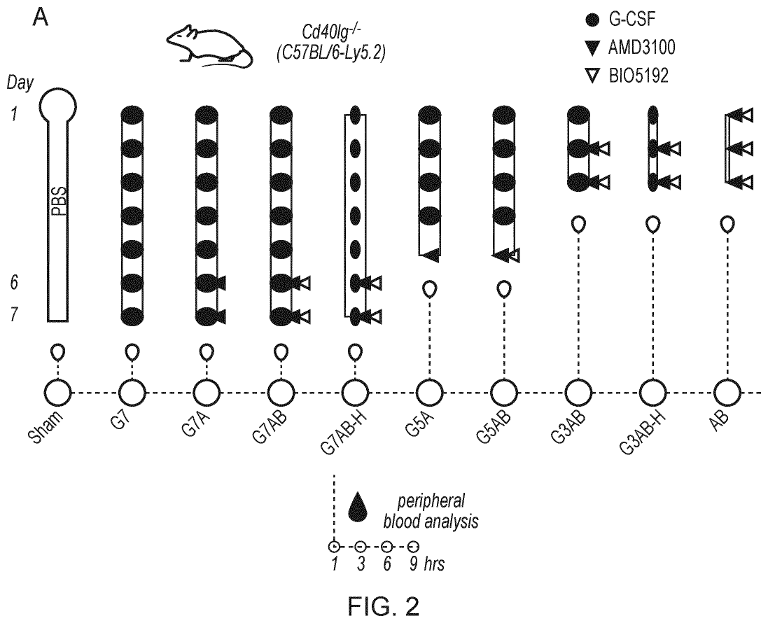

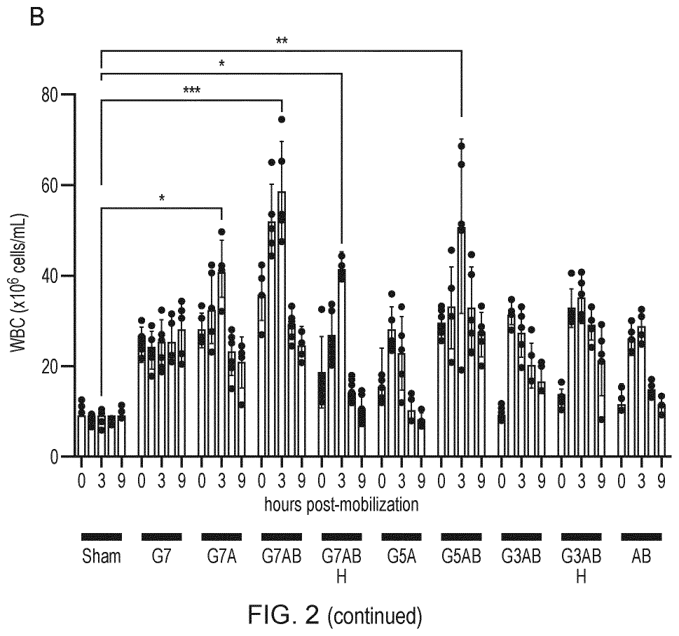

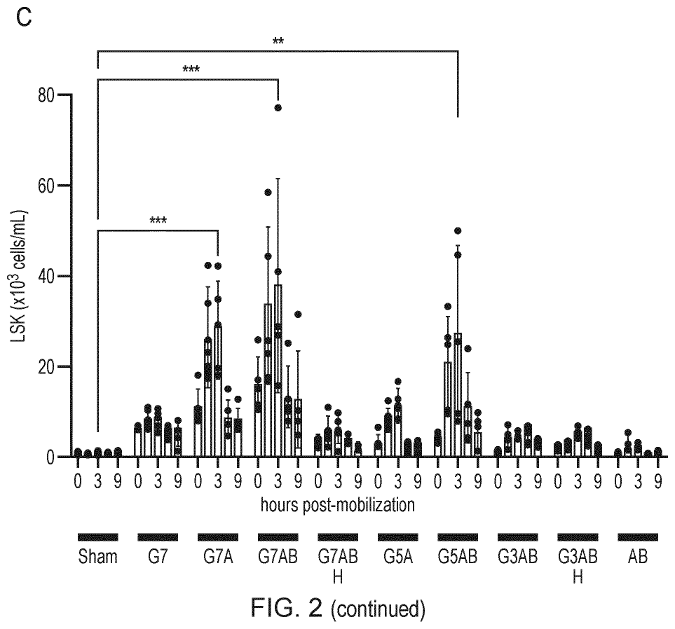

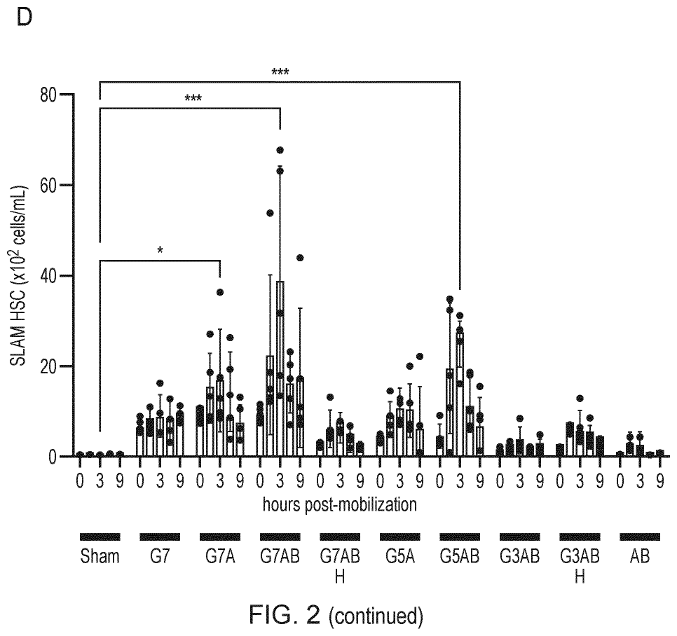

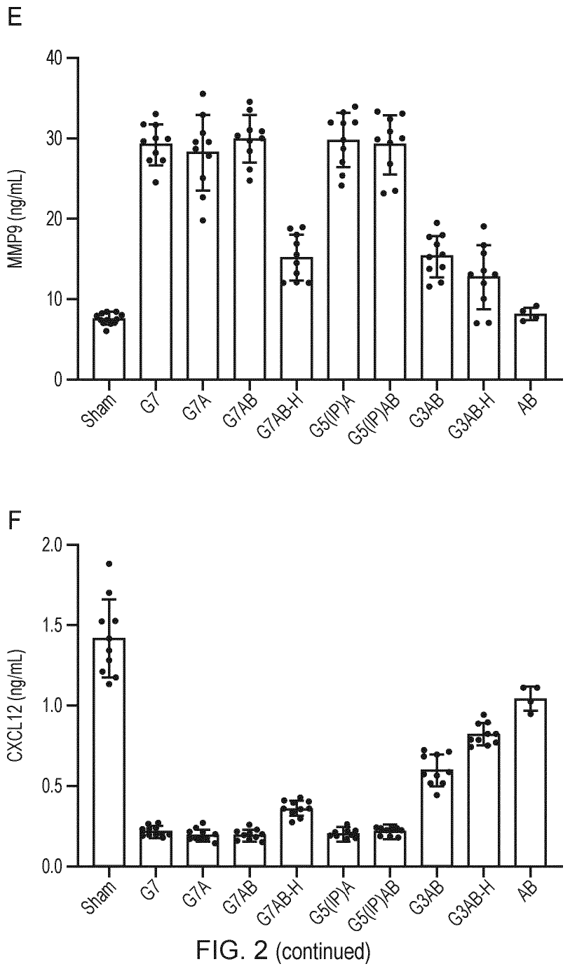

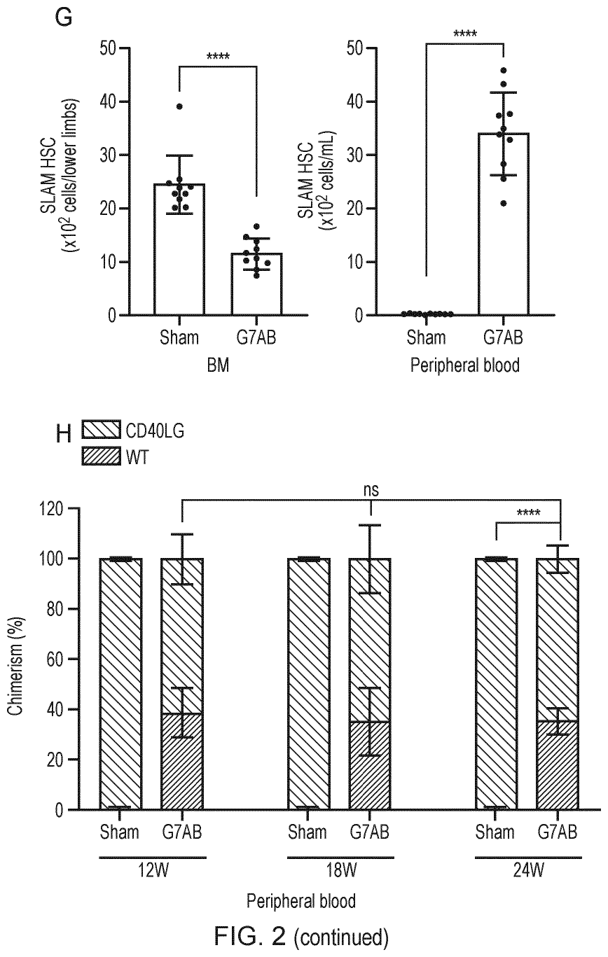

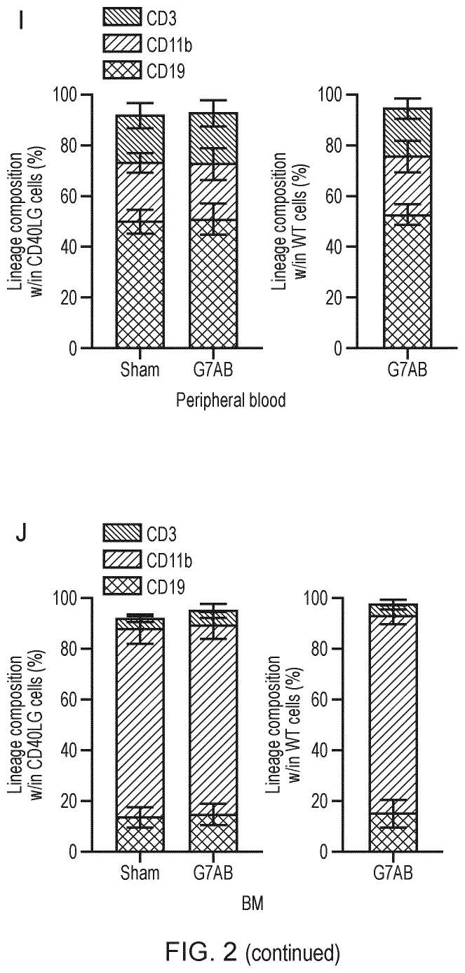

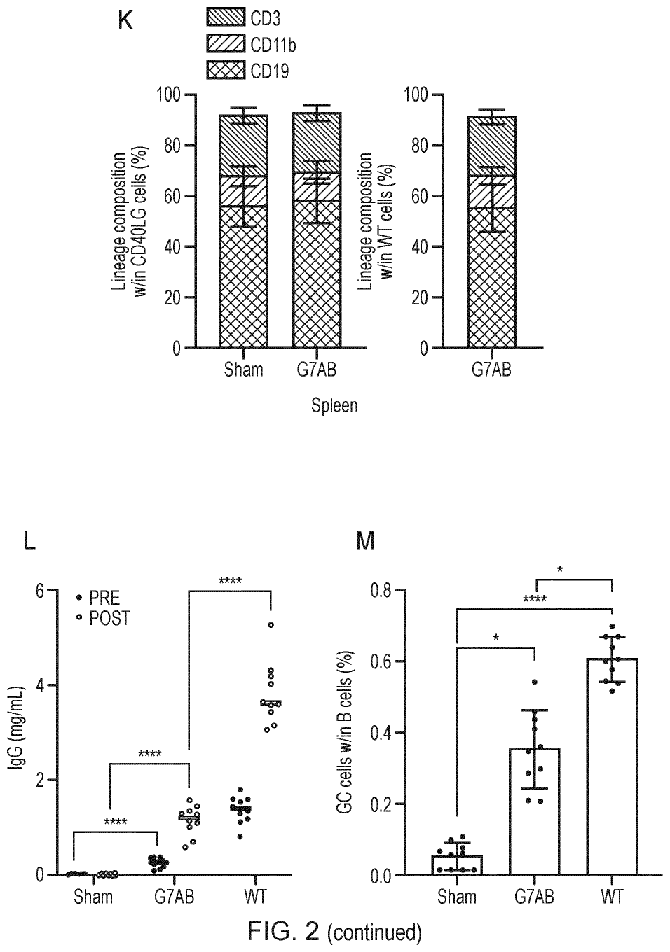

FIG. 2. M-HSCT allows establishing sufficient donor chimerism to rescue the HIGM1 phenotype
(A) Schematic of different mobilization protocols tested and the times of analysis in Cd40Ig^{-/-} mice.
(B-D) Counts of mobilized WBC (B), LSK (C) and SLAM HSC (D) per mL in the PB of Cd40Ig^{-/-} mice treated with PBS (Sham), G-CSF for 7 days (G7), G-CSF for 7 days and AMD3100 (G7A), G-CSF for 7 days, AMD3100 and BIO5192 (G7AB), half-dose of G-CSF for 7 days, AMD3100 and BIO5192 (G7AB-H), G-CSF for 5 days and AMD3100 (G5A), G-CSF for 5 days, AMD3100 and BIO5192 (G5AB), G-CSF for 3 days, AMD3100 and BIO5192 (G3AB), half-dose of G-CSF for 3 days, AMD3100 and BIO5192 (G3AB-H) and only AMD3100 and BIO5192 (AB) at 0, 1, 3, 6 and 9 hours after the last injection of A or AB. Kruskal-Wallis test performed for the 3-hour timepoint, followed by post hoc analysis with Dunn's test.
(E-F) MMP9 (E) and CXCL12 (F) concentration in the BM extracellular extracts of Cd40Ig^{-/-} mice mobilized with protocols described above.
(G) Total counts of SLAM HSC in the lower limbs (left panel) and in the PB per mL (right panel) of Cd40Ig^{-/-} mice treated with PBS or mobilized with G7AB. Mann-Whitney test performed.
(H) Long-term follow-up of the donor WT (blue) and recipient Cd40Ig^{-/-} (ochre) chimerism observed within total CD45+ cells in PB after transplanting 2×10⁶ Lin⁻ cells (collected from the BM) post-mobilization in recipient Cd40Ig^{-/-} mice. Longitudinal comparisons, performed by mixed-effects model (REML), followed by post hoc analysis with Sidak's test or by post hoc analysis with Dunnett's test (within groups).
(I-K) Myeloid and lymphoid lineage composition of Cd40Ig^{-/-} (left panel) and WT (right panel) cells in the PB (I), BM (J) and spleen (K) of Cd40Ig^{-/-} mice after M-HSCT. Comparison of lineages between WT and Cd40Ig^{-/-} cells performed by mixed-effects model (REML), followed by post hoc analysis with Dunnett's.
(L) TNP-KLH-specific IgG concentration in sera collected 7 days before (pre) and after (post) TNP-KLH vaccination of Cd40Ig^{-/-} mice after M-HSCT. Mixed-effects model (REML), followed by post hoc analysis with Tukey's test.
(M) Percentage of PNA⁺GL7⁺ splenic germinal centers B cells within the spleen of Cd40Ig^{-/-} mice after TNP-KLH vaccination of Cd40Ig^{-/-} mice treated by M-HSCT. Kruskal-Wallis test, followed by post hoc analysis with Dunn's test.

Results are mean±SEM, with n≥5 for the kinetic experiments, except for the AB group (n=4), and with n≥9 for the Cd40Ig−/− M-HSCT experiments. P-values were defined as such (*P<0.05, **P<0.01, ***P<0.001, ****P<0.0001. “ns” means non-significance).

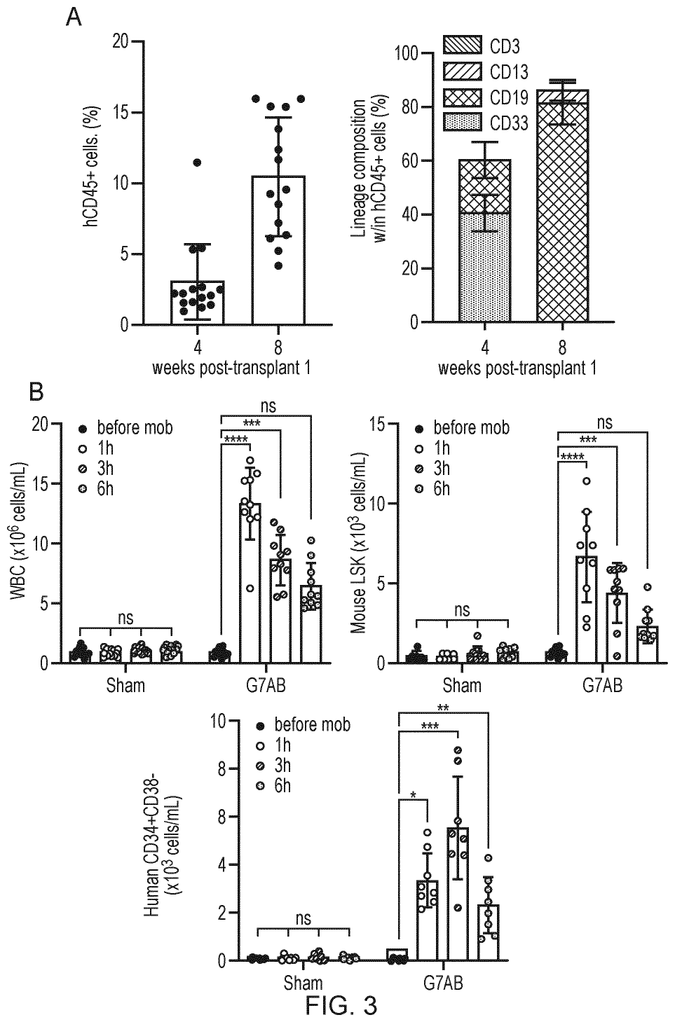

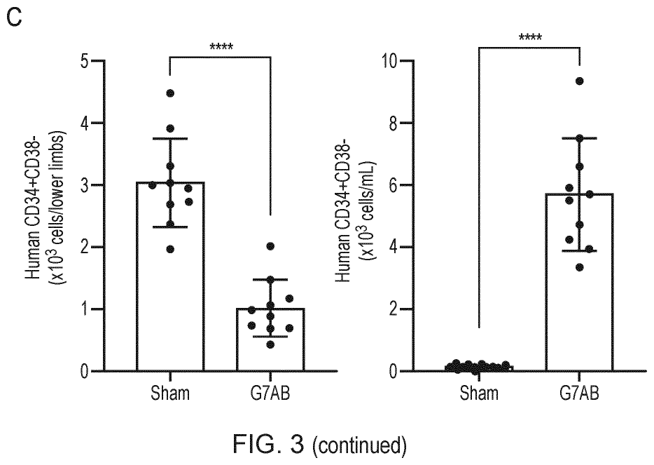

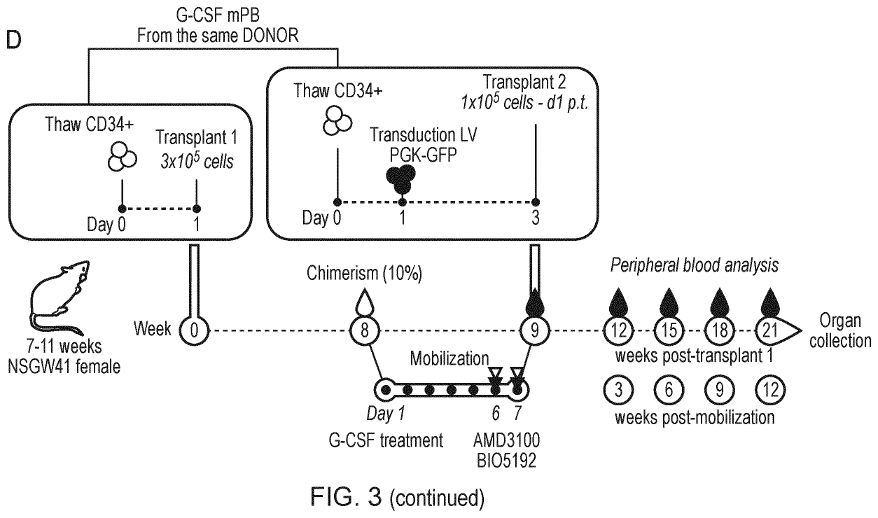

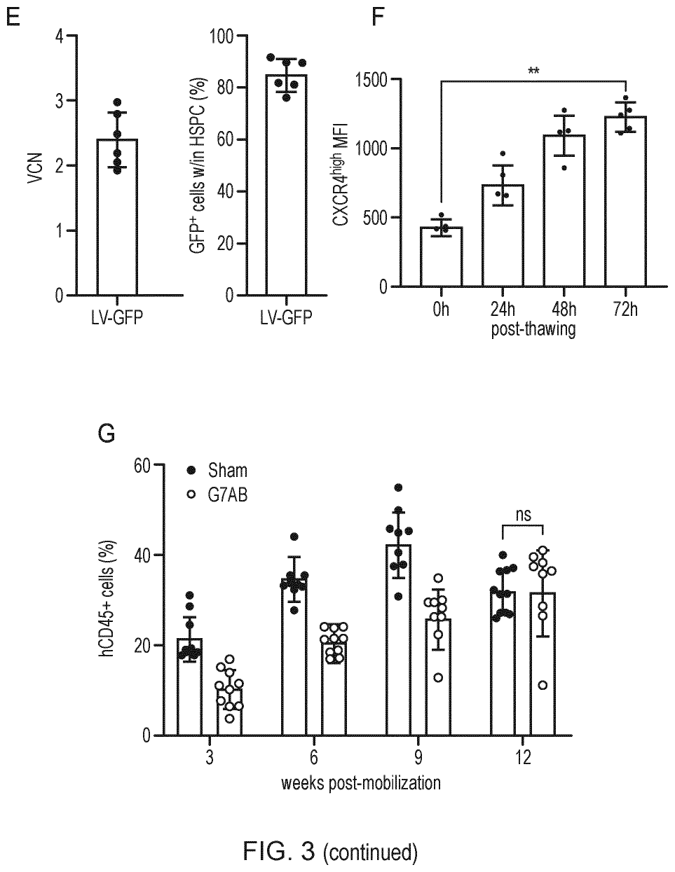

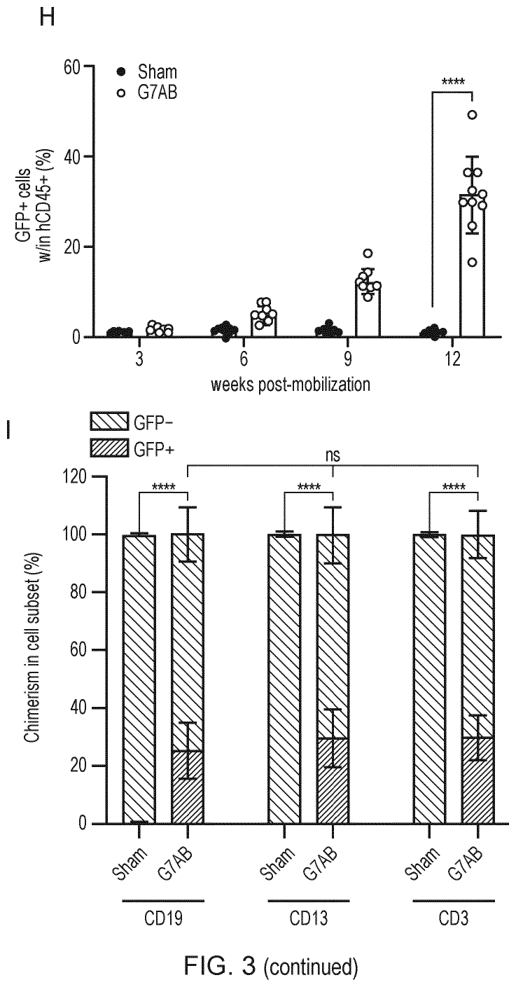

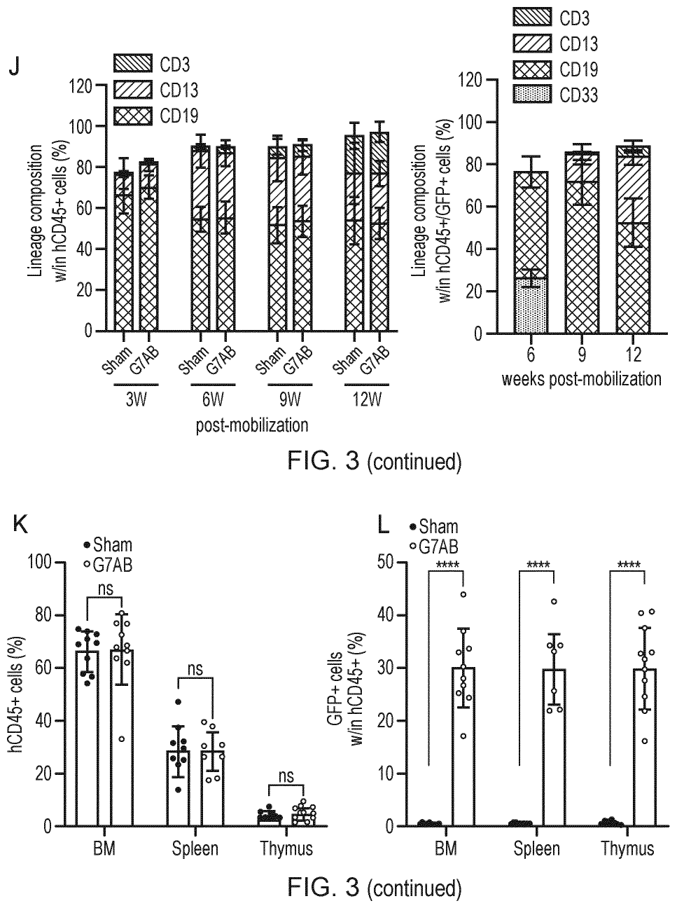

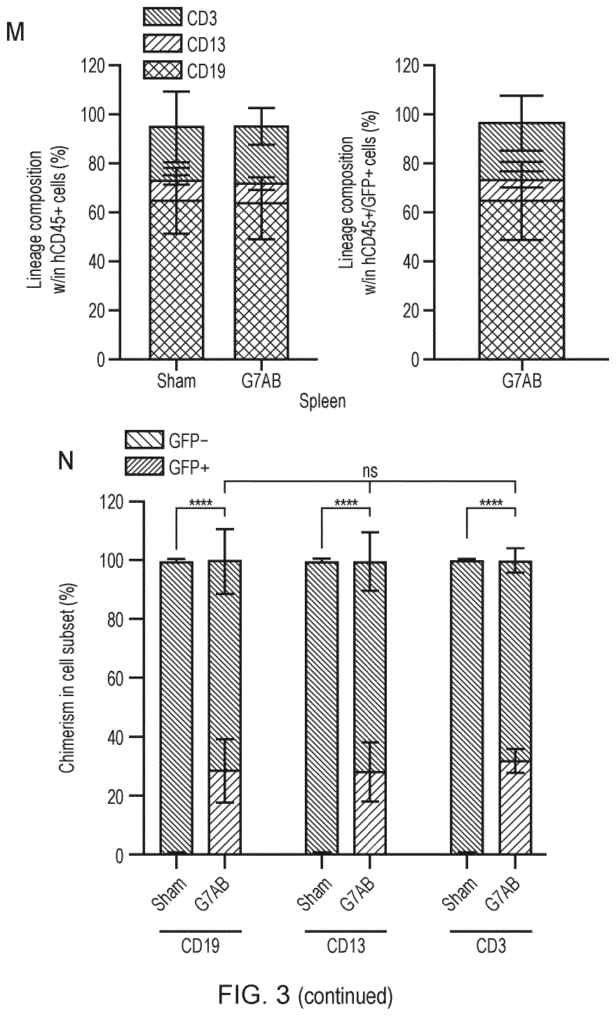

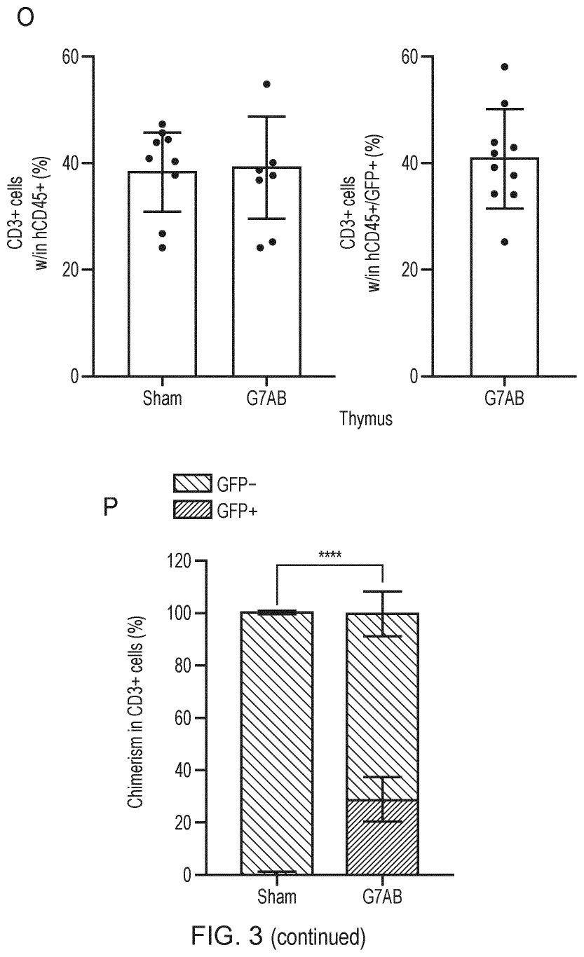

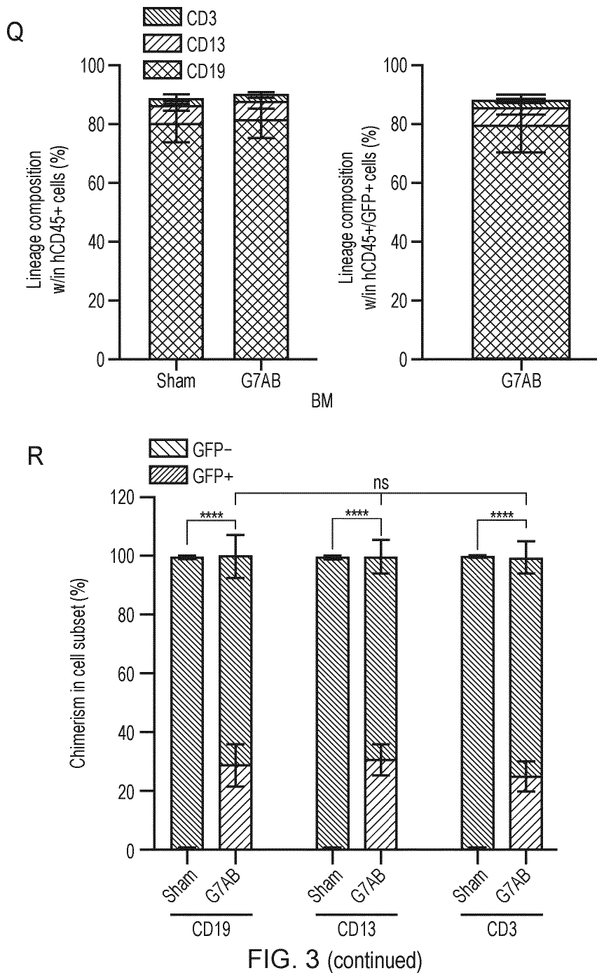

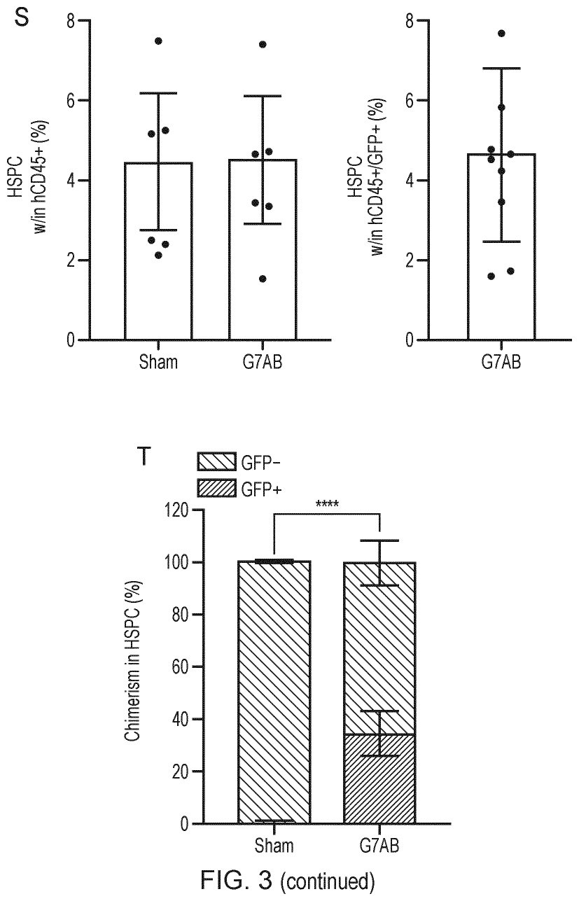

FIG. 3. M-HSCT allows efficient donor to recipient exchange of HSPCs within the human niche of hematochimeric mice
(A) Percentage of human chimerism (CD45⁺; left panel) and lymphoid/myeloid cell composition within human CD45⁺ population (right panel) overtime in NSGW41 mice, following the first transplant of human CD34⁺ cells.
(B) Counts of mobilized WBC (left panel), LSK cells (middle panel) and human CD34+CD38-cells (right panel) per mL in the PB of humanized NSGW41 mice non-mobilized or mobilized with G7AB at 0, 1, 3 and 6 hours after the last injection of AMD3100 and BIO5192. Kruskal-Wallis test, followed by post hoc analysis with Dunn's test.
(C) Total counts of CD34+CD38⁻ cells in the lower limbs (left panel) and the PB per mL (right panel) of humanized NSGW41 mice non-mobilized (Sham) or mobilized (G7AB). Mann-Whitney test performed.
(D) Schematic illustration of competitive transplantation, after G7AB mobilization, between human resident cells (initially transplanted with 3×10⁵ CD34⁺ G-CSF mPB cells, counted on day 1 post-thawing; d1 p.t.) and newly transplanted CD34+G-CSF mPB GFP-transduced cells (outgrowth of 1×10⁵ CD34⁺ cells, counted on d1 p.t., transplanted on d3 p.t.) in NSGW41 mice.
(E) Vector copy number (VCN) in HSPCs population (CD34⁺ CD133⁺ CD90⁺) (left panel) and percentage of GFP⁺ cells measured within CD34⁺ cells (right panel), in vitro, after transduction.
(F) CXCR4^{high} MFI overtime after thawing, stained with an antibody targeting the N-terminus epitope of CXCR4, on HSPC population (CD34⁺ CD133⁺ CD90⁺) mobilized with G-CSF.
(G-H) Long-term follow-up of human CD45⁺ (G) and GFP⁺/CD45⁺ (H) cells chimerism in PB after M-HSCT in NSGW41 mice. Mann-Whitney test performed.
(I) Chimerism of GFP⁺ cells observed within CD19⁺, CD13⁺ and CD3⁺ cells at the end of the experiment in PB after M-HSCT. Mixed-effects model (REML), followed by post hoc analysis with Sidak's test (between groups) or by post hoc analysis with Dunnett's test (within groups).
(J) Reconstitution of myeloid and lymphoid lineages overtime of human CD45⁺ (left panel) and CD45⁺/GFP⁺ cells (right panel) in PB after M-HSCT. Comparison of lineages between human CD45⁺ and GFP⁺ cells performed at the last time point by mixed-effects model (REML), followed by post hoc analysis with Dunnett's test (within groups).
(K-L) Human CD45⁺ (K) and GFP⁺/CD45⁺ (L) cells chimerism in BM, spleen and thymus after M-HSCT. Mixed-effects model (REML), followed by post hoc analysis with Sidak's test.
(M) Myeloid and lymphoid lineage composition of human CD45⁺ cells (left panel) and CD45⁺/GFP⁺ cells (right panel) in the spleen of NSGW41 mice after M-HSCT. Comparison of lineages between human CD45⁺ and CD45⁺/GFP⁺ cells by mixed-effects model (REML), followed by post hoc analysis with Dunnett's test (within groups).
(N) Chimerism of GFP⁺ cells observed within CD19⁺, CD13⁺ and CD3⁺ cells in the spleen after M-HSCT. Mixed-effects model (REML), followed by post hoc analysis with Sidak's test (between groups) or by post hoc analysis with Dunnett's test (within groups).
(O) Percentage of T cells within human CD45⁺ (left panel) and CD45⁺/GFP⁺ cells (right panel) in the thymus after M-HSCT. Kruskal-Wallis test was performed, followed by post hoc analysis with Dunn's test.
(P) Chimerism of GFP⁺ cells observed within T cells in thymus after M-HSCT. Mann-Whitney test performed.
(Q) Myeloid and lymphoid lineage composition of human CD45⁺ (left panel) and CD45⁺/GFP⁺ cells (right panel) in the BM of NSGW41 mice after M-HSCT. Comparison of lineages between human CD45⁺ and CD45⁺/GFP⁺ cells performed by mixed-effects model (REML), followed by post hoc analysis with Dunnett's test (within groups).
(R) Chimerism of GFP⁺ cells observed within CD19⁺, CD13⁺ and CD3⁺ cells in BM after M-HSCT. Mixed-effects model (REML), followed by post hoc analysis with Sidak's test (between groups) or by post hoc analysis with Dunnett's test (within groups).
(S) Percentage of HSPCs (CD34⁺ CD38⁻ CD90⁺) in human CD45⁺ (left panel) and CD45⁺/GFP⁺ cells (right panel) in the BM after M-HSCT. Kruskal-Wallis test was performed, followed by post hoc analysis with Dunn's test.
(T) Chimerism of GFP⁺ cells observed within HSPCs in BM after M-HSCT. Mann-Whitney test performed.

Results are mean±SEM, with n≥9. P-values were defined as such (*P<0.05, **P<0.01, ***P<0.001, ****P<0.0001. “ns” means non-significance).

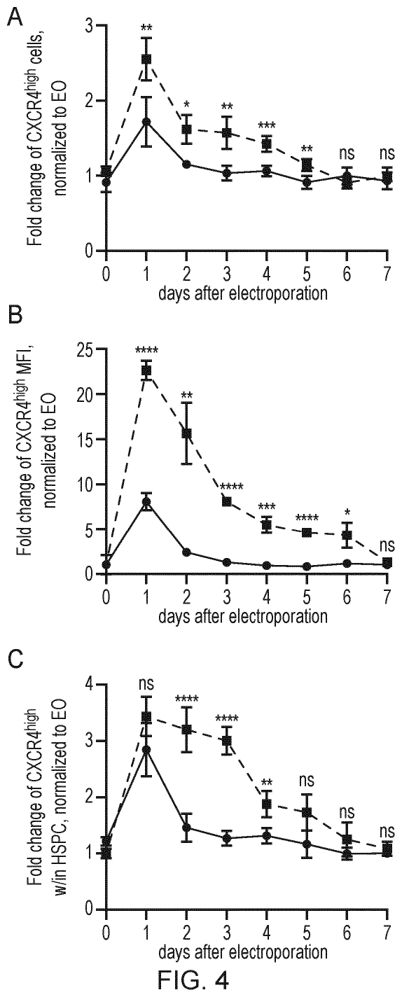

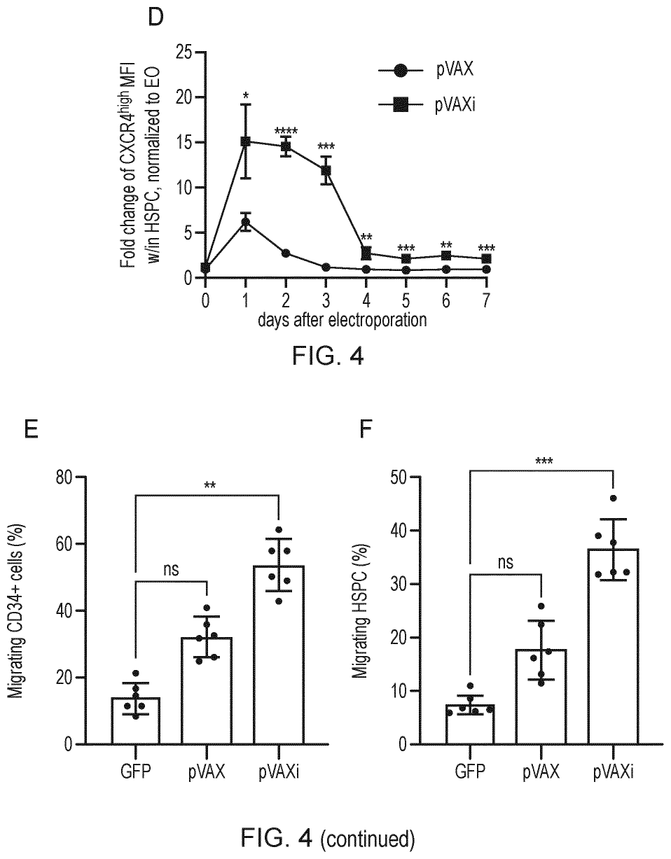

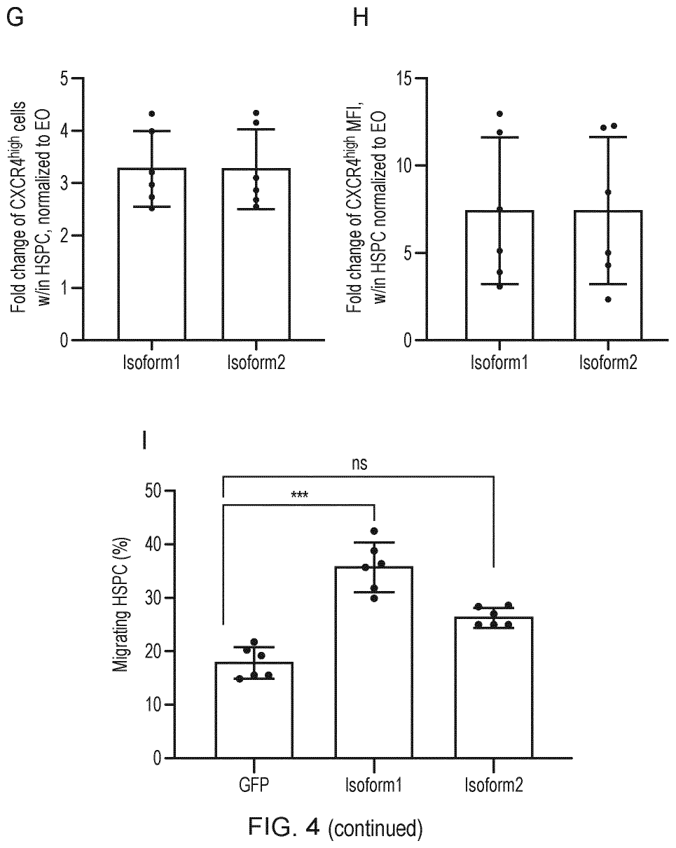

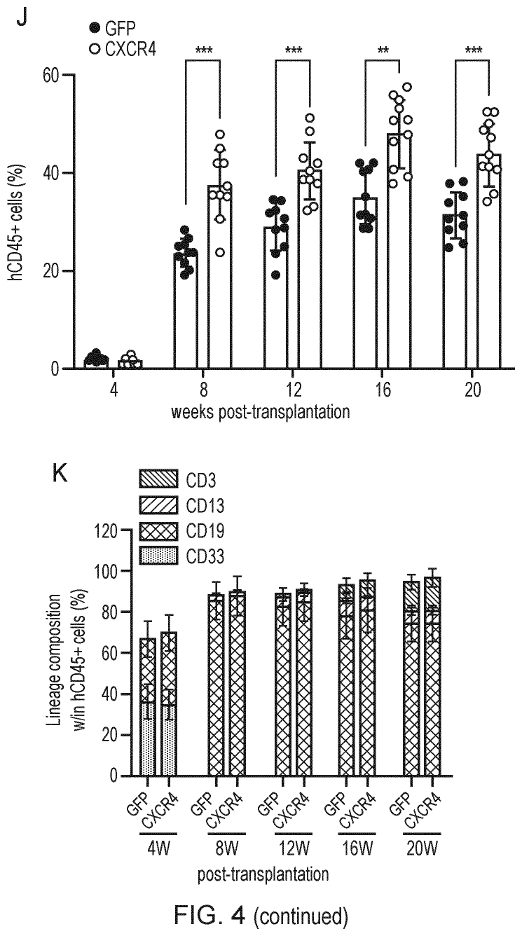

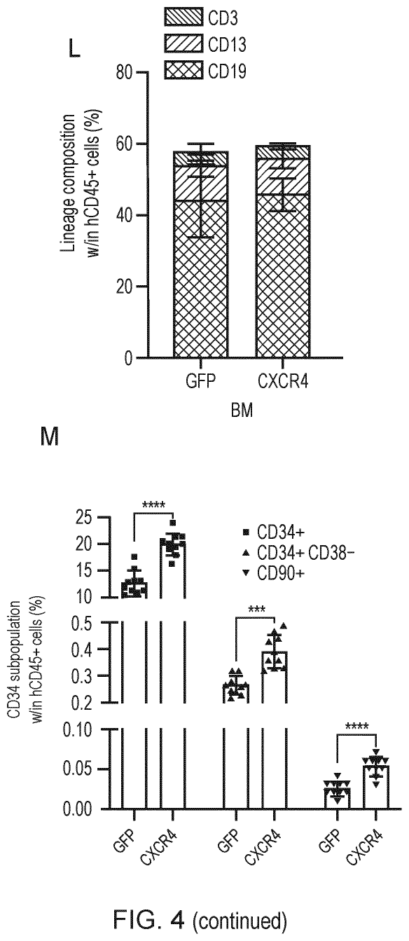

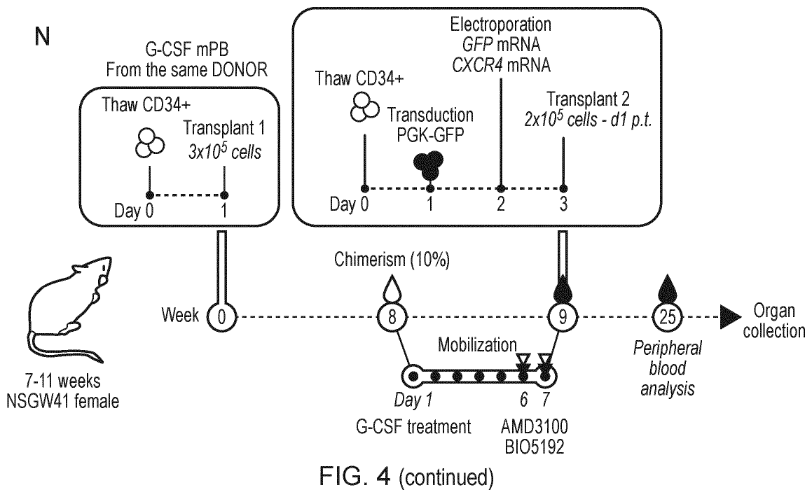

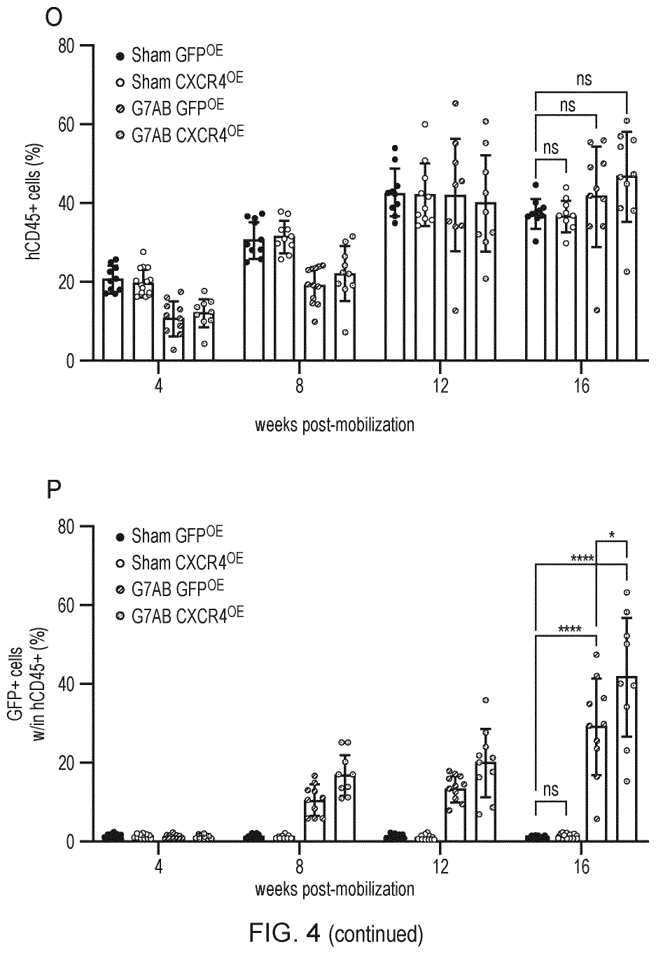

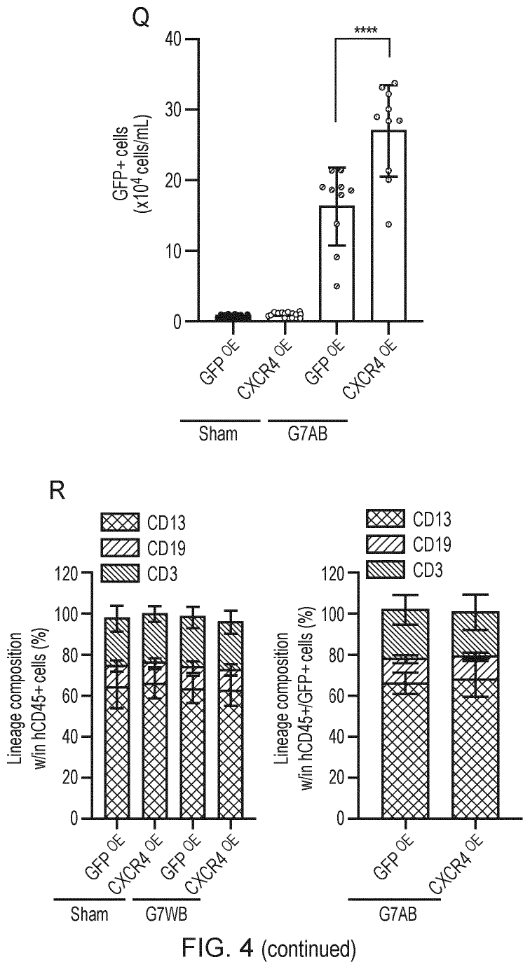

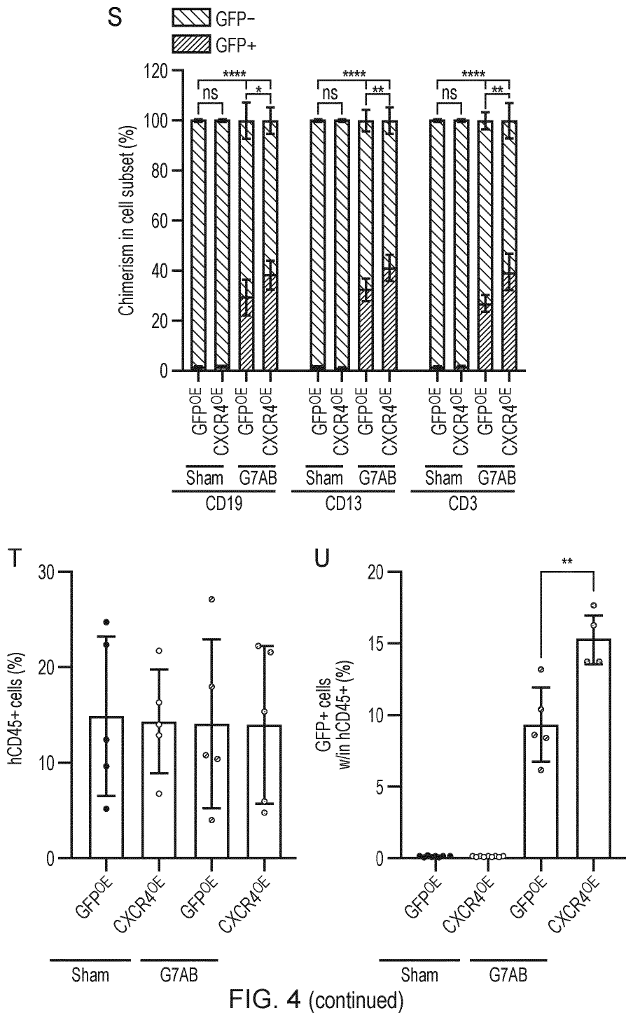

FIG. 4. Transient overexpression of CXCR4 increases chimerism in the humanized context, following M-HSCT
(A-B) Fold change of CXCR4^{high} cells (A) and CXCR4^{high} MFI (B) in bulk CD34⁺ cells, electroporated with CXCR4 mRNA (pVAX) or optimized CXCR4 mRNA (pVAXi), normalized to electroporated-only (EO) cells.
(C-D) Fold change of CXCR4^{high} cells (C) and CXCR4^{high} MFI (D) in CD34⁺ CD133⁺ CD90⁺ HSPCs, electroporated with CXCR4 mRNA or optimized CXCR4 mRNA, normalized to (electroporated only) EO cells.

Mixed-effects model (REML), followed by post hoc analysis with Sidak's test (A-D).
(E-F) Percentage of migrating bulk CD34⁺ cells (E) and HSPCs (F), electroporated with GFP, CXCR4 or optimized CXCR4 mRNA. Kruskal-Wallis test was performed, followed by post hoc analysis with Dunn's test.
(G-H) Fold change of CXCR4^{high} cells (G) and CXCR4^{high} MFI (H) in HSPCs, electroporated with CXCR4 isoform1 or isoform2 mRNA, normalized to EO cells.
(I) Percentage of migrating HSPCs, electroporated with GFP, CXCR4 isoform1 or isoform2 mRNA. Kruskal-Wallis test was performed, followed by post hoc analysis with Dunn's test.
(J) Long-term follow-up of human CD45⁺ in PB of NSG mice, following the transplantation of CD34⁺ cells electroporated with GFP or CXCR4 mRNA. Longitudinal comparisons, performed by mixed-effects model (REML), followed by post hoc analysis with Sidak's test.
(K) Reconstitution of myeloid and lymphoid lineages overtime within total CD45⁺ cells in PB of NSG mice, transplanted with CD34⁺ cells electroporated with GFP or CXCR4 mRNA. Comparison of lineages between human CD45⁺ and GFP⁺ cells performed at the last time point by mixed-effects model (REML), followed by post hoc analysis with Dunnett's test (within groups).
(L) Myeloid and lymphoid cell composition of human CD45⁺ cells in the BM of NSG mice, transplanted with CD34⁺ cells electroporated with GFP or CXCR4 mRNA. Comparison of lineages between human CD45⁺ cells by mixed-effects model (REML), followed by post hoc analysis with Dunnett's test (within groups).
(M) CD34⁺ subpopulation in the BM of NSG mice, transplanted with CD34⁺ cells electroporated with GFP or CXCR4 mRNA. Mixed-effects model (REML), followed by post hoc analysis with Sidak's test.
(N) Schematic illustration of competitive transplantation, after G7AB mobilization, between human resident cells (initially transplanted with 3×10⁵ CD34⁺ G-CSF mPB cells, counted on d1 p.t.) and newly transplanted CD34⁺ G-CSF mPB GFP-transduced and electroporated cells (outgrowth of 2×10⁵ CD34⁺ cells, counted on d1 p.t., transplanted on d3 p.t.) in NSGW41 mice.
(O-P) Long-term follow-up of human CD45⁺ (O) and CD45⁺/GFP⁺ (P) cells in PB after M-HSCT with CD34⁺ cells transiently overexpressing GFP or CXCR4 mRNA, in NSGW41 mice. Two-way ANOVA followed by post hoc analysis with Tukey's test.
(Q) Counts of GFP⁺ cells per mL in the PB after M-HSCT with CD34⁺ cells transiently overexpressing GFP or CXCR4 mRNA, in NSGW41 mice. Two-way ANOVA followed by post hoc analysis with Tukey's test.
(R) Myeloid and lymphoid lineage composition of human CD45⁺ (left panel) and CD45⁺/GFP⁺ cells (right panel) in the PB after M-HSCT, with CD34⁺ cells transiently overexpressing GFP or CXCR4, in NSGW41 mice. Comparison of lineages between human CD45⁺ and CD45⁺/GFP⁺ cells performed by mixed-effects model (REML), followed by post hoc analysis with Dunnett's test (within groups).
(S) Chimerism of GFP⁺ cells observed within CD19⁺, CD13⁺ and CD3⁺ cells in PB after M-HSCT, with CD34⁺ cells transiently overexpressing GFP or CXCR4, in NSGW41 mice. Mixed-effects model (REML), followed by post hoc analysis with Tukey's test (between groups) or by post hoc analysis with Dunnett's test (within groups).
(T) Percentage of human CD45⁺ cells in PB of NSG mice at 16 weeks, following secondary transplant of cells collected from groups described in FIG. 4O. Two-way ANOVA followed by post hoc analysis with Tukey's test.
(U) Percentage of human CD45⁺/GFP⁺ cells in PB of NSG mice at 16 weeks, following secondary transplant of cells collected from groups described in FIG. 4O. Two-way ANOVA followed by post hoc analysis with Tukey's test.

Results are mean±SEM, with n ≥10 for data collected in vivo, n≥4 for secondary transplant experiments, and n ≥5 for data collected in vitro (3 different donors). P-values were defined as such (*P<0.05, **P<0.01, ***P<0.001, ****P<0.0001. “ns” means non-significance).

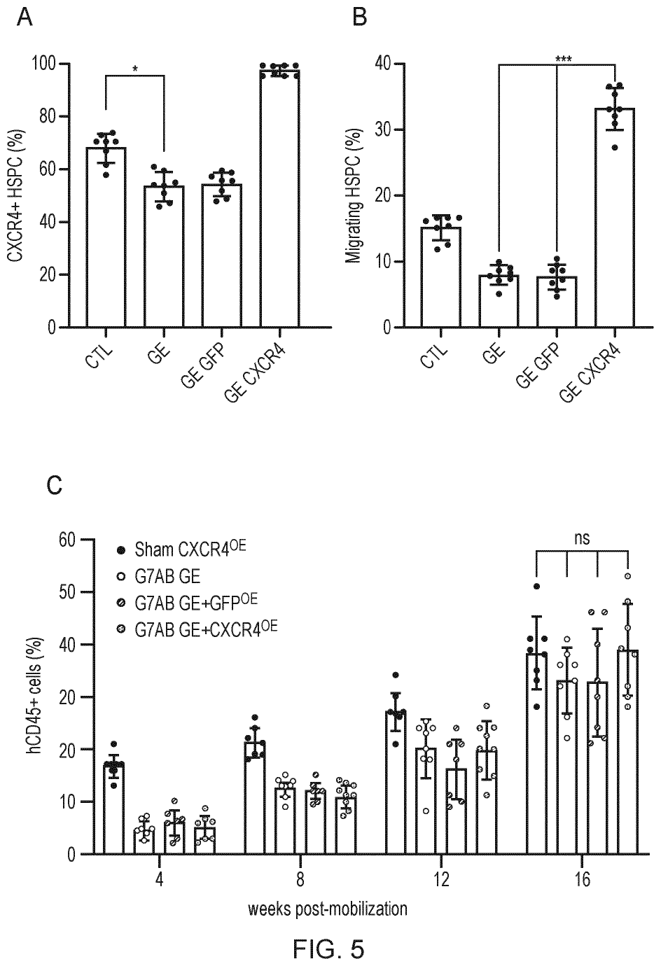

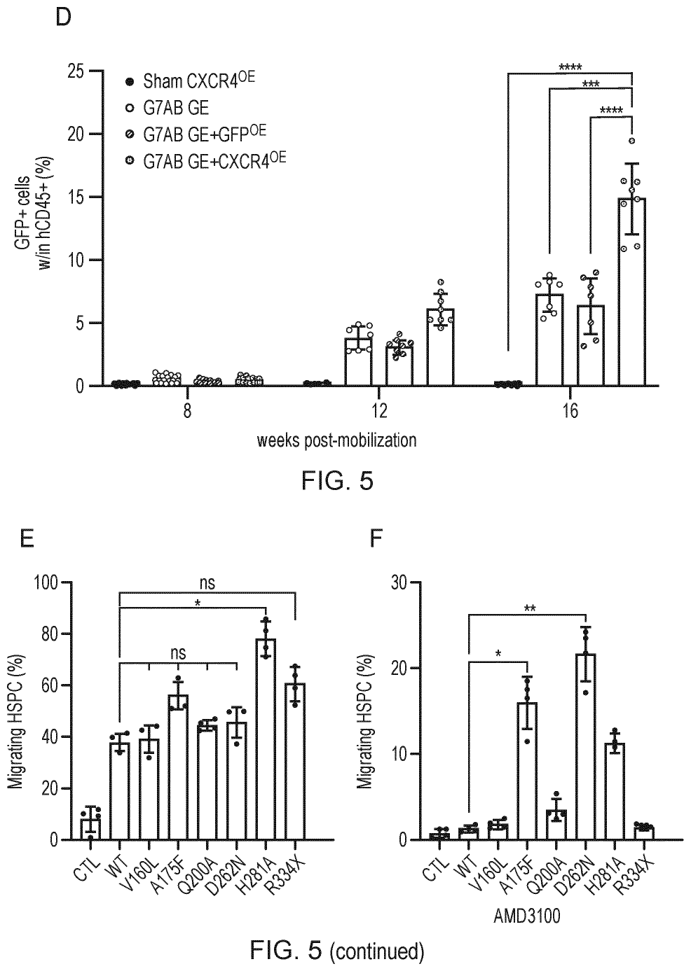

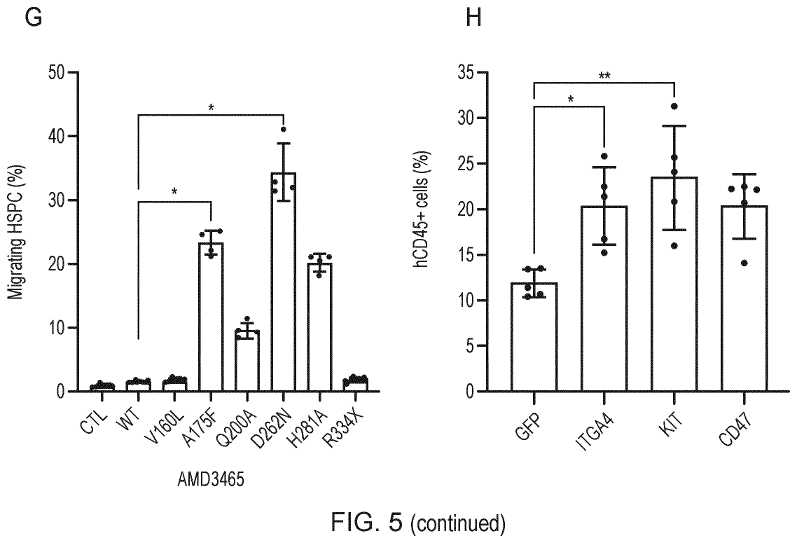

FIG. 5. M-HSCT confers significant advantage to gene edited cells when paired with an engraftment enhancer
(A-B) Percentage of CXCR4⁺ cells in HSPCs (CD34⁺ CD133⁺ CD90⁺) (A) and of migrating HSPCs (B), following gene editing (GE; Cas9 RNA, AAVS1 sgRNA, AAV6-GFP) combined or not with GFP (GE GFP) or CXCR4 (GE CXCR4) mRNA. Kruskal-Wallis test, followed by post hoc analysis with Dunn's test.
(C-D) Long-term follow-up of human CD45⁺ (C) and CD45⁺/GFP⁺ (D) cells in PB after M-HSCT (as in FIG. 3D) with CD34⁺ cells gene edited as in A, in NSGW41 mice. Mixed-effects model (REML), followed by post hoc analysis with Tukey's test.
(E-G) Percentage of migrating HSPCs without treatment (E), in presence of AMD3100 (F) or AMD3465 (G), electroporated with CXCR4 variants. Kruskal-Wallis test, followed by post hoc analysis with Dunn's test.
(H) Percentage of human CD45⁺ in PB of NSG mice at 16 weeks, following the transplantation of CD34⁺ cells electroporated with GFP, ITGA4, KIT and CD47 mRNA. Kruskal-Wallis test, followed by post hoc analysis with Dunn's test.

Results are mean±SEM, with n ≥8 for data collected in vivo on NSGW41 mice, with n ≥5 for data collected in vivo on NSG mice and n ≥5 for data collected in vitro (3 different donors).

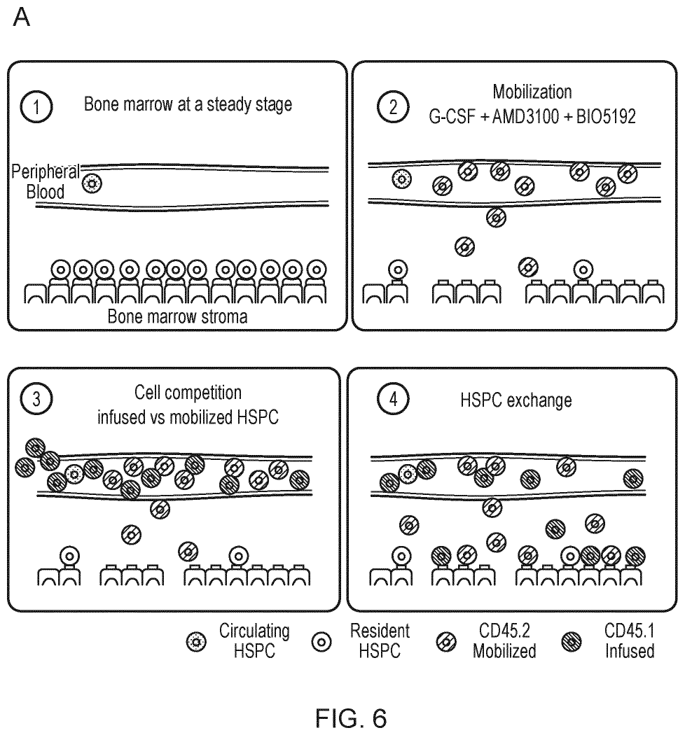

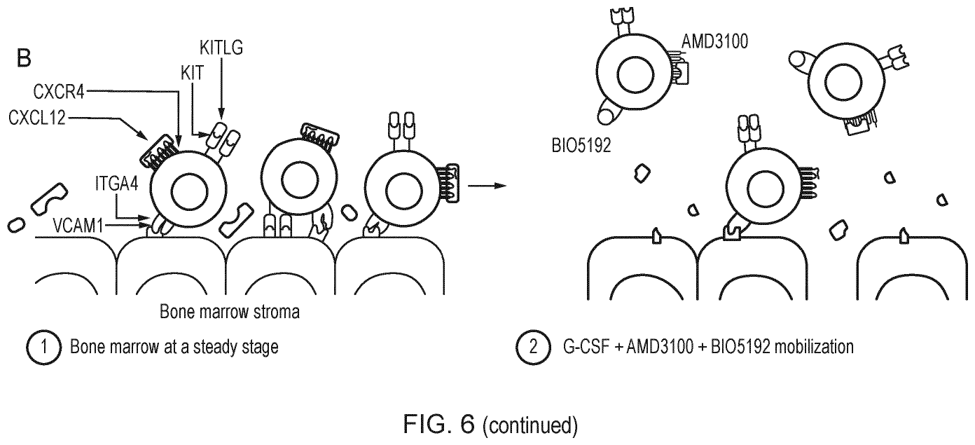

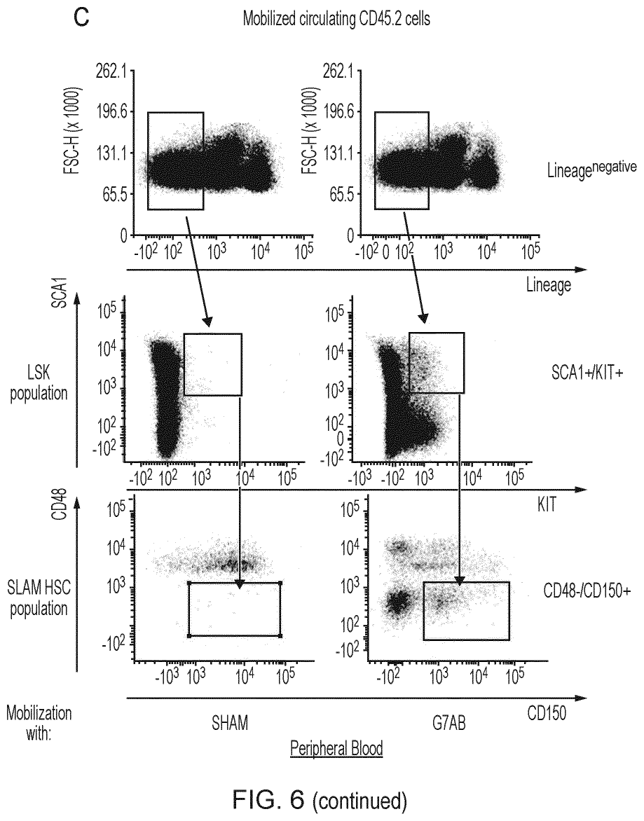

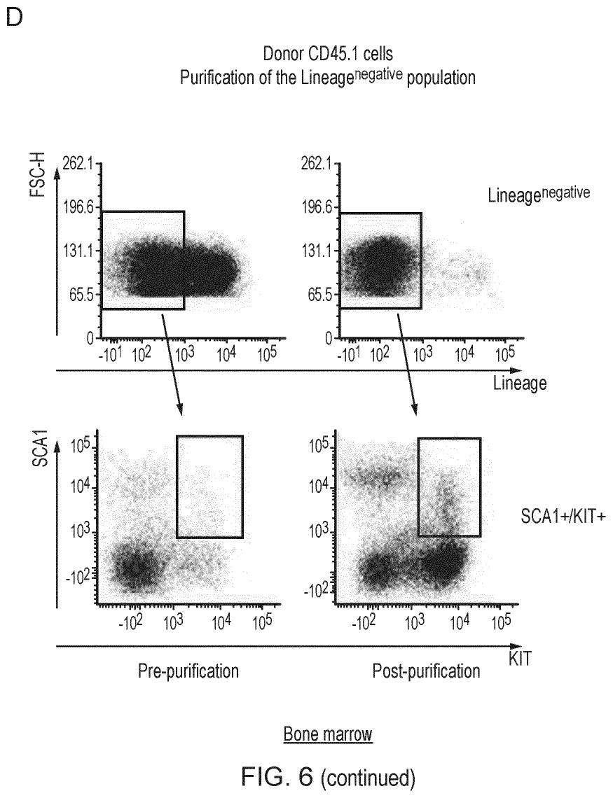

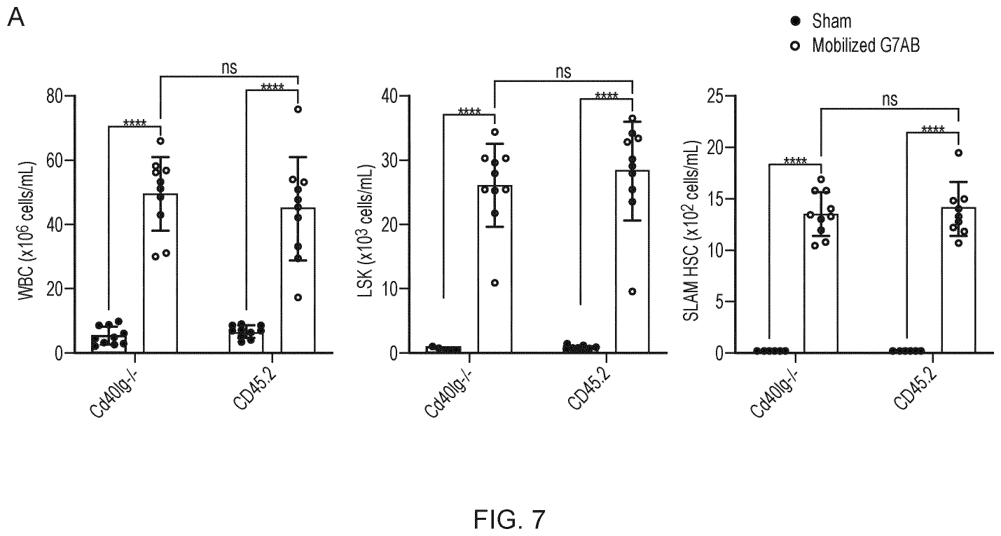

FIG. 6. Long-term donor chimerism is established by M-HSCT, related to FIG. 1
(A) Schematic of the M-HSCT strategy, illustrating the proposed exchange between mobilized recipient CD45.2 and donor CD45.1 cells.
(B) Schematic of the interaction between mobilization agents and their therapeutic targets within the BM microenvironment.
(C) Representative plots showing the gating strategy used to characterize LSK and SLAM HSC circulating in the peripheral blood, stained for Lineage markers, SCA1, KIT, CD48 and CD150.
(D) Representative plots showing the gating strategy used to characterize donor cells, extracted from the BM, stained for Lineage markers, SCA1, KIT, pre- and post-purification of Lineage negative cells.

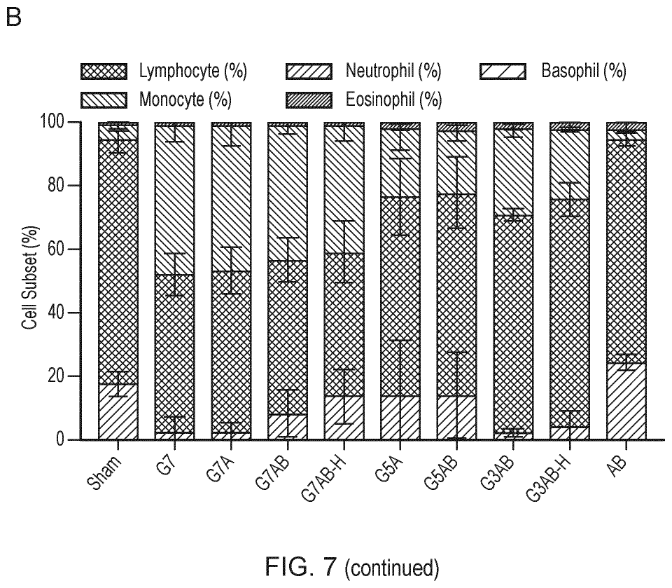

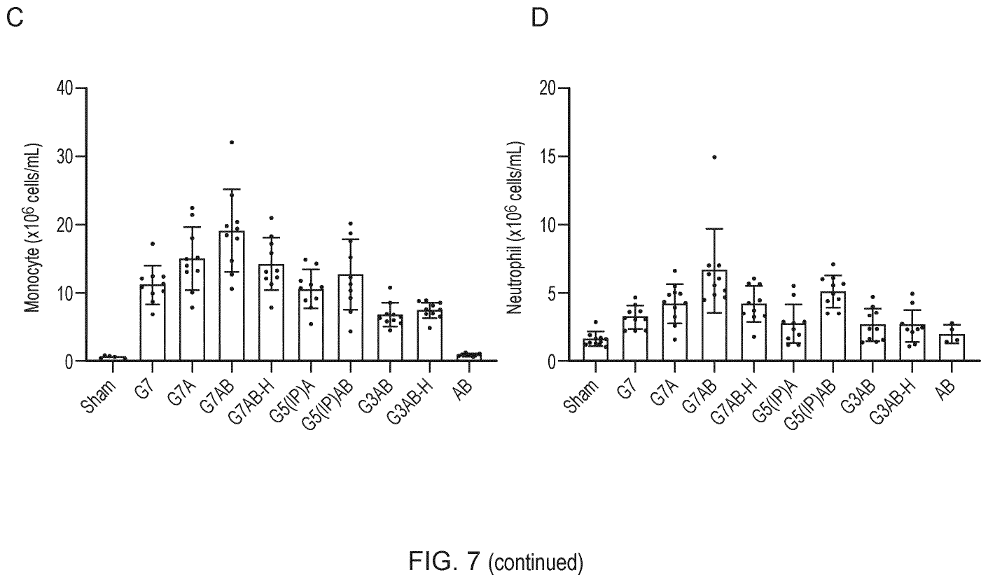

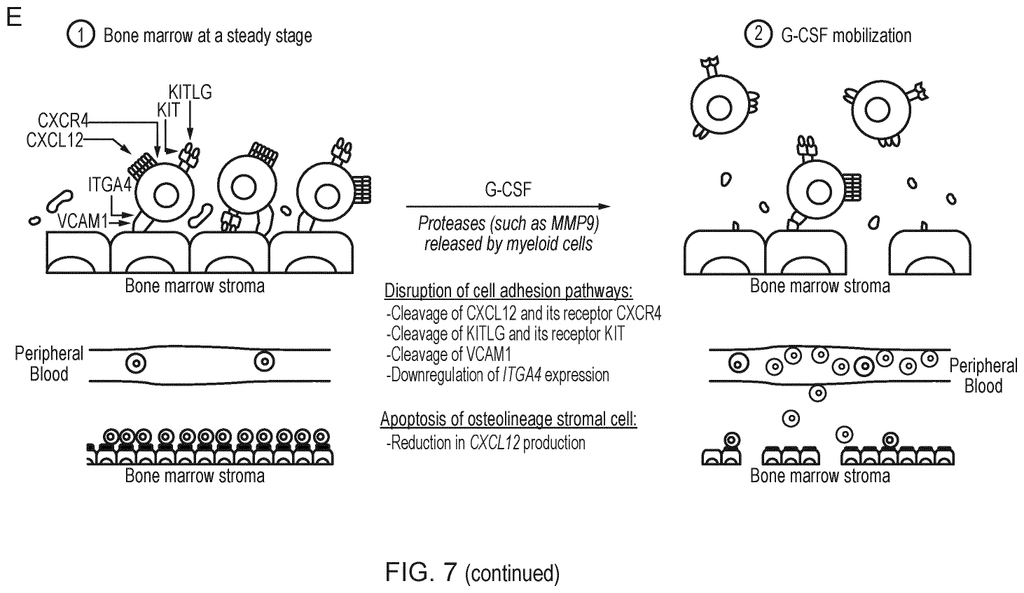

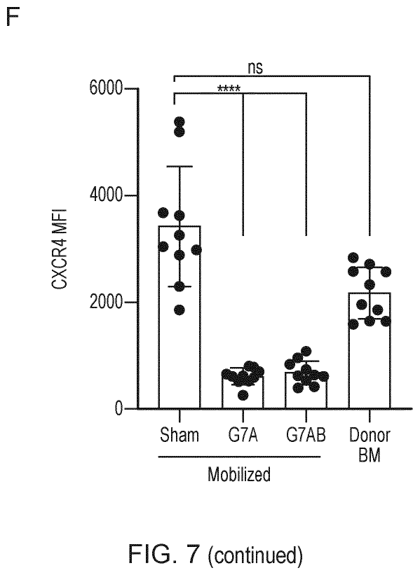

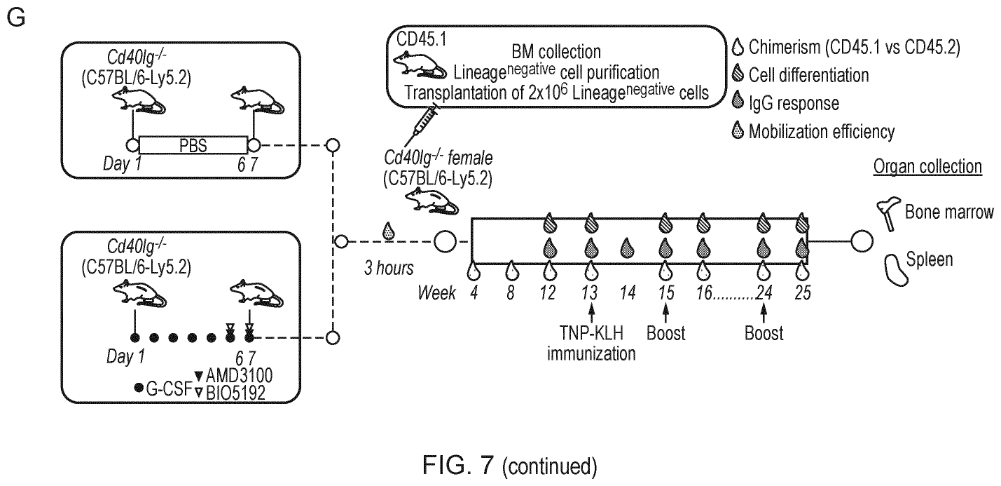

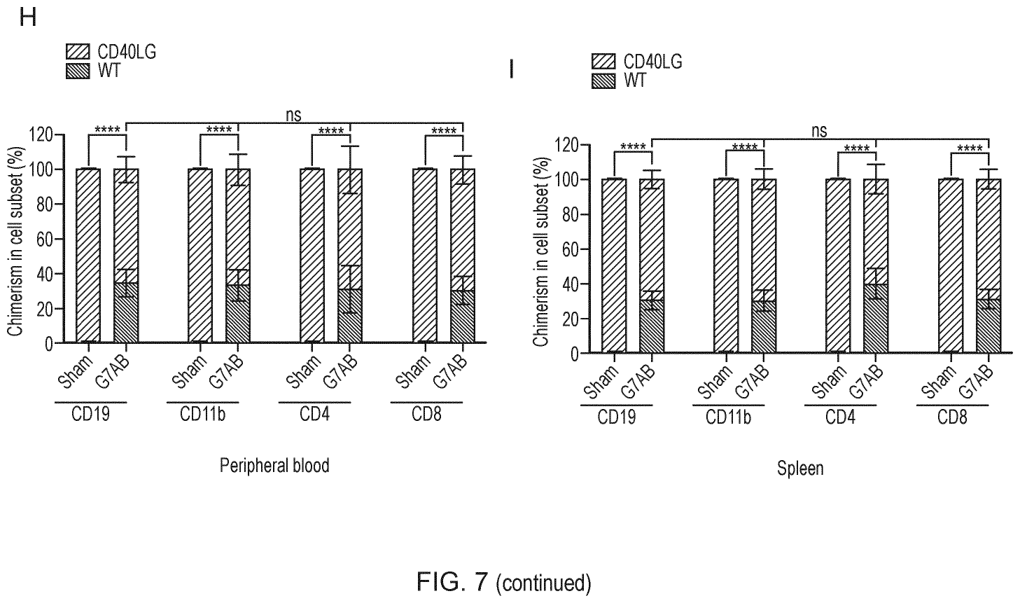

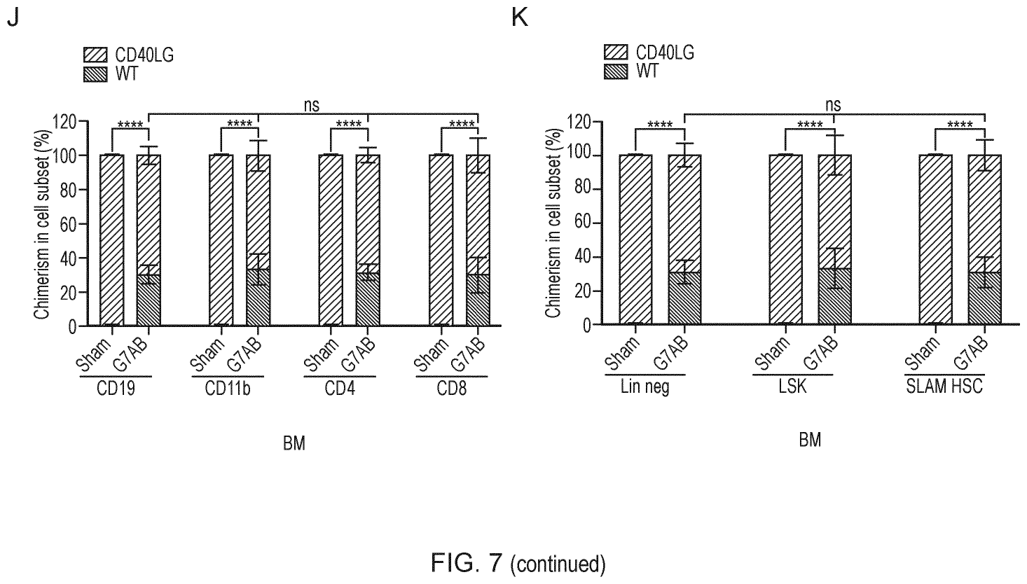

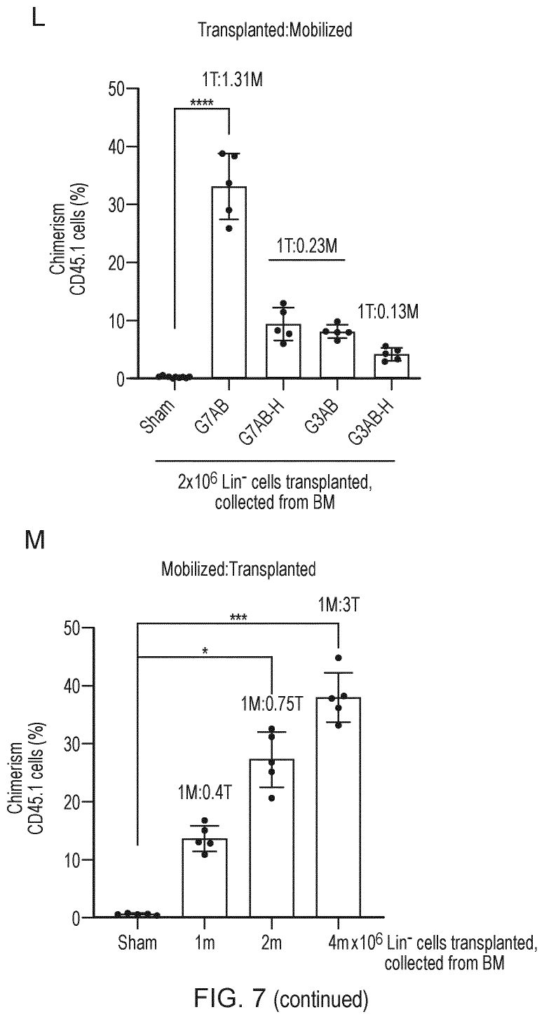

FIG. 7. M-HSCT allows establishing sufficient donor chimerism to rescue the HIGM1 phenotype, related to FIG. 2
(A) Counts of mobilized WBC (left panel), LSK (middle panel) and SLAM HSC (right panel) cells per mL in the PB of Cd40Ig^{-/-} mice and CD45.2 mice, after mobilization with PBS (Sham) or mobilized with G7AB. Mixed-effects model (REML), followed by post hoc analysis with Sidak's test.
(B) Percentage of neutrophils, lymphocytes, monocytes, eosinophils and basophils in the PB after different mobilization protocols.
(C-D) Counts of mobilized monocytes (C) and neutrophils (D) per mL in the PB after different mobilization protocols.
(E) Schematic of G-CSF impact on the BM.
(F) CXCR4 MFI on circulating LSK after treatments with PBS, G7A, G7AB and on donor cells purified from untreated bone marrow. Kruskal-Wallis test, followed by post hoc analysis with Dunn's test.
(G) Schematic of the M-HSCT protocol applied to Cd40Ig^{-/-} mice. Recipient Cd40Ig^{-/-} mice were mobilized with G-CSF (green dots), AMD3100 (red triangle) and BIO5192 (blue triangle) (G7AB), and subsequently transplanted with 2×10⁶ WT CD45.1 Lin⁻ cells, collected from the BM.
(H-K) Chimerism of WT cells observed within CD19⁺, CD11b⁺, CD4⁺ and CD8⁺ cells in PB (H), spleen (I) and in BM (J) and within Lin⁻, LSK and SLAM HSC in BM (K). Mixed-effects model (REML), followed by post hoc analysis with Tukey's test (between groups) or by post hoc analysis with Dunnett's test (within groups).
(L) Chimerism of CD45.1 cells at 20 weeks, following different M-HSCT protocol, subsequently transplanted with 2×10⁶ WT CD45.1 Lin⁻ cells, collected from the BM.
(M) Chimerism of CD45.1 cells at 20 weeks, following mobilization with G7AB and subsequently transplanted with different cell doses (WT CD45.1 Lin− cells, collected from the BM).

Results are mean±SEM, with n ≥5 for the kinetic experiments, except for the AB group (n-4), and with n≥9 for the Cd40Ig−/− M-HSCT experiments.

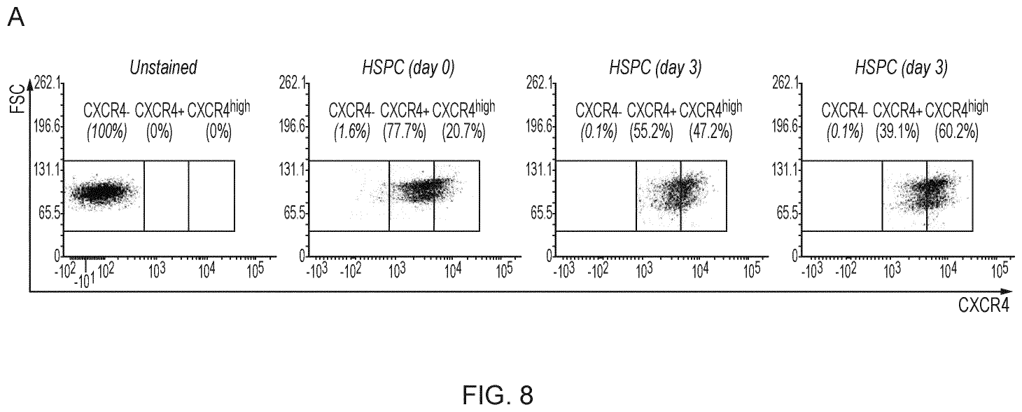

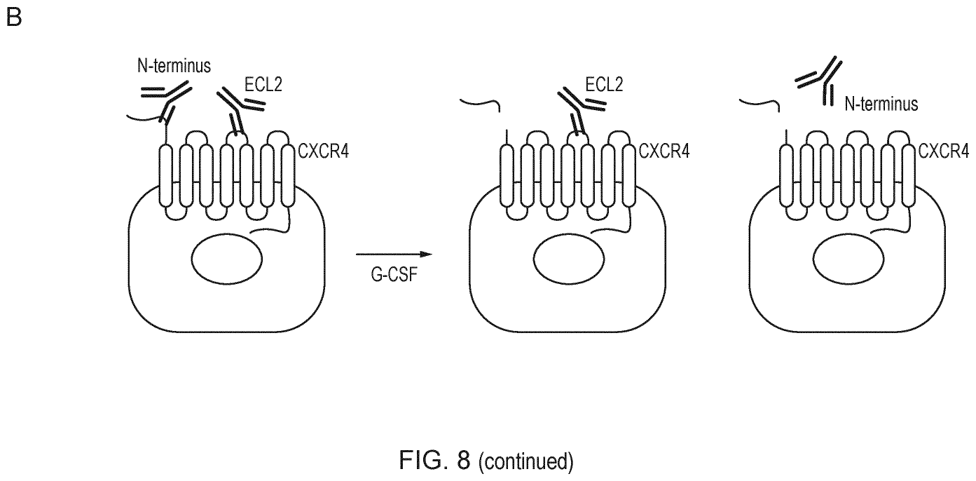

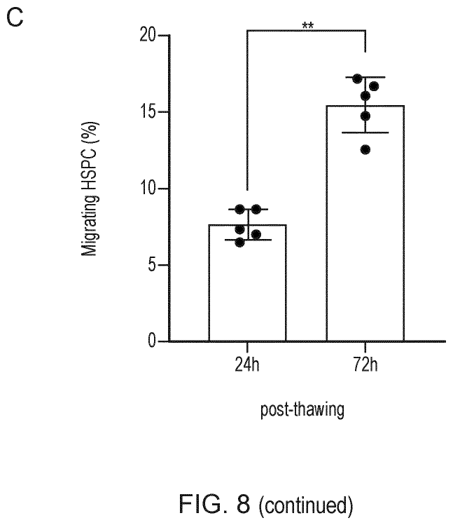

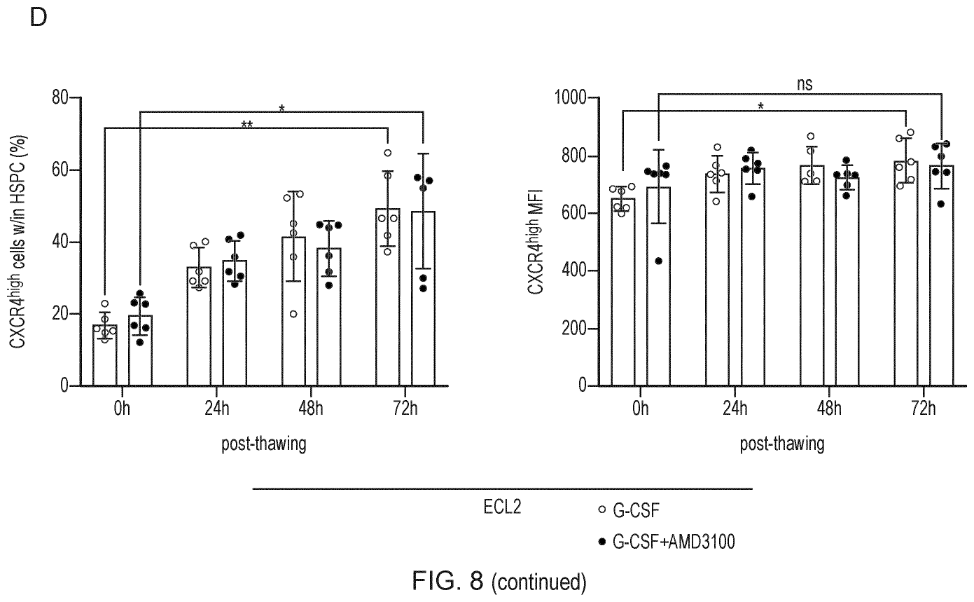

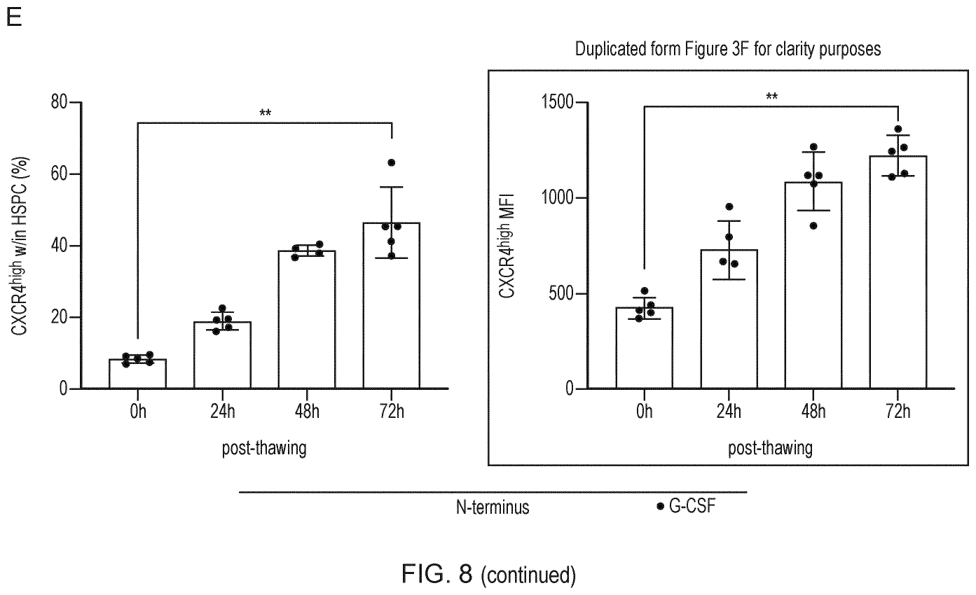

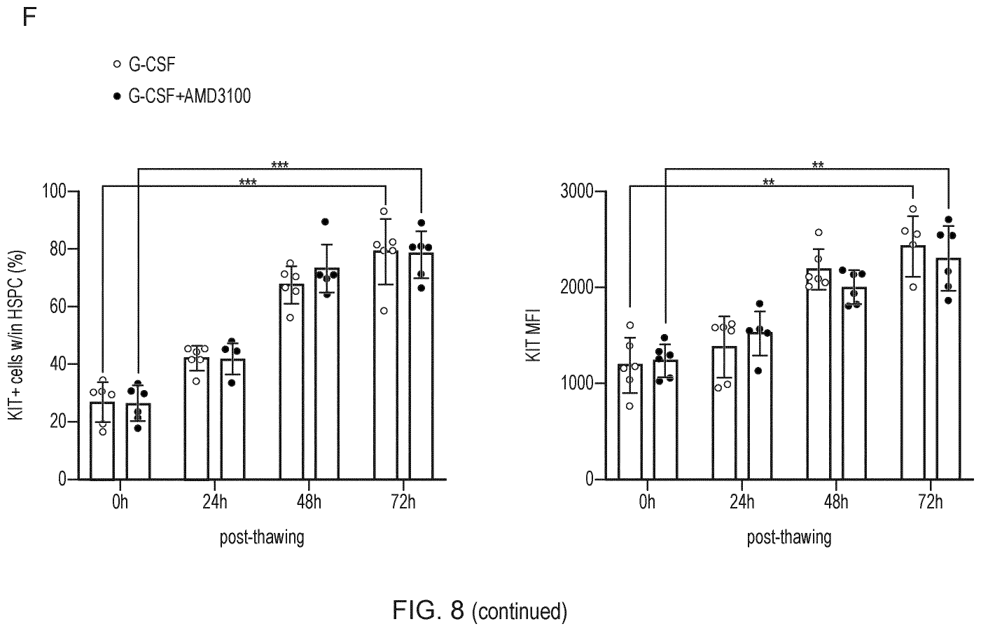

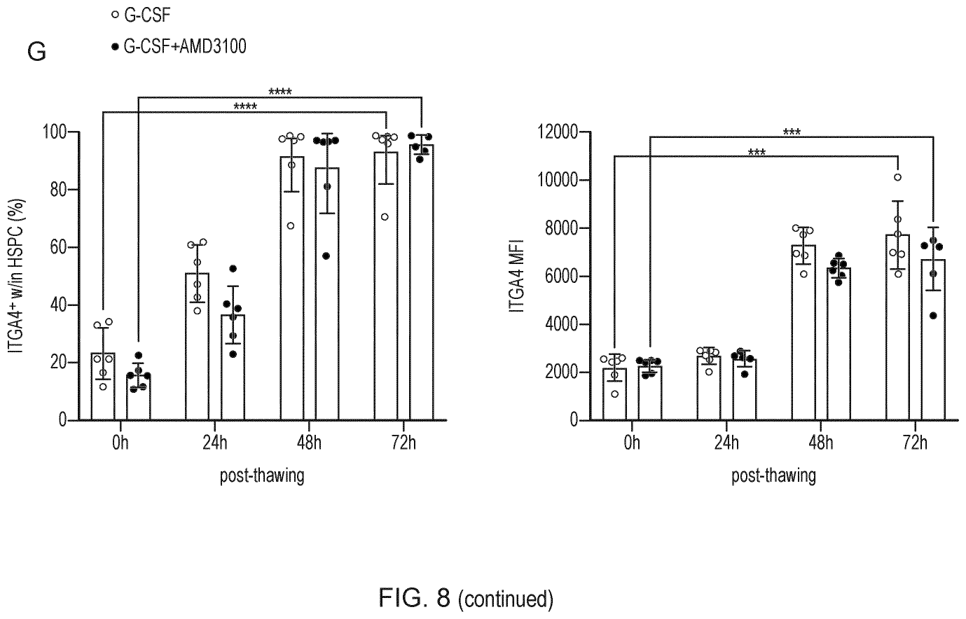

FIG. 8. M-HSCT allows efficient donor to recipient exchange of HSPCs within the human niche of hematochimeric mice, related to FIG. 3
(A) Representative plots showing the gating strategy used to characterize CXCR4^{high} population in HSPC (CD34⁺ CD133⁺ CD90⁺) population.
(B) Scheme of CXCR4 cleavage following G-CSF mobilization and related antibodies localization.
(C) Percentage of migrating HSPCs, performed at 24 hours and 72 hours post-thawing, previously collected with G-CSF. Kruskal-Wallis test was performed, followed by post hoc analysis with Dunn's test.
(D) Percentage of CXCR4^{high} cells (left panel) and MFI (right panel) overtime after thawing, stained with an antibody targeting the ECL2 epitope of CXCR4, on HSPC population mobilized with G-CSF or G-CSF/AMD3100.
(E) Percentage of CXCR4^{high} cells (left panel) and MFI (right panel) overtime after thawing, stained with an antibody targeting the N-terminus epitope of CXCR4, on HSPC population mobilized with G-CSF.
(F) Percentage of KIT⁺ cells (left panel) and MFI (right panel) overtime after thawing, on HSPC population mobilized with G-CSF or G-CSF/AMD3100.
(G) Percentage of ITGA4⁺ cells (left panel) and MFI (right panel) overtime after thawing, on HSPC population mobilized with G-CSF or G-CSF/AMD3100. Longitudinal comparisons, performed by mixed-effects model (REML), followed by post hoc analysis with Dunnett's test.

Results are mean±SEM, with n ≥5, with 3 different donors.

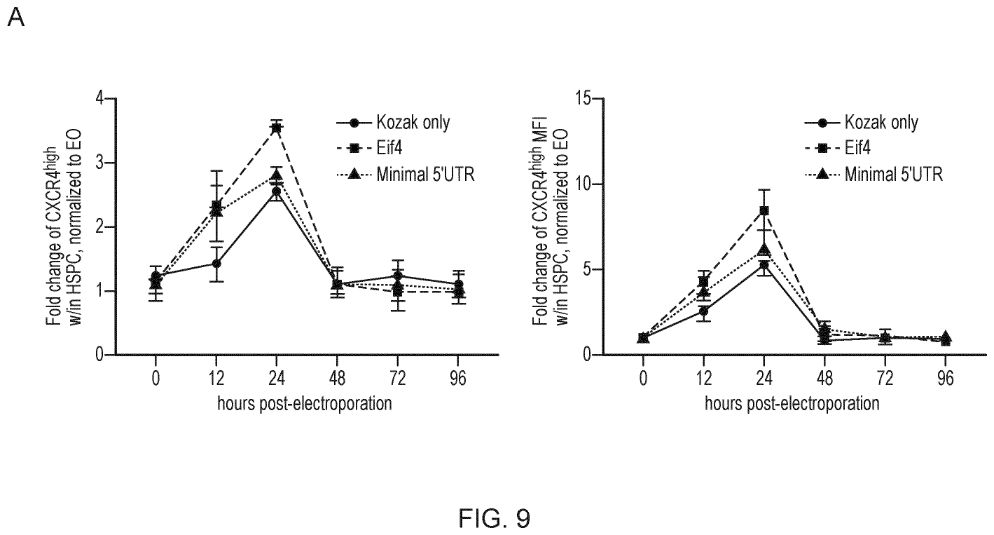

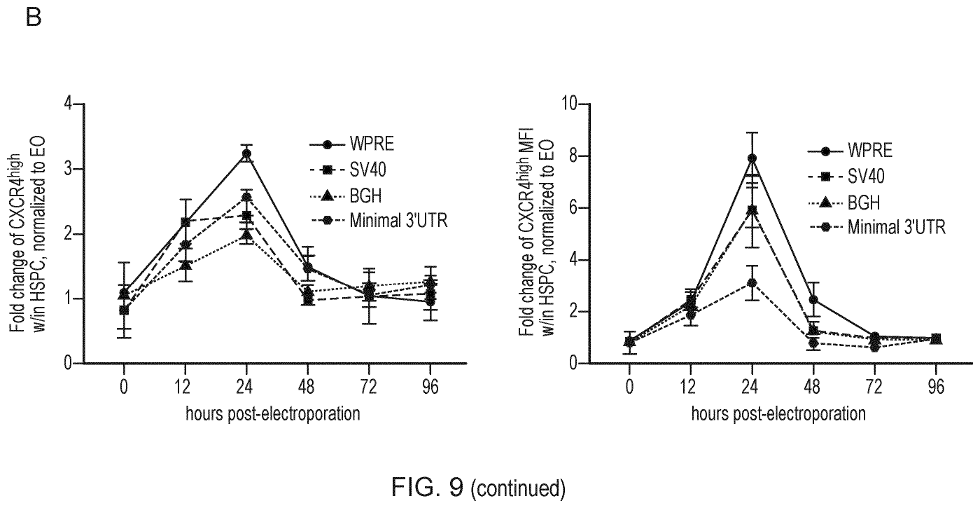

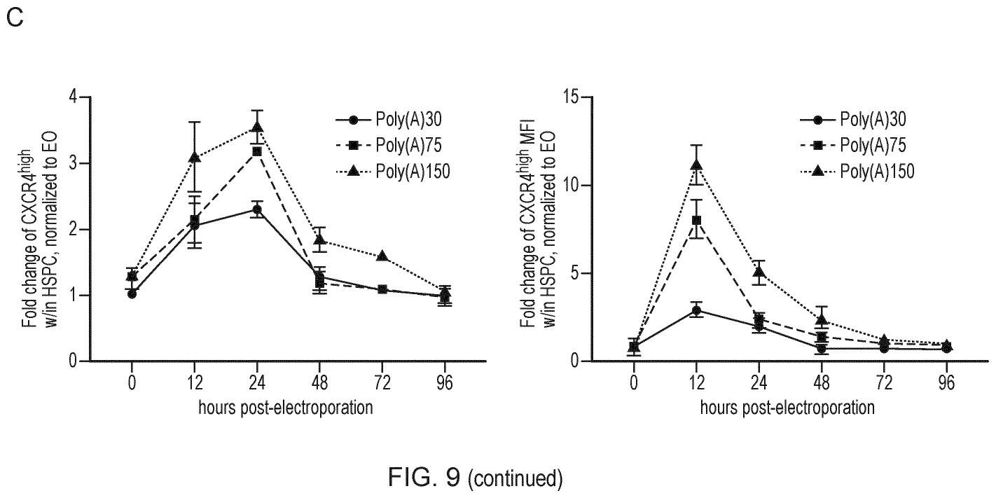

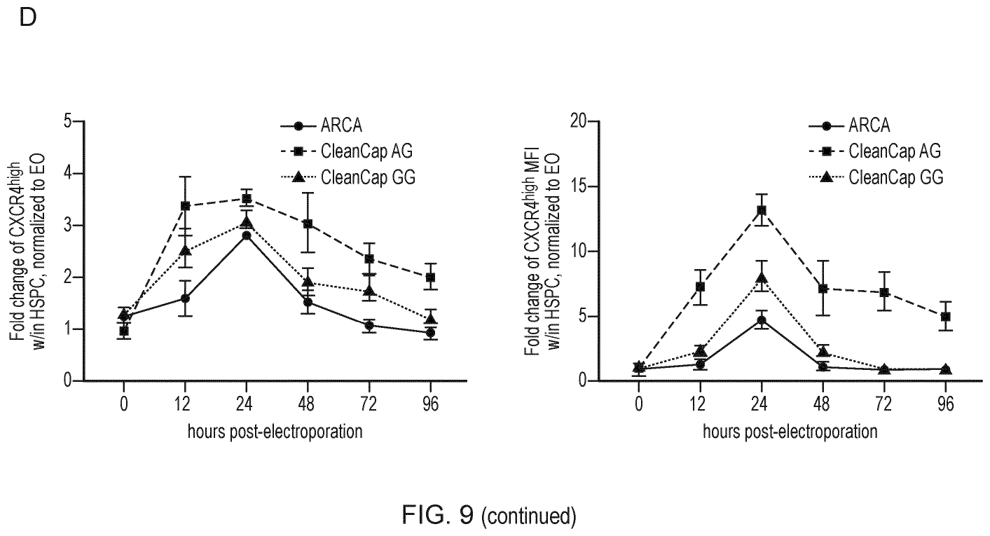

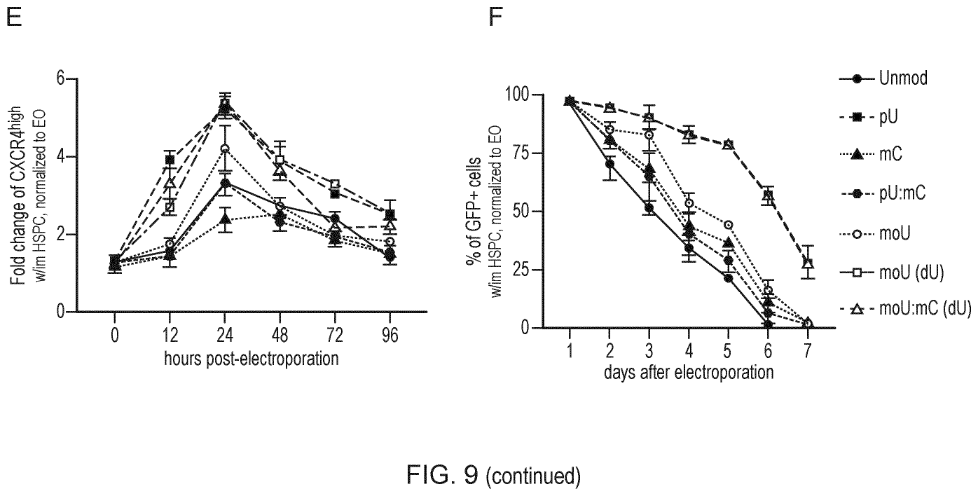

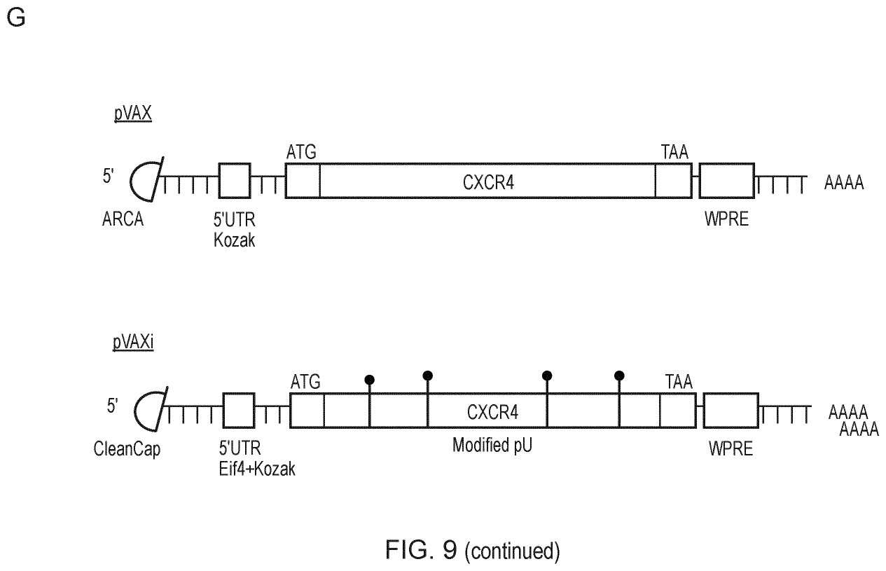

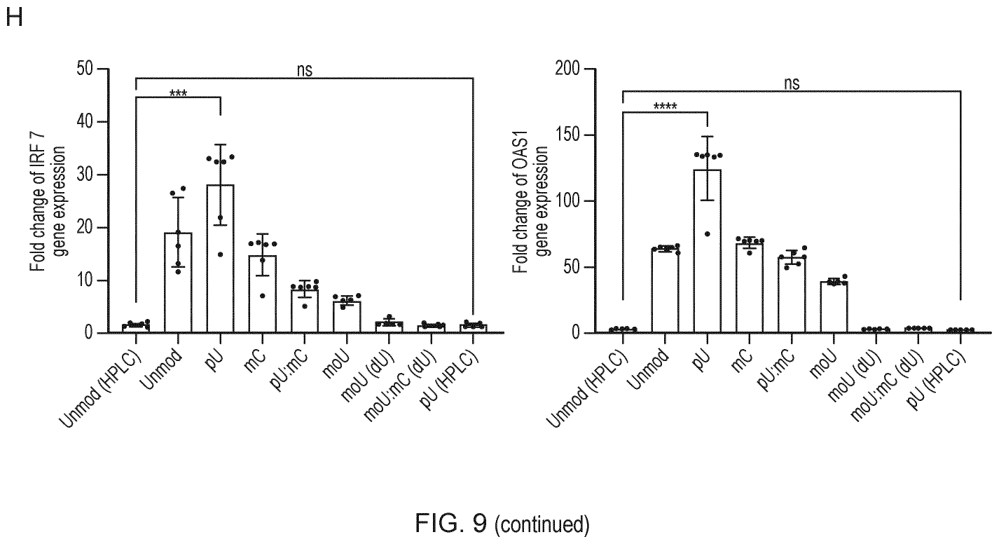

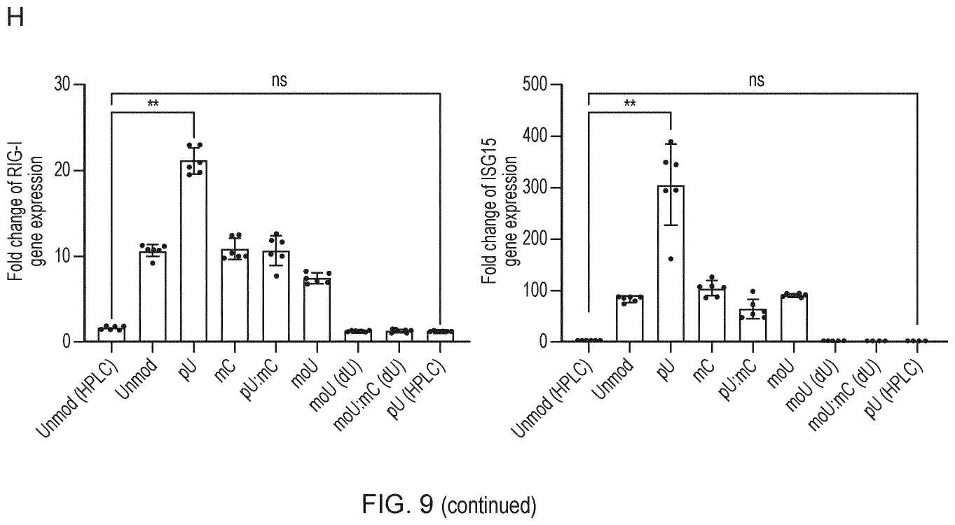

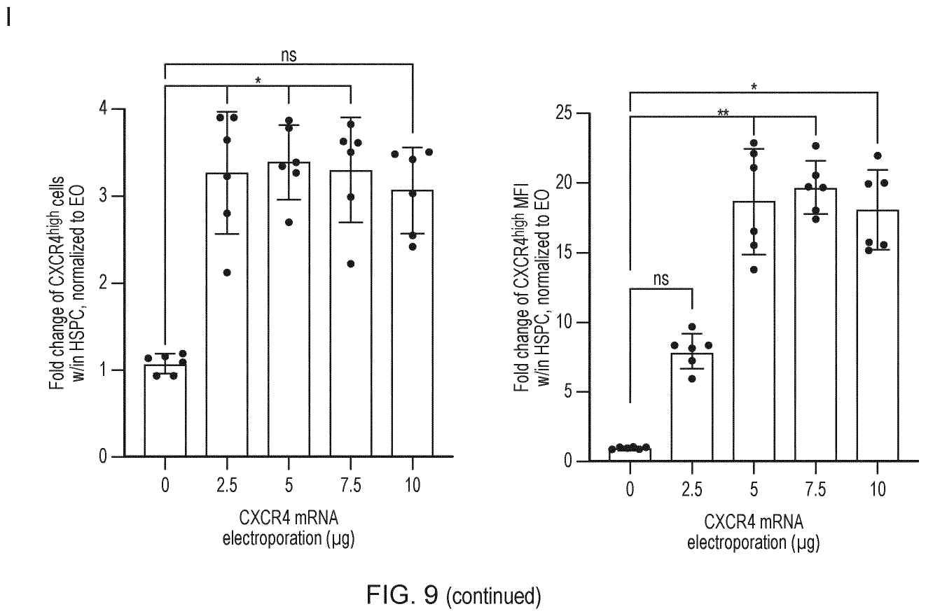

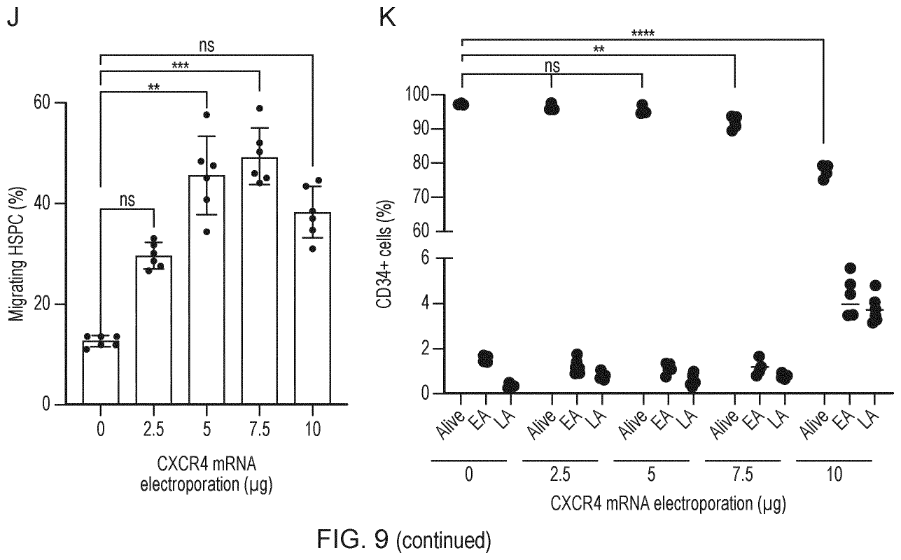

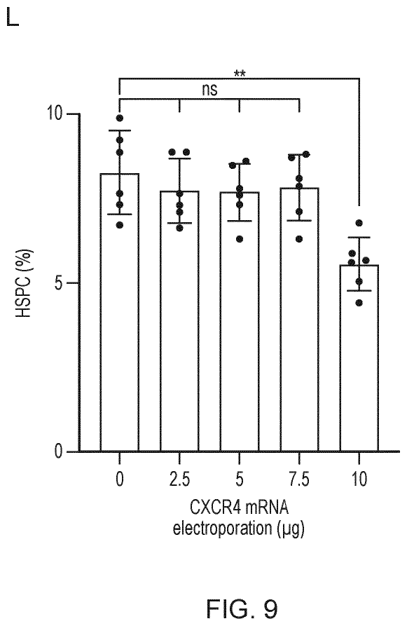

FIG. 9. Optimization of the mRNA delivery platform, related to FIG. 4
(A-D) Fold change of CXCR4^{high} cells (left panel) and CXCR4^{high} MFI (right panel) in HSPCs (CD34⁺ CD133⁺ CD90⁺) electroporated with CXCR4 mRNA differing for the 5′UTR sequence (A), the 3′UTR sequence (B), the polyA tail length (C) and the mRNA capping (D), normalized to EO cells.
(E) Fold change of CXCR4^{high} cells in HSPCs electroporated with CXCR4 mRNA differing for nucleotides used during mRNA synthesis, normalized to EO cells.
(F) Percentage of GFP⁺ cells in HSPCs, electroporated with GFP mRNA differing for nucleotides used for the mRNA synthesis, normalized to EO cells.
(G) Schematic of the pVAX CXCR4 mRNA and improved pVAXi CXCR4 mRNA.
(H) Fold change of IRF7, OAS1, RIG-I and ISG15 gene expression, in cells electroporated with CXCR4 mRNA differing for nucleotides used during mRNA synthesis, normalized to EO cells.
(I) Fold change of CXCR4^{high} cells (left panel) and CXCR4^{high} MFI (right panel) in HSPCs, electroporated with different quantities of CXCR4 mRNA, normalized to EO cells.
(J) Percentage of migrating HSPCs, electroporated with different quantities of CXCR4 mRNA.
(K) Early and late apoptosis (EA, LA) induced by the electroporation of different quantities of CXCR4 mRNA, in bulk CD34⁺ cells.
(L) Percentage of HSPCs (CD34⁺ CD133⁺ CD90⁺) in CD34⁺ cells electroporated with different quantities of CXCR4 mRNA.

Kruskal-Wallis test, followed by post hoc analysis with Dunn's test (G-K). Results are mean±SEM, with n ≥5 for data collected in vitro (3 different donors).

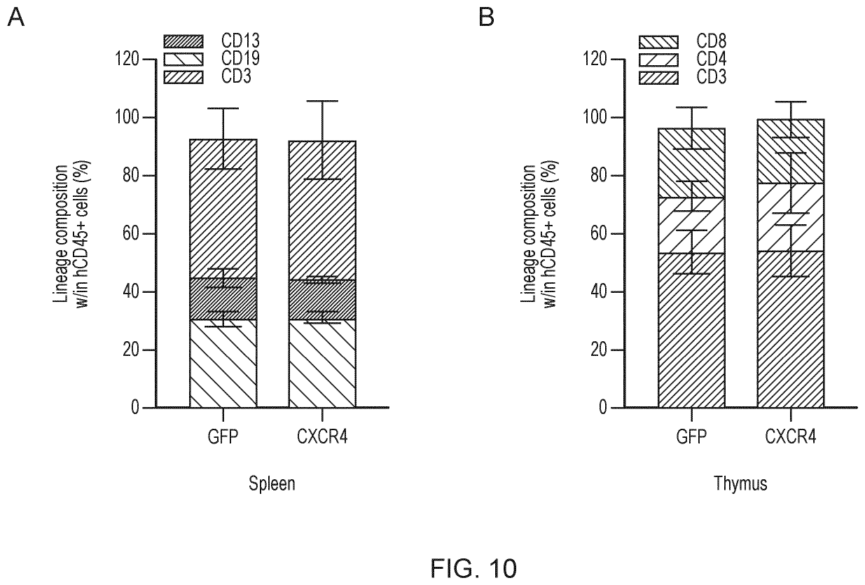

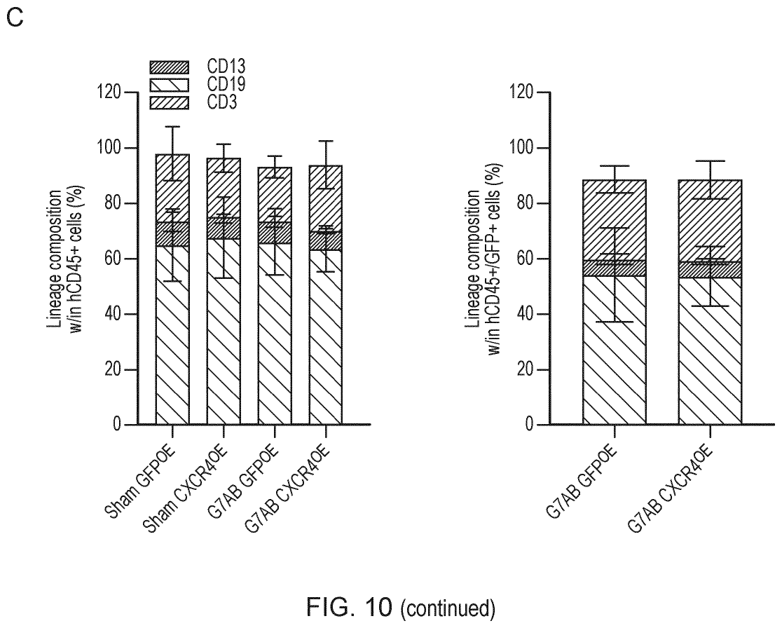

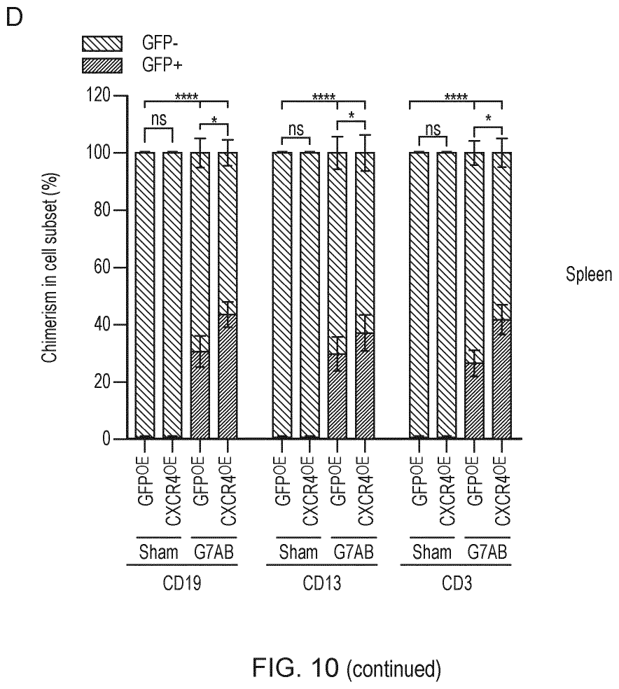

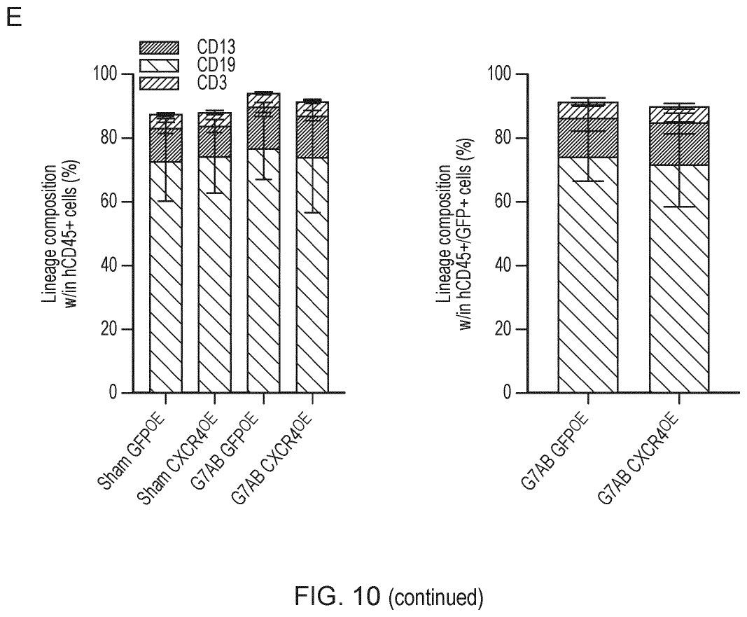

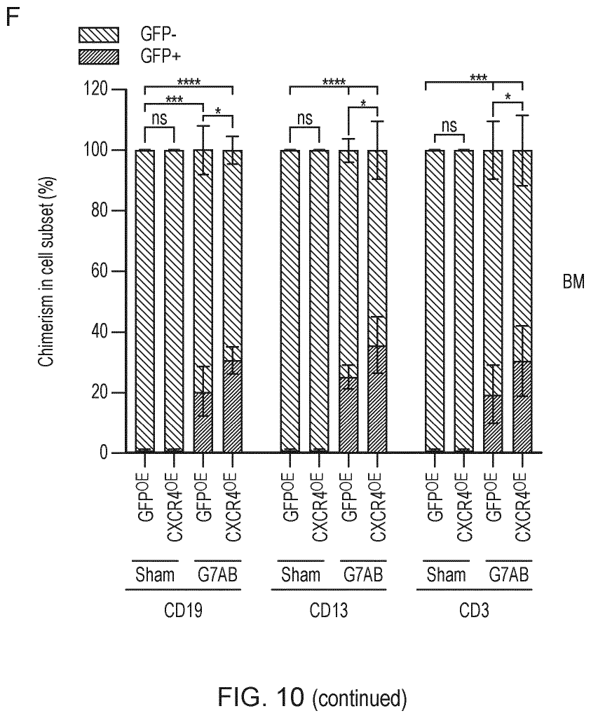

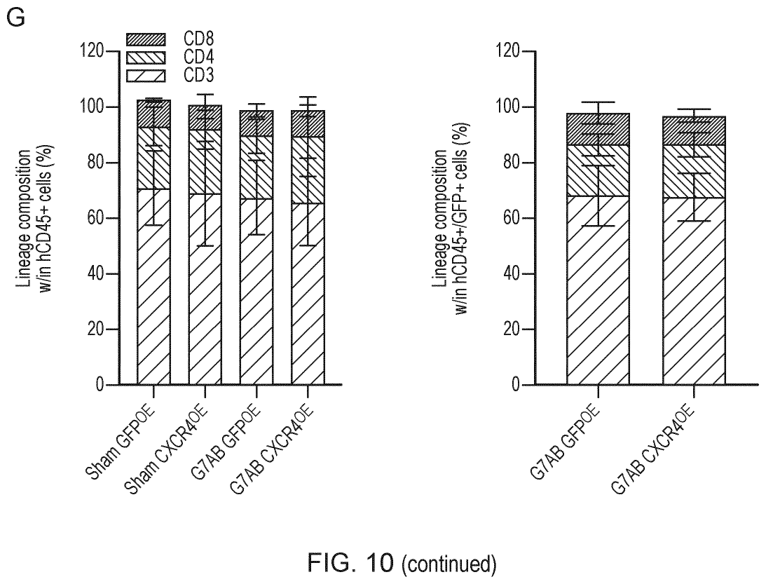

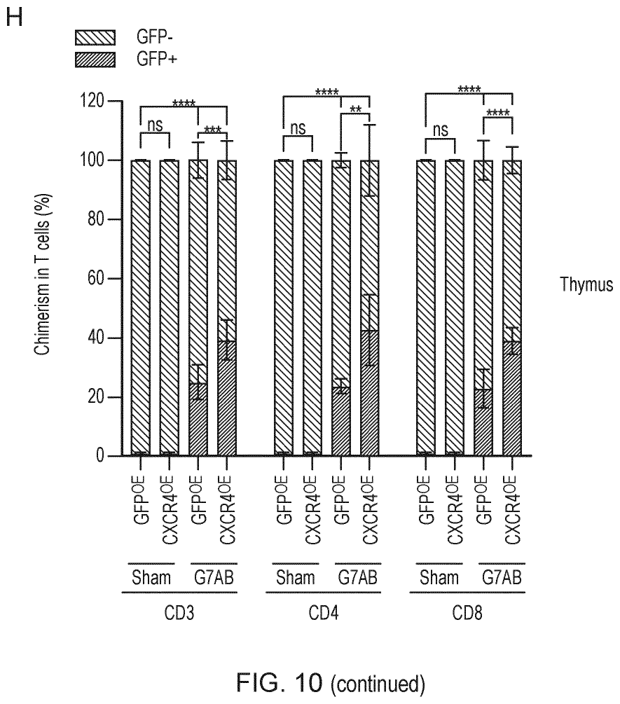

FIG. 10. Transient overexpression of CXCR4 increases chimerism in the humanized context, following M-HSCT, related to FIG. 4
(A-B) Myeloid and lymphoid cell composition of human CD45⁺ in the spleen (A) and thymus (B) of NSG mice, transplanted with CD34⁺ cells electroporated with GFP or CXCR4 mRNA. Comparison of lineages between human CD45⁺ and GFP⁺ cells by mixed-effects model (REML), followed by post hoc analysis with Dunnett's.
(C, E, G) Myeloid and lymphoid lineage composition of human CD45⁺ (left panel) and CD45⁺/GFP⁺ cells (right panel) in the spleen (C), BM (E) and thymus (G) after M-HSCT (as in FIG. 3D) with CD34⁺ cells transiently overexpressing GFP or CXCR4 in NSGW41 mice. Comparison of lineages between human CD45⁺ and CD45⁺/GFP⁺ cells performed by mixed-effects model (REML), followed by post hoc analysis with Dunnett's.
(D, F, H) Chimerism of GFP⁺ cells observed within CD19⁺, CD13⁺ and CD3⁺ cells in spleen (D), in BM (F) and within CD3⁺, CD4⁺, CD8⁺ T cells in thymus (H) after M-HSCT with CD34+ cells transiently overexpressing GFP or CXCR4 in NSGW41 mice. Mixed-effects model (REML), followed by post hoc analysis with Tukey's test (between groups) or by post hoc analysis with Dunnett's test (within groups).

Results are mean±SEM, with n ≥10.

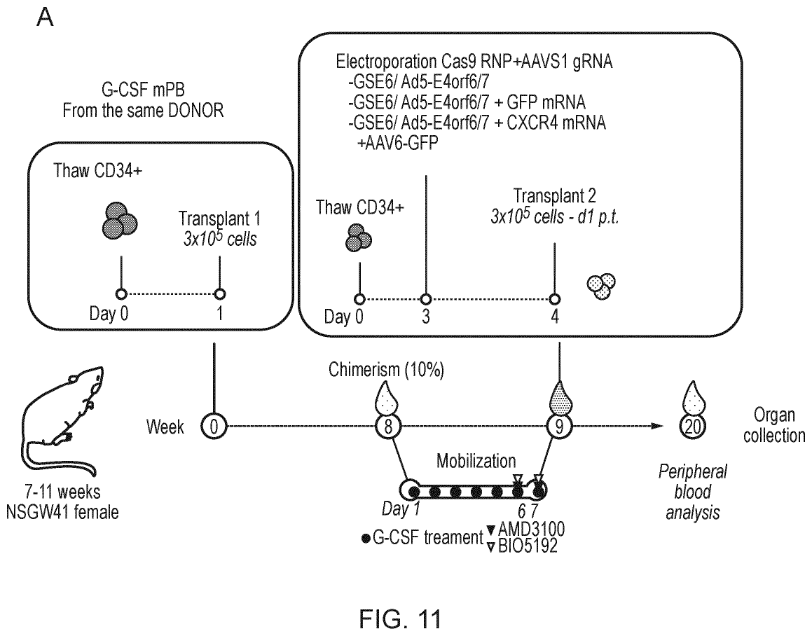

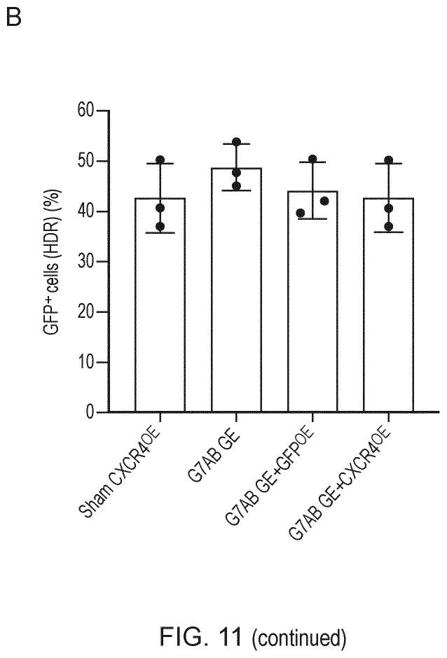

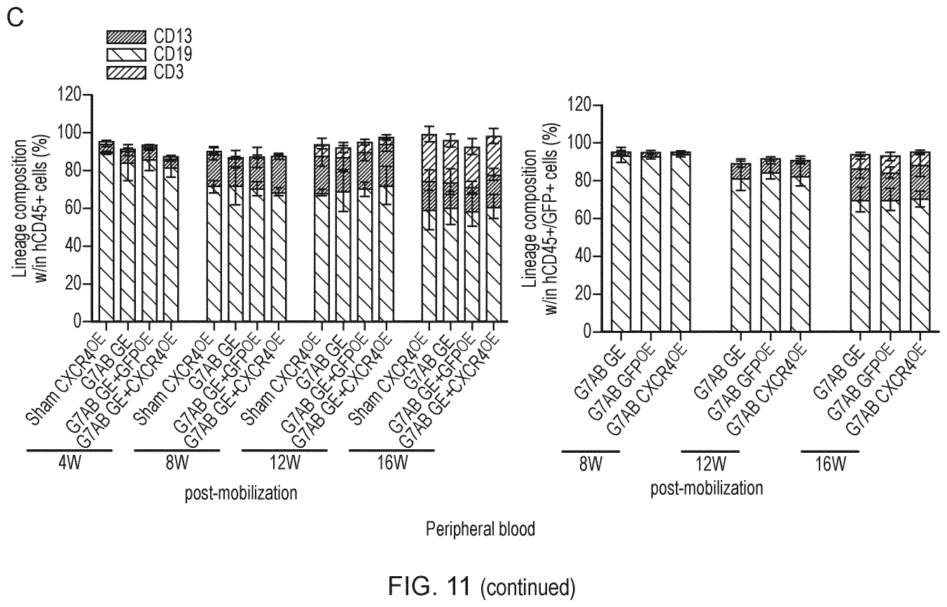

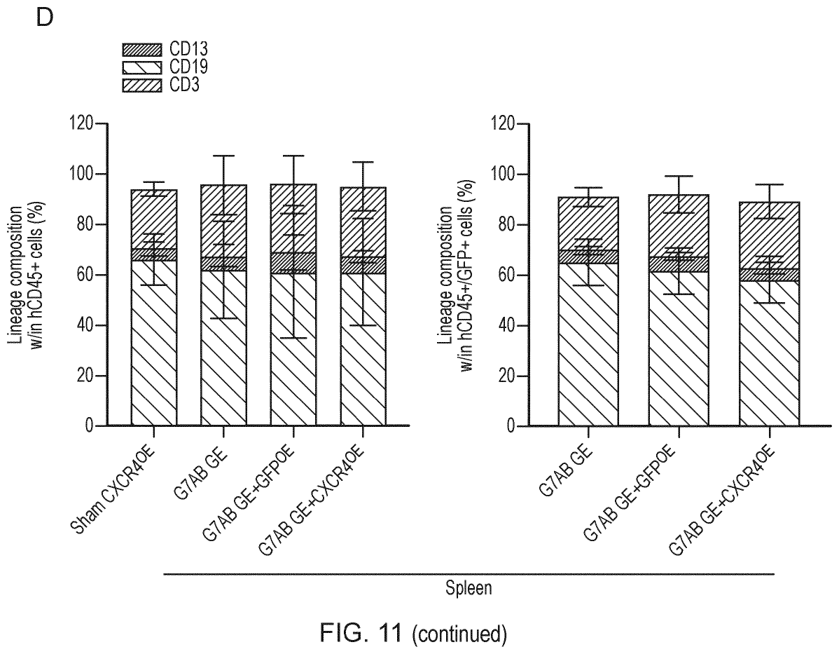

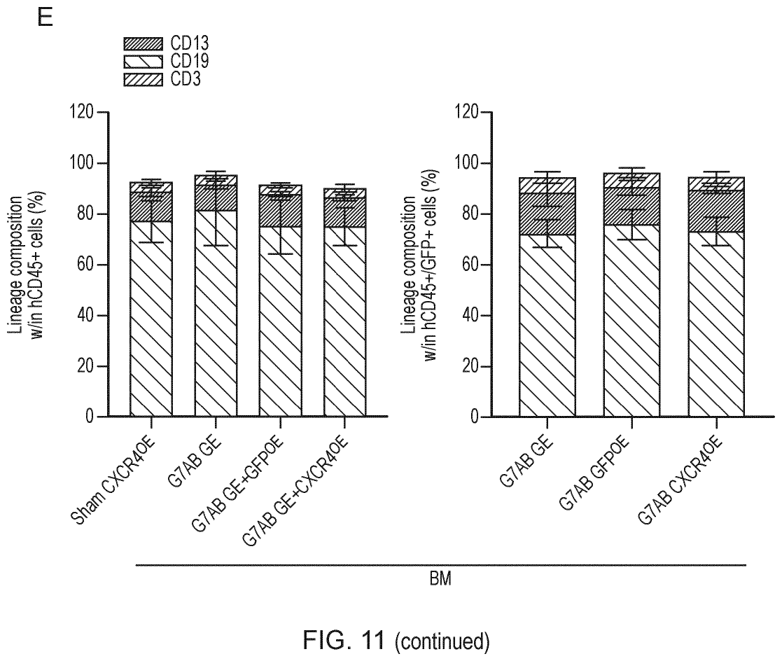

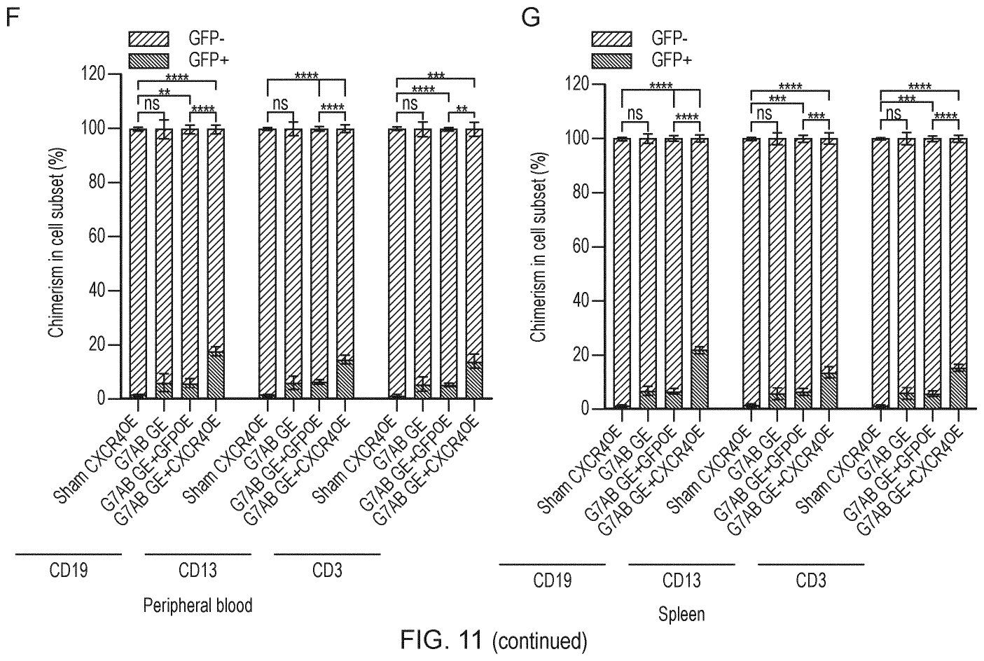

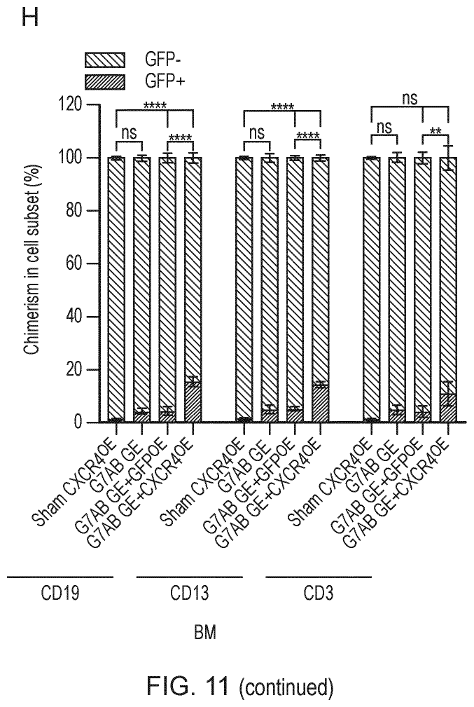

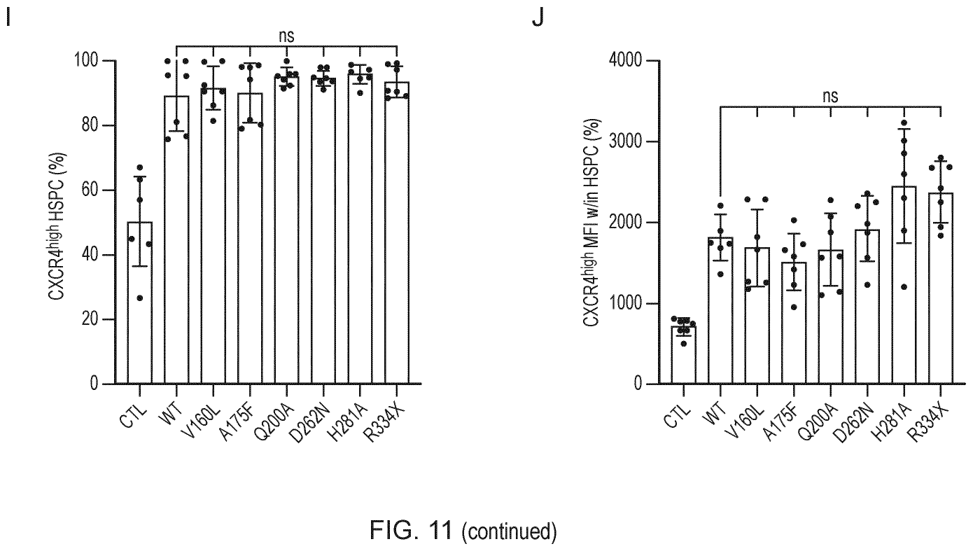

FIG. 11. M-HSCT confers a significant advantage to gene edited cells when paired with an engraftment enhancer, related to FIG. 5
(A) Schematic illustration of competitive transplantation, after G7AB mobilization, between human resident cells (initially transplanted with 3×10⁵ CD34⁺ G-CSF mPB cells, counted on d1 p.t.) and newly transplanted CD34⁺ G-CSF mPB gene-edited cells (outgrowth of 3×10⁵ CD34⁺ cells, counted on d1 p.t., transplanted on d4 p.t.) in NSGW41 mice.
(B) HDR efficiency in edited cells (GFP⁺) on the bulk CD34⁺ population, assessed in vitro, 15 days post-electroporation.
(C) Reconstitution of myeloid and lymphoid lineages overtime of human CD45⁺ (left panel) and CD45⁺/GFP⁺ cells (right panel) in PB after M-HSCT with CD34⁺ cells gene edited as in FIG. 5A, in NSGW41 mice. Comparison of lineages between human CD45⁺ and CD45⁺/GFP⁺ cells performed at the last time point by mixed-effects model (REML), followed by post hoc analysis with Dunnett's test (within groups).
(D, E) Myeloid and lymphoid lineage composition of human CD45⁺ (left panel) and CD45⁺/GFP⁺ cells (right panel) in the spleen (D) and BM (E) after M-HSCT with CD34⁺ cells gene edited as in FIG. 5A, in NSGW41 mice. Comparison of lineages between human CD45⁺ and CD45⁺/GFP⁺ cells performed by mixed-effects model (REML), followed by post hoc analysis with Dunnett's test (within groups).
(F-H) Chimerism of GFP⁺ cells observed within CD19⁺ B cells, CD13⁺ myeloid cells and CD3⁺ T cells at the end of the experiment in PB (F), in spleen (G) and in BM (H) after M-HSCT with CD34⁺ cells gene edited as in FIG. 5A, in NSGW41 mice. Mixed-effects model (REML), followed by post hoc analysis with Tukey's test (between groups) or by post hoc analysis with Dunnett's test (within groups).
(I) Percentage of CXCR4^{high} cells in HSPCs (CD34⁺ CD133⁺ CD90⁺), electroporated with CXCR4 variants mRNA. Kruskal-Wallis test, followed by post hoc analysis with Dunn's test.
(J) CXCR4^{high} MFI in HSPCs (CD34⁺ CD133⁺ CD90⁺), electroporated with CXCR4 variants mRNA. Kruskal-Wallis test, followed by post hoc analysis with Dunn's test.

Results are mean±SEM, with n ≥8 for data collected in vivo on NSGW41 mice, with n ≥5 for data collected in vivo on NSG mice and n ≥5 for data collected in vitro (3 different donors).

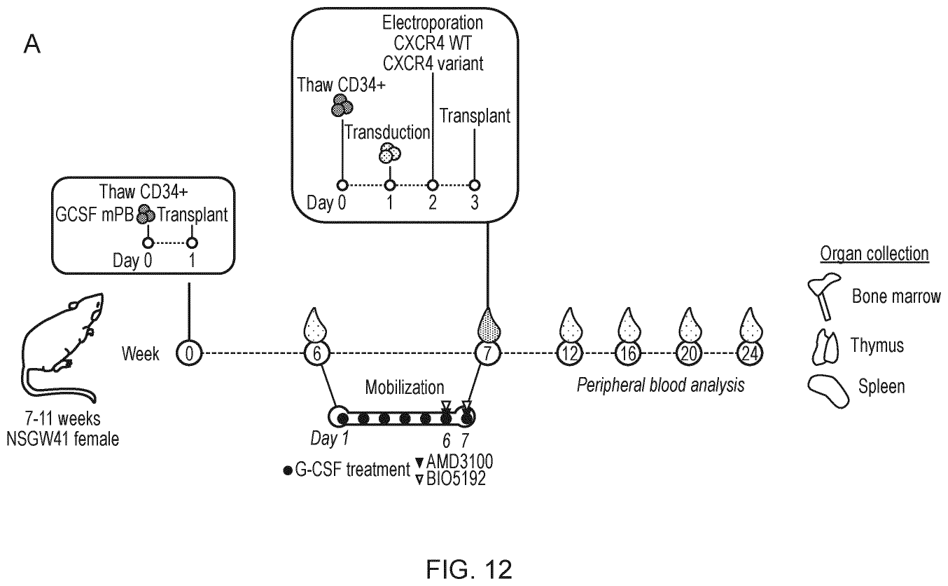

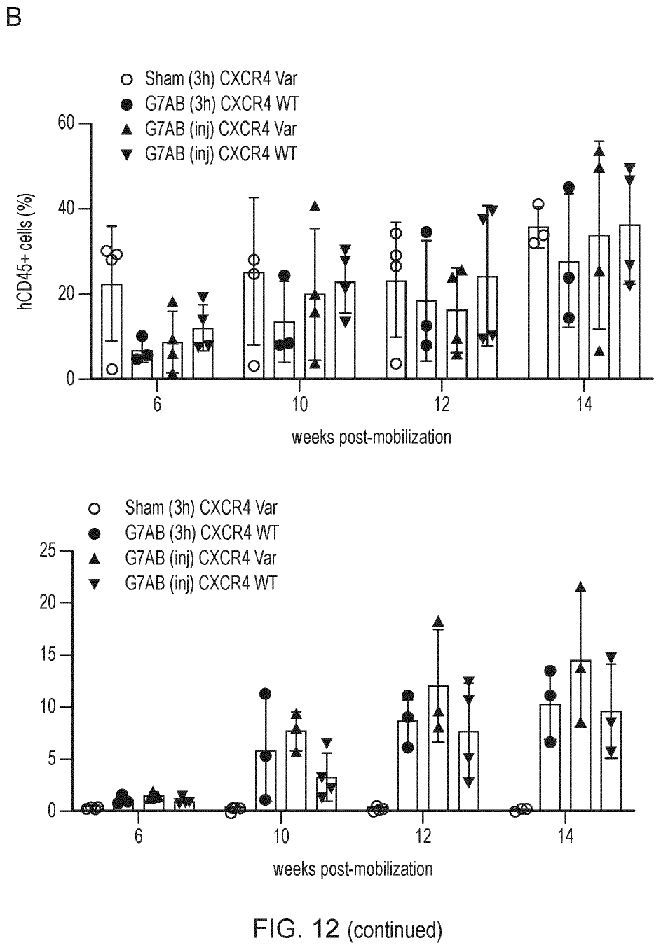

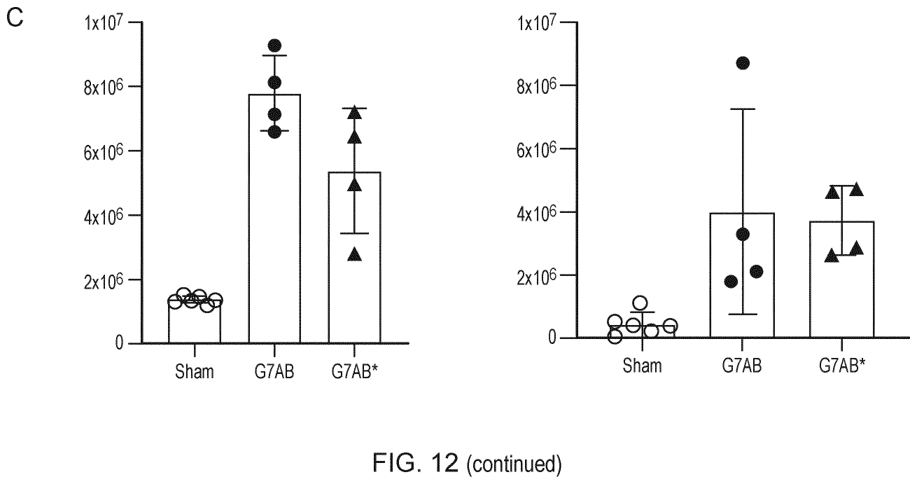

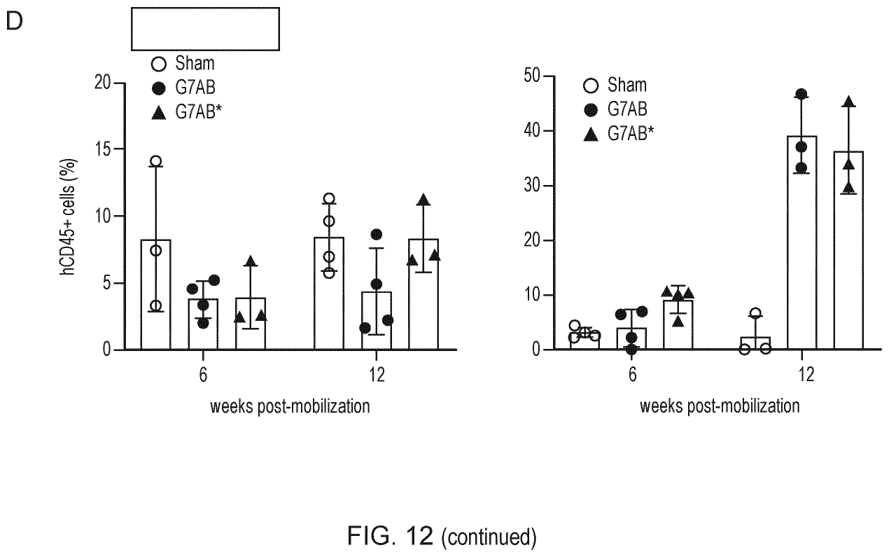

FIG. 12. Transient overexpression of drug-resistant CXCR4 variant confers an engraftment advantage in vivo.
(A) Schematic representation of the experimental design. NSGW41 or NBSGW mice were humanized with human mobilized peripheral blood CD34+ cells until 10% chimerism was achieved. Mice were then mobilized with G-CSF for 7 days through subcutaneous pump implantations and with two injections of AMD3100 and BIO5192 at day 6 and 7. For the second transplant, GFP lentiviral vector-transduced cells from the same donor used to humanize the mice were electroporated with either CXCR4 wild type or CXCR4^{D262N} variant. The donor cells were then transplanted either at 3 h or immediately after the last injection of AMD3100.
(B) Human chimerism over time after mobilization, expressed as % of human CD45+ cells present in peripheral blood (right panel), and exchange efficiency (left panel) expressed as % of GFP+ cells within the hCD45+ population. CXCR4 Var=CXCR4 D262N.
(C) Mobilization efficiency of the standard protocol (G7AB) or the modified protocol with AMD3465 (G7AB*) measured as white blood cell count (WBC, left panel), and human CD34+CD38− cells (right panel).
(D) Human chimerism over time after mobilization, expressed as % of hCD45+ cells in peripheral blood (right panel), and exchange efficiency (left panel) expressed as % of GFP+ cells within the hCD45+ population.

### DETAILED DESCRIPTION

Various preferred features and embodiments of the present invention will now be described by way of non-limiting examples. This disclosure is not limited by the exemplary methods and materials disclosed herein, and any methods and materials similar or equivalent to those described herein can be used in the practice or testing of embodiments of this disclosure. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The publications discussed herein are provided solely for their disclosure prior to the filing date of the present application. Nothing herein is to be construed as an admission that such publications constitute prior art to the claims appended hereto.

Haematopoietic Stem and/or Progenitor Cell Transplantation

In one aspect, the invention provides a population of haematopoietic stem and/or progenitor cells (HSPCs) for use in HSPC transplantation. In preferred embodiments, the subject is subjected to a regimen for mobilisation of endogenous HSPCs before and/or during the HSPC transplantation.

In one aspect, the invention provides a method for HSPC transplantation, the method comprising administering a population of HSPCs to a subject in need thereof. In preferred embodiments, the subject is subjected to a regimen for mobilisation of endogenous HSPCs before and/or during the administration of the population of HSPCs.

In one aspect, the invention provides a population of haematopoietic stem and/or progenitor cells (HSPCs) for use in HSPC transplantation, the HSPC transplantation comprising:
(a) administering one or more HSPC mobiliser to a subject to mobilise endogenous HSPCs from the subject's bone marrow; and
(b) administering the population of HSPCs to the subject.

In one aspect, the invention provides a method for haematopoietic stem and/or progenitor cell (HSPC) transplantation in a subject in need thereof, the method comprising:
(a) administering one or more HSPC mobiliser to the subject to mobilise the subject's endogenous HSPCs; and
(b) administering a population of HSPCs to the subject.

Haematopoietic stem/progenitor cell transplantation (HSCT) may refer to the transplantation of multipotent hematopoietic stem and/or progenitor cells, which are usually derived from bone marrow, peripheral blood, or umbilical cord blood. HSCT is a medical procedure in the fields of haematology and oncology, most often performed for people with diseases of the blood or bone marrow, or certain types of cancer.

In some embodiments, the HSCT is an autologous HSCT. By “autologous HSCT” it is to be understood that the population of HSPCs (which may then be cultured ex vivo and/or genetically engineered) is obtained from the same subject to which they are subsequently administered. Autologous transplant procedures are advantageous as they avoid problems associated with immunological incompatibility and are available to subjects irrespective of the availability of a genetically matched donor.

In some embodiments, the HSCT is an allogeneic HSCT. By “allogeneic HSCT” it is to be understood that the population of HSPCs (which may then be cultured ex vivo and/or genetically engineered) is obtained from a different subject as that to which they are subsequently administered. Preferably, the donor will be genetically matched to the subject to which the HSPCs are administered to minimise the risk of immunological incompatibility.

The subject may be subjected to multiple HSCTs. In some embodiments, the subject is subjected to two or more, three or more, four or more, or five or more HSCTs. In some embodiments, step (a) (or the regimen for mobilisation of endogenous HSPCs) is repeated one or more times, two or more times, three or more times, four or more times or five or more times. In some embodiments, step (b) (or the administration of the HSPCs) is repeated one or more times, two or more times, three or more times, four or more times or five or more times. In some embodiments, step (a) (or the regimen for mobilisation of endogenous HSPCs) and step (b) (or the administration of the HSPCs) are both repeated one or more times, two or more times, three or more times, four or more times or five or more times.

### Haematopoietic Stem and Proqenitor Cells (HSPCs)

Haematopoietic stem cells (HSCs) are multipotent stem cells that may be found, for example, in peripheral blood, bone marrow and umbilical cord blood. HSCs are capable of self-renewal and differentiation into any blood cell lineage. They are capable of recolonising the entire immune system, and the erythroid and myeloid lineages in all the haematopoietic tissues (such as bone marrow, spleen and thymus). They provide for life-long production of all lineages of haematopoietic cells.

Haematopoietic progenitor cells (HPCs) have the capacity to differentiate into a specific type of cell. In contrast to stem cells however, they are already far more specific: they are pushed to differentiate into their “target” cell. A difference between stem cells and progenitor cells is that stem cells can replicate indefinitely, whereas progenitor cells can only divide a limited number of times. Haematopoietic progenitor cells can be rigorously distinguished from HSCs only by functional in vivo assay (i.e. transplantation and demonstration of whether they can give rise to all blood lineages over prolonged time periods).

Haematopoietic Stem and/or Progenitor Cell (HSPC) Sources

A population of haematopoietic stem and/or progenitor cells (HSPCs) may be obtained from a tissue sample. For example, a population of haematopoietic stem and/or progenitor cells may be obtained from peripheral blood (e.g. adult and foetal peripheral blood), umbilical cord blood, bone marrow, liver or spleen. Preferably, these cells are obtained from peripheral blood or bone marrow.

A population of HSPCs may be obtained after the subject has been subjected to a regimen for mobilisation of endogenous HSPCs. The haematopoietic stem and/or progenitor cells may be mobilized peripheral blood (mPB) haematopoietic stem and/or progenitor cells. Mobilisation may be carried out using, for example, GCSF, Plerixafor, BIO5192, GROβ (GROβΔ4/CXCL2Δ4) (see e.g. Fukuda, et al. (2007) Blood 110: 860-869) or combinations thereof. Other agents, such as NSAIDs and dipeptidyl peptidase inhibitors, may also be useful as mobilizing agents.

With the availability of the stem cell growth factors GMCSF and GCSF, most HSCT procedures are now performed using stem cells collected from the peripheral blood, rather than from the bone marrow. Collecting peripheral blood stem cells provides a bigger graft, does not require that the donor be subjected to general anaesthesia to collect the graft, results in a shorter time to engraftment and may provide for a lower long-term relapse rate. Bone marrow may be collected by standard aspiration methods (either steady-state or after mobilisation), or by using next-generation harvesting tools (e.g. Marrow Miner).

HSPCs may also be derived from induced pluripotent stem cells.

### HSPC Characteristics

HSPCs are typically of low forward scatter and side scatter profile by flow cytometric procedures. Some are metabolically quiescent, as demonstrated by Rhodamine labelling which allows determination of mitochondrial activity. HSPCs may comprise certain cell surface markers such as CD34, CD45, CD133, CD90 and CD49f. They may also be defined as cells lacking the expression of the CD38 and CD45RA cell surface markers. However, expression of some of these markers is dependent upon the developmental stage and tissue-specific context of the HSPC. Some HSPCs called “side population cells” exclude the Hoechst 33342 dye as detected by flow cytometry. Thus, HSPCs have descriptive characteristics that allow for their identification and isolation.

CD34 and CD133 are the most useful positive markers for HSPCs. Some HSPCs are also positive for lineage markers such as CD90, CD49f and CD93. However, these markers may need to be used in combination for HSPC enrichment. By “positive marker” it is to be understood that human HSPCs express these markers. In some embodiments, HSPCs are CD34+.

CD38 is the most established and useful single negative marker for human HSPCs. Human HSPCs may also be negative for lineage markers such as CD2, CD3, CD14, CD16, CD19, CD20, CD24, CD36, CD56, CD66b, CD271 and CD45RA. However, these markers may need to be used in combination for HSPC enrichment. By “negative marker” it is to be understood that human HSPCs lack the expression of these markers. In some embodiments, HSPCs are CD34⁺ CD38−.

### Differentiated Cells

In contrast to HSPCs, a differentiated cell is a cell which has become more specialised. Differentiation occurs during the development of a multicellular organism as the organism changes from a single zygote to a complex system of tissues and cell types. Differentiation is also a common process in adults: adult stem cells divide and create fully-differentiated daughter cells during tissue repair and normal cell turnover. Differentiation dramatically changes a cell's size, shape, membrane potential, metabolic activity and responsiveness to signals. These changes are largely due to highly-controlled modifications in gene expression. In other words, a differentiated cell is a cell which has specific structures and performs certain functions due to a developmental process which involves the activation and deactivation of specific genes.

A differentiated cell includes differentiated cells of the haematopoietic lineage such as monocytes, macrophages, neutrophils, basophils, eosinophils, erythrocytes, megakaryocytes/platelets, dendritic cells, T-cells, B-cells and NK-cells. For example, differentiated cells of the haematopoietic lineage can be distinguished from HSPCs by detection of cell surface molecules which are not expressed or are expressed to a lesser degree on undifferentiated cells. Examples of suitable human lineage markers include CD33, CD13, CD14, CD15 (myeloid), CD19, CD20, CD22, CD79a (B), CD36, CD71, CD235a (erythroid), CD2, CD3, CD4, CD8 (T) and CD56 (NK).

### Mobilisation of Endogenous HSPCs

Any suitable regimen may be used for mobilisation of endogenous HSPCs from the subject's bone marrow.

In one aspect, the present invention provides a method of mobilizing endogenous HSPCs from a subject's bone marrow, the method comprising administering one or more HSPC mobiliser to the subject.

In one aspect, the present invention provides one or more HSPC mobiliser for use in mobilizing endogenous HSPCs from a subject's bone marrow, wherein the one or more HSPC mobiliser is administered to the subject.

In one aspect, the present invention provides one or more HSPC mobiliser for use in HSPC transplantation, the HSPC transplantation comprising:
(a) administering the one or more HSPC mobiliser to a subject to mobilise endogenous HSPCs from the subject's bone marrow; and
(b) administering a population of HSPCs to the subject.

As used herein, a “HSPC mobiliser” or “HSPC mobilization agent” may refer to an agent which, when administered to a subject, mobilizes endogenous HSPCs from their niche in bone marrow into circulation. Suitable HSPC mobilization agents will be known to those of skill in the art (see e.g. Domingues, M. J., et al., 2017. International journal of hematology, 105(2), pp. 141-152) and may include a granulocyte colony stimulating factor (GCSF), a CXCR4 antagonist (e.g. plerixafor (AMD3100), POL6326 (balixafortide), TG-0054 (burixafor), BKT140 (BL8040), LY2510924, ALX-0651), a VLA-4 antagonist (e.g. BIO5192, natalizumab), a SDF-1 antagonist (e.g. NOX-A12), a CXCR2 agonist (e.g. GROβ (SB-251353)), bortezomib, a PTH receptor agonist (e.g. PTH (teriparatide)), a FLT3 agonist (e.g. CDX-301 (rhFLT3L)), meloxicam, or a TPO receprot agonist (e.g. eltrombopag).

In some embodiments, the one or more HPSC mobiliser is selected from a granulocyte colony-stimulating factor (G-CSF), a CXCR4 antagonist and a VLA-4 antagonist, or any combination thereof. In some embodiments, the one or more HPSC mobiliser is a G-CSF and a CXCR4 antagonist. In some embodiments, the one or more HPSC mobiliser is a CXCR4 antagonist and a VLA-4 antagonist. In preferred embodiments, the one or more HPSC mobiliser is G-CSF, a CXCR4 antagonist and a VLA-4 antagonist.

In some embodiments, the regimen for mobilisation of endogenous HSPCs comprises:
(i) administering a G-CSF for at least about 3 days, at least about 4 days, at least about 5 days, at least about 6 days, or at least about 7 days;
(ii) administering a CXCR4 antagonist for at least about 1 day, at least about 2 days, or at least about 3 days; and/or
(iii) administering a VLA-4 antagonist for at least about 1 day, at least about 2 days, or at least about 3 days.

In some embodiments:
(i) the subject is administered a G-CSF for at least about 3 days, at least about 4 days, at least about 5 days, at least about 6 days, or at least about 7 days, optionally before the population of HSPCs is administered;
(ii) the subject is administered a CXCR4 antagonist for at least about 1 day, at least about 2 days, or at least about 3 days, optionally before the population of HSPCs is administered; and/or
(iii) the subject is administered a VLA-4 antagonist for at least about 1 day, at least about 2 days, or at least about 3 days, optionally before the population of HSPCs is administered.

In some embodiments:
(i) the subject is administered at least 3 daily doses, at least 4 daily doses, at least 5 daily doses, at least 6 daily doses, or at least 7 daily doses of G-CSF, optionally before the population of HSPCs is administered;
(ii) the subject is administered at least 1 daily dose, at least 2 daily doses, or at least 3 daily doses of a CXCR4 antagonist, optionally before the population of HSPCs is administered; and/or
(iii) the subject is administered at least 1 daily dose, at least 2 daily doses, or at least 3 daily doses of a VLA-4 antagonist, optionally before the population of HSPCs is administered.

In some embodiments, the regimen for mobilisation of endogenous HSPCs comprises:
(i) administering a G-CSF for about 7 days;
(ii) administering a CXCR4 antagonist for about 2 days; and
(iii) optionally, administering a VLA-4 antagonist for about 2 days.

In some embodiments:
(i) the subject is administered a G-CSF for about 7 days, before the population of HSPCs is administered;
(ii) the subject is administered a CXCR4 antagonist for about 2 days before the population of HSPCs is administered; and
(iii) optionally, the subject is administered a VLA-4 antagonist for about 2 days before the population of HSPCs is administered.

In some embodiments:
(i) the subject is administered 7 daily doses of a G-CSF before the population of HSPCs is administered; and
(ii) the subject is administered 2 daily doses of a CXCR4 antagonist before the population of HSPCs is administered; and
(iii) optionally, the subject is administered 2 daily doses of a VLA-4 antagonist before the population of HSPCs is administered.

The one or more HPSC mobiliser may be administered by any suitable route. Suitably, the one of more HSPC mobiliser is administered systemically, for example, the HPSC mobiliser may be administered intravenously, subcutaneously, or intraperitoneally.

The one or more HPSC mobiliser may be administered using any suitable dosage intervals. Suitably, the one of more HSPC mobiliser is administered as a single daily dose.

In some embodiments, the regimen for mobilisation of endogenous HSPCs is followed by a step of harvesting the mobilized endogenous HSPCs from the circulation, prior to administration of the population of HSPCs. Such a step may be used to deplete the endogenous HSPCs, thereby further enhancing engraftment of exogenous HSPCs. The mobilized endogenous HSPCs may be harvested by any suitable method described herein.

### Granulocyte Colony-Stimulating Factor (G-CSF)

In some embodiments, the subject is administered a G-CSF to mobilise endogenous HSPCs from the subject's bone marrow.

Granulocyte colony-stimulating factor (G-CSF) is also known as colony-stimulating factor 3 (CSF 3) and is a glycoprotein that stimulates the bone marrow to produce granulocytes and stem cells and release them into the bloodstream.

Suitable G-CSFs will be will be known to the skilled person. Recombinant granulocyte colony stimulating factor (filgrastim/lenograstim) is the most common mobilization agent and may be administered daily, either alone or in conjunction with chemotherapy (Bendall, L. J. and Bradstock, K. F., 2014. Cytokine & growth factor reviews, 25(4), pp. 355-367). Alternatively, use of the PEGylated variant of G-CSF (Pegfilgrastim), which has a significantly longer half-life, may eliminate the need for daily dosing (Piedmonte, D. M. and Treuheit, M. J., 2008. Advanced drug delivery reviews, 60(1), pp. 50-58).

Any suitable G-CSF mobilisation regimen may be used. Suitable doses of G-CSF will be known to the skilled person. Suitably, for human subjects G-CSF may be administered in a dose of from about 5 μg/kg/day to about 15 μg/kg/day, or about 10 μg/kg/day. Suitable routes of administration will be known to the skilled person. Suitably, the G-CSF may be administered by intravenous or subcutaneous injection.

### CXCR4 Antagonists

In some embodiments, the subject is administered a CXCR4 antagonist to mobilise endogenous HSPCs from the subject's bone marrow.

A “CXCR4 antagonist” or “CXCR4 inhibitor” may refer a substance that disrupts the interaction between CXCR4 and its ligand SDF-1. Suitable CXCR4 antagonists will be known to the skilled person and include plerixafor (AMD3100), POL6326 (balixafortide), TG-0054 (burixafor), BKT140 (BL8040), LY2510924, ALX-0651, and EPI-X4, and derivatives thereof (see e.g. Domingues, M. J., et al., 2017. International journal of hematology, 105(2), pp. 141-152).

In some embodiments, the CXCR4 antagonist is plerixafor (AMD3100). Plerixafor (AMD3100) is a small bicyclam molecule that reversibly binds and blocks CXCR4 and thereby inhibits the binding with its ligand stroma-cell-derived factor-1 (SCF-1). This process results in the release of HSPCs from its niches in the bone marrow stroma and to the circulation (Bilgin, Y. M. and de Greef, G. E., 2016. Current opinion in hematology, 23(1), pp. 67-71).

Any suitable CXCR4 antagonist mobilisation regimen may be used. Suitably, for human subjects AMD3100 may be administered in a dose of from about 100 μg/kg/day to about 500 μg/kg/day, about 160 μg/kg/day, about 240 μg/kg/day, or about 480 μg/kg/day (see e.g. De Clercq, E., Antiviral Chemistry and Chemotherapy, 27). Suitable routes of administration will be known to the skilled person. Suitably, the AMD3100 may be administered by subcutaneous injection.

### VLA-4 Antagonists

In some embodiments, the subject is administered a VLA-4 antagonist to mobilise endogenous HSPCs from the subject's bone marrow.

A “VLA-4 antagonist” or “VLA-4 inhibitor” may refer a substance that disrupts the interaction between VLA-4 and one or more of its natural ligands (e.g. VCAM-1). Suitable VLA-4 antagonists will be known to the skilled person and include BIO5192, firategrast (SB-683699), BOP and natalizumab, and derivatives thereof (see e.g. Domingues, M. J., et al., 2017. International journal of hematology, 105(2), pp. 141-152). In some embodiments, the VLA-4 antagonist is an ITGA4 antagonist.

In some embodiments, the VLA-4 antagonist is BIO5192. BIO5192 is a selective and potent small molecule inhibitor of VLA-4, with an affinity of 250- to 1000-fold higher than for the related α4β7 integrin (Ramirez, P., et al., 2009. Blood, 114(7), pp. 1340-1343).

In some embodiments, the VLA-4 antagonist is natalizumab. Natalizumab is a monoclonal antibody which targets the α4β1 integrin that is currently used for the treatment of multiple sclerosis (MS) and Crohn's disease (see e.g. Rudick, R., et al., 2013. JAMA neurology, 70(2), pp. 172-182).

### Population of HSPCs

Any suitable population of HSPCs may be administered to the subject. The population of HSPCs which is administered to the subject may be referred to as “exogenous” HSPCs to distinguish them from the “endogenous” HSPCs which are present in the subject's peripheral blood following mobilisation and prior to administration of the population of HSPCs.

In some embodiments, the population of HSPCs is autologous and/or allogenic. In some embodiments, the population of HSPCs is autologous or allogenic.

In some embodiments, the population of HSPCs is autologous. If the population of HSPCs is autologous, it may, for example, have been harvested from the subject's peripheral blood, bone marrow and/or cord blood. The population of HSPCs may have been harvested from the peripheral blood following mobilization, either performed by a prior mobilisation regimen or, e.g. in the case of concurrent administration, during the mobilisation regimen of the present invention, or from the bone marrow or the cord blood.

The population of HSPCs administered to the subject may be an HSPC according to the present invention. The inventors have found that the HSPCs of the present invention may be preferentially exchanged and/or selectively engrafted in subjects that have been subjected to mobilisation of their endogenous HSPCs.

In some embodiments, the population of HSPCs is genetically engineered. The term “genetically engineered” as used herein refers to the manipulation of a precursor cell, for example a natural cell, by the introduction of exogenous genetic material. Accordingly, in the context of the present invention a HSPC may be genetically engineered by the introduction of genetic material that encodes and enables the expression of one or more exogenous engraftment enhancer by the cell.

### Isolation and Enrichment of HSPCs

The population of HSPCs may be an isolated population of HSPCs. By “isolated population” of cells it is to be understood that the population of cells is not comprised within the body. An isolated population of cells may have been previously removed from a subject. An isolated population of cells may be cultured and manipulated ex vivo or in vitro using standard techniques known in the art. An isolated population of cells may later be reintroduced into a subject. Said subject may be the same subject from which the cells were originally isolated or a different subject.

A population of cells may be purified selectively for cells that exhibit a specific phenotype or characteristic, and from other cells which do not exhibit that phenotype or characteristic, or exhibit it to a lesser degree. For example, a population of cells that expresses a specific marker (such as CD34) may be purified from a starting population of cells. Alternatively, or in addition, a population of cells that does not express another marker (such as CD38) may be purified.

By “enriching” a population of cells for a certain type of cells it is to be understood that the concentration of that type of cells is increased within the population. The concentration of other types of cells may be concomitantly reduced.

Purification or enrichment may result in the population of cells being substantially pure of other types of cell. Purifying or enriching for a population of cells expressing a specific marker (e.g. CD34 or CD38) may be achieved by using an agent that binds to that marker, preferably substantially specifically to that marker.

An agent that binds to a cellular marker may be an antibody, for example an anti-CD34 or anti-CD38 antibody. The term “antibody” refers to complete antibodies or antibody fragments capable of binding to a selected target, and including Fv, ScFv, F(ab′) and F(ab′)₂, monoclonal and polyclonal antibodies, engineered antibodies including chimeric, CDR-grafted and humanised antibodies, and artificially selected antibodies produced using phage display or alternative techniques. In addition, alternatives to classical antibodies may also be used in the invention, for example “avibodies”, “avimers”, “anticalins”, “nanobodies” and “DARPins”.

The agents that bind to specific markers may be labelled so as to be identifiable using any of a number of techniques known in the art. The agent may be inherently labelled, or may be modified by conjugating a label thereto. By “conjugating” it is to be understood that the agent and label are operably linked. This means that the agent and label are linked together in a manner which enables both to carry out their function (e.g. binding to a marker, allowing fluorescent identification, or allowing separation when placed in a magnetic field) substantially unhindered. Suitable methods of conjugation are well known in the art and would be readily identifiable by the skilled person.

A label may allow, for example, the labelled agent and any cell to which it is bound to be purified from its environment (e.g. the agent may be labelled with a magnetic bead or an affinity tag, such as avidin), detected or both. Detectable markers suitable for use as a label include fluorophores (e.g. green, cherry, cyan and orange fluorescent proteins) and peptide tags (e.g. His tags, Myc tags, FLAG tags and HA tags).

A number of techniques for separating a population of cells expressing a specific marker are known in the art. These include magnetic bead-based separation technologies (e.g. closed-circuit magnetic bead-based separation), flow cytometry, fluorescence-activated cell sorting (FACS), affinity tag purification (e.g. using affinity columns or beads, such as biotin columns to separate avidin-labelled agents) and microscopy-based techniques.

It may also be possible to perform the separation using a combination of different techniques, such as a magnetic bead-based separation step followed by sorting of the resulting population of cells for one or more additional (positive or negative) markers by flow cytometry. Clinical grade separation may be performed, for example, using the CliniMACS® system (Miltenyi). This is an example of a closed-circuit magnetic bead-based separation technology.

It is also envisaged that dye exclusion properties (e.g. side population or rhodamine labelling) or enzymatic activity (e.g. ALDH activity) may be used to enrich for HSPCs.

### Culturing of HSPCs

In some embodiments, the population of HSPCs is cultured ex vivo prior to administration.

The present inventors have shown that ex vivo culture may endow HSPCs with a migration advantage by rescuing CXCR4 expression and therefore cultured HSPCs may outcompete mobilized endogenous HSPCs for engraftment in depleted BM niches.

Any suitable ex vivo culture conditions may be used. For example, ex vivo culture conditions which are standard in the art during lentivirus-based gene replacement protocols may allow recovery of surface molecules for homing/engraftment, whose expression is lowered by mobilization.

Suitably, the HSPCs are seeded at the concentration of about 1×10⁵ cells/ml to about 1×10⁶ cells per ml, e.g. about 1×10⁶ cells per ml. Suitably, the HSPCs are cultured in a 5% CO2 humidified atmosphere at 37° C. Suitably, the HSPCs are cultured for at least one day, at least two day, or at least three days. Suitably, the HSPCs are cultured for about one day, about two days, or about three days. In some embodiments, the HSPCs are cultured for about three days.

Any suitable culture medium may be used. Suitably, the HSPCs are cultured in serum-free cell culture medium. For example, commercially available medium such as StemSpan medium may be used, which contains bovine serum albumin, insulin, transferrin, and supplements in Iscove's MDM. The culture medium may be also supplemented with one or more antibiotic (e.g. penicillin, streptomycin).

The culture medium may be supplemented with one or more cytokines and/or growth factors. As used herein, a “cytokine” is any cell signalling substance and includes chemokines, interferons, interleukins, lymphokines, and tumour necrosis factors. As used herein, a “growth factor” is any substance capable of stimulating cell proliferation, wound healing, or cellular differentiation. The terms “cytokine” and “growth factor” may overlap.

The culture medium may comprise one or more early-acting cytokine, one or more transduction enhancer, and/or one or more expansion enhancer.

As used herein, an “early-acting cytokine” is a cytokine which stimulates HSPCs. Early-acting cytokines include thrombopoietin (TPO), stem cell factor (SCF), and Flt3-ligand (FLT3-L). In some embodiments, the culture medium comprises at least one early-acting cytokine. Any suitable concentration of early-acting cytokine may be used. For example, 1-1000 ng/ml, or 10-1000 ng/ml, or 10-500 ng/ml. In some embodiments, the culture medium comprises SCF. The concentration of SCF may be about 10-1000 ng/ml, about 50-500 ng/ml, or about 100-300 ng/ml. In some embodiments, the culture medium comprises FLT3-L. The concentration of FLT3-L may be about 10-1000 ng/ml, about 50-500 ng/ml, or about 100-300 ng/ml. In some embodiments, the culture medium comprises TPO. The concentration of TPO may be about 5-500 ng/ml, about 10-200 ng/ml, or about 20-100 ng/ml. In some embodiments, the culture medium comprises SCF (e.g. in a concentration of about 300 ng/ml), FLT3-L (e.g. in a concentration of about 300 ng/ml), and TPO (e.g. in a concentration of about 100 ng/ml).

As used herein, a “transduction enhancer” is a substance that is capable of improving viral transduction of HSPCs. Suitable transduction enhancers include LentiBOOST, prostaglandin E2 (PGE2) or a derivative thereof, protamine sulfate (PS), Vectofusin-1, ViraDuctin, RetroNectin, staurosporine (Stauro), 7-hydroxy-stauro, human serum albumin, and polyvinyl alcohol. In some embodiments, the culture medium comprises at least one transduction enhancer. Any suitable concentration of transduction enhancer may be used, for example as described in Schott, J. W., et al., 2019. Molecular Therapy-Methods & Clinical Development, 14, pp. 134-147 or Yang, H., et al., 2020. Molecular Therapy-Nucleic Acids, 20, pp. 451-458. In some embodiments, the culture medium comprises PGE2. Suitably, the PGE2 derivative is 16,16-dimethyl prostaglandin E2 (dmPGE2). The concentration of PGE2 or derivative thereof may be about 1-100 μM, about 5-20 μM, or about 10 μM.

As used herein, an “expansion enhancer” is a substance that is capable of improving expansion of HSPCs. Suitable expansion enhancers include UM171, UM729, StemRegenin1 (SR1), diethylaminobenzaldehyde (DEAB), LG1506, BIO (GSK3p inhibitor), NR-101, trichostatin A (TSA), garcinol (GAR), valproic acid (VPA), copper chelator, tetraethylenepentamine, and nicotinamide. In some embodiments, the culture medium comprises at least one expansion enhancer. Any suitable concentration of expansion enhancer may be used, for example as described in Huang, X., et al., 2019. F1000Research, 8, 1833. In some embodiments, the culture medium comprises UM171 or UM729. The concentration of UM171 may be about 10-200 nM, about 20-100 nM, or about 35 nM. In some embodiments, the culture medium comprises SR1. The concentration of SR1 may be about 0.1-10 μM, about 0.5-5 μM, or about 1 μM. In some embodiments, the culture medium comprises UM171 (e.g. in a concentration of about 35 nM) and SR1 (e.g. in a concentration of about 1 μM).

In some embodiments, the culture medium comprises SCF (e.g. in a concentration of about 300 ng/ml), FLT3-L (e.g. in a concentration of about 300 ng/ml), TPO (e.g. in a concentration of about 100 ng/ml), UM171 (e.g. in a concentration of about 35 nM) and SR1 (e.g. in a concentration of about 1 μM).

In some embodiments, the culture medium comprises SCF (e.g. in a concentration of about 300 ng/ml), FLT3-L (e.g. in a concentration of about 300 ng/ml), TPO (e.g. in a concentration of about 100 ng/ml), UM171 (e.g. in a concentration of about 35 nM), SR1 (e.g. in a concentration of about 1 μM), and PGE2 (e.g. in a concentration of about 10 μM).

### Engraftment Enhancers

In preferred embodiments, the population of HSPCs is genetically engineered to express one or more engraftment enhancer.

The present inventors have shown that expression of engraftment enhancers may endow HSPCs with a migration advantage and therefore exogenous HSPCs expressing engraftment enhancers may outcompete mobilized endogenous HSPCs for engraftment in depleted BM niches.

In preferred embodiments, the expression of the one or more engraftment enhancer is overexpression.

In some embodiments, the expression of the one or more engraftment enhancer is stable expression.

In preferred embodiments, the expression of the one or more engraftment enhancer is transient expression.

In one embodiment, the HSPCs are transduced or transfected with one or more polynucleotide encoding the one or more engraftment enhancer.

In one embodiment, the HSPCs are transduced or transfected with one or more vectors encoding the one or more engraftment enhancer.

In one embodiment, the HSPCs are transduced or transfected with one or more vectors encoding the one or more engraftment enhancer, wherein the vectors are selected from the group consisting of RNA vectors, integration-defective lentiviral vectors (IDLVs), adeno-associated viral (AAV) vectors and Sendai viral vectors. For example, an RNA polynucleotide encoding the one or more engraftment enhancer may be introduced into the HSPCs using RNA electroporation or by a non-viral delivery system. Each of these may enable transient expression.

In one embodiment, the HSPCs are transduced or transfected with one or more vectors encoding the one or more engraftment enhancer, wherein the vectors are RNA vectors. In one embodiment, the HSPCs are transduced or transfected with one or more vectors encoding the one or more engraftment enhancer, wherein the vectors are Sendai viral vectors.

In one embodiment, one or more engraftment enhancer is directly introduced into the HSPCs in the form of the protein, for example using protein electroporation. Direct protein introduction may enable the one or more engraftment enhancer to be introduced transiently to HSPCs.

In preferred embodiments, the HSPCs are transduced or transfected with one or more RNA polynucleotide encoding the one or more engraftment enhancer. Any RNA polynucleotide disclosed herein may be used.

Suitable engraftment enhancers are described herein and may include CXCR4, CD47, ITGA4, and KIT, or any combination thereof. In some embodiments, the one or more engraftment enhancer comprises or consists of CXCR4 (or a fragment or variant thereof). In some embodiments, the one or more engraftment enhancer comprises or consists of CD47 (or a fragment or variant thereof). In some embodiments, the one or more engraftment enhancer comprises or consists of ITGA4 (or a fragment or variant thereof). In some embodiments, the one or more engraftment enhancer comprises or consists of KIT (or a fragment or variant thereof).

In some embodiments, the one or more engraftment enhancer comprises two or more engraftment enhancers. In some embodiments, the one or more engraftment enhancer comprises three or more engraftment enhancers. In some embodiments, the one or more engraftment enhancer comprises four or more engraftment enhancers. In some embodiments, the one or more engraftment enhancer consists of one engraftment enhancer.

In some embodiments, the one or more engraftment enhancer consists of two engraftment enhancers. In some embodiments, the one or more engraftment enhancer consists of three engraftment enhancers. In some embodiments, the one or more engraftment enhancer consists of four engraftment enhancers.

In some embodiments, the one or more engraftment enhancer comprises or consists of CXCR4 (or a fragment or variant thereof) and CD47 (or a fragment or variant thereof). In some embodiments, the one or more engraftment enhancer comprises or consists of CXCR4 (or a fragment or variant thereof) and ITGA4 (or a fragment or variant thereof). In some embodiments, the one or more engraftment enhancer comprises or consists of CXCR4 (or a fragment or variant thereof) and KIT (or a fragment or variant thereof). In some embodiments, the one or more engraftment enhancer comprises or consists of CXCR4 (or a fragment or variant thereof), ITGA4 (or a fragment or variant thereof), and KIT (or a fragment or variant thereof). In some embodiments, the one or more engraftment enhancer comprises or consists of CXCR4 (or a fragment or variant thereof), CD47 (or a fragment or variant thereof), ITGA4 (or a fragment or variant thereof), and KIT (or a fragment or variant thereof). In some embodiments, the one or more engraftment enhancer comprises or consists of ITGA4 (or a fragment or variant thereof) and KIT (or a fragment or variant thereof).

### Gene Edited or Gene-Corrected

The population of HSPCs may be genetically engineered. Suitably, the genetic engineering is in addition to the genetic engineering to express one or more engraftment enhancer.

In some embodiments, the method of the present invention further comprises a step of genetically engineering the population of HSPCs prior to administering the population of HSPCs.

In some embodiments, the population of HSPCs are genetically engineered to express a transgene, gene-edited, and/or gene-corrected.

In some embodiments, in addition to the engraftment enhancer, the population of HSPCs is genetically engineered to express a transgene. In some embodiments, the method of the present invention further comprises a step of genetically engineering the population of HSPCs to express a transgene, prior to administering the population of HSPCs.

The transgene may be a nucleotide of interest (NOI). Preferably the nucleotide of interest gives rise to a therapeutic effect. Suitable NOIs include, but are not limited to, sequences encoding enzymes, cytokines, chemokines, hormones, antibodies, anti-oxidant molecules, engineered immunoglobulin-like molecules, single chain antibodies, fusion proteins, immune co-stimulatory molecules, immunomodulatory molecules, anti-sense RNA, microRNA, shRNA, siRNA, ribozymes, miRNA target sequences, a transdomain negative mutant of a target protein, toxins, conditional toxins, antigens, tumour suppressor proteins, growth factors, transcription factors, membrane proteins, surface receptors, anti-cancer molecules, vasoactive proteins and peptides, anti-viral proteins and ribozymes, and derivatives thereof (such as derivatives with an associated reporter group). The NOIs may also encode pro-drug activating enzymes.

An example of a NOI is the beta-globin chain which may be used for gene therapy of thalassemia/sickle cell disease. NOIs also include those useful for the treatment of other diseases requiring non-urgent/elective gene correction in the myeloid lineage such as: chronic granulomatous disease (CGD, e.g. the gp91phox transgene), leukocyte adhesion defects, other phagocyte disorders in patients without ongoing severe infections and inherited bone marrow failure syndromes (e.g. Fanconi anaemia), as well as primary immunodeficiencies (SCIDs). NOIs also include those useful in the treatment of lysosomal storage disorders and immunodeficiencies.

In some embodiments, the population of HSPCs is gene-edited. In some embodiments, the method of the present invention further comprises a step of gene editing the population of HSPCs, prior to administering the population of HSPCs.

The term “gene editing” refers to a type of genetic engineering in which a nucleic acid is inserted, deleted or replaced in a cell. Gene editing may be achieved using engineered nucleases, which may be targeted to a desired site in a polynucleotide (e.g. a genome). Such nucleases may create site-specific double-strand breaks at desired locations, which may then be repaired through non-homologous end-joining (NHEJ) or homologous recombination (HR), resulting in targeted mutations. Such nucleases may be delivered to a target cell using vectors, such as viral vectors.

Examples of suitable nucleases known in the art include zinc finger nucleases (ZFNs), transcription activator like effector nucleases (TALENs), and the clustered regularly interspaced short palindromic repeats (CRISPR)/Cas system (Gaj, T. et al. (2013) Trends Biotechnol. 31: 397-405; Sander, J. D. et al. (2014) Nat. Biotechnol. 32: 347-55). Meganucleases (Silve, G. et al. (2011) Cur. Gene Ther. 11: 11-27) may also be employed as suitable nucleases for gene editing.

The CRISPR/Cas system is an RNA-guided DNA binding system (van der Oost et al. (2014) Nat. Rev. Microbiol. 12: 479-92), wherein the guide RNA (gRNA) may be selected to enable a Cas9 domain to be targeted to a specific sequence. Methods for the design of gRNAs are known in the art. Furthermore, fully orthogonal Cas9 proteins, as well as Cas9/gRNA ribonucleoprotein complexes and modifications of the gRNA structure/composition to bind different proteins, have been recently developed to simultaneously and directionally target different effector domains to desired genomic sites of the cells (Esvelt et al. (2013) Nat. Methods 10: 1116-21; Zetsche, B. et al. (2015) Cell pii: S0092-8674(15)01200-3; Dahlman, J. E. et al. (2015) Nat. Biotechnol. 2015 Oct. 5. doi: 10.1038/nbt.3390; Zalatan, J. G. et al. (2015) Cell 160: 339-50; Paix, A. et al. (2015) Genetics 201: 47-54), and are suitable for use in the invention.

In some embodiments, the population of HSPCs is gene-corrected. In some embodiments, the method of the present invention further comprises a step of gene correcting the population of HSPCs, prior to administering the population of HSPCs.

As used herein “gene-corrected” cells may refer to cells in which disease-causing mutations have been corrected. Site-specific genome editing using programmable endonuclease platforms such as CRISPR-Cas9, transcription activator-like effector nucleases or zinc-finger nucleases can inactivate harmful alleles, disable transcriptional repressor expression or their binding sites, precisely correct mutations or insert healthy gene copies into a genomic ‘safe harbour’. Alternatively, cells may be subject to gene transfer, for example by using viral vectors such as gammaretroviruses and lentiviruses to integrate a therapeutic gene into the genome of the recipient cell (Ferrari, G., et al. (2021). Nat Rev Genet 22, 216-234).

### Administration of HSPCs

The subject may be administered the population of HSPCs during and/or after mobilisation of endogenous HSPCs.

The population of HSPCs may be administered in any suitable dose and via any suitable route of administration. Suitably, from about 1×10⁶ to about 1×10¹⁰ cells/kg may be administered by intravenous infusion.

Suitable doses of transduced cell populations are such as to be therapeutically and/or prophylactically effective. The skilled person can readily determine an appropriate dose of one of the agents of the invention to administer to a subject without undue experimentation. Typically, a physician may determine the actual dosage which will be most suitable for an individual patient and it will depend on a variety of factors including the activity of the specific agent employed, the metabolic stability and length of action of that agent, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

Although the agents for use in the invention (in particular, the populations of cells) can be administered alone, they will generally be administered in admixture with a pharmaceutical carrier, excipient or diluent, particularly for human therapy.

In some embodiments, prior to the administration of the population of HSPCs, the mobilized endogenous HSPC are harvested. The mobilized endogenous HSPCs may be harvested during and/or after mobilisation of endogenous HSPCs. Such a step may be used to deplete the endogenous HSPCs, thereby further enhancing engraftment of exogenous HSPCs. The mobilized endogenous HSPCs may be harvested by any suitable method described herein.

Administration after Mobilisation

The population of HSPCs may be administered at and/or after the peak of mobilisation.

The “peak of mobilisation” may refer to the time at which the highest number of endogenous HSPCs are mobilized in the peripheral blood. The peak of mobilisation may be determined by any suitable method. In some embodiments, the peak of mobilisation is the time of highest count of mobilized white blood cells (WBC), Lin-SCA1+KIT+ cells (LSK), and/or Lin− SCA1+KIT+CD150+CD48−(SLAM HSC) in the subject's peripheral blood.

In some embodiments, the population of HSPCs is administered at about the peak of mobilisation. In this context, “about” may refer to administration within 1 hour, within 50 minutes, within 40 minutes, within 30 minutes, within 20 minutes, or within 10 minutes of the peak of mobilisation. In some embodiments, the population of HSPCs is administered at the peak of mobilisation±1 hour. In some embodiments, the population of HSPCs is administered at the peak of mobilisation±30 minutes. In some embodiments, the population of HSPCs is administered at the peak of mobilisation±20 minutes. In some embodiments, the population of HSPCs is administered at the peak of mobilisation±10 minutes.

In some embodiments, the population of HSPCs is administered after the peak of mobilisation. In some embodiments, the population of HSPCs is administered within about 10 minutes, within about 20 minutes, within about 30 minutes, within about 40 minutes, within about 50 minutes, within about 1 hour, within about 2 hours, or within about 3 hours after the peak of mobilisation.

The present inventors have shown that the peak of mobilisation may be from about 1 hour to about 9 hours after the last administration of a HSPC mobiliser. In this context, the “last administration” refers to the last administration of a HSPC mobiliser in the regimen for mobilisation of endogenous HSPCs, which occurs prior to administration of the population of HSPCs. Thus, this does not exclude that further HSPC mobilisers may be administered to the subject after the population of HSPCs has been administered. For example, the subject may undergo multiple HSCTs, each comprising a separate step of administering one or more HSPC mobiliser to the subject.

In some embodiments, the population of HSPCs is administered within about 9 hours, within about 8 hours, within about 7 hours, within about 6 hours, within about 5 hours, within about 4 hours, within about 3 hours, within about 2 hours, or within about 1 hour after the last administration of a HSPC mobiliser. In some embodiments, the population of HSPCs is administered within about 3 hours, within about 2 hours, or within about 1 hour after the last administration of a HSPC mobiliser. In some embodiments, the population of HSPCs is administered from about 1 to about 9 hours, from about 1 to about 6 hours, from about 1 to about 5 hours, from about 1 to about 4 hours, from about 2 to about 4 hours, or from about 2 to about 3 hours after the last administration of a HSPC mobiliser. In some embodiments, the population of HSPCs is administered from about 2 to about 4 hours after the last administration of a HSPC mobiliser. In some embodiments, the population of HSPCs is administered about 3 hours, about 2 hours, or about 1 hour after the last administration of a HSPC mobiliser. In some embodiments, the population of HSPCs is administered about 3 hours after the last administration of a HSPC mobiliser.

In some embodiments, the population of HSPCs is administered within about 9 hours, within about 8 hours, within about 7 hours, within about 6 hours, within about 5 hours, within about 4 hours, within about 3 hours, within about 2 hours, or within about 1 hour, after the regimen for mobilisation of endogenous HSPCs is completed. In some embodiments, the population of HSPCs is administered within about 3 hours, within about 2 hours, or within about 1 hour, after the regimen for mobilisation of endogenous HSPCs is completed. In some embodiments, the population of HSPCs is administered from about 1 to about 9 hours, from about 1 to about 6 hours, from about 1 to about 5 hours, from about 1 to about 4 hours, from about 2 to about 4 hours, or from about 2 to about 3 hours after the regimen for mobilisation of endogenous HSPCs is completed. In some embodiments, the population of HSPCs is administered from about 2 to about 4 hours after the regimen for mobilisation of endogenous HSPCs is completed. In some embodiments, the population of HSPCs is administered about 3 hours, about 2 hours, or about 1 hour after the regimen for mobilisation of endogenous HSPCs is completed. In some embodiments, the population of HSPCs is administered about 3 hours after the regimen for mobilisation of endogenous HSPCs is completed.

In some embodiments, the population of HSPCs is administered within about 9 hours, within about 8 hours, within about 7 hours, within about 6 hours, within about 5 hours, within about 4 hours, within about 3 hours, within about 2 hours, or within about 1 hour, after the one or more HSPC mobiliser is administered to the subject. In some embodiments, the population of HSPCs is administered within about 3 hours, within about 2 hours, or within about 1 hour, after the one or more HSPC mobiliser is administered to the subject. In some embodiments, the population of HSPCs is administered from about 1 to about 9 hours, from about 1 to about 6 hours, from about 1 to about 5 hours, from about 1 to about 4 hours, from about 2 to about 4 hours, or from about 2 to about 3 hours after the one or more HSPC mobiliser is administered to the subject. In some embodiments, the population of HSPCs is administered from about 2 to about 4 hours after the one or more HSPC mobiliser is administered to the subject. In some embodiments, the population of HSPCs is administered about 3 hours, about 2 hours, or about 1 hour after the one or more HSPC mobiliser is administered to the subject. In some embodiments, the population of HSPCs is administered about 3 hours after the one or more HSPC mobiliser is administered to the subject.

### Administration During Mobilisation

The population of HSPCs may be administered concurrently with mobilisation of the endogenous HSPCs. In particular, when the population of HSPCs is genetically engineered to express one or more engraftment enhancer, the population of HSPCs may engraft in depleted BM niches despite the administration of mobilization agents. For example, the present inventors have shown that some CXCR4 variants are resistant to the CXCR4 antagonist AMD3100.

In some embodiments, the population of HSPCs is administered during mobilisation of endogenous HSPCs. In some embodiments, the population of HSPCs is administered before the last administration of a HSPC mobiliser. In some embodiments, the population of HSPCs is administered before the regimen for mobilisation of endogenous HSPCs is completed. In some embodiments, the population of HSPCs is administered before the one or more HSPC mobiliser is administered to the subject.

In some embodiments, the population of HSPCs is genetically engineered to express an engraftment enhancer that is resistant to a mobilisation agent.

In some embodiments, the population of HSPCs is genetically engineered to express a CXCR4 variant which is resistant to a CXCR4 antagonist. In some embodiments, the population of HSPCs is genetically engineered to express a CXCR4 variant comprising one or more amino acid substitution selected from A175F, Q200A, D262N, and H281A. In some embodiments, the population of HSPCs is genetically engineered to express a CXCR4 variant comprising one or more amino acid substitution selected from A175F and D262N.

In some embodiments, the population of HSPCs is genetically engineered to express an IGA4 variant which is resistant to a VLA-4 antagonist.

In some embodiments, the population of HSPCs is genetically engineered to express a KIT variant that has increased resistance to a KIT-directed antibody or immunotoxin.

### HSPC Engraftment and Chimerism

The administered population of HSPCs may engraft in the subject's bone marrow after transplantation.

The term “engraftment” as used herein refers to the ability of the haematopoietic stem and/or progenitor cells to populate and survive in a subject following their transplantation, i.e. in the short and/or long term after transplantation. For example, engraftment may refer to the number and/or percentages of haematopoietic cells descended from the transplanted haematopoietic stem and/or progenitor cells (e.g. graft-derived cells) that are detected about 1 day to 24 weeks, 1 day to 10 weeks, or 1-30 days or 10-30 days after transplantation. In the xenograft model of human haematopoietic stem and/or progenitor cell engraftment and repopulation, engraftment may be evaluated in the peripheral blood as the percentage of cells deriving from the human xenograft (e.g. positive for the CD45 surface marker), for example. In one embodiment, engraftment is assessed at about 1, 2, 3, 4, 5, 6, 7, 8, 9, 10, 15, 20, 25 or 30 days after transplantation. In another embodiment, engraftment is assessed at about 4, 5, 6, 7, 8, 9, 10, 11, 12, 13, 14, 15, 16, 17, 18, 19, 20, 21, 22, 23 or 24 weeks after transplantation. In another embodiment, engraftment is assessed at about 16-24 weeks, preferably 20 weeks, after transplantation.

Engraftment may be readily analysed by the skilled person. For example, the transplanted haematopoietic stem and/or progenitor cells may be engineered to comprise a marker (e.g. a reporter protein, such as a fluorescent protein), which can be used to quantify the graft-derived cells. Samples for analysis may be extracted from relevant tissues and analysed ex vivo (e.g. using flow cytometry).

The administered population of HSPCs may outcompete mobilized endogenous HSPCs for engraftment in the subject's BM. The relative engraftment may be determined by any suitable method and can be reported as the chimerism level (see e.g. Zimmerman, C., and Shenoy, S. (2020). Front Immunol 11, 1791). A chimerism of 100% means that all HSPCs in the subject's BM post-transplantation originate from the exogenous HSPCs. A chimerism of 0% means that all HSPCs in the subject's BM post-transplantation originate from the endogenous HSPCs. Donor chimerism requirements to achieve disease control are widely variable in NMD and can range from as low as 10% to >90%.

In some embodiments, the chimerism level of the population of HSPCs in the subject's bone marrow is at least about 10%, at least about 15%, at least about 20%, at least about 25%, at least about 30%, at least about 35%, or at least about 40%. In some embodiments, the chimerism level of the population of HSPCs in the subject's bone marrow is at least about 30%.

In some embodiments, the chimerism level is stable. In this context, a “stable chimerism” may mean that the chimerism level does not significantly change (e.g. there is no statistically significant change). In some embodiments, the chimerism decreases by about 5% or less, about 4% or less, about 3% or less, about 2% or less, or about 1% or less. In some embodiments, the chimerism level is stable for at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23 or at least about 24 weeks.

In some embodiments, the chimerism level of the population of HSPCs in the subject's bone marrow is at least about 10%, at least about 15%, at least about 20%, at least about 25%, at least about 30%, at least about 35%, or at least about 40% after 24 weeks. In some embodiments, the chimerism level of the population of HSPCs in the subject's bone marrow is at least about 30% after 24 weeks.

### Subject

The subject may be any suitable subject, for example a human or non-human animal.

In preferred embodiments, the subject is a human. The subject may be an infant, a child, an adolescent, an adult, or elderly.

Examples of non-human animals include vertebrates, for example mammals, such as non-human primates (particularly higher primates), dogs, rodents (e.g. mice, rats or guinea pigs), pigs and cats. The non-human animal may be a companion animal.

Many recipients of HSCTs are multiple myeloma or leukaemia patients who would not benefit from prolonged treatment with, or are already resistant to, chemotherapy. Candidates for HSCTs include paediatric cases where the patient has an inborn defect such as severe combined immunodeficiency or congenital neutropenia with defective stem cells, and also children or adults with aplastic anaemia who have lost their stem cells after birth. Other conditions treated with HSCT include sickle-cell disease, myelodysplastic syndrome, neuroblastoma, lymphoma, Ewing's Sarcoma, Desmoplastic small round cell tumour and Hodgkin's disease. More recently non-myeloablative, or so-called “mini transplant”, procedures have been developed that require smaller doses of preparative chemotherapy and radiation. This has allowed HSCT to be conducted in the elderly and other patients who would otherwise be considered too weak to withstand a conventional treatment regimen.

The subject may have any disorder which can benefit from HSCT, including cancers and non-malignant disorders. For example, the subject may have a disease selected from the group consisting of mucopolysaccharidosis type I (MPS-1), chronic granulomatous disorder (CGD), Fanconi anaemia (FA), sickle cell disease, Pyruvate kinase deficiency (PKD), Leukocyte adhesion deficiency (LAD), metachromatic leukodystrophy (MLD), globoid cell leukodystrophy (GLD), GM2 gangliosidosis, thalassemia, cancer, a genetic disease and a blood disease.

In some embodiments, the subject has cancer, a primary immunodeficiency, a lysosomal storage disorder, or a haemoglobinopathy. In some embodiments, the subject has a primary immunodeficiency, a lysosomal storage disorder, or a haemoglobinopathy. In some embodiment, the subject has a primary immunodeficiency, such as human primary combined immunodeficiency Hyper IgM Syndrome 1 (HIGM-1).

### Pre-Conditioning

In preferred embodiments, the subject does not undergo chemotherapy or radiotherapy conditioning before administration of the HSPCs.

In one embodiment, the subject is subjected to a mild myeloablative, reduced intensity or non-myeloablative conditioning regimen before administration of the HSPCs.

In one embodiment, the subject is subjected to a mild myeloablative conditioning regimen before administration of the HSPCs.

In one embodiment, the subject is subjected to a reduced intensity conditioning regimen before administration of the HSPCs.

In one embodiment, the subject is subjected to a non-myeloablative conditioning regimen before administration of the HSPCs.

The invention may also utilise conditioning regimens that are based on the administration of toxins that targeted to HSPCs. Such methods may enable selective depletion or ablation of endogenous HSPC populations, and include those disclosed in US 2016/324982 for example. Such methods may be non-myeloablative. These methods may utilise one or more markers on the HSPC cell surface to target a toxin, such that the toxin is internalised by the HSPC. The methods may avoid toxicities associated with traditional conditioning methods.

In one embodiment, the subject subjected to conditioning with one or more HSPC-specific immunotoxins. Preferably, the one or more HSPC-specific immunotoxins are administered before administration of the HSPCs.

In one embodiment, the subject is administered an antibody conjugated to a toxin before administration of the HSPCs.

Suitable toxins include, but are not limited to saporin, diphtheria toxin, pseudomonas exotoxin A, Ricin A chain derivatives, a small molecule toxin, RNA polymerase II and/or III inhibitors (e.g. an amatoxin, such as α-amanitin, β-amanitin, γ-amanitin, ε-amanitin, amanin, amaninamide, amanullin or amanullinic acid), a DNA-damaging molecule (e.g. an anti-tubulin agent, a DNA crosslinking agent, a DNA alkylating agent or a mitotic disrupting agent; such as, maytansine) and combinations thereof.

Suitable antibodies include antibodies that bind to a cell surface protein selected from the group consisting of CD45, CD49d (VLA-4), CD49f (VLA-6), CD51, CD84, CD90, CD117, CD133, CD134 and CD184 (CXCR4).

In one embodiment, the immunotoxin is an anti-cKit immunotoxin. An anti-cKit immunotoxin may comprise an anti-cKit antibody conjugated to a toxin (see, for example, Czechowicz, A. et al. (2018) Biol Bone Marrow Transplant 24: S60 Abstract 54).

In one embodiment, the immunotoxin comprises an anti-cKit antibody. In one embodiment, the immunotoxin comprises a protein synthesis toxin, preferably a saporin. In a preferred embodiment, the immunotoxin is an anti-cKit-saporin immunotoxin.

In one embodiment, the immunotoxin comprises an anti-CD45 antibody. In one embodiment, the immunotoxin comprises a protein synthesis toxin, preferably a saporin. In a preferred embodiment, the immunotoxin is an anti-CD45-saporin immunotoxin.

Preferably, the HSPCs are administered to the subject after the toxin has dissipated from the bone marrow of the subject.

### Exemplary Methods

In some embodiments, the method of the present invention comprises:
(a) administering one or more HSPC mobiliser to a subject to mobilise endogenous HSPCs from the subject's bone marrow; and
(b) administering a population of HSPCs to the subject at the peak of mobilisation, wherein the population of HSPCs is cultured ex vivo prior to administration and genetically engineered to express one or more engraftment enhancer.

In some embodiments, the method of the present invention comprises:
(a) administering one or more HSPC mobiliser to a subject to mobilise endogenous HSPCs from the subject's bone marrow; and
(b) administering a population of HSPCs to the subject at the peak of mobilisation, wherein the population of HSPCs is cultured ex vivo prior to administration and genetically engineered to express one or more engraftment enhancer,
   wherein the subject does not undergo chemotherapy or radiotherapy conditioning before administration of the HSPCs.

In some embodiments, the method of the present invention comprises:
(a) (i) administering a G-CSF for about 7 days; (ii) administering a CXCR4 antagonist for about 2 days; and (iii) optionally, administering a VLA-4 antagonist for about 2 days to a subject to mobilise endogenous HSPCs from the subject's bone marrow; and
(b) administering a population of HSPCs to the subject at the peak of mobilisation, wherein the population of HSPCs is cultured ex vivo prior to administration and genetically engineered to express one or more engraftment enhancer,
   wherein the subject does not undergo chemotherapy or radiotherapy conditioning before administration of the HSPCs.

In some embodiments, the method of the present invention comprises:
(a) (i) administering a G-CSF for about 7 days; (ii) administering a CXCR4 antagonist for about 2 days; and (iii) optionally, administering a VLA-4 antagonist for about 2 days to a subject to mobilise endogenous HSPCs from the subject's bone marrow; and
(b) administering a population of HSPCs to the subject at the peak of mobilisation, wherein the population of HSPCs is cultured ex vivo prior to administration and an RNA polynucleotide encoding one or more engraftment enhancer is introduced into the HSPCs prior to administration,
   wherein the subject does not undergo chemotherapy or radiotherapy conditioning before administration of the HSPCs.

In some embodiments, the method of the present invention comprises:
(a) (i) administering a G-CSF for about 7 days; (ii) administering a CXCR4 antagonist for about 2 days; and (iii) optionally, administering a VLA-4 antagonist for about 2 days to a subject to mobilise endogenous HSPCs from the subject's bone marrow; and
(b) administering a population of HSPCs to the subject at the peak of mobilisation, wherein the population of HSPCs is cultured ex vivo prior to administration and an RNA polynucleotide encoding one or more engraftment enhancer selected from CXCR4 (or a fragment or variant thereof), CD47 (or a fragment or variant thereof), ITGA4 (or a fragment or variant thereof), and KIT (or a fragment or variant thereof) is introduced into the HSPCs prior to administration,
   wherein the subject does not undergo chemotherapy or radiotherapy conditioning before administration of the HSPCs.

In some embodiments, the method of the present invention comprises:
(a) (i) administering a G-CSF for about 7 days; (ii) administering a CXCR4 antagonist for about 2 days; and (iii) optionally, administering a VLA-4 antagonist for about 2 days to a subject to mobilise endogenous HSPCs from the subject's bone marrow; and
(b) administering a population of autologous genetically engineered HSPCs to the subject at the peak of mobilisation, wherein the population of HSPCs is cultured ex vivo prior to administration and an RNA polynucleotide encoding one or more engraftment enhancer selected from CXCR4 (or a fragment or variant thereof), CD47 (or a fragment or variant thereof), ITGA4 (or a fragment or variant thereof), and KIT (or a fragment or variant thereof) is introduced into the HSPCs prior to administration,
   wherein the subject does not undergo chemotherapy or radiotherapy conditioning before administration of the HSPCs.

### Engraftment Enhancers

In one aspect, the present invention provides use of one or more engraftment enhancer for increasing engraftment by haematopoietic stem and/or progenitor cells (HSPCs). In one embodiment, the use is an ex vivo use. In one embodiment, the use is an in vitro use.

In one aspect, the present invention provides a method for increasing engraftment by haematopoietic stem and/or progenitor cells (HSPCs), wherein the method comprises the step of genetically engineering the HSPCs to express one or more engraftment enhancer. In one embodiment, the method is an ex vivo method. In one embodiment, the method is an in vitro method.

In one aspect, the present invention provides a population of genetically engineered haematopoietic stem and/or progenitor cells (HSPCs) obtainable or obtained by the method of the present invention.

In one aspect, the present invention provides a population of genetically engineered haematopoietic stem and/or progenitor cells (HSPCs), wherein the HSPCs are genetically engineered to express one or more engraftment enhancer.

In one aspect, the present invention provides a method for haematopoietic stem and/or progenitor cell (HSPC) transplantation comprising the steps:
(a) providing a population of HSPCs which are genetically engineered to express one or more engraftment enhancer; and
(b) administering the HSPCs to a subject.

An “engraftment enhancer” may refer to any agent that enhances the ability of exogenous HSPCs to engraft in a subject's bone marrow. The increased engraftment may be in comparison to HSPCs that have not been genetically engineered to express the engraftment enhancer. Engraftment may be increased, for example, by at least about 10%, at least about 20%, at least about 30%, at least about 40%, at least about 50%, at least about 75%, at least about 100%, at least about 200% or at least about 500% in comparison to HSPCs that have not been genetically engineered to express the engraftment enhancer. This ability may be determined by any suitable assay. For example, using a transmigration assay (see e.g. Henschler, R. and Richter, R., 2019. In Stem Cell Mobilization (pp. 59-70). Humana, New York, NY) or by transplanting the exogenous HSPCs and determining the extent of engraftment following transplantation.

In some embodiments, the one or more engraftment enhancer comprises or consists of CXCR4 (or a fragment or variant thereof), CD47 (or a fragment or variant thereof), ITGA4 (or a fragment or variant thereof), and/or KIT (or a fragment or variant thereof).

In some embodiments, the one or more engraftment enhancer comprises two or more engraftment enhancers. In some embodiments, the one or more engraftment enhancer comprises three or more engraftment enhancers. In some embodiments, the one or more engraftment enhancer comprises four or more engraftment enhancers. In some embodiments, the one or more engraftment enhancer consists of one engraftment enhancer. In some embodiments, the one or more engraftment enhancer consists of two engraftment enhancers. In some embodiments, the one or more engraftment enhancer consists of three engraftment enhancers. In some embodiments, the one or more engraftment enhancer consists of four engraftment enhancers.

In some embodiments, the one or more engraftment enhancer comprises or consists of CXCR4 (or a fragment or variant thereof) and CD47 (or a fragment or variant thereof). In some embodiments, the one or more engraftment enhancer comprises or consists of CXCR4 (or a fragment or variant thereof) and ITGA4 (or a fragment or variant thereof). In some embodiments, the one or more engraftment enhancer comprises or consists of CXCR4 (or a fragment or variant thereof) and KIT (or a fragment or variant thereof). In some embodiments, the one or more engraftment enhancer comprises or consists of ITGA4 (or a fragment or variant thereof) and CD47 (or a fragment or variant thereof). In some embodiments, the one or more engraftment enhancer comprises or consists of KIT (or a fragment or variant thereof) and CD47 (or a fragment or variant thereof). In some embodiments, the one or more engraftment enhancer comprises or consists of ITGA4 (or a fragment or variant thereof) and KIT (or a fragment or variant thereof).

In some embodiments, the one or more engraftment enhancer comprises or consists of CXCR4 (or a fragment or variant thereof), ITGA4 (or a fragment or variant thereof), and KIT (or a fragment or variant thereof). In some embodiments, the one or more engraftment enhancer comprises or consists of CXCR4 (or a fragment or variant thereof), ITGA4 (or a fragment or variant thereof), and CD47 (or a fragment or variant thereof). In some embodiments, the one or more engraftment enhancer comprises or consists of CXCR4 (or a fragment or variant thereof), KIT (or a fragment or variant thereof), and CD47 (or a fragment or variant thereof).

In some embodiments, the one or more engraftment enhancer comprises or consists of CXCR4 (or a fragment or variant thereof), CD47 (or a fragment or variant thereof), ITGA4 (or a fragment or variant thereof), and KIT (or a fragment or variant thereof).

In one embodiment, the engraftment enhancer is a fusion with a destabilising domain protein. In one embodiment, each engraftment enhancer is individually a fusion with destabilising domain proteins. Use of an example destabilising domain strategy is disclosed in Banaszynski et al. (2012) Cell, 126(5): 995-1004. Typically, the engraftment enhancer is operably linked to a destabilising domain protein (DD) that is tuneable through the use of a stabilising agent (e.g. a small molecule). For example, the destabilising domain may be stable when bound to its stabilising agent, but may cause the fusion protein to be unstable in the absence thereof. For example, the engraftment enhancer may be operably linked to a destabilising domain protein and delivered using a vector. The engraftment enhancer in this form may be normally unstable, but expression of the engraftment enhancer may then be induced (e.g. in vivo) for a time period of interest by the delivery of a stabilising agent.

### C-X-C Chemokine Receptor Type 4 (CXCR4)

In one aspect, the present invention provides use of CXCR4 (or a fragment or variant thereof) for increasing engraftment by haematopoietic stem and/or progenitor cells (HSPCs).

In one aspect, the present invention provides a method for increasing engraftment by haematopoietic stem and/or progenitor cells (HSPCs), wherein the method comprises the step of genetically engineering the HSPCs to express CXCR4 (or a fragment or variant thereof).

In one aspect, the present invention provides a population of genetically engineered haematopoietic stem and/or progenitor cells (HSPCs), wherein the HSPCs are genetically engineered to express CXCR4 (or a fragment or variant thereof).

In one aspect, the present invention provides a method for haematopoietic stem and/or progenitor cell (HSPC) transplantation comprising the steps:
(a) providing a population of HSPCs which are genetically engineered to express CXCR4 (or a fragment or variant thereof); and
(b) administering the HSPCs to a subject.

C-X-C chemokine receptor type 4 (CXCR4) is a receptor expressed on the surface of HSPCs. The interaction of CXCR4 with CXCL12 is one of the major mechanisms that directs migration to the bone marrow. CXCR4 may also known as fusin or CD184. Mouse and human CXCR4 have been cloned and show about 91% overall amino acid identity (see e.g. Heesen, M., et al., 1996. The Journal of Immunology, 157(12), pp. 5455-5460).

In a preferred embodiment, the CXCR4 is human CXCR4. A human CXCR4 may have an amino acid sequence of UniProtKB P61073. In some embodiments, the CXCR4 is isoform I.

Exemplary CXCR4 polypeptides are provided by SEQ ID NOs: 1 and 2. In one embodiment, the CXCR4 comprises or consists of an amino acid sequence that has at least 70%, at least 80%, at least 85%, at least 90%, at least 95%, at least 96%, at least 97%, at least 98%, at least 99% or 100% identity to SEQ ID NO: 1 or 2, preferably wherein the amino acid sequence substantially retains the engraftment enhancing activity of the protein represented by SEQ ID NO: 1 or 2, respectively.

In one embodiment, the CXCR4 comprises or consists of an amino acid sequence that has at least 70%, at least 80%, at least 85%, at least 90%, at least 95%, at least 96%, at least 97%, at least 98%, at least 99% or 100% identity to SEQ ID NO: 2, preferably wherein the amino acid sequence substantially retains the engraftment enhancing activity of the protein represented by SEQ ID NO: 2.

In one embodiment, the amino acid sequence of CXCR4 isoform II is:

Exemplary CXCR4 isoform II
(SEQ ID NO: 1)
MSIPLPLLQIYTSDNYTEEMGSGDYDSMKEPCFREENANENKIFLPTIYS
IIFLTGIVGNGLVILVMGYQKKLRSMTDKYRLHLSVADLLFVITLPFWAV
DAVANWYFGNFLCKAVHVIYTVNLYSSVLILAFISLDRYLAIVHATNSQR
PRKLLAEKVVYVGVWIPALLLTIPDFIFANVSEADDRYICDRFYPNDLWV
VVFQFQHIMVGLILPGIVILSCYCIIISKLSHSKGHQKRKALKTTVILIL
AFFACWLPYYIGISIDSFILLEIIKQGCEFENTVHKWISITEALAFFHCC
LNPILYAFLGAKFKTSAQHALTSVSRGSSLKILSKGKRGGHSSVSTESES
SSFHSS

In one embodiment, the amino acid sequence of CXCR4 isoform I is:

Exemplary CXCR4 isoform I
(SEQ ID NO: 2)
MEGISIYTSDNYTEEMGSGDYDSMKEPCFREENANENKIFLPTIYSIIFL
TGIVGNGLVILVMGYQKKLRSMTDKYRLHLSVADLLFVITLPFWAVDAVA
NWYFGNFLCKAVHVIYTVNLYSSVLILAFISLDRYLAIVHATNSQRPRKL
LAEKVVYVGVWIPALLLTIPDFIFANVSEADDRYICDRFYPNDLWVVVFQ
FQHIMVGLILPGIVILSCYCIIISKLSHSKGHQKRKALKTTVILILAFFA
CWLPYYIGISIDSFILLEIIKQGCEFENTVHKWISITEALAFFHCCLNPI
LYAFLGAKFKTSAQHALTSVSRGSSLKILSKGKRGGHSSVSTESESSSFH
SS

In a preferred embodiment, the nucleotide sequence encoding the CXCR4 is codon optimised.

In one embodiment, the CXCR4 is encoded by a nucleotide sequence that has at least 70%, at least 80%, at least 85%, at least 90%, at least 95%, at least 96%, at least 97%, at least 98%, at least 99% or 100% identity to any of SEQ ID NOs: 10-11 or 12-13, preferably wherein the protein encoded by the nucleotide sequence substantially retains the engraftment enhancing activity of the protein represented by SEQ ID NO: 1 or 2, respectively.

In one embodiment, the CXCR4 is encoded by a nucleotide sequence that has at least 70%, at least 80%, at least 85%, at least 90%, at least 95%, at least 96%, at least 97%, at least 98%, at least 99% or 100% identity to SEQ ID NO: 12, preferably wherein the protein encoded by the nucleotide sequence substantially retains the engraftment enhancing activity of the protein represented by SEQ ID NO: 2.

In one embodiment, the nucleotide sequence encoding CXCR4 isoform II is:

Exemplary nucleotide sequence encoding CXCR4
isoform II
(SEQ ID NO: 10)
ATGTCTATTCCTCTGCCCCTGCTGCAGATCTACACCAGCGACAACTACAC
CGAGGAAATGGGCAGCGGCGACTACGACAGCATGAAGGAACCCTGCTTCC
GGGAAGAGAACGCCAACTTCAACAAGATCTTCCTGCCCACAATCTACAGC
ATCATCTTTCTGACCGGCATCGTGGGCAACGGACTCGTGATCCTCGTGAT
GGGCTACCAGAAAAAGCTGCGGAGCATGACCGACAAGTACCGGCTGCACC
TGAGCGTGGCCGACCTGCTGTTCGTGATCACCCTGCCTTTCTGGGCCGTG
GACGCCGTGGCCAATTGGTACTTCGGCAACTTCCTGTGCAAGGCCGTGCA
CGTGATCTACACAGTGAACCTGTACAGCAGCGTGCTGATCCTGGCCTTCA
TCAGCCTGGACAGATACCTGGCCATCGTGCACGCCACCAACAGCCAGCGG
CCTAGAAAGCTGCTGGCCGAGAAGGTGGTGTACGTGGGCGTGTGGATTCC
CGCCCTGCTGCTGACCATCCCCGACTTCATCTTCGCCAACGTGTCCGAGG
CCGACGACCGGTACATCTGCGACCGGTTCTACCCCAACGACCTGTGGGTG
GTGGTGTTCCAGTTCCAGCACATCATGGTGGGACTGATCCTGCCTGGCAT
CGTGATTCTGAGCTGCTACTGCATCATCATCAGCAAGCTGAGCCACAGCA
AGGGCCACCAGAAGCGGAAGGCCCTGAAAACCACCGTGATCCTGATTCTG
GCTTTCTTCGCCTGCTGGCTGCCCTACTACATCGGCATCAGCATCGACAG
CTTCATCCTGCTGGAAATCATCAAGCAGGGCTGCGAGTTCGAGAACACCG
TGCACAAGTGGATCAGCATTACCGAGGCCCTGGCCTTTTTCCACTGCTGC
CTGAACCCTATCCTGTACGCCTTCCTGGGCGCCAAGTTCAAGACCTCTGC
CCAGCACGCCCTGACCAGCGTGTCCAGAGGAAGCAGCCTGAAGATCCTGA
GCAAGGGCAAGAGAGGCGGCCACAGCTCCGTGTCTACAGAGAGCGAGAGC
AGCAGCTTCCACAGCAGC

In another embodiment, the nucleotide sequence encoding CXCR4 isoform II is:

Exemplary nucleotide sequence encoding CXCR4
isoform II
(SEQ ID NO: 11)
ATGTCCATTCCTTTGCCTCTTTTGCAGATATACACTTCAGATAACTACAC
CGAGGAAATGGGCTCAGGGGACTATGACTCCATGAAGGAACCCTGTTTCC
GTGAAGAAAATGCTAATTTCAATAAAATCTTCCTGCCCACCATCTACTCC
ATCATCTTCTTAACTGGCATTGTGGGCAATGGATTGGTCATCCTGGTCAT
GGGTTACCAGAAGAAACTGAGAAGCATGACGGACAAGTACAGGCTGCACC
TGTCAGTGGCCGACCTCCTCTTTGTCATCACGCTTCCCTTCTGGGCAGTT
GATGCCGTGGCAAACTGGTACTTTGGGAACTTCCTATGCAAGGCAGTCCA
TGTCATCTACACAGTCAACCTCTACAGCAGTGTCCTCATCCTGGCCTTCA
TCAGTCTGGACCGCTACCTGGCCATCGTCCACGCCACCAACAGTCAGAGG
CCAAGGAAGCTGTTGGCTGAAAAGGTGGTCTATGTTGGCGTCTGGATCCC
TGCCCTCCTGCTGACTATTCCCGACTTCATCTTTGCCAACGTCAGTGAGG
CAGATGACAGATATATCTGTGACCGCTTCTACCCCAATGACTTGTGGGTG
GTTGTGTTCCAGTTTCAGCACATCATGGTTGGCCTTATCCTGCCTGGTAT
TGTCATCCTGTCCTGCTATTGCATTATCATCTCCAAGCTGTCACACTCCA
AGGGCCACCAGAAGCGCAAGGCCCTCAAGACCACAGTCATCCTCATCCTG
GCTTTCTTCGCCTGTTGGCTGCCTTACTACATTGGGATCAGCATCGACTC
CTTCATCCTCCTGGAAATCATCAAGCAAGGGTGTGAGTTTGAGAACACTG
TGCACAAGTGGATTTCCATCACCGAGGCCCTAGCTTTCTTCCACTGTTGT
CTGAACCCCATCCTCTATGCTTTCCTTGGAGCCAAATTTAAAACCTCTGC
CCAGCACGCACTCACCTCTGTGAGCAGAGGGTCCAGCCTCAAGATCCTCT
CCAAAGGAAAGCGAGGTGGACATTCATCTGTTTCCACTGAGTCTGAGTCT
TCAAGTTTTCACTCCAGC

In another embodiment, the nucleotide sequence encoding CXCR4 isoform I is:

Exemplary nucleotide sequence encoding CXCR4
isoform I
(SEQ ID NO: 12)
ATGGAAGGCATCAGCATCTACACCAGCGACAACTACACCGAGGAAATGGG
CAGCGGCGACTACGACAGCATGAAGGAACCCTGCTTCCGGGAAGAGAACG
CCAACTTCAACAAGATCTTCCTGCCCACAATCTACAGCATCATCTTTCTG
ACCGGCATCGTGGGCAACGGACTCGTGATCCTCGTGATGGGCTACCAGAA
AAAGCTGCGGAGCATGACCGACAAGTACCGGCTGCACCTGAGCGTGGCCG
ACCTGCTGTTCGTGATCACCCTGCCTTTCTGGGCCGTGGACGCCGTGGCC
AATTGGTACTTCGGCAACTTCCTGTGCAAGGCCGTGCACGTGATCTACAC
AGTGAACCTGTACAGCAGCGTGCTGATCCTGGCCTTCATCAGCCTGGACA
GATACCTGGCCATCGTGCACGCCACCAACAGCCAGCGGCCTAGAAAGCTG
CTGGCCGAGAAGGTGGTGTACGTGGGCGTGTGGATTCCCGCCCTGCTGCT
GACCATCCCCGACTTCATCTTCGCCAACGTGTCCGAGGCCGACGACCGGT
ACATCTGCGACCGGTTCTACCCCAACGACCTGTGGGTGGTGGTGTTCCAG
TTCCAGCACATCATGGTGGGACTGATCCTGCCTGGCATCGTGATTCTGAG
CTGCTACTGCATCATCATCAGCAAGCTGAGCCACAGCAAGGGCCACCAGA
AGCGGAAGGCCCTGAAAACCACCGTGATCCTGATTCTGGCTTTCTTCGCC
TGCTGGCTGCCCTACTACATCGGCATCAGCATCGACAGCTTCATCCTGCT
GGAAATCATCAAGCAGGGCTGCGAGTTCGAGAACACCGTGCACAAGTGGA
TCAGCATTACCGAGGCCCTGGCCTTTTTCCACTGCTGCCTGAACCCTATC
CTGTACGCCTTCCTGGGCGCCAAGTTCAAGACCTCTGCCCAGCACGCCCT
GACCAGCGTGTCCAGAGGAAGCAGCCTGAAGATCCTGAGCAAGGGCAAGA
GAGGCGGCCACAGCTCCGTGTCTACAGAGAGCGAGAGCAGCAGCTTCCAC
AGCAGC

In another embodiment, the nucleotide sequence encoding CXCR4 isoform I is:

Exemplary nucleotide sequence encoding CXCR4
isoform I
(SEQ ID NO: 13)
ATGGAGGGGATCAGTATATACACTTCAGATAACTACACCGAGGAAATGGG
CTCAGGGGACTATGACTCCATGAAGGAACCCTGTTTCCGTGAAGAAAATG
CTAATTTCAATAAAATCTTCCTGCCCACCATCTACTCCATCATCTTCTTA
ACTGGCATTGTGGGCAATGGATTGGTCATCCTGGTCATGGGTTACCAGAA
GAAACTGAGAAGCATGACGGACAAGTACAGGCTGCACCTGTCAGTGGCCG
ACCTCCTCTTTGTCATCACGCTTCCCTTCTGGGCAGTTGATGCCGTGGCA
AACTGGTACTTTGGGAACTTCCTATGCAAGGCAGTCCATGTCATCTACAC
AGTCAACCTCTACAGCAGTGTCCTCATCCTGGCCTTCATCAGTCTGGACC
GCTACCTGGCCATCGTCCACGCCACCAACAGTCAGAGGCCAAGGAAGCTG
TTGGCTGAAAAGGTGGTCTATGTTGGCGTCTGGATCCCTGCCCTCCTGCT
GACTATTCCCGACTTCATCTTTGCCAACGTCAGTGAGGCAGATGACAGAT
ATATCTGTGACCGCTTCTACCCCAATGACTTGTGGGTGGTTGTGTTCCAG
TTTCAGCACATCATGGTTGGCCTTATCCTGCCTGGTATTGTCATCCTGTC
CTGCTATTGCATTATCATCTCCAAGCTGTCACACTCCAAGGGCCACCAGA
AGCGCAAGGCCCTCAAGACCACAGTCATCCTCATCCTGGCTTTCTTCGCC
TGTTGGCTGCCTTACTACATTGGGATCAGCATCGACTCCTTCATCCTCCT
GGAAATCATCAAGCAAGGGTGTGAGTTTGAGAACACTGTGCACAAGTGGA
TTTCCATCACCGAGGCCCTAGCTTTCTTCCACTGTTGTCTGAACCCCATC
CTCTATGCTTTCCTTGGAGCCAAATTTAAAACCTCTGCCCAGCACGCACT
CACCTCTGTGAGCAGAGGGTCCAGCCTCAAGATCCTCTCCAAAGGAAAGC
GAGGTGGACATTCATCTGTTTCCACTGAGTCTGAGTCTTCAAGTTTTCAC
TCCAGC

A person skilled in the art would be able to generate variants and/or fragments retaining the engraftment enhancing activity of CXCR4 based on conservative substitutions and/or the known structural and functional features of CXCR4. These are described, for instance in Qin, L., et al., 2015. Science, 347(6226), pp. 1117-1122.

### CXCR4 Fragments

In one embodiment, the CXCR4 is a truncated CXCR4. In one embodiment, the CXCR4 is a CXCR4 Whim isoform (Kawai T. et al. (2005) Experimental Hematology; the CXCR4 Whim isoform I may be naturally expressed in subjects with Whim syndrome). In one embodiment, the CXCR4 is a CXCR4 Whim isoform 1. In one embodiment, the CXCR4 is a CXCR4 Whim isoform II. CXCR4 Whim isoform I may also be referred to as a CXCR4 R334X mutant.

Exemplary truncated CXCR4 polypeptides are provided by SEQ ID NOs: 3 and 4. In one embodiment, the CXCR4 comprises or consists of an amino acid sequence that has at least 70%, at least 80%, at least 85%, at least 90%, at least 95%, at least 96%, at least 97%, at least 98%, at least 99% or 100% identity to SEQ ID NO: 3 or 4, preferably wherein the amino acid sequence substantially retains the engraftment enhancing activity of the protein represented by SEQ ID NO: 3 or 4, respectively.

In one embodiment, the CXCR4 comprises or consists of an amino acid sequence that has at least 70%, at least 80%, at least 85%, at least 90%, at least 95%, at least 96%, at least 97%, at least 98%, at least 99% or 100% identity to SEQ ID NO: 4, preferably wherein the amino acid sequence substantially retains the engraftment enhancing activity of the protein represented by SEQ ID NO: 4.

An example amino acid sequence of CXCR4 Whim isoform II is:

Exemplary CXCR4 Whim isoform II
(SEQ ID NO: 3)
MSIPLPLLQIYTSDNYTEEMGSGDYDSMKEPCFREENANFNKIFLPTIYS
IIFLTGIVGNGLVILVMGYQKKLRSMTDKYRLHLSVADLLFVITLPFWAV
DAVANWYFGNFLCKAVHVIYTVNLYSSVLILAFISLDRYLAIVHATNSQR
PRKLLAEKVVYVGVWIPALLLTIPDFIFANVSEADDRYICDRFYPNDLWV
VVFQFQHIMVGLILPGIVILSCYCIIISKLSHSKGHQKRKALKTTVILIL
AFFACWLPYYIGISIDSFILLEIIKQGCEFENTVHKWISITEALAFFHCC
LNPILYAFLGAKFKTSAQHALTSVSRGSSLKILSKGK

An example amino acid sequence of CXCR4 Whim isoform I is:

Exemplary CXCR4 Whim isoform I
(SEQ ID NO: 4)
MEGISIYTSDNYTEEMGSGDYDSMKEPCFREENANFNKIFLPTIYSIIFL
TGIVGNGLVILVMGYQKKLRSMTDKYRLHLSVADLLFVITLPFWAVDAVA
NWYFGNFLCKAVHVIYTVNLYSSVLILAFISLDRYLAIVHATNSQRPRKL
LAEKVVYVGVWIPALLLTIPDFIFANVSEADDRYICDRFYPNDLWVVVFQ
FQHIMVGLILPGIVILSCYCIIISKLSHSKGHQKRKALKTTVILILAFFA
CWLPYYIGISIDSFILLEIIKQGCEFENTVHKWISITEALAFFHCCLNPI
LYAFLGAKFKTSAQHALTSVSRGSSLKILSKGK

In one embodiment, the CXCR4 is encoded by a nucleotide sequence that has at least 70%, at least 80%, at least 85%, at least 90%, at least 95%, at least 96%, at least 97%, at least 98%, at least 99% or 100% identity to any of SEQ ID NOs: 14-15 or 16-17, preferably wherein the protein encoded by the nucleotide sequence substantially retains the engraftment enhancing activity of the protein represented by SEQ ID NO: 3 or 4, respectively.

In one embodiment, the CXCR4 is encoded by a nucleotide sequence that has at least 70%, at least 80%, at least 85%, at least 90%, at least 95%, at least 96%, at least 97%, at least 98%, at least 99% or 100% identity to SEQ ID NO: 17, preferably wherein the protein encoded by the nucleotide sequence substantially retains the engraftment enhancing activity of the protein represented by SEQ ID NO: 4.

An example nucleotide sequence encoding CXCR4 Whim isoform II is:

Exemplary nucleotide sequence encoding CXCR4 Whim
isoform II
(SEQ ID NO: 14)
ATGTCCATTCCTTTGCCTCTTTTGCAGATATACACTTCAGATAACTACAC
CGAGGAAATGGGCTCAGGGGACTATGACTCCATGAAGGAACCCTGTTTCC
GTGAAGAAAATGCTAATTTCAATAAAATCTTCCTGCCCACCATCTACTCC
ATCATCTTCTTAACTGGCATTGTGGGCAATGGATTGGTCATCCTGGTCAT
GGGTTACCAGAAGAAACTGAGAAGCATGACGGACAAGTACAGGCTGCACC
TGTCAGTGGCCGACCTCCTCTTTGTCATCACGCTTCCCTTCTGGGCAGTT
GATGCCGTGGCAAACTGGTACTTTGGGAACTTCCTATGCAAGGCAGTCCA
TGTCATCTACACAGTCAACCTCTACAGCAGTGTCCTCATCCTGGCCTTCA
TCAGTCTGGACCGCTACCTGGCCATCGTCCACGCCACCAACAGTCAGAGG
CCAAGGAAGCTGTTGGCTGAAAAGGTGGTCTATGTTGGCGTCTGGATCCC
TGCCCTCCTGCTGACTATTCCCGACTTCATCTTTGCCAACGTCAGTGAGG
CAGATGACAGATATATCTGTGACCGCTTCTACCCCAATGACTTGTGGGTG
GTTGTGTTCCAGTTTCAGCACATCATGGTTGGCCTTATCCTGCCTGGTAT
TGTCATCCTGTCCTGCTATTGCATTATCATCTCCAAGCTGTCACACTCCA
AGGGCCACCAGAAGCGCAAGGCCCTCAAGACCACAGTCATCCTCATCCTG
GCTTTCTTCGCCTGTTGGCTGCCTTACTACATTGGGATCAGCATCGACTC
CTTCATCCTCCTGGAAATCATCAAGCAAGGGTGTGAGTTTGAGAACACTG
TGCACAAGTGGATTTCCATCACCGAGGCCCTAGCTTTCTTCCACTGTTGT
CTGAACCCCATCCTCTATGCTTTCCTTGGAGCCAAATTTAAAACCTCTGC
CCAGCACGCACTCACCTCTGTGAGCAGAGGGTCCAGCCTCAAGATCCTCT
CCAAAGGAAAGTGAGGTGGACATTCATCTGTTTCCACTGAGTCTGAGTCT
TCAAGTTTTCACTCCAGC

Another example nucleotide sequence encoding CXCR4 Whim isoform II is:

Exemplary nucleotide sequence encoding CXCR4 Whim
isoform II
(SEQ ID NO: 15)
ATGTCTATTCCTCTGCCCCTGCTGCAGATCTACACCAGCGACAACTACAC
CGAGGAAATGGGCAGCGGCGACTACGACAGCATGAAGGAACCCTGCTTCC
GGGAAGAGAACGCCAACTTCAACAAGATCTTCCTGCCCACAATCTACAGC
ATCATCTTTCTGACCGGCATCGTGGGCAACGGACTCGTGATCCTCGTGAT
GGGCTACCAGAAAAAGCTGCGGAGCATGACCGACAAGTACCGGCTGCACC
TGAGCGTGGCCGACCTGCTGTTCGTGATCACCCTGCCTTTCTGGGCCGTG
GACGCCGTGGCCAATTGGTACTTCGGCAACTTCCTGTGCAAGGCCGTGCA
CGTGATCTACACAGTGAACCTGTACAGCAGCGTGCTGATCCTGGCCTTCA
TCAGCCTGGACAGATACCTGGCCATCGTGCACGCCACCAACAGCCAGCGG
CCTAGAAAGCTGCTGGCCGAGAAGGTGGTGTACGTGGGCGTGTGGATTCC
CGCCCTGCTGCTGACCATCCCCGACTTCATCTTCGCCAACGTGTCCGAGG
CCGACGACCGGTACATCTGCGACCGGTTCTACCCCAACGACCTGTGGGTG
GTGGTGTTCCAGTTCCAGCACATCATGGTGGGACTGATCCTGCCTGGCAT
CGTGATTCTGAGCTGCTACTGCATCATCATCAGCAAGCTGAGCCACAGCA
AGGGCCACCAGAAGCGGAAGGCCCTGAAAACCACCGTGATCCTGATTCTG
GCTTTCTTCGCCTGCTGGCTGCCCTACTACATCGGCATCAGCATCGACAG
CTTCATCCTGCTGGAAATCATCAAGCAGGGCTGCGAGTTCGAGAACACCG
TGCACAAGTGGATCAGCATTACCGAGGCCCTGGCCTTTTTCCACTGCTGC
CTGAACCCTATCCTGTACGCCTTCCTGGGCGCCAAGTTCAAGACCTCTGC
CCAGCACGCCCTGACCAGCGTGTCCAGAGGAAGCAGCCTGAAGATCCTGA
GCAAGGGCAAGTGAGGCGGCCACAGCTCCGTGTCTACAGAGAGCGAGAGC
AGCAGCTTCCACAGCAGC

An example nucleotide sequence encoding CXCR4 Whim isoform I is:

Exemplary nucleotide sequence encoding CXCR4 Whim
isoform I
(SEQ ID NO: 16)
ATGGAGGGGATCAGTATATACACTTCAGATAACTACACCGAGGAAATGGG
CTCAGGGGACTATGACTCCATGAAGGAACCCTGTTTCCGTGAAGAAAATG
CTAATTTCAATAAAATCTTCCTGCCCACCATCTACTCCATCATCTTCTTA
ACTGGCATTGTGGGCAATGGATTGGTCATCCTGGTCATGGGTTACCAGAA
GAAACTGAGAAGCATGACGGACAAGTACAGGCTGCACCTGTCAGTGGCCG
ACCTCCTCTTTGTCATCACGCTTCCCTTCTGGGCAGTTGATGCCGTGGCA
AACTGGTACTTTGGGAACTTCCTATGCAAGGCAGTCCATGTCATCTACAC
AGTCAACCTCTACAGCAGTGTCCTCATCCTGGCCTTCATCAGTCTGGACC
GCTACCTGGCCATCGTCCACGCCACCAACAGTCAGAGGCCAAGGAAGCTG
TTGGCTGAAAAGGTGGTCTATGTTGGCGTCTGGATCCCTGCCCTCCTGCT
GACTATTCCCGACTTCATCTTTGCCAACGTCAGTGAGGCAGATGACAGAT
ATATCTGTGACCGCTTCTACCCCAATGACTTGTGGGTGGTTGTGTTCCAG
TTTCAGCACATCATGGTTGGCCTTATCCTGCCTGGTATTGTCATCCTGTC
CTGCTATTGCATTATCATCTCCAAGCTGTCACACTCCAAGGGCCACCAGA
AGCGCAAGGCCCTCAAGACCACAGTCATCCTCATCCTGGCTTTCTTCGCC
TGTTGGCTGCCTTACTACATTGGGATCAGCATCGACTCCTTCATCCTCCT
GGAAATCATCAAGCAAGGGTGTGAGTTTGAGAACACTGTGCACAAGTGGA
TTTCCATCACCGAGGCCCTAGCTTTCTTCCACTGTTGTCTGAACCCCATC
CTCTATGCTTTCCTTGGAGCCAAATTTAAAACCTCTGCCCAGCACGCACT
CACCTCTGTGAGCAGAGGGTCCAGCCTCAAGATCCTCTCCAAAGGAAAGT
GAGGTGGACATTCATCTGTTTCCACTGAGTCTGAGTCTTCAAGTTTTCAC
TCCAGC

Another example nucleotide sequence encoding CXCR4 Whim isoform I is:

Exemplary nucleotide sequence encoding CXCR4 Whim
isoform I
(SEQ ID NO: 17)
ATGGAAGGCATCAGCATCTACACCAGCGACAACTACACCGAGGAAATGGG
CAGCGGCGACTACGACAGCATGAAGGAACCCTGCTTCCGGGAAGAGAACG
CCAACTTCAACAAGATCTTCCTGCCCACAATCTACAGCATCATCTTTCTG
ACCGGCATCGTGGGCAACGGACTCGTGATCCTCGTGATGGGCTACCAGAA
AAAGCTGCGGAGCATGACCGACAAGTACCGGCTGCACCTGAGCGTGGCCG
ACCTGCTGTTCGTGATCACCCTGCCTTTCTGGGCCGTGGACGCCGTGGCC
AATTGGTACTTCGGCAACTTCCTGTGCAAGGCCGTGCACGTGATCTACAC
AGTGAACCTGTACAGCAGCGTGCTGATCCTGGCCTTCATCAGCCTGGACA
GATACCTGGCCATCGTGCACGCCACCAACAGCCAGCGGCCTAGAAAGCTG
CTGGCCGAGAAGGTGGTGTACGTGGGCGTGTGGATTCCCGCCCTGCTGCT
GACCATCCCCGACTTCATCTTCGCCAACGTGTCCGAGGCCGACGACCGGT
ACATCTGCGACCGGTTCTACCCCAACGACCTGTGGGTGGTGGTGTTCCAG
TTCCAGCACATCATGGTGGGACTGATCCTGCCTGGCATCGTGATTCTGAG
CTGCTACTGCATCATCATCAGCAAGCTGAGCCACAGCAAGGGCCACCAGA
AGCGGAAGGCCCTGAAAACCACCGTGATCCTGATTCTGGCTTTCTTCGCC
TGCTGGCTGCCCTACTACATCGGCATCAGCATCGACAGCTTCATCCTGCT
GGAAATCATCAAGCAGGGCTGCGAGTTCGAGAACACCGTGCACAAGTGGA
TCAGCATTACCGAGGCCCTGGCCTTTTTCCACTGCTGCCTGAACCCTATC
CTGTACGCCTTCCTGGGCGCCAAGTTCAAGACCTCTGCCCAGCACGCCCT
GACCAGCGTGTCCAGAGGAAGCAGCCTGAAGATCCTGAGCAAGGGCAAGT
GAGGCGGCCACAGCTCCGTGTCTACAGAGAGCGAGAGCAGCAGCTTCCAC
AGCAGC

### CXCR4 Variants

In one aspect, the present invention provides use of a CXCR4 variant (or fragment thereof) for increasing engraftment by haematopoietic stem and/or progenitor cells (HSPCs).

In one aspect, the present invention provides a method for increasing engraftment by haematopoietic stem and/or progenitor cells (HSPCs), wherein the method comprises the step of genetically engineering the HSPCs to express a CXCR4 variant (or fragment thereof).

In one aspect, the present invention provides a population of genetically engineered haematopoietic stem and/or progenitor cells (HSPCs), wherein the HSPCs are genetically engineered to express a CXCR4 variant (or fragment thereof).

In one aspect, the present invention provides a method for haematopoietic stem and/or progenitor cell (HSPC) transplantation comprising the steps:
(a) providing a population of HSPCs which are genetically engineered to express a CXCR4 variant (or fragment thereof); and
(b) administering the HSPCs to a subject.

The CXCR4 variant may be one which enhances engraftment to the same or a greater level than CXCR4. This may be determined by any suitable assay. For example, using a transmigration assay or by transplanting genetically engineered HSPCs and determining the extent of engraftment following transplantation.

The CXCR4 variant may have a maintained or increased response to SDF-1 compared to CXCR4. This may be determined by any suitable assay. For example, by using a calcium mobilisation assay, an antibody binding assay, or a PCR-based virus entry assay e.g. as described in Hatse, S., et al., 2001. Molecular pharmacology, 60(1), pp. 164-173. In some embodiments, the CXCR4 variant has the same or greater binding affinity for SDF-1 compared to CXCR4. This may be determined by any suitable assay. For example, using the binding assay described in e.g. Zhang, W. B., et al., 2002. Journal of Biological Chemistry, 277(27), pp. 24515-24521.

In some embodiments, the CXCR4 variant has increased resistance to a CXCR4 antagonist compared to CXCR4. In some embodiments, the CXCR4 variant has increased resistance to AMD3100 compared to CXCR4. In some embodiments, the CXCR4 variant has a reduced binding affinity for a CXCR4 antagonist (e.g. AMD3100) compared to CXCR4. This may be determined by any suitable assay. For example, using a transmigration assay in the absence or presence of a CXCR4 antagonist or using a calcium mobilisation assay, an antibody binding assay, or a PCR-based virus entry assay e.g. as described in Hatse, S., et al., 2001. Molecular pharmacology, 60(1), pp. 164-173.

In some embodiments, the CXCR4 variant comprises one or more amino acid substitution selected from: V160L, A175F, Q200A, D262N, and H281A. These amino acid positions refer to the positions in the CXCR4 shown in SEQ ID NO: 2. These amino acid positions can be converted to the corresponding position in other isoforms, variants, and/or fragments. For example, these correspond to: V164L, A179F, Q204A, D262N, and H285A in the CXCR4 shown in SEQ ID NO: 1.

In some embodiments, the CXCR4 variant comprises one or more amino acid substitution selected from: A175F, Q200A, D262N, and H281A. In some embodiments, the CXCR4 variant comprises one or more amino acid substitution selected from: A175F or D262N. In some embodiments, the CXCR4 variant comprises the amino acid substitution A175F. In some embodiments, the CXCR4 variant comprises the amino acid substitution Q200A. In some embodiments, the CXCR4 variant comprises the amino acid substitution D262N. In some embodiments, the CXCR4 variant comprises the amino acid substitution H281A.

In some embodiments, the CXCR4 variant comprises or consists of an amino acid sequence that has at least 70%, at least 80%, at least 85%, at least 90%, at least 95%, at least 96%, at least 97%, at least 98%, at least 99% or 100% identity to SEQ ID NO: 2 and comprises one or more amino acid substitution selected from: V160L, A175F, Q200A, D262N, and H281A, preferably wherein the amino acid sequence substantially retains the engraftment enhancing activity of the protein represented by SEQ ID NO: 2.

In some embodiments, the CXCR4 variant comprises or consists of an amino acid sequence that has at least 70%, at least 80%, at least 85%, at least 90%, at least 95%, at least 96%, at least 97%, at least 98%, at least 99% or 100% identity to SEQ ID NO: 2 and comprises one or more amino acid substitution selected from: A175F, Q200A, D262N, and H281A, preferably wherein the amino acid sequence substantially retains the engraftment enhancing activity of the protein represented by SEQ ID NO: 2.

Exemplary CXCR4 variants are provided by SEQ ID NOs: 5-9. In one embodiment, the CXCR4 variant comprises or consists of an amino acid sequence that has at least 70%, at least 80%, at least 85%, at least 90%, at least 95%, at least 96%, at least 97%, at least 98%, at least 99% or 100% identity to any of SEQ ID NOs: 5-9, preferably wherein the amino acid sequence substantially retains the engraftment enhancing activity of the protein represented by SEQ ID NO: 2.

In one embodiment, the CXCR4 variant comprises or consists of an amino acid sequence that has at least 70%, at least 80%, at least 85%, at least 90%, at least 95%, at least 96%, at least 97%, at least 98%, at least 99% or 100% identity to any of SEQ ID NOs: 6-9, preferably wherein the amino acid sequence substantially retains the engraftment enhancing activity of the protein represented by SEQ ID NO: 2.

In one embodiment, the amino acid sequence of a CXCR4 V160L variant is:

Exemplary CXCR4 V160L variant
(SEQ ID NO: 5)
MEGISIYTSDNYTEEMGSGDYDSMKEPCFREENANENKIFLPTIYSIIFL
TGIVGNGLVILVMGYQKKLRSMTDKYRLHLSVADLLFVITLPFWAVDAVA
NWYFGNFLCKAVHVIYTVNLYSSVLILAFISLDRYLAIVHATNSQRPRKL
LAEKVVYVGLWIPALLLTIPDFIFANVSEADDRYICDRFYPNDLWVVVFQ
FQHIMVGLILPGIVILSCYCIIISKLSHSKGHQKRKALKTTVILILAFFA
CWLPYYIGISIDSFILLEIIKQGCEFENTVHKWISITEALAFFHCCLNPI
LYAFLGAKFKTSAQHALTSVSRGSSLKILSKGKRGGHSSVSTESESSSFH
SS

In one embodiment, the amino acid sequence of a CXCR4 A175F variant is:

Exemplary CXCR4 A175F variant
(SEQ ID NO: 6)
MEGISIYTSDNYTEEMGSGDYDSMKEPCFREENANFNKIFLPTIYSIIFL
TGIVGNGLVILVMGYQKKLRSMTDKYRLHLSVADLLFVITLPFWAVDAVA
NWYFGNFLCKAVHVIYTVNLYSSVLILAFISLDRYLAIVHATNSQRPRKL
LAEKVVYVGVWIPALLLTIPDFIFFNVSEADDRYICDRFYPNDLWVVVFQ
FQHIMVGLILPGIVILSCYCIIISKLSHSKGHQKRKALKTTVILILAFFA
CWLPYYIGISIDSFILLEIIKQGCEFENTVHKWISITEALAFFHCCLNPI
LYAFLGAKFKTSAQHALTSVSRGSSLKILSKGKRGGHSSVSTESESSSFH
SS

In one embodiment, the amino acid sequence of a CXCR4 Q200A variant is:

Exemplary CXCR4 Q200A variant
(SEQ ID NO: 7)
MEGISIYTSDNYTEEMGSGDYDSMKEPCFREENANFNKIFLPTIYSIIFL
TGIVGNGLVILVMGYQKKLRSMTDKYRLHLSVADLLFVITLPFWAVDAVA
NWYFGNFLCKAVHVIYTVNLYSSVLILAFISLDRYLAIVHATNSQRPRKL
LAEKVVYVGVWIPALLLTIPDFIFANVSEADDRYICDRFYPNDLWVVVFA
FQHIMVGLILPGIVILSCYCIIISKLSHSKGHQKRKALKTTVILILAFFA
CWLPYYIGISIDSFILLEIIKQGCEFENTVHKWISITEALAFFHCCLNPI
LYAFLGAKFKTSAQHALTSVSRGSSLKILSKGKRGGHSSVSTESESSSFH
SS

In one embodiment, the amino acid sequence of a CXCR4 H281A variant is:

Exemplary CXCR4 D262N variant
(SEQ ID NO: 8)
MEGISIYTSDNYTEEMGSGDYDSMKEPCFREENANFNKIFLPTIYSIIFL
TGIVGNGLVILVMGYQKKLRSMTDKYRLHLSVADLLFVITLPFWAVDAVA
NWYFGNFLCKAVHVIYTVNLYSSVLILAFISLDRYLAIVHATNSQRPRKL
LAEKVVYVGVWIPALLLTIPDFIFANVSEADDRYICDRFYPNDLWVVVFQ
FQHIMVGLILPGIVILSCYCIIISKLSHSKGHQKRKALKTTVILILAFFA
CWLPYYIGISINSFILLEIIKQGCEFENTVHKWISITEALAFFHCCLNPI
LYAFLGAKFKTSAQHALTSVSRGSSLKILSKGKRGGHSSVSTESESSSFH
SS

Exemplary CXCR4 H281A variant
(SEQ ID NO: 9)
MEGISIYTSDNYTEEMGSGDYDSMKEPCFREENANFNKIFLPTIYSIIFL
TGIVGNGLVILVMGYQKKLRSMTDKYRLHLSVADLLFVITLPFWAVDAVA
NWYFGNFLCKAVHVIYTVNLYSSVLILAFISLDRYLAIVHATNSQRPRKL
LAEKVVYVGVWIPALLLTIPDFIFANVSEADDRYICDRFYPNDLWVVVFQ
FQHIMVGLILPGIVILSCYCIIISKLSHSKGHQKRKALKTTVILILAFFA
CWLPYYIGISIDSFILLEIIKQGCEFENTVAKWISITEALAFFHCCLNPI
LYAFLGAKFKTSAQHALTSVSRGSSLKILSKGKRGGHSSVSTESESSSFH
SS

In one embodiment, the CXCR4 variant is encoded by a nucleotide sequence that has at least 70%, at least 80%, at least 85%, at least 90%, at least 95%, at least 96%, at least 97%, at least 98%, at least 99% or 100% identity to any of SEQ ID NOs: 18-22, preferably wherein the protein encoded by the nucleotide sequence substantially retains the engraftment enhancing activity of the protein represented by SEQ ID NO: 2.

In one embodiment, the CXCR4 variant is encoded by a nucleotide sequence that has at least 70%, at least 80%, at least 85%, at least 90%, at least 95%, at least 96%, at least 97%, at least 98%, at least 99% or 100% identity to any of SEQ ID NOs: 19-22, preferably wherein the protein encoded by the nucleotide sequence substantially retains the engraftment enhancing activity of the protein represented by SEQ ID NO: 2.

In one embodiment, the nucleotide sequence encoding a CXCR4 V160L variant is:

Exemplary nucleotide encoding a CXCR4 V160L
variant
(SEQ ID NO: 18)
ATGGAAGGCATCAGCATCTACACCAGCGACAACTACACCGAGGAAATGGG
CAGCGGCGACTACGACAGCATGAAGGAACCCTGCTTCCGGGAAGAGAACG
CCAACTTCAACAAGATCTTCCTGCCCACAATCTACAGCATCATCTTTCTG
ACCGGCATCGTGGGCAACGGACTCGTGATCCTCGTGATGGGCTACCAGAA
AAAGCTGCGGAGCATGACCGACAAGTACCGGCTGCACCTGAGCGTGGCCG
ACCTGCTGTTCGTGATCACCCTGCCTTTCTGGGCCGTGGACGCCGTGGCC
AATTGGTACTTCGGCAACTTCCTGTGCAAGGCCGTGCACGTGATCTACAC
AGTGAACCTGTACAGCAGCGTGCTGATCCTGGCCTTCATCAGCCTGGACA
GATACCTGGCCATCGTGCACGCCACCAACAGCCAGCGGCCTAGAAAGCTG
CTGGCCGAGAAGGTGGTGTACGTGGGCCTGTGGATTCCCGCCCTGCTGCT
GACCATCCCCGACTTCATCTTCGCCAACGTGTCCGAGGCCGACGACCGGT
ACATCTGCGACCGGTTCTACCCCAACGACCTGTGGGTGGTGGTGTTCCAG
TTCCAGCACATCATGGTGGGACTGATCCTGCCTGGCATCGTGATTCTGAG
CTGCTACTGCATCATCATCAGCAAGCTGAGCCACAGCAAGGGCCACCAGA
AGCGGAAGGCCCTGAAAACCACCGTGATCCTGATTCTGGCTTTCTTCGCC
TGCTGGCTGCCCTACTACATCGGCATCAGCATCGACAGCTTCATCCTGCT
GGAAATCATCAAGCAGGGCTGCGAGTTCGAGAACACCGTGCACAAGTGGA
TCAGCATTACCGAGGCCCTGGCCTTTTTCCACTGCTGCCTGAACCCTATC
CTGTACGCCTTCCTGGGCGCCAAGTTCAAGACCTCTGCCCAGCACGCCCT
GACCAGCGTGTCCAGAGGAAGCAGCCTGAAGATCCTGAGCAAGGGCAAGA
GAGGCGGCCACAGCTCCGTGTCTACAGAGAGCGAGAGCAGCAGCTTCCAC
AGCAGC

In one embodiment, the nucleotide sequence encoding a CXCR4 A175F variant is:

Exemplary nucleotide encoding a CXCR4 A175F
variant
(SEQ ID NO: 19)
ATGGAAGGCATCAGCATCTACACCAGCGACAACTACACCGAGGAAATGGG
CAGCGGCGACTACGACAGCATGAAGGAACCCTGCTTCCGGGAAGAGAACG
CCAACTTCAACAAGATCTTCCTGCCCACAATCTACAGCATCATCTTTCTG
ACCGGCATCGTGGGCAACGGACTCGTGATCCTCGTGATGGGCTACCAGAA
AAAGCTGCGGAGCATGACCGACAAGTACCGGCTGCACCTGAGCGTGGCCG
ACCTGCTGTTCGTGATCACCCTGCCTTTCTGGGCCGTGGACGCCGTGGCC
AATTGGTACTTCGGCAACTTCCTGTGCAAGGCCGTGCACGTGATCTACAC
AGTGAACCTGTACAGCAGCGTGCTGATCCTGGCCTTCATCAGCCTGGACA
GATACCTGGCCATCGTGCACGCCACCAACAGCCAGCGGCCTAGAAAGCTG
CTGGCCGAGAAGGTGGTGTACGTGGGCGTGTGGATTCCCGCCCTGCTGCT
GACCATCCCCGACTTCATCTTCTTCAACGTGTCCGAGGCCGACGACCGGT
ACATCTGCGACCGGTTCTACCCCAACGACCTGTGGGTGGTGGTGTTCCAG
TTCCAGCACATCATGGTGGGACTGATCCTGCCTGGCATCGTGATTCTGAG
CTGCTACTGCATCATCATCAGCAAGCTGAGCCACAGCAAGGGCCACCAGA
AGCGGAAGGCCCTGAAAACCACCGTGATCCTGATTCTGGCTTTCTTCGCC
TGCTGGCTGCCCTACTACATCGGCATCAGCATCGACAGCTTCATCCTGCT
GGAAATCATCAAGCAGGGCTGCGAGTTCGAGAACACCGTGCACAAGTGGA
TCAGCATTACCGAGGCCCTGGCCTTTTTCCACTGCTGCCTGAACCCTATC
CTGTACGCCTTCCTGGGCGCCAAGTTCAAGACCTCTGCCCAGCACGCCCT
GACCAGCGTGTCCAGAGGAAGCAGCCTGAAGATCCTGAGCAAGGGCAAGA
GAGGCGGCCACAGCTCCGTGTCTACAGAGAGCGAGAGCAGCAGCTTCCAC
AGCAGC

In one embodiment, the nucleotide sequence encoding a CXCR4 Q200A variant is:

Exemplary nucleotide encoding a CXCR4 Q200A
variant
(SEQ ID NO: 20)
ATGGAAGGCATCAGCATCTACACCAGCGACAACTACACCGAGGAAATGGG
CAGCGGCGACTACGACAGCATGAAGGAACCCTGCTTCCGGGAAGAGAACG
CCAACTTCAACAAGATCTTCCTGCCCACAATCTACAGCATCATCTTTCTG
ACCGGCATCGTGGGCAACGGACTCGTGATCCTCGTGATGGGCTACCAGAA
AAAGCTGCGGAGCATGACCGACAAGTACCGGCTGCACCTGAGCGTGGCCG
ACCTGCTGTTCGTGATCACCCTGCCTTTCTGGGCCGTGGACGCCGTGGCC
AATTGGTACTTCGGCAACTTCCTGTGCAAGGCCGTGCACGTGATCTACAC
AGTGAACCTGTACAGCAGCGTGCTGATCCTGGCCTTCATCAGCCTGGACA
GATACCTGGCCATCGTGCACGCCACCAACAGCCAGCGGCCTAGAAAGCTG
CTGGCCGAGAAGGTGGTGTACGTGGGCGTGTGGATTCCCGCCCTGCTGCT
GACCATCCCCGACTTCATCTTCGCCAACGTGTCCGAGGCCGACGACCGGT
ACATCTGCGACCGGTTCTACCCCAACGACCTGTGGGTGGTGGTGTTCGCG
TTCCAGCACATCATGGTGGGACTGATCCTGCCTGGCATCGTGATTCTGAG
CTGCTACTGCATCATCATCAGCAAGCTGAGCCACAGCAAGGGCCACCAGA
AGCGGAAGGCCCTGAAAACCACCGTGATCCTGATTCTGGCTTTCTTCGCC
TGCTGGCTGCCCTACTACATCGGCATCAGCATCGACAGCTTCATCCTGCT
GGAAATCATCAAGCAGGGCTGCGAGTTCGAGAACACCGTGCACAAGTGGA
TCAGCATTACCGAGGCCCTGGCCTTTTTCCACTGCTGCCTGAACCCTATC
CTGTACGCCTTCCTGGGCGCCAAGTTCAAGACCTCTGCCCAGCACGCCCT
GACCAGCGTGTCCAGAGGAAGCAGCCTGAAGATCCTGAGCAAGGGCAAGA
GAGGCGGCCACAGCTCCGTGTCTACAGAGAGCGAGAGCAGCAGCTTCCAC
AGCAGC

In one embodiment, the nucleotide sequence encoding a CXCR4 D262N variant is:

Exemplary nucleotide encoding a CXCR4 D262N
variant
(SEQ ID NO: 21)
ATGGAAGGCATCAGCATCTACACCAGCGACAACTACACCGAGGAAATGGG
CAGCGGCGACTACGACAGCATGAAGGAACCCTGCTTCCGGGAAGAGAACG
CCAACTTCAACAAGATCTTCCTGCCCACAATCTACAGCATCATCTTTCTG
ACCGGCATCGTGGGCAACGGACTCGTGATCCTCGTGATGGGCTACCAGAA
AAAGCTGCGGAGCATGACCGACAAGTACCGGCTGCACCTGAGCGTGGCCG
ACCTGCTGTTCGTGATCACCCTGCCTTTCTGGGCCGTGGACGCCGTGGCC
AATTGGTACTTCGGCAACTTCCTGTGCAAGGCCGTGCACGTGATCTACAC
AGTGAACCTGTACAGCAGCGTGCTGATCCTGGCCTTCATCAGCCTGGACA
GATACCTGGCCATCGTGCACGCCACCAACAGCCAGCGGCCTAGAAAGCTG
CTGGCCGAGAAGGTGGTGTACGTGGGCGTGTGGATTCCCGCCCTGCTGCT
GACCATCCCCGACTTCATCTTCGCCAACGTGTCCGAGGCCGACGACCGGT
ACATCTGCGACCGGTTCTACCCCAACGACCTGTGGGTGGTGGTGTTCCAG
TTCCAGCACATCATGGTGGGACTGATCCTGCCTGGCATCGTGATTCTGAG
CTGCTACTGCATCATCATCAGCAAGCTGAGCCACAGCAAGGGCCACCAGA
AGCGGAAGGCCCTGAAAACCACCGTGATCCTGATTCTGGCTTTCTTCGCC
TGCTGGCTGCCCTACTACATCGGCATCAGCATCAACAGCTTCATCCTGCT
GGAAATCATCAAGCAGGGCTGCGAGTTCGAGAACACCGTGCACAAGTGGA
TCAGCATTACCGAGGCCCTGGCCTTTTTCCACTGCTGCCTGAACCCTATC
CTGTACGCCTTCCTGGGCGCCAAGTTCAAGACCTCTGCCCAGCACGCCCT
GACCAGCGTGTCCAGAGGAAGCAGCCTGAAGATCCTGAGCAAGGGCAAGA
GAGGCGGCCACAGCTCCGTGTCTACAGAGAGCGAGAGCAGCAGCTTCCAC
AGCAGC

In one embodiment, the nucleotide sequence encoding a CXCR4 H281A variant is:

Exemplary nucleotide encoding a CXCR4 H281A
variant
(SEQ ID NO: 22)
ATGGAAGGCATCAGCATCTACACCAGCGACAACTACACCGAGGAAATGGG
CAGCGGCGACTACGACAGCATGAAGGAACCCTGCTTCCGGGAAGAGAACG
CCAACTTCAACAAGATCTTCCTGCCCACAATCTACAGCATCATCTTTCTG
ACCGGCATCGTGGGCAACGGACTCGTGATCCTCGTGATGGGCTACCAGAA
AAAGCTGCGGAGCATGACCGACAAGTACCGGCTGCACCTGAGCGTGGCCG
ACCTGCTGTTCGTGATCACCCTGCCTTTCTGGGCCGTGGACGCCGTGGCC
AATTGGTACTTCGGCAACTTCCTGTGCAAGGCCGTGCACGTGATCTACAC
AGTGAACCTGTACAGCAGCGTGCTGATCCTGGCCTTCATCAGCCTGGACA
GATACCTGGCCATCGTGCACGCCACCAACAGCCAGCGGCCTAGAAAGCTG
CTGGCCGAGAAGGTGGTGTACGTGGGCGTGTGGATTCCCGCCCTGCTGCT
GACCATCCCCGACTTCATCTTCGCCAACGTGTCCGAGGCCGACGACCGGT
ACATCTGCGACCGGTTCTACCCCAACGACCTGTGGGTGGTGGTGTTCCAG
TTCCAGCACATCATGGTGGGACTGATCCTGCCTGGCATCGTGATTCTGAG
CTGCTACTGCATCATCATCAGCAAGCTGAGCCACAGCAAGGGCCACCAGA
AGCGGAAGGCCCTGAAAACCACCGTGATCCTGATTCTGGCTTTCTTCGCC
TGCTGGCTGCCCTACTACATCGGCATCAGCATCGACAGCTTCATCCTGCT
GGAAATCATCAAGCAGGGCTGCGAGTTCGAGAACACCGTGGCCAAGTGGA
TCAGCATTACCGAGGCCCTGGCCTTTTTCCACTGCTGCCTGAACCCTATC
CTGTACGCCTTCCTGGGCGCCAAGTTCAAGACCTCTGCCCAGCACGCCCT
GACCAGCGTGTCCAGAGGAAGCAGCCTGAAGATCCTGAGCAAGGGCAAGA
GAGGCGGCCACAGCTCCGTGTCTACAGAGAGCGAGAGCAGCAGCTTCCAC
AGCAGC

Integrin alpha-4 (ITGA4)

In one aspect, the present invention provides use of ITGA4 (or a fragment or variant thereof) for increasing engraftment by haematopoietic stem and/or progenitor cells (HSPCs).

In one aspect, the present invention provides a method for increasing engraftment by haematopoietic stem and/or progenitor cells (HSPCs), wherein the method comprises the step of genetically engineering the HSPCs to express ITGA4 (or a fragment or variant thereof).

In one aspect, the present invention provides a population of genetically engineered haematopoietic stem and/or progenitor cells (HSPCs), wherein the HSPCs are genetically engineered to express ITGA4 (or a fragment or variant thereof).

In one aspect, the present invention provides a method for haematopoietic stem and/or progenitor cell (HSPC) transplantation comprising the steps:
(a) providing a population of HSPCs which are genetically engineered to express ITGA4 (or a fragment or variant thereof); and
(b) administering the HSPCs to a subject.

Integrin alpha-4 (ITGA4) is also known as CD49 antigen-like family member D, Integrin alpha-IV, and VLA-4 subunit alpha and makes up half of the a4p1 lymphocyte homing receptor.

In a preferred embodiment, the ITGA4 is human ITGA4. A human ITGA4 may have an amino acid sequence of UniProtKB P13612.

An exemplary ITGA4 polypeptide is provided by SEQ ID NO: 29. In one embodiment, the ITGA4 comprises or consists of an amino acid sequence that has at least 70%, at least 80%, at least 85%, at least 90%, at least 95%, at least 96%, at least 97%, at least 98%, at least 99% or 100% identity to SEQ ID NO: 29, preferably wherein the amino acid sequence substantially retains the engraftment enhancing activity of the protein represented by SEQ ID NO: 29.

In one embodiment, the amino acid sequence of ITGA4 is:

Exemplary ITGA4
(SEQ ID NO: 29)
MAWEARREPGPRRAAVRETVMLLLCLGVPTGRPYNVDTESALLYQGPHNT
LFGYSVVLHSHGANRWLLVGAPTANWLANASVINPGAIYRCRIGKNPGQT
CEQLQLGSPNGEPCGKTCLEERDNQWLGVTLSRQPGENGSIVTCGHRWKN
IFYIKNENKLPTGGCYGVPPDLRTELSKRIAPCYQDYVKKFGENFASCQA
GISSFYTKDLIVMGAPGSSYWTGSLFVYNITTNKYKAFLDKQNQVKFGSY
LGYSVGAGHFRSQHTTEVVGGAPQHEQIGKAYIFSIDEKELNILHEMKGK
KLGSYFGASVCAVDLNADGFSDLLVGAPMQSTIREEGRVFVYINSGSGAV
MNAMETNLVGSDKYAARFGESIVNLGDIDNDGFEDVAIGAPQEDDLQGAI
YIYNGRADGISSTFSQRIEGLQISKSLSMFGQSISGQIDADNNGYVDVAV
GAFRSDSAVLLRTRPVVIVDASLSHPESVNRTKFDCVENGWPSVCIDLTL
CFSYKGKEVPGYIVLFYNMSLDVNRKAESPPRFYFSSNGTSDVITGSIQV
SSREANCRTHQAFMRKDVRDILTPIQIEAAYHLGPHVISKRSTEEFPPLQ
PILQQKKEKDIMKKTINFARFCAHENCSADLQVSAKIGFLKPHENKTYLA
VGSMKTLMLNVSLFNAGDDAYETTLHVKLPVGLYFIKILELEEKQINCEV
TDNSGVVQLDCSIGYIYVDHLSRIDISFLLDVSSLSRAEEDLSITVHATC
ENEEEMDNLKHSRVTVAIPLKYEVKLTVHGFVNPTSFVYGSNDENEPETC
MVEKMNLTFHVINTGNSMAPNVSVEIMVPNSFSPQTDKLFNILDVQTTTG
ECHFENYQRVCALEQQKSAMQTLKGIVRFLSKTDKRLLYCIKADPHCLNF
LCNFGKMESGKEASVHIQLEGRPSILEMDETSALKFEIRATGFPEPNPRV
IELNKDENVAHVLLEGLHHQRPKRYFTIVIISSSLLLGLIVLLLISYVMW
KAGFFKRQYKSILQEENRRDSWSYINSKSNDD

In a preferred embodiment, the nucleotide sequence encoding the ITGA4 is codon optimised.

In one embodiment, the ITGA4 is encoded by a nucleotide sequence that has at least 70%, 80%, 90%, 95%, 96%, 97%, 98% 99% or 100% identity to SEQ ID NO: 30, preferably wherein the protein encoded by the nucleotide sequence substantially retains the engraftment enhancing activity of the protein represented by SEQ ID NO: 29.

In one embodiment, the nucleotide sequence encoding ITGA4 is:

Exemplary nucleotide sequence encoding ITGA4
(SEQ ID NO: 30)
ATGGCTTGGGAGGCTCGGAGAGAACCTGGACCTAGAAGAGCTGCCGTGCGGGAGACTGTCATGCTGCTGCTG
TGCCTGGGGGTGCCCACAGGCAGACCTTACAACGTGGATACCGAGAGCGCCCTGCTGTATCAGGGCCCCCAC
AACACCCTGTTTGGCTACTCTGTGGTGCTGCACAGCCACGGCGCCAACCGCTGGCTGCTGGTGGGCGCCCCC
ACCGCCAATTGGCTGGCCAATGCCTCCGTGATCAACCCAGGCGCCATCTACAGATGTCGGATCGGCAAGAAT
CCTGGCCAGACATGCGAGCAGCTGCAGCTGGGCTCCCCCAACGGCGAGCCTTGTGGCAAGACATGCCTGGAG
GAGAGGGACAATCAGTGGCTGGGCGTGACACTGAGCAGACAGCCCGGCGAGAACGGCTCCATCGTGACATGC
GGCCACAGATGGAAGAACATCTTTTACATCAAGAACGAGAATAAGCTGCCAACAGGCGGCTGCTATGGCGTG
CCCCCAGACCTGAGAACAGAGCTGAGCAAGCGGATCGCCCCATGCTACCAGGATTATGTGAAGAAGTTTGGC
GAGAATTTTGCCTCTTGCCAGGCCGGCATCTCCTCCTTCTACACCAAGGATCTGATCGTGATGGGCGCCCCT
GGCTCTTCCTATTGGACAGGCTCTCTGTTCGTGTATAATATCACAACCAACAAGTACAAGGCCTTCCTGGAC
AAGCAGAACCAGGTGAAGTTCGGCTCCTATCTGGGCTACAGCGTGGGCGCCGGCCACTTTCGGTCTCAGCAC
ACCACAGAGGTGGTGGGCGGCGCCCCCCAGCACGAGCAGATCGGCAAGGCCTACATCTTCTCCATCGACGAG
AAGGAGCTGAATATCCTGCACGAGATGAAGGGCAAGAAGCTGGGCTCCTACTTTGGCGCCTCCGTGTGCGCC
GTGGACCTGAATGCCGACGGCTTTTCCGACCTGCTGGTGGGCGCCCCAATGCAGTCCACAATCAGAGAGGAG
GGCAGAGTGTTCGTGTATATCAATTCCGGCAGCGGCGCCGTGATGAATGCCATGGAGACCAATCTGGTGGGC
TCCGACAAGTATGCCGCCAGATTCGGCGAGAGCATCGTGAATCTGGGCGACATCGACAACGATGGCTTCGAG
GACGTGGCCATCGGCGCCCCACAGGAGGATGACCTGCAGGGCGCCATCTATATCTATAACGGCCGGGCCGAC
GGCATCTCTAGCACCTTCTCCCAGAGAATCGAGGGCCTGCAGATCAGCAAGTCCCTGAGCATGTTCGGCCAG
AGCATCTCCGGCCAGATCGACGCCGATAATAACGGCTACGTGGATGTGGCCGTGGGCGCCTTTAGAAGCGAC
TCCGCCGTGCTGCTGAGAACAAGGCCCGTGGTGATCGTGGATGCCTCCCTGTCTCACCCCGAGTCCGTGAAT
CGGACAAAGTTTGACTGCGTGGAGAATGGCTGGCCAAGCGTGTGCATCGATCTGACACTGTGCTTTTCCTAT
AAGGGCAAGGAGGTGCCAGGCTATATCGTGCTGTTTTACAACATGTCTCTGGATGTGAACAGAAAGGCCGAG
TCCCCCCCAAGATTCTACTTTTCCTCTAACGGCACCTCTGATGTGATCACCGGCTCTATCCAGGTGTCCAGC
AGGGAGGCCAATTGCAGAACCCACCAGGCCTTTATGCGGAAGGATGTGCGCGACATCCTGACCCCAATCCAG
ATCGAGGCCGCCTATCACCTGGGCCCCCACGTGATCTCCAAGCGGTCCACCGAGGAGTTCCCTCCACTGCAG
CCAATCCTGCAGCAGAAGAAGGAGAAGGACATCATGAAGAAGACAATCAACTTCGCCAGGTTTTGCGCCCAC
GAGAACTGTTCCGCCGACCTGCAGGTGTCTGCCAAGATCGGCTTCCTGAAGCCCCACGAGAACAAGACATAT
CTGGCCGTGGGCTCCATGAAGACCCTGATGCTGAACGTGAGCCTGTTTAACGCCGGCGACGATGCCTACGAG
ACAACACTGCACGTGAAGCTGCCAGTGGGCCTGTACTTCATCAAGATCCTGGAGCTGGAGGAGAAGCAGATC
AACTGTGAGGTGACCGATAACTCCGGCGTGGTGCAGCTGGATTGCAGCATCGGCTATATCTACGTGGACCAC
CTGTCCCGCATCGACATCTCTTTTCTGCTGGACGTGTCCAGCCTGTCCCGGGCCGAGGAGGACCTGTCCATC
ACAGTGCACGCCACCTGCGAGAATGAGGAGGAGATGGACAACCTGAAGCACTCCAGAGTGACAGTGGCCATC
CCACTGAAGTACGAGGTGAAGCTGACAGTGCACGGCTTTGTGAATCCAACCTCCTTCGTGTACGGCTCCAAT
GACGAGAATGAGCCAGAGACATGTATGGTGGAGAAGATGAACCTGACATTTCACGTGATCAATACAGGCAAT
TCTATGGCCCCTAACGTGAGCGTGGAGATCATGGTGCCAAATTCTTTCAGCCCACAGACAGACAAGCTGTTT
AACATCCTGGACGTGCAGACAACCACAGGCGAGTGTCACTTTGAGAACTACCAGAGAGTGTGCGCCCTGGAG
CAGCAGAAGTCCGCCATGCAGACACTGAAGGGCATCGTGAGATTTCTGAGCAAGACAGATAAGAGGCTGCTG
TACTGCATCAAGGCCGATCCCCACTGCCTGAATTTTCTGTGCAACTTCGGCAAGATGGAGTCTGGCAAGGAG
GCCTCCGTGCACATCCAGCTGGAGGGCAGACCCTCCATCCTGGAGATGGACGAGACCAGCGCCCTGAAGTTC
GAGATCAGAGCCACAGGCTTCCCAGAGCCCAACCCCCGGGTGATCGAGCTGAACAAGGATGAGAACGTGGCC
CACGTGCTGCTGGAGGGCCTGCACCACCAGCGGCCCAAGAGATATTTCACCATCGTGATCATCTCCAGCTCT
CTGCTGCTGGGCCTGATCGTGCTGCTGCTGATCTCCTATGTGATGTGGAAGGCCGGCTTCTTTAAGCGGCAG
TACAAGTCCATCCTGCAGGAAGAAAATCGACGCGATTCATGGTCTTACATTAATTCTAAATCAAACGACGAC

The ITGA4 variant may be one which enhances engraftment to the same or a greater level than ITGA4. This may be determined by any suitable assay. For example, using a transmigration assay or by transplanting genetically engineered HSPCs and determining the extent of engraftment following transplantation.

The ITGA4 variant may have maintained or increased response to natural ligands (e.g. MAdCAM and VCAM) compared to ITGA4. In some embodiments, the ITGA4 variant has the same or greater binding affinity for natural ligands (e.g. MAdCAM and VCAM) compared to ITGA4. This may be determined by any suitable assay. For example, by using a binding assay e.g. as described in Darc, M., et al., 2011. PloS one, 6(9), p.e24461.

A person skilled in the art would be able to generate variants and/or fragments retaining the engraftment enhancing activity of ITGA4 based on conservative substitutions and/or the known structural and functional features of ITGA4. These are described, for instance in Yu, Y., et al., 2012. Journal of Cell Biology, 196(1), pp. 131-146.

In some embodiments, the ITGA4 variant has increased resistance to a VLA-4 antagonist compared to ITGA4. In some embodiments, the ITGA4 variant has a reduced binding affinity for a VLA-4 antagonist (e.g. natalizumab). This may be determined by any suitable assay. For example, using a transmigration assay in the absence or presence of a VLA-4 antagonist or using a binding assay e.g. as described in Darc, M., et al., 2011. PloS one, 6(9), p.e24461.

### Tyrosine-Protein Kinase KIT (KIT)

In one aspect, the present invention provides use of KIT (or a fragment or variant thereof) for increasing engraftment by haematopoietic stem and/or progenitor cells (HSPCs).

In one aspect, the present invention provides a method for increasing engraftment by haematopoietic stem and/or progenitor cells (HSPCs), wherein the method comprises the step of genetically engineering the HSPCs to express KIT (or a fragment or variant thereof).

In one aspect, the present invention provides a population of genetically engineered haematopoietic stem and/or progenitor cells (HSPCs), wherein the HSPCs are genetically engineered to express KIT (or a fragment or variant thereof).

In one aspect, the present invention provides a method for haematopoietic stem and/or progenitor cell (HSPC) transplantation comprising the steps:
(a) providing a population of HSPCs which are genetically engineered to express KIT (or a fragment or variant thereof); and
(b) administering the HSPCs to a subject.

Tyrosine-protein kinase KIT (KIT) is also known as mast/stem cell growth factor receptor Kit (EC: 2.7.10.1), SCFR, Piebald trait protein (PBT), Proto-oncogene c-Kit, Tyrosine-protein kinase Kit and CD117. KIT is a tyrosine-protein kinase that acts as cell-surface receptor for the cytokine KITLG/SCF and plays an essential role in the regulation of cell survival and proliferation, hematopoiesis, stem cell maintenance, gametogenesis, mast cell development, migration and function, and in melanogenesis.

In a preferred embodiment, the KIT is human KIT. A human KIT may have an amino acid sequence of UniProtKB P10721.

An exemplary KIT polypeptide is provided by SEQ ID NOs: 31 and 47. In one embodiment, the KIT comprises or consists of an amino acid sequence that has at least 70%, at least 80%, at least 85%, at least 90%, at least 95%, at least 96%, at least 97%, at least 98%, at least 99% or 100% identity to SEQ ID NO: 31 or 47, preferably wherein the amino acid sequence substantially retains the engraftment enhancing activity of the protein represented by SEQ ID NO: 31 or 47, respectively.

In one embodiment, the KIT comprises or consists of an amino acid sequence that has at least 70%, at least 80%, at least 85%, at least 90%, at least 95%, at least 96%, at least 97%, at least 98%, at least 99% or 100% identity to SEQ ID NO: 31, preferably wherein the amino acid sequence substantially retains the engraftment enhancing activity of the protein represented by SEQ ID NO: 31.

In one embodiment, the amino acid sequence of KIT is:

Exemplary KIT
(SEQ ID NO: 31)
MRGARGAWDFLCVLLLLLRVQTGSSQPSVSPGEPSPPSIHPGKSDLIVRVGDEIRLLCTDPGFVKWTFEILD
ETNENKQNEWITEKAEATNTGKYTCTNKHGLSNSIYVFVRDPAKLFLVDRSLYGKEDNDTLVRCPLTDPEVT
NYSLKGCQGKPLPKDLRFIPDPKAGIMIKSVKRAYHRLCLHCSVDQEGKSVLSEKFILKVRPAFKAVPVVSV
SKASYLLREGEEFTVTCTIKDVSSSVYSTWKRENSQTKLQEKYNSWHHGDFNYERQATLTISSARVNDSGVF
MCYANNTFGSANVTTTLEVVDKGFINIFPMINTTVFVNDGENVDLIVEYEAFPKPEHQQWIYMNRTFTDKWE
DYPKSENESNIRYVSELHLTRLKGTEGGTYTFLVSNSDVNAAIAFNVYVNTKPEILTYDRLVNGMLQCVAAG
FPEPTIDWYFCPGTEQRCSASVLPVDVQTLNSSGPPFGKLVVQSSIDSSAFKHNGTVECKAYNDVGKTSAYF
NFAFKGNNKEQIHPHTLFTPLLIGFVIVAGMMCIIVMILTYKYLQKPMYEVQWKVVEEINGNNYVYIDPTQL
PYDHKWEFPRNRLSFGKTLGAGAFGKVVEATAYGLIKSDAAMTVAVKMLKPSAHLTEREALMSELKVLSYLG
NHMNIVNLLGACTIGGPTLVITEYCCYGDLLNFLRRKRDSFICSKQEDHAEAALYKNLLHSKESSCSDSTNE
YMDMKPGVSYVVPTKADKRRSVRIGSYIERDVTPAIMEDDELALDLEDLLSFSYQVAKGMAFLASKNCIHRD
LAARNILLTHGRITKICDFGLARDIKNDSNYVVKGNARLPVKWMAPESIFNCVYTFESDVWSYGIFLWELFS
LGSSPYPGMPVDSKFYKMIKEGFRMLSPEHAPAEMYDIMKTCWDADPLKRPTFKQIVQLIEKQISESTNHIY
SNLANCSPNRQKPVVDHSVRINSVGSTASSSQPLLVHDDV

In one embodiment, the amino acid sequence of KIT is:

Exemplary KIT
(SEQ ID NO: 47)
MRGARGAWDFLCVLLLLLRVQTGSSQPSVSPGEPSPPSIHPGKSDLIVRVGDEIRLLCTDPGFVKWTFEILD
ETNENKQNEWITEKAEATNTGKYTCTNKHGLSNSIYVFVRDPAKLFLVDRSLYGKEDNDTLVRCPLTDPEVT
NYSLKGCQGKPLPKDLRFIPDPKAGIMIKSVKRAYHRLCLHCSVDQEGKSVLSEKFILKVRPAFKAVPVVSV
SKASYLLREGEEFTVTCTIKDVSSSVYSTWKRENSQTKLQEKYNSWHHGDFNYERQATLTISSARVNDSGVF
MCYANNTFGSANVTTTLEVVDKGFINIFPMINTTVFVNDGENVDLIVEYEAFPKPEHQQWIYMNRTFTDKWE
DYPKSENESNIRYVSELHLTRLKGTEGGTYTFLVSNSDVNAAIAFNVYVNTKPEILTYDRLVNGMLQCVAAG
FPEPTIDWYFCPGTEQRCSASVLPVDVQTLNSSGPPFGKLVVQSSIDSSAFKHNGTVECKAYNDVGKTSAYF
NFAFKEQIHPHTLFTPLLIGFVIVAGMMCIIVMILTYKYLQKPMYEVQWKVVEEINGNNYVYIDPTQLPYDH
KWEFPRNRLSFGKTLGAGAFGKVVEATAYGLIKSDAAMTVAVKMLKPSAHLTEREALMSELKVLSYLGNHMN
IVNLLGACTIGGPTLVITEYCCYGDLLNFLRRKRDSFICSKQEDHAEAALYKNLLHSKESSCSDSTNEYMDM
KPGVSYVVPTKADKRRSVRIGSYIERDVTPAIMEDDELALDLEDLLSFSYQVAKGMAFLASKNCIHRDLAAR
NILLTHGRITKICDFGLARDIKNDSNYVVKGNARLPVKWMAPESIFNCVYTFESDVWSYGIFLWELFSLGSS
PYPGMPVDSKFYKMIKEGFRMLSPEHAPAEMYDIMKTCWDADPLKRPTFKQIVQLIEKQISESTNHIYSNLA
NCSPNRQKPVVDHSVRINSVGSTASSSQPLLVHDDV

In a preferred embodiment, the nucleotide sequence encoding the KIT is codon optimised.

In one embodiment, the KIT is encoded by a nucleotide sequence that has at least 70%, 80%, 90%, 95%, 96%, 97%, 98% 99% or 100% identity to SEQ ID NO: 32, preferably wherein the protein encoded by the nucleotide sequence substantially retains the 40 engraftment enhancing activity of the protein represented by SEQ ID NO: 31.

In one embodiment, the nucleotide sequence encoding KIT is:

Exemplary nucleotide sequence encoding KIT
(SEQ ID NO: 32)
ATGAGAGGCGCTCGCGGCGCCTGGGATTTTCTCTGCGTTCTGCTCCTACTGCTTCGCGTCCAGACAGGCTCT
TCTCAACCATCTGTGAGTCCAGGGGAACCGTCTCCACCATCCATCCATCCAGGAAAATCAGACTTAATAGTC
CGCGTGGGCGACGAGATTAGGCTGTTATGCACTGATCCGGGCTTTGTCAAATGGACTTTTGAGATCCTGGAT
GAAACGAATGAGAATAAGCAGAATGAATGGATCACGGAAAAGGCAGAAGCCACCAACACCGGCAAATACACG
TGCACCAACAAACACGGCTTAAGCAATTCCATTTATGTGTTTGTTAGAGATCCTGCCAAGCTTTTCCTTGTT
GACCGCTCCTTGTATGGGAAAGAAGACAACGACACGCTGGTCCGCTGTCCTCTCACAGACCCAGAAGTGACC
AATTATTCCCTCAAGGGGTGCCAGGGGAAGCCTCTTCCCAAGGACTTGAGGTTTATTCCTGACCCCAAGGCG
GGCATCATGATCAAAAGTGTGAAACGCGCCTACCATCGGCTCTGTCTGCATTGTTCTGTGGACCAGGAGGGC
AAGTCAGTGCTGTCGGAAAAATTCATCCTGAAAGTGAGGCCAGCCTTCAAAGCTGTGCCTGTTGTGTCTGTG
TCCAAAGCAAGCTATCTTCTTAGGGAAGGGGAAGAATTCACAGTGACGTGCACAATAAAAGATGTGTCTAGT
TCTGTGTACTCAACGTGGAAAAGAGAAAACAGTCAGACTAAACTACAGGAGAAATATAATAGCTGGCATCAC
GGTGACTTCAATTATGAACGTCAGGCAACGTTGACTATCAGTTCAGCGAGAGTTAATGATTCTGGAGTGTTC
ATGTGTTATGCCAATAATACTTTTGGATCAGCAAATGTCACAACAACCTTGGAAGTAGTAGATAAAGGATTC
ATTAATATCTTCCCCATGATAAACACTACAGTATTTGTAAACGATGGAGAAAATGTAGATTTGATTGTTGAA
TATGAAGCATTCCCCAAACCTGAACACCAGCAGTGGATCTATATGAACAGAACCTTCACTGATAAATGGGAA
GATTATCCCAAGTCTGAGAATGAAAGTAATATCAGATACGTAAGTGAACTTCATCTAACGAGATTAAAAGGC
ACCGAAGGAGGCACTTACACATTCCTAGTGTCCAATTCTGACGTCAATGCTGCCATAGCATTTAATGTTTAT
GTGAATACAAAACCAGAAATCCTGACTTACGACAGGCTCGTGAATGGCATGCTCCAATGTGTGGCAGCAGGA
TTCCCAGAGCCCACAATAGATTGGTATTTTTGTCCAGGAACTGAGCAGAGATGCTCTGCTTCTGTACTGCCA
GTGGATGTGCAGACACTAAACTCATCTGGGCCACCGTTTGGAAAGCTAGTGGTTCAGAGTTCTATAGATTCT
AGTGCATTCAAGCACAATGGCACGGTTGAATGTAAGGCTTACAACGATGTGGGCAAGACTTCTGCCTATTTT
AACTTTGCATTTAAAGGTAACAACAAAGAGCAAATCCATCCCCACACCCTGTTCACTCCTTTGCTGATTGGT
TTCGTAATCGTAGCTGGCATGATGTGCATTATTGTGATGATTCTGACCTACAAATATTTACAGAAACCCATG
TATGAAGTACAGTGGAAGGTTGTTGAGGAGATAAATGGAAACAATTATGTTTACATAGACCCAACACAACTT
CCTTATGATCACAAATGGGAGTTTCCCAGAAACAGGCTGAGTTTTGGGAAAACCCTGGGTGCTGGAGCTTTC
GGGAAGGTTGTTGAGGCAACTGCTTATGGCTTAATTAAGTCAGATGCGGCCATGACTGTCGCTGTAAAGATG
CTCAAGCCGAGTGCCCATTTGACAGAACGGGAAGCCCTCATGTCTGAACTCAAAGTCCTGAGTTACCTTGGT
AATCACATGAATATTGTGAATCTACTTGGAGCCTGCACCATTGGAGGGCCCACCCTGGTCATTACAGAATAT
TGTTGCTATGGTGATCTTTTGAATTTTTTGAGAAGAAAACGTGATTCATTTATTTGTTCAAAGCAGGAAGAT
CATGCAGAAGCTGCACTTTATAAGAATCTTCTGCATTCAAAGGAGTCTTCCTGCAGCGATAGTACTAATGAG
TACATGGACATGAAACCTGGAGTTTCTTATGTTGTCCCAACCAAGGCCGACAAAAGGAGATCTGTGAGAATA
GGCTCATACATAGAAAGAGATGTGACTCCCGCCATCATGGAGGATGACGAGTTGGCCCTAGACTTAGAAGAC
TTGCTGAGCTTTTCTTACCAGGTGGCAAAGGGCATGGCTTTCCTCGCCTCCAAGAATTGTATTCACAGAGAC
TTGGCAGCCAGAAATATCCTCCTTACTCATGGTCGGATCACAAAGATTTGTGATTTTGGTCTAGCCAGAGAC
ATCAAGAATGATTCTAATTATGTGGTTAAAGGAAACGCTCGACTACCTGTGAAGTGGATGGCACCTGAAAGC
ATTTTCAACTGTGTATACACGTTTGAAAGTGACGTCTGGTCCTATGGGATTTTTCTTTGGGAGCTGTTCTCT
TTAGGAAGCAGCCCCTATCCTGGAATGCCGGTCGATTCTAAGTTCTACAAGATGATCAAGGAAGGCTTCCGG
ATGCTCAGCCCTGAACACGCACCTGCTGAAATGTATGACATAATGAAGACTTGCTGGGATGCAGATCCCCTA
AAAAGACCAACATTCAAGCAAATTGTTCAGCTAATTGAGAAGCAGATTTCAGAGAGCACCAATCATATTTAC
TCCAACTTAGCAAACTGCAGCCCCAACCGACAGAAGCCCGTGGTAGACCATTCTGTGCGGATCAATTCTGTC
GGCAGCACCGCTTCCTCCTCCCAGCCTCTGCTTGTGCACGACGATGTC

The KIT variant may be one which enhances engraftment to the same or a greater level than KIT. This may be determined by any suitable assay. For example, using a transmigration assay or by transplanting genetically engineered HSPCs and determining the extent of engraftment following transplantation.

The KIT variant may have maintained or increased response to SCF compared to KIT. In some embodiments, the KIT variant has the same or greater binding affinity for SCF compared to KIT. This may be determined by any suitable assay. For example, by using SCF binding and dimerization assays e.g. as described in Lemmon, M. A., et al., 1997. Journal of Biological Chemistry, 272(10), pp. 6311-6317.

A person skilled in the art would be able to generate variants and/or fragments retaining the engraftment enhancing activity of KIT based on conservative substitutions and/or the known structural and functional features of KIT. These are described, for instance in Liu, H., et al., 2007. The EMBO journal, 26(3), pp. 891-901.

In some embodiments, the KIT variant has increased resistance to a KIT-directed antibody or immunotoxin compared to KIT. In some embodiments, the KIT variant has a reduced binding affinity for a KIT-directed antibody or immunotoxin compared to KIT. This may be determined by any suitable assay. For example, using a transmigration assay in the absence or presence of a KIT-directed antibody or immunotoxin or using cell proliferation and c-KIT autophosphorylation assays e.g. as described in Roberts, K. G., et al., 2007. Molecular cancer therapeutics, 6(3), pp. 1159-1166.

### Cluster of Differentiation 47 (CD47)

In one aspect, the present invention provides use of CD47 (or a fragment or variant thereof) for increasing engraftment by haematopoietic stem and/or progenitor cells (HSPCs).

In one aspect, the present invention provides a method for increasing engraftment by haematopoietic stem and/or progenitor cells (HSPCs), wherein the method comprises the step of genetically engineering the HSPCs to express CD47 (or a fragment or variant thereof).

In one aspect, the present invention provides a population of genetically engineered haematopoietic stem and/or progenitor cells (HSPCs), wherein the HSPCs are genetically engineered to express CD47 (or a fragment or variant thereof).

In one aspect, the present invention provides a method for haematopoietic stem and/or progenitor cell (HSPC) transplantation comprising the steps:
(a) providing a population of HSPCs which are genetically engineered to express CD47 (or a fragment or variant thereof); and
(b) administering the HSPCs to a subject.

Cluster of differentiation 47 (CD47; also known as integrin-associated protein, IAP) is a transmembrane protein belonging to the immunoglobulin superfamily. CD47 binds thrombospondin-1 (TSP-1) and signal-regulatory protein alpha (SIRPa), and functions as a signal to macrophages. Mouse, rat, bovine and human CD47 have been cloned and show about 70% overall amino acid identity (see e.g. Brown, E. J. and Frazier, W. A., 2001. Trends in cell biology, 11(3), pp. 130-135).

In a preferred embodiment, the CD47 is human CD47. A human CD47 may have an amino acid sequence of UniProtKB Q08722.

Exemplary CD47 polypeptides are provided by SEQ ID NOs: 23-26. In one embodiment, the CD47 comprises or consists of an amino acid sequence that has at least 70%, at least 80%, at least 85%, at least 90%, at least 95%, at least 96%, at least 97%, at least 98%, at least 99% or 100% identity to any of SEQ ID NOs: 23-26, preferably wherein the amino acid sequence substantially retains the engraftment enhancing activity of the protein represented by SEQ ID NOs: 23-26, respectively.

In one embodiment, the amino acid sequence of CD47 is:

Exemplary CD47
(SEQ ID NO: 23)
MWPLVAALLLGSACCGSAQLLFNKTKSVEFTFCNDTVVIPCFVTNMEAQNTTEVYVKWKFKGRDIYTFDGAL
NKSTVPTDESSAKIEVSQLLKGDASLKMDKSDAVSHTGNYTCEVTELTREGETIIELKYRVVSWESPNENIL
IVIFPIFAILLFWGQFGIKTLKYRSGGMDEKTIALLVAGLVITVIVIVGAILFVPGEYSLKNATGLGLIVTS
TGILILLHYYVFSTAIGLTSFVIAILVIQVIAYILAVVGLSLCIAACIPMHGPLLISGLSILALAQLLGLVY
MKFVASNQKTIQPPRKAVEEPLNAFKESKGMMNDE

In another embodiment, the amino acid sequence of CD47 is:

Exemplary CD47
(SEQ ID NO: 24)
MWPLVAALLLGSACCGSAQLLENKTKSVEFTFCNDTVVIPCFVTNMEAQNTTEVYVKWKFKGRDIYTFDGAL
NKSTVPTDESSAKIEVSQLLKGDASLKMDKSDAVSHTGNYTCEVTELTREGETIIELKYRVVSWFSPNENIL
IVIFPIFAILLFWGQFGIKTLKYRSGGMDEKTIALLVAGLVITVIVIVGAILFVPGEYSLKNATGLGLIVTS
TGILILLHYYVFSTAIGLTSFVIAILVIQVIAYILAVVGLSLCIAACIPMHGPLLISGLSILALAQLLGLVY
MKFV

In another embodiment, the amino acid sequence of CD47 is:

Exemplary CD47
(SEQ ID NO: 25)
MWPLVAALLLGSACCGSAQLLFNKTKSVEFTFCNDTVVIPCFVTNMEAQNTTEVYVKWKFKGRDIYTEDGAL
NKSTVPTDESSAKIEVSQLLKGDASLKMDKSDAVSHTGNYTCEVTELTREGETIIELKYRVVSWFSPNENIL
IVIFPIFAILLFWGQFGIKTLKYRSGGMDEKTIALLVAGLVITVIVIVGAILFVPGEYSLKNATGLGLIVTS
TGILILLHYYVESTAIGLTSFVIAILVIQVIAYILAVVGLSLCIAACIPMHGPLLISGLSILALAQLLGLVY
MKFVASNQKTIQPPRNN

In another embodiment, the amino acid sequence of CD47 is:

Exemplary CD47
(SEQ ID NO: 26)
MWPLVAALLLGSACCGSAQLLFNKTKSVEFTFCNDTVVIPCFVTNMEAQNTTEVYVKWKFKGRDIYTEDGAL
NKSTVPTDESSAKIEVSQLLKGDASLKMDKSDAVSHTGNYTCEVTELTREGETIIELKYRVVSWESPNENIL
IVIFPIFAILLFWGQFGIKTLKYRSGGMDEKTIALLVAGLVITVIVIVGAILFVPGEYSLKNATGLGLIVTS
TGILILLHYYVFSTAIGLTSFVIAILVIQVIAYILAVVGLSLCIAACIPMHGPLLISGLSILALAQLLGLVY
MKFVASNQKTIQPPRKAVEEPLN

In a preferred embodiment, the nucleotide sequence encoding the CD47 is codon optimised.

In one embodiment, the CD47 is encoded by a nucleotide sequence that has at least 70%, 80%, 90%, 95%, 96%, 97%, 98% 99% or 100% identity to SEQ ID NO: 27 or 28, preferably wherein the protein encoded by the nucleotide sequence substantially retains the engraftment enhancing activity of the protein represented by SEQ ID NO: 23.

In one embodiment, the nucleotide sequence encoding CD47 is:

Exemplary nucleotide sequence encoding CD47
(SEQ ID NO: 27)
ATGTGGCCTCTCGTGGCCGCTCTGCTGCTCGGGAGCGCTTGTTGCGGCAGCGCCCAGCTGCTGTTCAACAAA
ACCAAGTCCGTCGAGTTCACCTTCTGCAACGACACAGTGGTGATCCCCTGCTTCGTCACCAACATGGAGGCT
CAGAATACCACCGAGGTCTACGTCAAGTGGAAATTCAAGGGCAGAGACATCTACACCTTCGACGGAGCCCTC
AACAAGAGCACAGTGCCTACCGACTTTTCCAGCGCCAAGATTGAGGTGAGCCAACTCCTGAAGGGAGACGCC
AGCCTGAAGATGGACAAGAGCGATGCCGTCAGCCACACAGGAAACTACACCTGCGAGGTGACAGAGCTCACC
AGAGAGGGCGAGACCATCATCGAGCTCAAATACAGAGTGGTGTCCTGGTTCTCCCCCAACGAGAACATCCTC
ATCGTGATCTTCCCCATCTTCGCCATCCTGCTGTTCTGGGGCCAGTTCGGCATCAAAACCCTGAAGTATAGA
TCCGGCGGCATGGACGAGAAAACAATCGCCCTGCTGGTGGCCGGCCTCGTGATTACCGTGATCGTCATCGTG
GGCGCCATCCTCTTCGTGCCCGGAGAGTACAGCCTCAAGAACGCCACCGGCCTGGGCCTGATTGTGACCTCC
ACAGGCATTCTGATCCTGCTGCACTACTACGTGTTCAGCACAGCCATTGGCCTCACAAGCTTCGTGATCGCC
ATCCTGGTCATCCAGGTGATCGCCTACATCCTCGCCGTGGTCGGACTCAGCCTCTGTATTGCCGCTTGCATC
CCCATGCACGGACCCCTCCTGATCTCCGGCCTCAGCATTCTGGCTCTCGCTCAGCTGCTCGGCCTGGTGTAC
ATGAAGTTCGTCGCCAGCAACCAGAAGACCATCCAACCCCCCAGAAAGGCCGTCGAAGAGCCTCTGAACGCC
TTTAAGGAGAGCAAGGGCATGATGAACGACGAG

In another embodiment, the nucleotide sequence encoding CD47 is:

Exemplary nucleotide sequence encoding CD47
(SEQ ID NO: 28)
ATGTGGCCCCTGGTAGCGGCGCTGTTGCTGGGCTCGGCGTGCTGCGGATCAGCTCAGCTACTATTTAATAAA
ACAAAATCTGTAGAATTCACGTTTTGTAATGACACTGTCGTCATTCCATGCTTTGTTACTAATATGGAGGCA
CAAAACACTACTGAAGTATACGTAAAGTGGAAATTTAAAGGAAGAGATATTTACACCTTTGATGGAGCTCTA
AACAAGTCCACTGTCCCCACTGACTTTAGTAGTGCAAAAATTGAAGTCTCACAATTACTAAAAGGAGATGCC
TCTTTGAAGATGGATAAGAGTGATGCTGTCTCACACACAGGAAACTACACTTGTGAAGTAACAGAATTAACC
AGAGAAGGTGAAACGATCATCGAGCTAAAATATCGTGTTGTTTCATGGTTTTCTCCAAATGAAAATATTCTT
ATTGTTATTTTCCCAATTTTTGCTATACTCCTGTTCTGGGGACAGTTTGGTATTAAAACACTTAAATATAGA
TCCGGTGGTATGGATGAGAAAACAATTGCTTTACTTGTTGCTGGACTAGTGATCACTGTCATTGTCATTGTT
GGAGCCATTCTTTTCGTCCCAGGTGAATATTCATTAAAGAATGCTACTGGCCTTGGTTTAATTGTGACTTCT
ACAGGGATATTAATATTACTTCACTACTATGTGTTTAGTACAGCGATTGGATTAACCTCCTTCGTCATTGCC
ATATTGGTTATTCAGGTGATAGCCTATATCCTCGCTGTGGTTGGACTGAGTCTCTGTATTGCGGCGTGTATA
CCAATGCATGGCCCTCTTCTGATTTCAGGTTTGAGTATCTTAGCTCTAGCACAATTACTTGGACTAGTTTAT
ATGAAATTTGTGGCTTCCAATCAGAAGACTATACAACCTCCTAGGAAAGCTGTAGAGGAACCCCTTAATGCA
TTCAAAGAATCAAAAGGAATGATGAATGATGAA

The CD47 variant may be one which enhances engraftment to the same or a greater level than CD47. This may be determined by any suitable assay. For example, using a transmigration assay or by transplanting genetically engineered HSPCs and determining the extent of engraftment following transplantation.

The CD47 variant may have maintained or increased response to its natural ligands (e.g. TSP-1, SIRPα, and/or integrins) compared to CD47. In some embodiments, the CD47 variant has the same or greater binding affinity for its natural ligands compared to CD47. This may be determined by any suitable assay. For example, by using a binding assay.

A person skilled in the art would be able to generate variants and/or fragments retaining the engraftment enhancing activity of CD47 based on conservative substitutions and/or the known structural and functional features of CD47. These are described, for instance in Fenalti, G., et al., 2021. Nature communications, 12(1), pp. 1-14.

### RNA Polynucleotide

In one aspect, the present invention provides an RNA polynucleotide comprising a protein-coding sequence. Preferably, the protein-coding sequence encodes an engraftment enhancer.

As used herein, an “RNA polynucleotide” may refer to a polynucleotide which consists substantially of ribonucleotides, which are nucleotides containing ribose as its pentose component. The RNA polynucleotide may be messenger RNA (mRNA).

### Structural Elements

The RNA polynucleotide may comprise one or more structural elements for improving stability and translation efficiency.

Any suitable structural elements may be used. Modifying mRNA structural elements, particularly the 5′ cap, 5′- and 3′-untranslated regions (UTRs), the coding region, and polyadenylation tail, may help improve its intracellular stability and translational efficiency.

The structural elements may be operably linked to the protein-coding sequence, when appropriate. The term “operably linked” may mean that the components described are in a relationship permitting them to function in their intended manner.

### Kozak Sequence

The RNA polynucleotide of the present invention may comprise a Kozak sequence. Suitably, the protein-coding sequence is operably linked to a Kozak sequence. A Kozak sequence may be inserted before the start codon to improve the initiation of translation.

Suitable Kozak sequences will be well known to those of skill in the art (see e.g. Kozak, M., 1987. Nucleic acids research, 15(20), pp. 8125-8148).

In some embodiments, the Kozak sequence comprises or consists of a nucleotide sequence which is at least 80% identical to SEQ ID NO: 38 or a fragment thereof. In some embodiments, the Kozak sequence comprises or consists of the nucleotide sequence SEQ ID NO: 38 or a fragment thereof.

Exemplary Kozak sequence
(SEQ ID NO: 38)
CCACC

eIF4F Aptamer

The RNA polynucleotide of the present invention may comprise a translation non-blocking eIF4F aptamer. Suitably, the protein-coding sequence is operably linked a translation non-blocking eIF4F aptamer. A translation non-blocking eIF4F aptamer may be inserted in the 5′-UTR to improve the initiation of translation.

Eukaryotic initiation factor 4F (eIF4F) is a heterotrimeric protein complex that binds the 5′ cap of mRNAs to promote translation initiation. The eIF4F complex is composed of three non-identical subunits: the DEAD-box RNA helicase eIF4A, the cap-binding protein eIF4E, and the large “scaffold” protein eIF4G.

A “translation non-blocking eIF4F aptamer” may refer to an aptamer sequence which binds to eIF4F complex but does not inhibit translation. In some embodiments, a translation non-blocking eIF4F aptamer promotes initiation of translation. Suitable translation non-blocking eIF4F aptamers are described in WO 2019/081383A1.

In preferred embodiments, the translation non-blocking eIF4F aptamer is a translation non-blocking eIF4G aptamer (i.e. the aptamer binds to subunit eIF4G). In some embodiments, the translation non-blocking eIF4F aptamer is a translation non-blocking eIF4A aptamer (i.e. the aptamer binds to subunit eIF4A). In some embodiments, the translation non-blocking eIF4F aptamer is a translation non-blocking eIF4E aptamer (i.e. the aptamer binds to subunit eIF4E).

Exemplary translation non-blocking eIF4F aptamers are shown in SEQ ID NOs: 33-36.

In some embodiments, the translation non-blocking eIF4F aptamer comprises or consists of a nucleotide sequence having at least 90%, at least 92%, at least 94%, at least 96%, at least 98%, or 100% identity to any of SEQ ID NOs: 33-36. In some embodiments, the translation non-blocking eIF4F aptamer comprises or consists of the nucleotide sequence of any of SEQ ID NOs: 33-36.

In some embodiments, the translation non-blocking eIF4F aptamer comprises or consists of a nucleotide sequence having at least 90%, at least 92%, at least 94%, at least 96%, at least 98%, or 100% identity to SEQ ID NO: 34. In some embodiments, the translation non-blocking eIF4F aptamer comprises or consists of the nucleotide sequence of SEQ ID NO: 34.

Exemplary translation non-blocking eIF4G aptamer
(SEQ ID NO: 33)
ACUCACUAUUUGUUUUCGCGCCCAGUUGCAAAAAGUGUCG
Exemplary translation non-blocking eIF4G aptamer
(SEQ ID NO: 34)
GACUCACUAUUUGUUUUCGCGCCCAGUUGCAAAAAGUGUCG
Exemplary translation non-blocking eIF4G aptamer
(SEQ ID NO: 35)
UCCGCGGCGCCAUCUCAUGUUUAGUUGUCCUAUGUCGAGC
Exemplary translation non-blocking eIF4G aptamer
(SEQ ID NO: 36)
UCCGUAGAAACGCGUUAAGGUGAAAGUUUGAGGGCUCCUCA

### Woodchuck Hepatitis Virus Post-Transcriptional Regulatory Element (WPRE)

The RNA polynucleotide of the present invention may comprise a woodchuck hepatitis virus post-transcriptional regulatory element (WPRE). Suitably, the protein-coding sequence is operably linked to a WPRE. A WPRE may be inserted in the 3′UTR to improve the initiation of translation.

Suitable WPRE sequences will be well known to those of skill in the art (see e.g. Zufferey, R., et al., 1999. Journal of virology, 73(4), pp. 2886-2892; and Zanta-Boussif, M. A. et al., 2009. Gene therapy, 16(5), pp. 605-619). Suitably, the WPRE is a wild-type WPRE or is a mutant WPRE. For example, the WPRE may be mutated to abrogate translation of the woodchuck hepatitis virus X protein (WHX) e.g. by mutating the WHX ORF translation start codon.

In some embodiments, the WPRE comprises or consists of a nucleotide sequence which is at least 70% identical to SEQ ID NO: 37 or a fragment thereof. Suitably, the WPRE comprises or consists of a nucleotide sequence which is at least 75%, at least 80%, at least 85%, at least 90%, at least 95%, at least 96%, at least 97%, at least 98%, at least 99%, or 100% identical to SEQ ID NO: 37 or a fragment thereof.

In some embodiments, the WPRE comprises or consists of the nucleotide sequence SEQ ID NO: 37 or a fragment thereof.

Exemplary WPRE
(SEQ ID NO: 37)
AAUCAACCUCUGGAUUACAAAAUUUGUGAAAGAUUGACUGGUAUUCUUAACUAUGUUGCUCCUUUUACG
CUAUGUGGAUACGCUGCUUUAAUGCCUUUGUAUCAUGCUAUUGCUUCCCGUAUGGCUUUCAUUUUCUCC
UCCUUGUAUAAAUCCUGGUUGCUGUCUCUUUAUGAGGAGUUGUGGCCCGUUGUCAGGCAACGUGGCGUG
GUGUGCACUGUGUUUGCUGACGCAACCCCCACUGGUUGGGGCAUUGCCACCACCUGUCAGCUCCUUUCC
GGGACUUUCGCUUUCCCCCUCCCUAUUGCCACGGCGGAACUCAUCGCCGCCUGCCUUGCCCGCUGCUGG
ACAGGGGCUCGGCUGUUGGGCACUGACAAUUCCGUGGUGUUGUCGGGGAAAUCAUCGUCCUUUCCUUGG
CUGCUCGCCUGUGUUGCCACCUGGAUUCUGCGCGGGACGUCCUUCUGCUACGUCCCUUCGGCCCUCAAU
CCAGCGGACCUUCCUUCCCGCGGCCUGCUGCCGGCUCUGCGGCCUCUUCCGCGUCUUCGCCUUCGCCCU
CAGACGAGUCGGAUCUCCCUUUGGGCCGCCUCCCCGCCUG

### Polyadenylation Tail

The RNA polynucleotide of the present invention may comprise a polyadenylation (poly(A)) tail. Suitably, the protein-coding sequence is operably linked to a poly(A) tail.

A poly(A) tail typically consists of multiple adenosine monophosphates and is found at the 3′ end of mRNA. A poly(A) tail may be important for the nuclear export, translation and stability of mRNA. Although a poly(A) tail typically consists of multiple adenosine monophosphates, other adenosine monophosphate derivatives may be present (see e.g. Strzelecka, D., et al., 2020. RNA, 26(12), pp. 1815-1837).

In some embodiments, the polyA tail is at least about 100, at least about 110, at least about 120, at least about 130, at least about 140, or at least about 150 nucleotides in length. In some embodiments, the polyA tail is about 100 to about 150, about 110 to about 150, or about 120 to about 150 nucleotides in length. In some embodiments, the polyA tail is about 120 nucleotides in length.

In some embodiments, the polyA tail consists of (A)ₓ wherein x≥100, x≥110, x≥120, x≥130, x≥140, or x≥150. In some embodiments, the polyA tail consists of (A)ₓ wherein x=100±5, x=110±5, x=120±5, x=130±5, x=140±5, or x=150±5. In some embodiments, x=120±5.

### 5′ Cap

The RNA polynucleotide of the present invention may comprise a 5′ cap.

A 5′ cap is a specially altered nucleotide on the 5′ end of some primary transcripts such as precursor messenger RNA that may allow for stable and mature messenger RNA to undergo translation during protein synthesis.

Typically, a 5′ cap may consist of a guanine nucleotide connected to mRNA via an unusual 5′ to 5′ triphosphate linkage. This guanosine may be methylated on the 7 position directly after capping and is referred to as a 7-methylguanylate cap, abbreviated m7G. Further modifications exist including the methylation of the 2′ hydroxy-groups of the first 2 ribose sugars of the 5′ end of the mRNA. RNA polynucleotides may be capped co-transcriptionally by using a cap analog or separately using a capping enzyme.

In some embodiments, the RNA polynucleotide of the present invention comprises a 5′ cap analogue. Cap analogues can allow for: (i) high incorporation efficiencies when added to IVT, (ii) correct orientation when incorporated into RNA, (iii) strong binding to the cap-binding protein eIF4E, (iv) inhibitory potential when added as competitor in an in vitro translation assay and (v) high translation efficiency of resulting capped RNA. Standard cap analogues include m⁷GpppG and GpppG (see e.g. Muttach, F., et al., 2017. Beilstein journal of organic chemistry, 13(1), pp. 2819-2832). Suitable 5′ cap analogues include G(5′)ppp(5′)G, m⁷G(5′)ppp(5′)G, 3′-O-Me-m⁷G(5′)ppp(5′)G, m₃^{2,2,7}G(5′)ppp(5′)G, m₂^{7,3}′° G(5′)ppp(5′)G, G(5′)ppp(5′)A, m⁷G(5′)ppp(5′)A, m⁷G(5′)ppp(5′)(2′OMeA)pG, and the like.

In some embodiments, the RNA polynucleotide of the present invention comprises a 5′ cap comprising or consisting of m⁷G(5′)ppp(5′)(2′OMeA)pG. This 5′ cap may be commercially available as the CleanCap® Reagent AG.

### Modified Nucleobases

The RNA polynucleotide of the present invention may comprise one or more modified nucleobases.

RNA polynucleotides typically comprise the four canonical bases guanine, uracil, adenine, and cytosine, but these bases and attached sugars can be modified in numerous ways. There are more than 100 naturally occurring modified nucleosides (see e.g. Cantara, W. A., et al., 2010. Nucleic acids research, 39(suppl_1), pp. D195-D201). Modified bases can be introduced during in vitro transcription using modified nucleotides in place of canonical nucleotides.

In some embodiments, the RNA polynucleotide of the present invention comprises one or more modified nucleobase. Suitably, all of the guanines are replaced with modified guanine (i.e. the only guanine present in the RNA polynucleotide is modified), all of the uracils are replaced with modified uracil (i.e. the only uracil present in the RNA polynucleotide is modified), all of the adenines are replaced with modified adenine (i.e. the only adenine present in the RNA polynucleotide is modified), and/or all of the cytosines are replace with modified cytosine (i.e. the only cytosine present in the RNA polynucleotide is modified).

In some embodiments, the RNA polynucleotide of the present invention comprises one or more modified nucleoside. Suitably, all of the guanosines are replaced with modified guanosine (i.e. the guanosine uracil present in the RNA polynucleotide is modified), all of the uridines are replaced with modified uridine (i.e. the only uridine present in the RNA polynucleotide is modified), all of the adenosines are replaced with modified adenosine (i.e. the only adenosine present in the RNA polynucleotide is modified), and/or all of the cytidines are replace with modified cytidine (i.e. the only cytidine present in the RNA polynucleotide is modified).

In some embodiments, the RNA polynucleotide of the present invention comprises modified guanine. In some embodiments, all of the guanines are replaced with modified guanine. In some embodiments, the RNA polynucleotide of the present invention comprises modified guanosine. In some embodiments, all of the guanosines are replaced with modified guanosine. Suitable modified guanosines will be known to those of skill in the art.

In some embodiments, the RNA polynucleotide of the present invention comprises modified uracil. In some embodiments, all of the uracils are replaced with modified uracil. In some embodiments, the RNA polynucleotide of the present invention comprises modified uridine.

In some embodiments, all of the uridines are replaced with modified uridine. Suitable modified uridines will be known to those of skill in the art and include, 5-Methyluridine, 5-Methoxyuridine, Pseudouridine, N1-Methyl-pseudouridine, and 2-thiouridine.

In some embodiments, the RNA polynucleotide of the present invention comprises modified adenine. In some embodiments, all of the adenines are replaced with modified adenine. In some embodiments, the RNA polynucleotide of the present invention comprises modified adenosine. In some embodiments, all of the adenosines are replaced with modified adenosine. Suitable modified adenosines will be known to those of skill in the art and include N1-methyl-adenosine and N6-methyl-adenosine.

In some embodiments, the RNA polynucleotide of the present invention comprises modified cytosine. In some embodiments, all of the cytosines are replaced with modified cytosine. In some embodiments, the RNA polynucleotide of the present invention comprises modified cytidine. In some embodiments, all of the cytidines are replaced with modified cytidine.

Suitable modified cytidines will be known to those of skill in the art and include 5′-methyl-cytidine.

In some embodiments, the RNA polynucleotide of the present invention comprises pseudouridine. In some embodiments, all of the uridine is replaced by pseudouridine (i.e. the only uridine present in the RNA polynucleotide is pseudouridine).

In some embodiments, the RNA polynucleotide of the present invention is nucleoside-modified mRNA. In some embodiments, the RNA polynucleotide of the present invention is pseudouridine-modified mRNA.

### Protein-Coding Sequence

The protein-coding sequence is not particularly limited and may encode any protein of interest.

The protein-coding sequence may be codon-optimised. For example, the protein-coding sequence may be codon-optimised for expression in a mammalian (e.g. human) cell. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Codon usage tables are known in the art for mammalian cells (e.g. humans), as well as for a variety of other organisms.

In preferred embodiments, the protein-coding sequence encodes an engraftment enhancer.

The engraftment enhancer may be any disclosed herein. In some embodiments, the engraftment enhancer is selected from CXCR4 (or a fragment or variant thereof), CD47 (or a fragment or variant thereof), ITGA4 (or a fragment or variant thereof), and KIT (or a fragment or variant thereof). In some embodiments, the engraftment enhancer comprises or consists of an amino acid sequence that has at least 70%, at least 80%, at least 85%, at least 90%, at least 95%, at least 96%, at least 97%, at least 98%, at least 99% or 100% identity to any of SEQ ID NOs: 2-9, 23-26, 29 or 31.

The protein-coding sequence may be any protein coding sequence disclosed herein. In some embodiments, the protein-coding sequence comprises or consists of a nucleotide sequence that has at least 70%, at least 80%, at least 85%, at least 90%, at least 95%, at least 96%, at least 97%, at least 98%, at least 99% or 100% identity to any of SEQ ID NOs: 10-22, 27, 28, 30 or 32 (e.g. wherein “T” is replaced with “U”).

### C-X-C Chemokine Receptor Type 4 (CXCR4)

In some embodiments, the engraftment enhancer is CXCR4.

In some embodiments, the engraftment enhancer comprises or consists of an amino acid sequence that has at least 70%, at least 80%, at least 85%, at least 90%, at least 95%, at least 96%, at least 97%, at least 98%, at least 99% or 100% identity to any of SEQ ID NOs: 2-9.

In some embodiments, the engraftment enhancer comprises or consists of an amino acid sequence that has at least 70%, at least 80%, at least 85%, at least 90%, at least 95%, at least 96%, at least 97%, at least 98%, at least 99% or 100% identity to SEQ ID NO: 2.

In some embodiments, the engraftment enhancer comprises or consists of an amino acid sequence that has at least 70%, at least 80%, at least 85%, at least 90%, at least 95%, at least 96%, at least 97%, at least 98%, at least 99% or 100% identity to any of SEQ ID NOs: 3-9.

In some embodiments, the engraftment enhancer comprises or consists of an amino acid sequence that has at least 70%, at least 80%, at least 85%, at least 90%, at least 95%, at least 96%, at least 97%, at least 98%, at least 99% or 100% identity to any of SEQ ID NOs: 6-9.

In some embodiments, the protein-coding sequence comprises or consists of a nucleotide sequence that has at least 70%, at least 80%, at least 85%, at least 90%, at least 95%, at least 96%, at least 97%, at least 98%, at least 99% or 100% identity to any of SEQ ID NOs: 10-22 (wherein “T” is replaced with “U”).

In some embodiments, the protein-coding sequence comprises or consists of a nucleotide sequence that has at least 70%, at least 80%, at least 85%, at least 90%, at least 95%, at least 96%, at least 97%, at least 98%, at least 99% or 100% identity to SEQ ID NO: 12 (wherein “T” is replaced with “U”).

In some embodiments, the protein-coding sequence comprises or consists of a nucleotide sequence that has at least 70%, at least 80%, at least 85%, at least 90%, at least 95%, at least 96%, at least 97%, at least 98%, at least 99% or 100% identity to any of SEQ ID NOs: 14-22 (wherein “T” is replaced with “U”).

In some embodiments, the protein-coding sequence comprises or consists of a nucleotide sequence that has at least 70%, at least 80%, at least 85%, at least 90%, at least 95%, at least 96%, at least 97%, at least 98%, at least 99% or 100% identity to any of SEQ ID NOs: 19-22 (wherein “T” is replaced with “U”).

### Integrin Alpha-4 (ITGA4)

In some embodiments, the engraftment enhancer is ITGA4.

In some embodiments, the engraftment enhancer comprises or consists of an amino acid sequence that has at least 70%, at least 80%, at least 85%, at least 90%, at least 95%, at least 96%, at least 97%, at least 98%, at least 99% or 100% identity to SEQ ID NO: 29.

In some embodiments, the protein-coding sequence comprises or consists of a nucleotide sequence that has at least 70%, at least 80%, at least 85%, at least 90%, at least 95%, at least 96%, at least 97%, at least 98%, at least 99% or 100% identity to SEQ ID NO: 30 (wherein “T” is replaced with “U”).

### Tyrosine-Protein Kinase KIT (KIT)

In some embodiments, the engraftment enhancer is KIT.

In some embodiments, the engraftment enhancer comprises or consists of an amino acid sequence that has at least 70%, at least 80%, at least 85%, at least 90%, at least 95%, at least 96%, at least 97%, at least 98%, at least 99% or 100% identity to SEQ ID NO: 31 or 47.

In some embodiments, the engraftment enhancer comprises or consists of an amino acid sequence that has at least 70%, at least 80%, at least 85%, at least 90%, at least 95%, at least 96%, at least 97%, at least 98%, at least 99% or 100% identity to SEQ ID NO: 31.

In some embodiments, the protein-coding sequence comprises or consists of a nucleotide sequence that has at least 70%, at least 80%, at least 85%, at least 90%, at least 95%, at least 96%, at least 97%, at least 98%, at least 99% or 100% identity to SEQ ID NO: 32 (wherein “T” is replaced with “U”).

### Cluster of Differentiation 47 (CD47)

In some embodiments, the engraftment enhancer is CD47.

In some embodiments, the engraftment enhancer comprises or consists of an amino acid sequence that has at least 70%, at least 80%, at least 85%, at least 90%, at least 95%, at least 96%, at least 97%, at least 98%, at least 99% or 100% identity to any of SEQ ID NOs: 23-26.

In some embodiments, the protein-coding sequence comprises or consists of a nucleotide sequence that has at least 70%, at least 80%, at least 85%, at least 90%, at least 95%, at least 96%, at least 97%, at least 98%, at least 99% or 100% identity to SEQ ID NO: 27 or 28 (wherein “T” is replaced with “U”).

### Method of Production

In one aspect, the present invention provides a method for producing the RNA polynucleotide according to the present invention. In preferred embodiments, the method comprises the step of in vitro transcribing a DNA polynucleotide encoding the RNA polynucleotide according to the present invention.

In one aspect, the present invention provides a method for producing the RNA polynucleotide according to the present invention, the method comprising:
(i) providing an in vitro transcription reaction mixture comprising a DNA polynucleotide encoding the RNA polynucleotide, nucleoside triphosphates, and optionally a synthetic cap analog;
(ii) incubating the in vitro transcription reaction mixture to provide an RNA polynucleotide; and
(iii) purifying the RNA polynucleotide.

In one aspect, the present invention provides an in vitro transcription reaction mixture comprising a DNA polynucleotide encoding the RNA polynucleotide of the present invention.

The in vitro transcription reaction mixture may comprise a DNA polynucleotide encoding the RNA polynucleotide as a template, an RNA polymerase, and nucleoside triphosphates. The DNA polynucleotide may be in the form of a linearised plasmid. The RNA polymerase may be a T7 RNA polymerase. The nucleoside triphosphates may consist of adenosine triphosphate, guanosine triphosphate, cytidine triphosphate and uridine triphosphate, or modified versions thereof.

In some embodiments, the in vitro transcription reaction mixture comprises a modified nucleoside triphosphate. In some embodiments, the in vitro transcription reaction mixture comprises a modified uridine, preferably pseudouridine. In some embodiments, the nucleoside triphosphates consist of adenosine triphosphate, guanosine triphosphate, cytidine triphosphate and pseudouridine triphosphate.

In some embodiments, the method comprises a step of capping the RNA polynucleotide. The RNA polynucleotide may be capped enzymatically at the end of the in vitro transcription reaction or as a synthetic cap analog during the in vitro transcription reaction. In some embodiments, the in vitro transcription reaction mixture comprises a synthetic cap analog. In some embodiments, the synthetic cap analog is m⁷G(5′)ppp(5′)(2′OMeA)pG.

The in vitro transcription reaction mixture may comprise any other suitable reagents such as an RNase inhibitor, an inorganic pyrophosphatase, a transcription buffer, and the like.

The in vitro transcription reaction mixture may be incubated under conditions suitable for transcribing the DNA polynucleotide to produce the RNA polynucleotide. Any suitable conditions may be used, for example about 37° C. for about 1-4 hours or about 37° C. for about 2-3 hours.

In some embodiments, the method comprises a step of purifying the RNA polynucleotide. Any suitable method for purifying the RNA polynucleotide may be used, including silica-based purification and/or high-performance liquid chromatography purification.

### Exemplary RNA Polynucleotides

In one aspect, the present invention provides a RNA polynucleotide comprising from 5′ to 3′: a translation non-blocking eIF4F aptamer; optionally a Kozak sequence; a protein-coding sequence; and a WPRE.

In one aspect, the present invention provides a RNA polynucleotide comprising from 5′ to 3′: a translation non-blocking eIF4F aptamer; optionally a Kozak sequence; a protein-coding sequence; a WPRE; and a polyA tail comprising at least about 100 nucleotides.

In one aspect, the present invention provides a RNA polynucleotide comprising from 5′ to 3′: a m7G(5′)ppp(5′)(2′OMeA)pG cap; a translation non-blocking eIF4F aptamer; optionally a Kozak sequence; a protein-coding sequence; a WPRE; and a polyA tail comprising at least about 100 nucleotides.

In one aspect, the present invention provides a RNA polynucleotide comprising from 5′ to 3′: a translation non-blocking eIF4F aptamer; optionally a Kozak sequence; a protein-coding sequence encoding an engraftment enhancer; and a WPRE.

In one aspect, the present invention provides a RNA polynucleotide comprising from 5′ to 3′: a translation non-blocking eIF4F aptamer; optionally a Kozak sequence; a protein-coding sequence encoding an engraftment enhancer; a WPRE; and a polyA tail comprising at least about 100 nucleotides.

In one aspect, the present invention provides a RNA polynucleotide comprising from 5′ to 3′: a m7G(5′)ppp(5′)(2′OMeA)pG cap; a translation non-blocking eIF4F aptamer; optionally a Kozak sequence; a protein-coding sequence encoding an engraftment enhancer; a WPRE; and a polyA tail comprising at least about 100 nucleotides.

The RNA polynucleotide may comprise any other suitable elements, for example linker sequences. In some embodiments, the RNA polynucleotide comprises one or more linker sequences. Suitably a linker sequence is from 1 to 100, from 1 to 50, from 1 to 40, from 1 to 30, from 1 to 20, or from 1 to 10 nucleotides in length.

Exemplary RNA polynucleotides are provided below in SEQ ID NOs: 39-46. In some embodiments, the RNA polynucleotide comprises or consists of a nucleotide sequence having least 70%, at least 75%, at least 80%, at least 85%, at least 90%, at least 95%, at least 96%, at least 97%, at least 98%, at least 99% or 100% identity to any of SEQ ID NOs: 39-46. In some embodiments, the RNA polynucleotide comprises or consists of the nucleotide sequence of any of SEQ ID NOs: 39-46.

In one aspect, the present invention provides a RNA polynucleotide comprising or consisting a nucleotide sequence having least 70%, at least 75%, at least 80%, at least 85%, at least 90%, at least 95%, at least 96%, at least 97%, at least 98%, at least 99% or 100% identity to any of SEQ ID NOs: 39-46.

In one aspect, the present invention provides a RNA polynucleotide comprising or consisting of the nucleotide sequence of any of SEQ ID NOs: 39-46.

Exemplary RNA polynucleotide encoding CXCR4 WT
(SEQ ID NO: 39)
UAAUACGACUCACUAUAAGGACUCACUAUUUGUUUUCGCGCCCAGUUGCAAAAAGUGUCGCCACCAUGG
AAGGCAUCAGCAUCUACACCAGCGACAACUACACCGAGGAAAUGGGCAGCGGCGACUACGACAGCAUGA
AGGAACCCUGCUUCCGGGAAGAGAACGCCAACUUCAACAAGAUCUUCCUGCCCACAAUCUACAGCAUCA
UCUUUCUGACCGGCAUCGUGGGCAACGGACUCGUGAUCCUCGUGAUGGGCUACCAGAAAAAGCUGCGGA
GCAUGACCGACAAGUACCGGCUGCACCUGAGCGUGGCCGACCUGCUGUUCGUGAUCACCCUGCCUUUCU
GGGCCGUGGACGCCGUGGCCAAUUGGUACUUCGGCAACUUCCUGUGCAAGGCCGUGCACGUGAUCUACA
CAGUGAACCUGUACAGCAGCGUGCUGAUCCUGGCCUUCAUCAGCCUGGACAGAUACCUGGCCAUCGUGC
ACGCCACCAACAGCCAGCGGCCUAGAAAGCUGCUGGCCGAGAAGGUGGUGUACGUGGGCGUGUGGAUUC
CCGCCCUGCUGCUGACCAUCCCCGACUUCAUCUUCGCCAACGUGUCCGAGGCCGACGACCGGUACAUCU
GCGACCGGUUCUACCCCAACGACCUGUGGGUGGUGGUGUUCCAGUUCCAGCACAUCAUGGUGGGACUGA
UCCUGCCUGGCAUCGUGAUUCUGAGCUGCUACUGCAUCAUCAUCAGCAAGCUGAGCCACAGCAAGGGCC
ACCAGAAGCGGAAGGCCCUGAAAACCACCGUGAUCCUGAUUCUGGCUUUCUUCGCCUGCUGGCUGCCCU
ACUACAUCGGCAUCAGCAUCGACAGCUUCAUCCUGCUGGAAAUCAUCAAGCAGGGCUGCGAGUUCGAGA
ACACCGUGCACAAGUGGAUCAGCAUUACCGAGGCCCUGGCCUUUUUCCACUGCUGCCUGAACCCUAUCC
UGUACGCCUUCCUGGGCGCCAAGUUCAAGACCUCUGCCCAGCACGCCCUGACCAGCGUGUCCAGAGGAA
GCAGCCUGAAGAUCCUGAGCAAGGGCAAGAGAGGCGGCCACAGCUCCGUGUCUACAGAGAGCGAGAGCA
GCAGCUUCCACAGCAGCUGAAAUCAACCUCUGGAUUACAAAAUUUGUGAAAGAUUGACUGGUAUUCUUA
ACUAUGUUGCUCCUUUUACGCUAUGUGGAUACGCUGCUUUAAUGCCUUUGUAUCAUGCUAUUGCUUCCC
GUAUGGCUUUCAUUUUCUCCUCCUUGUAUAAAUCCUGGUUGCUGUCUCUUUAUGAGGAGUUGUGGCCCG
UUGUCAGGCAACGUGGCGUGGUGUGCACUGUGUUUGCUGACGCAACCCCCACUGGUUGGGGCAUUGCCA
CCACCUGUCAGCUCCUUUCCGGGACUUUCGCUUUCCCCCUCCCUAUUGCCACGGCGGAACUCAUCGCCG
CCUGCCUUGCCCGCUGCUGGACAGGGGCUCGGCUGUUGGGCACUGACAAUUCCGUGGUGUUGUCGGGGA
AAUCAUCGUCCUUUCCUUGGCUGCUCGCCUGUGUUGCCACCUGGAUUCUGCGCGGGACGUCCUUCUGCU
ACGUCCCUUCGGCCCUCAAUCCAGCGGACCUUCCUUCCCGCGGCCUGCUGCCGGCUCUGCGGCCUCUUC
CGCGUCUUCGCCUUCGCCCUCAGACGAGUCGGAUCUCCCUUUGGGCCGCCUCCCCGCCUGUUAAUUAAA
AAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAA
Exemplary RNA polynucleotide encoding CXCR4 V160L
(SEQ ID NO: 40)
UAAUACGACUCACUAUAAGGACUCACUAUUUGUUUUCGCGCCCAGUUGCAAAAAGUGUCGCCACCAUGG
AAGGCAUCAGCAUCUACACCAGCGACAACUACACCGAGGAAAUGGGCAGCGGCGACUACGACAGCAUGA
AGGAACCCUGCUUCCGGGAAGAGAACGCCAACUUCAACAAGAUCUUCCUGCCCACAAUCUACAGCAUCA
UCUUUCUGACCGGCAUCGUGGGCAACGGACUCGUGAUCCUCGUGAUGGGCUACCAGAAAAAGCUGCGGA
GCAUGACCGACAAGUACCGGCUGCACCUGAGCGUGGCCGACCUGCUGUUCGUGAUCACCCUGCCUUUCU
GGGCCGUGGACGCCGUGGCCAAUUGGUACUUCGGCAACUUCCUGUGCAAGGCCGUGCACGUGAUCUACA
CAGUGAACCUGUACAGCAGCGUGCUGAUCCUGGCCUUCAUCAGCCUGGACAGAUACCUGGCCAUCGUGC
ACGCCACCAACAGCCAGCGGCCUAGAAAGCUGCUGGCCGAGAAGGUGGUGUACGUGGGCCUGUGGAUUC
CCGCCCUGCUGCUGACCAUCCCCGACUUCAUCUUCGCCAACGUGUCCGAGGCCGACGACCGGUACAUCU
GCGACCGGUUCUACCCCAACGACCUGUGGGUGGUGGUGUUCCAGUUCCAGCACAUCAUGGUGGGACUGA
UCCUGCCUGGCAUCGUGAUUCUGAGCUGCUACUGCAUCAUCAUCAGCAAGCUGAGCCACAGCAAGGGCC
ACCAGAAGCGGAAGGCCCUGAAAACCACCGUGAUCCUGAUUCUGGCUUUCUUCGCCUGCUGGCUGCCCU
ACUACAUCGGCAUCAGCAUCGACAGCUUCAUCCUGCUGGAAAUCAUCAAGCAGGGCUGCGAGUUCGAGA
ACACCGUGCACAAGUGGAUCAGCAUUACCGAGGCCCUGGCCUUUUUCCACUGCUGCCUGAACCCUAUCC
UGUACGCCUUCCUGGGCGCCAAGUUCAAGACCUCUGCCCAGCACGCCCUGACCAGCGUGUCCAGAGGAA
GCAGCCUGAAGAUCCUGAGCAAGGGCAAGAGAGGCGGCCACAGCUCCGUGUCUACAGAGAGCGAGAGCA
GCAGCUUCCACAGCAGCUGAAAUCAACCUCUGGAUUACAAAAUUUGUGAAAGAUUGACUGGUAUUCUUA
ACUAUGUUGCUCCUUUUACGCUAUGUGGAUACGCUGCUUUAAUGCCUUUGUAUCAUGCUAUUGCUUCCC
GUAUGGCUUUCAUUUUCUCCUCCUUGUAUAAAUCCUGGUUGCUGUCUCUUUAUGAGGAGUUGUGGCCCG
UUGUCAGGCAACGUGGCGUGGUGUGCACUGUGUUUGCUGACGCAACCCCCACUGGUUGGGGCAUUGCCA
CCACCUGUCAGCUCCUUUCCGGGACUUUCGCUUUCCCCCUCCCUAUUGCCACGGCGGAACUCAUCGCCG
CCUGCCUUGCCCGCUGCUGGACAGGGGCUCGGCUGUUGGGCACUGACAAUUCCGUGGUGUUGUCGGGGA
AAUCAUCGUCCUUUCCUUGGCUGCUCGCCUGUGUUGCCACCUGGAUUCUGCGCGGGACGUCCUUCUGCU
ACGUCCCUUCGGCCCUCAAUCCAGCGGACCUUCCUUCCCGCGGCCUGCUGCCGGCUCUGCGGCCUCUUC
CGCGUCUUCGCCUUCGCCCUCAGACGAGUCGGAUCUCCCUUUGGGCCGCCUCCCCGCCUGUUAAUUAAA
AAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAA
Exemplary RNA polynucleotide encoding CXCR4 A175F
(SEQ ID NO: 41)
UAAUACGACUCACUAUAAGGACUCACUAUUUGUUUUCGCGCCCAGUUGCAAAAAGUGUCGCCACCAUGG
AAGGCAUCAGCAUCUACACCAGCGACAACUACACCGAGGAAAUGGGCAGCGGCGACUACGACAGCAUGA
AGGAACCCUGCUUCCGGGAAGAGAACGCCAACUUCAACAAGAUCUUCCUGCCCACAAUCUACAGCAUCA
UCUUUCUGACCGGCAUCGUGGGCAACGGACUCGUGAUCCUCGUGAUGGGCUACCAGAAAAAGCUGCGGA
GCAUGACCGACAAGUACCGGCUGCACCUGAGCGUGGCCGACCUGCUGUUCGUGAUCACCCUGCCUUUCU
GGGCCGUGGACGCCGUGGCCAAUUGGUACUUCGGCAACUUCCUGUGCAAGGCCGUGCACGUGAUCUACA
CAGUGAACCUGUACAGCAGCGUGCUGAUCCUGGCCUUCAUCAGCCUGGACAGAUACCUGGCCAUCGUGC
ACGCCACCAACAGCCAGCGGCCUAGAAAGCUGCUGGCCGAGAAGGUGGUGUACGUGGGCGUGUGGAUUC
CCGCCCUGCUGCUGACCAUCCCCGACUUCAUCUUCUUCAACGUGUCCGAGGCCGACGACCGGUACAUCU
GCGACCGGUUCUACCCCAACGACCUGUGGGUGGUGGUGUUCCAGUUCCAGCACAUCAUGGUGGGACUGA
UCCUGCCUGGCAUCGUGAUUCUGAGCUGCUACUGCAUCAUCAUCAGCAAGCUGAGCCACAGCAAGGGCC
ACCAGAAGCGGAAGGCCCUGAAAACCACCGUGAUCCUGAUUCUGGCUUUCUUCGCCUGCUGGCUGCCCU
ACUACAUCGGCAUCAGCAUCGACAGCUUCAUCCUGCUGGAAAUCAUCAAGCAGGGCUGCGAGUUCGAGA
ACACCGUGCACAAGUGGAUCAGCAUUACCGAGGCCCUGGCCUUUUUCCACUGCUGCCUGAACCCUAUCC
UGUACGCCUUCCUGGGCGCCAAGUUCAAGACCUCUGCCCAGCACGCCCUGACCAGCGUGUCCAGAGGAA
GCAGCCUGAAGAUCCUGAGCAAGGGCAAGAGAGGCGGCCACAGCUCCGUGUCUACAGAGAGCGAGAGCA
GCAGCUUCCACAGCAGCUGAAAUCAACCUCUGGAUUACAAAAUUUGUGAAAGAUUGACUGGUAUUCUUA
ACUAUGUUGCUCCUUUUACGCUAUGUGGAUACGCUGCUUUAAUGCCUUUGUAUCAUGCUAUUGCUUCCC
GUAUGGCUUUCAUUUUCUCCUCCUUGUAUAAAUCCUGGUUGCUGUCUCUUUAUGAGGAGUUGUGGCCCG
UUGUCAGGCAACGUGGCGUGGUGUGCACUGUGUUUGCUGACGCAACCCCCACUGGUUGGGGCAUUGCCA
CCACCUGUCAGCUCCUUUCCGGGACUUUCGCUUUCCCCCUCCCUAUUGCCACGGCGGAACUCAUCGCCG
CCUGCCUUGCCCGCUGCUGGACAGGGGCUCGGCUGUUGGGCACUGACAAUUCCGUGGUGUUGUCGGGGA
AAUCAUCGUCCUUUCCUUGGCUGCUCGCCUGUGUUGCCACCUGGAUUCUGCGCGGGACGUCCUUCUGCU
ACGUCCCUUCGGCCCUCAAUCCAGCGGACCUUCCUUCCCGCGGCCUGCUGCCGGCUCUGCGGCCUCUUC
CGCGUCUUCGCCUUCGCCCUCAGACGAGUCGGAUCUCCCUUUGGGCCGCCUCCCCGCCUGUUAAUUAAA
AAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAA
Exemplary RNA polynucleotide encoding CXCR4 Q200A
(SEQ ID NO: 42)
UAAUACGACUCACUAUAAGGACUCACUAUUUGUUUUCGCGCCCAGUUGCAAAAAGUGUCGCCACCAUGG
AAGGCAUCAGCAUCUACACCAGCGACAACUACACCGAGGAAAUGGGCAGCGGCGACUACGACAGCAUGA
AGGAACCCUGCUUCCGGGAAGAGAACGCCAACUUCAACAAGAUCUUCCUGCCCACAAUCUACAGCAUCA
UCUUUCUGACCGGCAUCGUGGGCAACGGACUCGUGAUCCUCGUGAUGGGCUACCAGAAAAAGCUGCGGA
GCAUGACCGACAAGUACCGGCUGCACCUGAGCGUGGCCGACCUGCUGUUCGUGAUCACCCUGCCUUUCU
GGGCCGUGGACGCCGUGGCCAAUUGGUACUUCGGCAACUUCCUGUGCAAGGCCGUGCACGUGAUCUACA
CAGUGAACCUGUACAGCAGCGUGCUGAUCCUGGCCUUCAUCAGCCUGGACAGAUACCUGGCCAUCGUGC
ACGCCACCAACAGCCAGCGGCCUAGAAAGCUGCUGGCCGAGAAGGUGGUGUACGUGGGCGUGUGGAUUC
CCGCCCUGCUGCUGACCAUCCCCGACUUCAUCUUCGCCAACGUGUCCGAGGCCGACGACCGGUACAUCU
GCGACCGGUUCUACCCCAACGACCUGUGGGUGGUGGUGUUCGCGUUCCAGCACAUCAUGGUGGGACUGA
UCCUGCCUGGCAUCGUGAUUCUGAGCUGCUACUGCAUCAUCAUCAGCAAGCUGAGCCACAGCAAGGGCC
ACCAGAAGCGGAAGGCCCUGAAAACCACCGUGAUCCUGAUUCUGGCUUUCUUCGCCUGCUGGCUGCCCU
ACUACAUCGGCAUCAGCAUCGACAGCUUCAUCCUGCUGGAAAUCAUCAAGCAGGGCUGCGAGUUCGAGA
ACACCGUGCACAAGUGGAUCAGCAUUACCGAGGCCCUGGCCUUUUUCCACUGCUGCCUGAACCCUAUCC
UGUACGCCUUCCUGGGCGCCAAGUUCAAGACCUCUGCCCAGCACGCCCUGACCAGCGUGUCCAGAGGAA
GCAGCCUGAAGAUCCUGAGCAAGGGCAAGAGAGGCGGCCACAGCUCCGUGUCUACAGAGAGCGAGAGCA
GCAGCUUCCACAGCAGCUGAAAUCAACCUCUGGAUUACAAAAUUUGUGAAAGAUUGACUGGUAUUCUUA
ACUAUGUUGCUCCUUUUACGCUAUGUGGAUACGCUGCUUUAAUGCCUUUGUAUCAUGCUAUUGCUUCCC
GUAUGGCUUUCAUUUUCUCCUCCUUGUAUAAAUCCUGGUUGCUGUCUCUUUAUGAGGAGUUGUGGCCCG
UUGUCAGGCAACGUGGCGUGGUGUGCACUGUGUUUGCUGACGCAACCCCCACUGGUUGGGGCAUUGCCA
CCACCUGUCAGCUCCUUUCCGGGACUUUCGCUUUCCCCCUCCCUAUUGCCACGGCGGAACUCAUCGCCG
CCUGCCUUGCCCGCUGCUGGACAGGGGCUCGGCUGUUGGGCACUGACAAUUCCGUGGUGUUGUCGGGGA
AAUCAUCGUCCUUUCCUUGGCUGCUCGCCUGUGUUGCCACCUGGAUUCUGCGCGGGACGUCCUUCUGCU
ACGUCCCUUCGGCCCUCAAUCCAGCGGACCUUCCUUCCCGCGGCCUGCUGCCGGCUCUGCGGCCUCUUC
CGCGUCUUCGCCUUCGCCCUCAGACGAGUCGGAUCUCCCUUUGGGCCGCCUCCCCGCCUGUUAAUUAAA
AAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAA
Exemplary RNA polynucleotide encoding CXCR4 D262N
(SEQ ID NO: 43)
UAAUACGACUCACUAUAAGGACUCACUAUUUGUUUUCGCGCCCAGUUGCAAAAAGUGUCGCCACCAUGG
AAGGCAUCAGCAUCUACACCAGCGACAACUACACCGAGGAAAUGGGCAGCGGCGACUACGACAGCAUGA
AGGAACCCUGCUUCCGGGAAGAGAACGCCAACUUCAACAAGAUCUUCCUGCCCACAAUCUACAGCAUCA
UCUUUCUGACCGGCAUCGUGGGCAACGGACUCGUGAUCCUCGUGAUGGGCUACCAGAAAAAGCUGCGGA
GCAUGACCGACAAGUACCGGCUGCACCUGAGCGUGGCCGACCUGCUGUUCGUGAUCACCCUGCCUUUCU
GGGCCGUGGACGCCGUGGCCAAUUGGUACUUCGGCAACUUCCUGUGCAAGGCCGUGCACGUGAUCUACA
CAGUGAACCUGUACAGCAGCGUGCUGAUCCUGGCCUUCAUCAGCCUGGACAGAUACCUGGCCAUCGUGC
ACGCCACCAACAGCCAGCGGCCUAGAAAGCUGCUGGCCGAGAAGGUGGUGUACGUGGGCGUGUGGAUUC
CCGCCCUGCUGCUGACCAUCCCCGACUUCAUCUUCGCCAACGUGUCCGAGGCCGACGACCGGUACAUCU
GCGACCGGUUCUACCCCAACGACCUGUGGGUGGUGGUGUUCCAGUUCCAGCACAUCAUGGUGGGACUGA
UCCUGCCUGGCAUCGUGAUUCUGAGCUGCUACUGCAUCAUCAUCAGCAAGCUGAGCCACAGCAAGGGCC
ACCAGAAGCGGAAGGCCCUGAAAACCACCGUGAUCCUGAUUCUGGCUUUCUUCGCCUGCUGGCUGCCCU
ACUACAUCGGCAUCAGCAUCAACAGCUUCAUCCUGCUGGAAAUCAUCAAGCAGGGCUGCGAGUUCGAGA
ACACCGUGCACAAGUGGAUCAGCAUUACCGAGGCCCUGGCCUUUUUCCACUGCUGCCUGAACCCUAUCC
UGUACGCCUUCCUGGGCGCCAAGUUCAAGACCUCUGCCCAGCACGCCCUGACCAGCGUGUCCAGAGGAA
GCAGCCUGAAGAUCCUGAGCAAGGGCAAGAGAGGCGGCCACAGCUCCGUGUCUACAGAGAGCGAGAGCA
GCAGCUUCCACAGCAGCUGAAAUCAACCUCUGGAUUACAAAAUUUGUGAAAGAUUGACUGGUAUUCUUA
ACUAUGUUGCUCCUUUUACGCUAUGUGGAUACGCUGCUUUAAUGCCUUUGUAUCAUGCUAUUGCUUCCC
GUAUGGCUUUCAUUUUCUCCUCCUUGUAUAAAUCCUGGUUGCUGUCUCUUUAUGAGGAGUUGUGGCCCG
UUGUCAGGCAACGUGGCGUGGUGUGCACUGUGUUUGCUGACGCAACCCCCACUGGUUGGGGCAUUGCCA
CCACCUGUCAGCUCCUUUCCGGGACUUUCGCUUUCCCCCUCCCUAUUGCCACGGCGGAACUCAUCGCCG
CCUGCCUUGCCCGCUGCUGGACAGGGGCUCGGCUGUUGGGCACUGACAAUUCCGUGGUGUUGUCGGGGA
AAUCAUCGUCCUUUCCUUGGCUGCUCGCCUGUGUUGCCACCUGGAUUCUGCGCGGGACGUCCUUCUGCU
ACGUCCCUUCGGCCCUCAAUCCAGCGGACCUUCCUUCCCGCGGCCUGCUGCCGGCUCUGCGGCCUCUUC
CGCGUCUUCGCCUUCGCCCUCAGACGAGUCGGAUCUCCCUUUGGGCCGCCUCCCCGCCUGUUAAUUAAA
AAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAA
Exemplary RNA polynucleotide encoding CXCR4 H281A
(SEQ ID NO: 44)
UAAUACGACUCACUAUAAGGACUCACUAUUUGUUUUCGCGCCCAGUUGCAAAAAGUGUCGCCACCAUGG
AAGGCAUCAGCAUCUACACCAGCGACAACUACACCGAGGAAAUGGGCAGCGGCGACUACGACAGCAUGA
AGGAACCCUGCUUCCGGGAAGAGAACGCCAACUUCAACAAGAUCUUCCUGCCCACAAUCUACAGCAUCA
UCUUUCUGACCGGCAUCGUGGGCAACGGACUCGUGAUCCUCGUGAUGGGCUACCAGAAAAAGCUGCGGA
GCAUGACCGACAAGUACCGGCUGCACCUGAGCGUGGCCGACCUGCUGUUCGUGAUCACCCUGCCUUUCU
GGGCCGUGGACGCCGUGGCCAAUUGGUACUUCGGCAACUUCCUGUGCAAGGCCGUGCACGUGAUCUACA
CAGUGAACCUGUACAGCAGCGUGCUGAUCCUGGCCUUCAUCAGCCUGGACAGAUACCUGGCCAUCGUGC
ACGCCACCAACAGCCAGCGGCCUAGAAAGCUGCUGGCCGAGAAGGUGGUGUACGUGGGCGUGUGGAUUC
CCGCCCUGCUGCUGACCAUCCCCGACUUCAUCUUCGCCAACGUGUCCGAGGCCGACGACCGGUACAUCU
GCGACCGGUUCUACCCCAACGACCUGUGGGUGGUGGUGUUCCAGUUCCAGCACAUCAUGGUGGGACUGA
UCCUGCCUGGCAUCGUGAUUCUGAGCUGCUACUGCAUCAUCAUCAGCAAGCUGAGCCACAGCAAGGGCC
ACCAGAAGCGGAAGGCCCUGAAAACCACCGUGAUCCUGAUUCUGGCUUUCUUCGCCUGCUGGCUGCCCU
ACUACAUCGGCAUCAGCAUCGACAGCUUCAUCCUGCUGGAAAUCAUCAAGCAGGGCUGCGAGUUCGAGA
ACACCGUGGCCAAGUGGAUCAGCAUUACCGAGGCCCUGGCCUUUUUCCACUGCUGCCUGAACCCUAUCC
UGUACGCCUUCCUGGGCGCCAAGUUCAAGACCUCUGCCCAGCACGCCCUGACCAGCGUGUCCAGAGGAA
GCAGCCUGAAGAUCCUGAGCAAGGGCAAGAGAGGCGGCCACAGCUCCGUGUCUACAGAGAGCGAGAGCA
GCAGCUUCCACAGCAGCUGAAAUCAACCUCUGGAUUACAAAAUUUGUGAAAGAUUGACUGGUAUUCUUA
ACUAUGUUGCUCCUUUUACGCUAUGUGGAUACGCUGCUUUAAUGCCUUUGUAUCAUGCUAUUGCUUCCC
GUAUGGCUUUCAUUUUCUCCUCCUUGUAUAAAUCCUGGUUGCUGUCUCUUUAUGAGGAGUUGUGGCCCG
UUGUCAGGCAACGUGGCGUGGUGUGCACUGUGUUUGCUGACGCAACCCCCACUGGUUGGGGCAUUGCCA
CCACCUGUCAGCUCCUUUCCGGGACUUUCGCUUUCCCCCUCCCUAUUGCCACGGCGGAACUCAUCGCCG
CCUGCCUUGCCCGCUGCUGGACAGGGGCUCGGCUGUUGGGCACUGACAAUUCCGUGGUGUUGUCGGGGA
AAUCAUCGUCCUUUCCUUGGCUGCUCGCCUGUGUUGCCACCUGGAUUCUGCGCGGGACGUCCUUCUGCU
ACGUCCCUUCGGCCCUCAAUCCAGCGGACCUUCCUUCCCGCGGCCUGCUGCCGGCUCUGCGGCCUCUUC
CGCGUCUUCGCCUUCGCCCUCAGACGAGUCGGAUCUCCCUUUGGGCCGCCUCCCCGCCUGUUAAUUAAA
AAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAA
Exemplary RNA polynucleotide encoding ITGA4
(SEQ ID NO: 45)
UAAUACGACUCACUAUAAGGACUCACUAUUUGUUUUCGCGCCCAGUUGCAAAAAGUGUCGCCACCAUGG
CUUGGGAGGCUCGGAGAGAACCUGGACCUAGAAGAGCUGCCGUGCGGGAGACUGUCAUGCUGCUGCUGU
GCCUGGGGGUGCCCACAGGCAGACCUUACAACGUGGAUACCGAGAGCGCCCUGCUGUAUCAGGGCCCCC
ACAACACCCUGUUUGGCUACUCUGUGGUGCUGCACAGCCACGGCGCCAACCGCUGGCUGCUGGUGGGCG
CCCCCACCGCCAAUUGGCUGGCCAAUGCCUCCGUGAUCAACCCAGGCGCCAUCUACAGAUGUCGGAUCG
GCAAGAAUCCUGGCCAGACAUGCGAGCAGCUGCAGCUGGGCUCCCCCAACGGCGAGCCUUGUGGCAAGA
CAUGCCUGGAGGAGAGGGACAAUCAGUGGCUGGGCGUGACACUGAGCAGACAGCCCGGCGAGAACGGCU
CCAUCGUGACAUGCGGCCACAGAUGGAAGAACAUCUUUUACAUCAAGAACGAGAAUAAGCUGCCAACAG
GCGGCUGCUAUGGCGUGCCCCCAGACCUGAGAACAGAGCUGAGCAAGCGGAUCGCCCCAUGCUACCAGG
AUUAUGUGAAGAAGUUUGGCGAGAAUUUUGCCUCUUGCCAGGCCGGCAUCUCCUCCUUCUACACCAAGG
AUCUGAUCGUGAUGGGCGCCCCUGGCUCUUCCUAUUGGACAGGCUCUCUGUUCGUGUAUAAUAUCACAA
CCAACAAGUACAAGGCCUUCCUGGACAAGCAGAACCAGGUGAAGUUCGGCUCCUAUCUGGGCUACAGCG
UGGGCGCCGGCCACUUUCGGUCUCAGCACACCACAGAGGUGGUGGGCGGCGCCCCCCAGCACGAGCAGA
UCGGCAAGGCCUACAUCUUCUCCAUCGACGAGAAGGAGCUGAAUAUCCUGCACGAGAUGAAGGGCAAGA
AGCUGGGCUCCUACUUUGGCGCCUCCGUGUGCGCCGUGGACCUGAAUGCCGACGGCUUUUCCGACCUGC
UGGUGGGCGCCCCAAUGCAGUCCACAAUCAGAGAGGAGGGCAGAGUGUUCGUGUAUAUCAAUUCCGGCA
GCGGCGCCGUGAUGAAUGCCAUGGAGACCAAUCUGGUGGGCUCCGACAAGUAUGCCGCCAGAUUCGGCG
AGAGCAUCGUGAAUCUGGGCGACAUCGACAACGAUGGCUUCGAGGACGUGGCCAUCGGCGCCCCACAGG
AGGAUGACCUGCAGGGCGCCAUCUAUAUCUAUAACGGCCGGGCCGACGGCAUCUCUAGCACCUUCUCCC
AGAGAAUCGAGGGCCUGCAGAUCAGCAAGUCCCUGAGCAUGUUCGGCCAGAGCAUCUCCGGCCAGAUCG
ACGCCGAUAAUAACGGCUACGUGGAUGUGGCCGUGGGCGCCUUUAGAAGCGACUCCGCCGUGCUGCUGA
GAACAAGGCCCGUGGUGAUCGUGGAUGCCUCCCUGUCUCACCCCGAGUCCGUGAAUCGGACAAAGUUUG
ACUGCGUGGAGAAUGGCUGGCCAAGCGUGUGCAUCGAUCUGACACUGUGCUUUUCCUAUAAGGGCAAGG
AGGUGCCAGGCUAUAUCGUGCUGUUUUACAACAUGUCUCUGGAUGUGAACAGAAAGGCCGAGUCCCCCC
CAAGAUUCUACUUUUCCUCUAACGGCACCUCUGAUGUGAUCACCGGCUCUAUCCAGGUGUCCAGCAGGG
AGGCCAAUUGCAGAACCCACCAGGCCUUUAUGCGGAAGGAUGUGCGCGACAUCCUGACCCCAAUCCAGA
UCGAGGCCGCCUAUCACCUGGGCCCCCACGUGAUCUCCAAGCGGUCCACCGAGGAGUUCCCUCCACUGC
AGCCAAUCCUGCAGCAGAAGAAGGAGAAGGACAUCAUGAAGAAGACAAUCAACUUCGCCAGGUUUUGCG
CCCACGAGAACUGUUCCGCCGACCUGCAGGUGUCUGCCAAGAUCGGCUUCCUGAAGCCCCACGAGAACA
AGACAUAUCUGGCCGUGGGCUCCAUGAAGACCCUGAUGCUGAACGUGAGCCUGUUUAACGCCGGCGACG
AUGCCUACGAGACAACACUGCACGUGAAGCUGCCAGUGGGCCUGUACUUCAUCAAGAUCCUGGAGCUGG
AGGAGAAGCAGAUCAACUGUGAGGUGACCGAUAACUCCGGCGUGGUGCAGCUGGAUUGCAGCAUCGGCU
AUAUCUACGUGGACCACCUGUCCCGCAUCGACAUCUCUUUUCUGCUGGACGUGUCCAGCCUGUCCCGGG
CCGAGGAGGACCUGUCCAUCACAGUGCACGCCACCUGCGAGAAUGAGGAGGAGAUGGACAACCUGAAGC
ACUCCAGAGUGACAGUGGCCAUCCCACUGAAGUACGAGGUGAAGCUGACAGUGCACGGCUUUGUGAAUC
CAACCUCCUUCGUGUACGGCUCCAAUGACGAGAAUGAGCCAGAGACAUGUAUGGUGGAGAAGAUGAACC
UGACAUUUCACGUGAUCAAUACAGGCAAUUCUAUGGCCCCUAACGUGAGCGUGGAGAUCAUGGUGCCAA
AUUCUUUCAGCCCACAGACAGACAAGCUGUUUAACAUCCUGGACGUGCAGACAACCACAGGCGAGUGUC
ACUUUGAGAACUACCAGAGAGUGUGCGCCCUGGAGCAGCAGAAGUCCGCCAUGCAGACACUGAAGGGCA
UCGUGAGAUUUCUGAGCAAGACAGAUAAGAGGCUGCUGUACUGCAUCAAGGCCGAUCCCCACUGCCUGA
AUUUUCUGUGCAACUUCGGCAAGAUGGAGUCUGGCAAGGAGGCCUCCGUGCACAUCCAGCUGGAGGGCA
GACCCUCCAUCCUGGAGAUGGACGAGACCAGCGCCCUGAAGUUCGAGAUCAGAGCCACAGGCUUCCCAG
AGCCCAACCCCCGGGUGAUCGAGCUGAACAAGGAUGAGAACGUGGCCCACGUGCUGCUGGAGGGCCUGC
ACCACCAGCGGCCCAAGAGAUAUUUCACCAUCGUGAUCAUCUCCAGCUCUCUGCUGCUGGGCCUGAUCG
UGCUGCUGCUGAUCUCCUAUGUGAUGUGGAAGGCCGGCUUCUUUAAGCGGCAGUACAAGUCCAUCCUGC
AGGAAGAAAAUCGACGCGAUUCAUGGUCUUACAUUAAUUCUAAAUCAAACGACGACUAAAAUCAACCUC
UGGAUUACAAAAUUUGUGAAAGAUUGACUGGUAUUCUUAACUAUGUUGCUCCUUUUACGCUAUGUGGAU
ACGCUGCUUUAAUGCCUUUGUAUCAUGCUAUUGCUUCCCGUAUGGCUUUCAUUUUCUCCUCCUUGUAUA
AAUCCUGGUUGCUGUCUCUUUAUGAGGAGUUGUGGCCCGUUGUCAGGCAACGUGGCGUGGUGUGCACUG
UGUUUGCUGACGCAACCCCCACUGGUUGGGGCAUUGCCACCACCUGUCAGCUCCUUUCCGGGACUUUCG
CUUUCCCCCUCCCUAUUGCCACGGCGGAACUCAUCGCCGCCUGCCUUGCCCGCUGCUGGACAGGGGCUC
GGCUGUUGGGCACUGACAAUUCCGUGGUGUUGUCGGGGAAAUCAUCGUCCUUUCCUUGGCUGCUCGCCU
GUGUUGCCACCUGGAUUCUGCGCGGGACGUCCUUCUGCUACGUCCCUUCGGCCCUCAAUCCAGCGGACC
UUCCUUCCCGCGGCCUGCUGCCGGCUCUGCGGCCUCUUCCGCGUCUUCGCCUUCGCCCUCAGACGAGUC
GGAUCUCCCUUUGGGCCGCCUCCCCGCCUGUUAAUUAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAA
Exemplary RNA polynucleotide encoding KIT
(SEQ ID NO: 46)
UAAUACGACUCACUAUAAGGACUCACUAUUUGUUUUCGCGCCCAGUUGCAAAAAGUGUCGCCACCAUGA
GAGGCGCUCGCGGCGCCUGGGAUUUUCUCUGCGUUCUGCUCCUACUGCUUCGCGUCCAGACAGGCUCUU
CUCAACCAUCUGUGAGUCCAGGGGAACCGUCUCCACCAUCCAUCCAUCCAGGAAAAUCAGACUUAAUAG
UCCGCGUGGGCGACGAGAUUAGGCUGUUAUGCACUGAUCCGGGCUUUGUCAAAUGGACUUUUGAGAUCC
UGGAUGAAACGAAUGAGAAUAAGCAGAAUGAAUGGAUCACGGAAAAGGCAGAAGCCACCAACACCGGCA
AAUACACGUGCACCAACAAACACGGCUUAAGCAAUUCCAUUUAUGUGUUUGUUAGAGAUCCUGCCAAGC
UUUUCCUUGUUGACCGCUCCUUGUAUGGGAAAGAAGACAACGACACGCUGGUCCGCUGUCCUCUCACAG
ACCCAGAAGUGACCAAUUAUUCCCUCAAGGGGUGCCAGGGGAAGCCUCUUCCCAAGGACUUGAGGUUUA
UUCCUGACCCCAAGGCGGGCAUCAUGAUCAAAAGUGUGAAACGCGCCUACCAUCGGCUCUGUCUGCAUU
GUUCUGUGGACCAGGAGGGCAAGUCAGUGCUGUCGGAAAAAUUCAUCCUGAAAGUGAGGCCAGCCUUCA
AAGCUGUGCCUGUUGUGUCUGUGUCCAAAGCAAGCUAUCUUCUUAGGGAAGGGGAAGAAUUCACAGUGA
CGUGCACAAUAAAAGAUGUGUCUAGUUCUGUGUACUCAACGUGGAAAAGAGAAAACAGUCAGACUAAAC
UACAGGAGAAAUAUAAUAGCUGGCAUCACGGUGACUUCAAUUAUGAACGUCAGGCAACGUUGACUAUCA
GUUCAGCGAGAGUUAAUGAUUCUGGAGUGUUCAUGUGUUAUGCCAAUAAUACUUUUGGAUCAGCAAAUG
UCACAACAACCUUGGAAGUAGUAGAUAAAGGAUUCAUUAAUAUCUUCCCCAUGAUAAACACUACAGUAU
UUGUAAACGAUGGAGAAAAUGUAGAUUUGAUUGUUGAAUAUGAAGCAUUCCCCAAACCUGAACACCAGC
AGUGGAUCUAUAUGAACAGAACCUUCACUGAUAAAUGGGAAGAUUAUCCCAAGUCUGAGAAUGAAAGUA
AUAUCAGAUACGUAAGUGAACUUCAUCUAACGAGAUUAAAAGGCACCGAAGGAGGCACUUACACAUUCC
UAGUGUCCAAUUCUGACGUCAAUGCUGCCAUAGCAUUUAAUGUUUAUGUGAAUACAAAACCAGAAAUCC
UGACUUACGACAGGCUCGUGAAUGGCAUGCUCCAAUGUGUGGCAGCAGGAUUCCCAGAGCCCACAAUAG
AUUGGUAUUUUUGUCCAGGAACUGAGCAGAGAUGCUCUGCUUCUGUACUGCCAGUGGAUGUGCAGACAC
UAAACUCAUCUGGGCCACCGUUUGGAAAGCUAGUGGUUCAGAGUUCUAUAGAUUCUAGUGCAUUCAAGC
ACAAUGGCACGGUUGAAUGUAAGGCUUACAACGAUGUGGGCAAGACUUCUGCCUAUUUUAACUUUGCAU
UUAAAGGUAACAACAAAGAGCAAAUCCAUCCCCACACCCUGUUCACUCCUUUGCUGAUUGGUUUCGUAA
UCGUAGCUGGCAUGAUGUGCAUUAUUGUGAUGAUUCUGACCUACAAAUAUUUACAGAAACCCAUGUAUG
AAGUACAGUGGAAGGUUGUUGAGGAGAUAAAUGGAAACAAUUAUGUUUACAUAGACCCAACACAACUUC
CUUAUGAUCACAAAUGGGAGUUUCCCAGAAACAGGCUGAGUUUUGGGAAAACCCUGGGUGCUGGAGCUU
UCGGGAAGGUUGUUGAGGCAACUGCUUAUGGCUUAAUUAAGUCAGAUGCGGCCAUGACUGUCGCUGUAA
AGAUGCUCAAGCCGAGUGCCCAUUUGACAGAACGGGAAGCCCUCAUGUCUGAACUCAAAGUCCUGAGUU
ACCUUGGUAAUCACAUGAAUAUUGUGAAUCUACUUGGAGCCUGCACCAUUGGAGGGCCCACCCUGGUCA
UUACAGAAUAUUGUUGCUAUGGUGAUCUUUUGAAUUUUUUGAGAAGAAAACGUGAUUCAUUUAUUUGUU
CAAAGCAGGAAGAUCAUGCAGAAGCUGCACUUUAUAAGAAUCUUCUGCAUUCAAAGGAGUCUUCCUGCA
GCGAUAGUACUAAUGAGUACAUGGACAUGAAACCUGGAGUUUCUUAUGUUGUCCCAACCAAGGCCGACA
AAAGGAGAUCUGUGAGAAUAGGCUCAUACAUAGAAAGAGAUGUGACUCCCGCCAUCAUGGAGGAUGACG
AGUUGGCCCUAGACUUAGAAGACUUGCUGAGCUUUUCUUACCAGGUGGCAAAGGGCAUGGCUUUCCUCG
CCUCCAAGAAUUGUAUUCACAGAGACUUGGCAGCCAGAAAUAUCCUCCUUACUCAUGGUCGGAUCACAA
AGAUUUGUGAUUUUGGUCUAGCCAGAGACAUCAAGAAUGAUUCUAAUUAUGUGGUUAAAGGAAACGCUC
GACUACCUGUGAAGUGGAUGGCACCUGAAAGCAUUUUCAACUGUGUAUACACGUUUGAAAGUGACGUCU
GGUCCUAUGGGAUUUUUCUUUGGGAGCUGUUCUCUUUAGGAAGCAGCCCCUAUCCUGGAAUGCCGGUCG
AUUCUAAGUUCUACAAGAUGAUCAAGGAAGGCUUCCGGAUGCUCAGCCCUGAACACGCACCUGCUGAAA
UGUAUGACAUAAUGAAGACUUGCUGGGAUGCAGAUCCCCUAAAAAGACCAACAUUCAAGCAAAUUGUUC
AGCUAAUUGAGAAGCAGAUUUCAGAGAGCACCAAUCAUAUUUACUCCAACUUAGCAAACUGCAGCCCCA
ACCGACAGAAGCCCGUGGUAGACCAUUCUGUGCGGAUCAAUUCUGUCGGCAGCACCGCUUCCUCCUCCC
AGCCUCUGCUUGUGCACGACGAUGUCUGAGCGGCCGCGUCGAUCGACAAUCAACCUCUGGAUUACAAAA
UUUGUGAAAGAUUGACUGGUAUUCUUAACUAUGUUGCUCCUUUUACGCUAUGUGGAUACGCUGCUUUAA
UGCCUUUGUAUCAUGCUAUUGCUUCCCGUAUGGCUUUCAUUUUCUCCUCCUUGUAUAAAUCCUGGUUGC
UGUCUCUUUAUGAGGAGUUGUGGCCCGUUGUCAGGCAACGUGGCGUGGUGUGCACUGUGUUUGCUGACG
CAACCCCCACUGGUUGGGGCAUUGCCACCACCUGUCAGCUCCUUUCCGGGACUUUCGCUUUCCCCCUCC
CUAUUGCCACGGCGGAACUCAUCGCCGCCUGCCUUGCCCGCUGCUGGACAGGGGCUCGGCUGUUGGGCA
CUGACAAUUCCGUGGUGUUGUCGGGGAAAUCAUCGUCCUUUCCUUGGCUGCUCGCCUGUGUUGCCACCU
GGAUUCUGCGCGGGACGUCCUUCUGCUACGUCCCUUCGGCCCUCAAUCCAGCGGACCUUCCUUCCCGCG
GCCUGCUGCCGGCUCUGCGGCCUCUUCCGCGUCUUCGCCUUCGCCCUCAGACGAGUCGGAUCUCCCUUU
GGGCCGCCUCCCCGCCUGGAAUUCGAGCUCGUAAAAAAAAAAAAAAAAAAAAAAAAAAAAAAAAAAAAA
AAAAAAAAAAAAAAAAAAAAAAAAAAA

### DNA Polynucleotide

In one aspect, the present invention provides a DNA polynucleotide encoding a RNA polynucleotide according to the present invention. Preferably, the RNA polynucleotide encodes an engraftment enhancer.

As used herein, a “DNA polynucleotide” may refer to a polynucleotide which consists substantially of deoxyribonucleotides, which are nucleotides containing deoxyribose as its pentose component.

Exemplary DNA polynucleotides are provided below in SEQ ID NOs: 48-55. In some embodiments, the DNA polynucleotide comprises or consists of a nucleotide sequence having least 70%, at least 75%, at least 80%, at least 85%, at least 90%, at least 95%, at least 96%, at least 97%, at least 98%, at least 99% or 100% identity to any of SEQ ID NOs: 48-55. In some embodiments, the DNA polynucleotide comprises or consists of the nucleotide sequence of any of SEQ ID NOs: 48-55.

In one aspect, the present invention provides a DNA polynucleotide comprising or consisting a nucleotide sequence having least 70%, at least 75%, at least 80%, at least 85%, at least 90%, at least 95%, at least 96%, at least 97%, at least 98%, at least 99% or 100% identity to any of SEQ ID NOs: 48-55.

In one aspect, the present invention provides a DNA polynucleotide comprising or consisting of the nucleotide sequence of any of SEQ ID NOs: 48-55.

Exemplary DNA polynucleotide encoding an RNA polynucleotide encoding
CXCR4 WT
(SEQ ID NO: 48)
TAATACGACTCACTATAAGGACTCACTATTTGTTTTCGCGCCCAGTTGCAAAAAGTGTCGCCACCATGG
AAGGCATCAGCATCTACACCAGCGACAACTACACCGAGGAAATGGGCAGCGGCGACTACGACAGCATGA
AGGAACCCTGCTTCCGGGAAGAGAACGCCAACTTCAACAAGATCTTCCTGCCCACAATCTACAGCATCA
TCTTTCTGACCGGCATCGTGGGCAACGGACTCGTGATCCTCGTGATGGGCTACCAGAAAAAGCTGCGGA
GCATGACCGACAAGTACCGGCTGCACCTGAGCGTGGCCGACCTGCTGTTCGTGATCACCCTGCCTTTCT
GGGCCGTGGACGCCGTGGCCAATTGGTACTTCGGCAACTTCCTGTGCAAGGCCGTGCACGTGATCTACA
CAGTGAACCTGTACAGCAGCGTGCTGATCCTGGCCTTCATCAGCCTGGACAGATACCTGGCCATCGTGC
ACGCCACCAACAGCCAGCGGCCTAGAAAGCTGCTGGCCGAGAAGGTGGTGTACGTGGGCGTGTGGATTC
CCGCCCTGCTGCTGACCATCCCCGACTTCATCTTCGCCAACGTGTCCGAGGCCGACGACCGGTACATCT
GCGACCGGTTCTACCCCAACGACCTGTGGGTGGTGGTGTTCCAGTTCCAGCACATCATGGTGGGACTGA
TCCTGCCTGGCATCGTGATTCTGAGCTGCTACTGCATCATCATCAGCAAGCTGAGCCACAGCAAGGGCC
ACCAGAAGCGGAAGGCCCTGAAAACCACCGTGATCCTGATTCTGGCTTTCTTCGCCTGCTGGCTGCCCT
ACTACATCGGCATCAGCATCGACAGCTTCATCCTGCTGGAAATCATCAAGCAGGGCTGCGAGTTCGAGA
ACACCGTGCACAAGTGGATCAGCATTACCGAGGCCCTGGCCTTTTTCCACTGCTGCCTGAACCCTATCC
TGTACGCCTTCCTGGGCGCCAAGTTCAAGACCTCTGCCCAGCACGCCCTGACCAGCGTGTCCAGAGGAA
GCAGCCTGAAGATCCTGAGCAAGGGCAAGAGAGGCGGCCACAGCTCCGTGTCTACAGAGAGCGAGAGCA
GCAGCTTCCACAGCAGCTGAAATCAACCTCTGGATTACAAAATTTGTGAAAGATTGACTGGTATTCTTA
ACTATGTTGCTCCTTTTACGCTATGTGGATACGCTGCTTTAATGCCTTTGTATCATGCTATTGCTTCCC
GTATGGCTTTCATTTTCTCCTCCTTGTATAAATCCTGGTTGCTGTCTCTTTATGAGGAGTTGTGGCCCG
TTGTCAGGCAACGTGGCGTGGTGTGCACTGTGTTTGCTGACGCAACCCCCACTGGTTGGGGCATTGCCA
CCACCTGTCAGCTCCTTTCCGGGACTTTCGCTTTCCCCCTCCCTATTGCCACGGCGGAACTCATCGCCG
CCTGCCTTGCCCGCTGCTGGACAGGGGCTCGGCTGTTGGGCACTGACAATTCCGTGGTGTTGTCGGGGA
AATCATCGTCCTTTCCTTGGCTGCTCGCCTGTGTTGCCACCTGGATTCTGCGCGGGACGTCCTTCTGCT
ACGTCCCTTCGGCCCTCAATCCAGCGGACCTTCCTTCCCGCGGCCTGCTGCCGGCTCTGCGGCCTCTTC
CGCGTCTTCGCCTTCGCCCTCAGACGAGTCGGATCTCCCTTTGGGCCGCCTCCCCGCCTGTTAATTAAA
AAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAA
Exemplary DNA polynucleotide encoding an RNA polynucleotide encoding
CXCR4 V160L
(SEQ ID NO: 49)
TAATACGACTCACTATAAGGACTCACTATTTGTTTTCGCGCCCAGTTGCAAAAAGTGTCGCCACCATGG
AAGGCATCAGCATCTACACCAGCGACAACTACACCGAGGAAATGGGCAGCGGCGACTACGACAGCATGA
AGGAACCCTGCTTCCGGGAAGAGAACGCCAACTTCAACAAGATCTTCCTGCCCACAATCTACAGCATCA
TCTTTCTGACCGGCATCGTGGGCAACGGACTCGTGATCCTCGTGATGGGCTACCAGAAAAAGCTGCGGA
GCATGACCGACAAGTACCGGCTGCACCTGAGCGTGGCCGACCTGCTGTTCGTGATCACCCTGCCTTTCT
GGGCCGTGGACGCCGTGGCCAATTGGTACTTCGGCAACTTCCTGTGCAAGGCCGTGCACGTGATCTACA
CAGTGAACCTGTACAGCAGCGTGCTGATCCTGGCCTTCATCAGCCTGGACAGATACCTGGCCATCGTGC
ACGCCACCAACAGCCAGCGGCCTAGAAAGCTGCTGGCCGAGAAGGTGGTGTACGTGGGCCTGTGGATTC
CCGCCCTGCTGCTGACCATCCCCGACTTCATCTTCGCCAACGTGTCCGAGGCCGACGACCGGTACATCT
GCGACCGGTTCTACCCCAACGACCTGTGGGTGGTGGTGTTCCAGTTCCAGCACATCATGGTGGGACTGA
TCCTGCCTGGCATCGTGATTCTGAGCTGCTACTGCATCATCATCAGCAAGCTGAGCCACAGCAAGGGCC
ACCAGAAGCGGAAGGCCCTGAAAACCACCGTGATCCTGATTCTGGCTTTCTTCGCCTGCTGGCTGCCCT
ACTACATCGGCATCAGCATCGACAGCTTCATCCTGCTGGAAATCATCAAGCAGGGCTGCGAGTTCGAGA
ACACCGTGCACAAGTGGATCAGCATTACCGAGGCCCTGGCCTTTTTCCACTGCTGCCTGAACCCTATCC
TGTACGCCTTCCTGGGCGCCAAGTTCAAGACCTCTGCCCAGCACGCCCTGACCAGCGTGTCCAGAGGAA
GCAGCCTGAAGATCCTGAGCAAGGGCAAGAGAGGCGGCCACAGCTCCGTGTCTACAGAGAGCGAGAGCA
GCAGCTTCCACAGCAGCTGAAATCAACCTCTGGATTACAAAATTTGTGAAAGATTGACTGGTATTCTTA
ACTATGTTGCTCCTTTTACGCTATGTGGATACGCTGCTTTAATGCCTTTGTATCATGCTATTGCTTCCC
GTATGGCTTTCATTTTCTCCTCCTTGTATAAATCCTGGTTGCTGTCTCTTTATGAGGAGTTGTGGCCCG
TTGTCAGGCAACGTGGCGTGGTGTGCACTGTGTTTGCTGACGCAACCCCCACTGGTTGGGGCATTGCCA
CCACCTGTCAGCTCCTTTCCGGGACTTTCGCTTTCCCCCTCCCTATTGCCACGGCGGAACTCATCGCCG
CCTGCCTTGCCCGCTGCTGGACAGGGGCTCGGCTGTTGGGCACTGACAATTCCGTGGTGTTGTCGGGGA
AATCATCGTCCTTTCCTTGGCTGCTCGCCTGTGTTGCCACCTGGATTCTGCGCGGGACGTCCTTCTGCT
ACGTCCCTTCGGCCCTCAATCCAGCGGACCTTCCTTCCCGCGGCCTGCTGCCGGCTCTGCGGCCTCTTC
CGCGTCTTCGCCTTCGCCCTCAGACGAGTCGGATCTCCCTTTGGGCCGCCTCCCCGCCTGTTAATTAAA
AAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAA
Exemplary DNA polynucleotide encoding an RNA polynucleotide encoding
CXCR4 A175F
(SEQ ID NO: 50)
TAATACGACTCACTATAAGGACTCACTATTTGTTTTCGCGCCCAGTTGCAAAAAGTGTCGCCACCATGG
AAGGCATCAGCATCTACACCAGCGACAACTACACCGAGGAAATGGGCAGCGGCGACTACGACAGCATGA
AGGAACCCTGCTTCCGGGAAGAGAACGCCAACTTCAACAAGATCTTCCTGCCCACAATCTACAGCATCA
TCTTTCTGACCGGCATCGTGGGCAACGGACTCGTGATCCTCGTGATGGGCTACCAGAAAAAGCTGCGGA
GCATGACCGACAAGTACCGGCTGCACCTGAGCGTGGCCGACCTGCTGTTCGTGATCACCCTGCCTTTCT
GGGCCGTGGACGCCGTGGCCAATTGGTACTTCGGCAACTTCCTGTGCAAGGCCGTGCACGTGATCTACA
CAGTGAACCTGTACAGCAGCGTGCTGATCCTGGCCTTCATCAGCCTGGACAGATACCTGGCCATCGTGC
ACGCCACCAACAGCCAGCGGCCTAGAAAGCTGCTGGCCGAGAAGGTGGTGTACGTGGGCGTGTGGATTC
CCGCCCTGCTGCTGACCATCCCCGACTTCATCTTCTTCAACGTGTCCGAGGCCGACGACCGGTACATCT
GCGACCGGTTCTACCCCAACGACCTGTGGGTGGTGGTGTTCCAGTTCCAGCACATCATGGTGGGACTGA
TCCTGCCTGGCATCGTGATTCTGAGCTGCTACTGCATCATCATCAGCAAGCTGAGCCACAGCAAGGGCC
ACCAGAAGCGGAAGGCCCTGAAAACCACCGTGATCCTGATTCTGGCTTTCTTCGCCTGCTGGCTGCCCT
ACTACATCGGCATCAGCATCGACAGCTTCATCCTGCTGGAAATCATCAAGCAGGGCTGCGAGTTCGAGA
ACACCGTGCACAAGTGGATCAGCATTACCGAGGCCCTGGCCTTTTTCCACTGCTGCCTGAACCCTATCC
TGTACGCCTTCCTGGGCGCCAAGTTCAAGACCTCTGCCCAGCACGCCCTGACCAGCGTGTCCAGAGGAA
GCAGCCTGAAGATCCTGAGCAAGGGCAAGAGAGGCGGCCACAGCTCCGTGTCTACAGAGAGCGAGAGCA
GCAGCTTCCACAGCAGCTGAAATCAACCTCTGGATTACAAAATTTGTGAAAGATTGACTGGTATTCTTA
ACTATGTTGCTCCTTTTACGCTATGTGGATACGCTGCTTTAATGCCTTTGTATCATGCTATTGCTTCCC
GTATGGCTTTCATTTTCTCCTCCTTGTATAAATCCTGGTTGCTGTCTCTTTATGAGGAGTTGTGGCCCG
TTGTCAGGCAACGTGGCGTGGTGTGCACTGTGTTTGCTGACGCAACCCCCACTGGTTGGGGCATTGCCA
CCACCTGTCAGCTCCTTTCCGGGACTTTCGCTTTCCCCCTCCCTATTGCCACGGCGGAACTCATCGCCG
CCTGCCTTGCCCGCTGCTGGACAGGGGCTCGGCTGTTGGGCACTGACAATTCCGTGGTGTTGTCGGGGA
AATCATCGTCCTTTCCTTGGCTGCTCGCCTGTGTTGCCACCTGGATTCTGCGCGGGACGTCCTTCTGCT
ACGTCCCTTCGGCCCTCAATCCAGCGGACCTTCCTTCCCGCGGCCTGCTGCCGGCTCTGCGGCCTCTTC
CGCGTCTTCGCCTTCGCCCTCAGACGAGTCGGATCTCCCTTTGGGCCGCCTCCCCGCCTGTTAATTAAA
AAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAA
Exemplary DNA polynucleotide encoding an RNA polynucleotide encoding
CXCR4 Q200A
(SEQ ID NO: 51)
TAATACGACTCACTATAAGGACTCACTATTTGTTTTCGCGCCCAGTTGCAAAAAGTGTCGCCACCATGG
AAGGCATCAGCATCTACACCAGCGACAACTACACCGAGGAAATGGGCAGCGGCGACTACGACAGCATGA
AGGAACCCTGCTTCCGGGAAGAGAACGCCAACTTCAACAAGATCTTCCTGCCCACAATCTACAGCATCA
TCTTTCTGACCGGCATCGTGGGCAACGGACTCGTGATCCTCGTGATGGGCTACCAGAAAAAGCTGCGGA
GCATGACCGACAAGTACCGGCTGCACCTGAGCGTGGCCGACCTGCTGTTCGTGATCACCCTGCCTTTCT
GGGCCGTGGACGCCGTGGCCAATTGGTACTTCGGCAACTTCCTGTGCAAGGCCGTGCACGTGATCTACA
CAGTGAACCTGTACAGCAGCGTGCTGATCCTGGCCTTCATCAGCCTGGACAGATACCTGGCCATCGTGC
ACGCCACCAACAGCCAGCGGCCTAGAAAGCTGCTGGCCGAGAAGGTGGTGTACGTGGGCGTGTGGATTC
CCGCCCTGCTGCTGACCATCCCCGACTTCATCTTCGCCAACGTGTCCGAGGCCGACGACCGGTACATCT
GCGACCGGTTCTACCCCAACGACCTGTGGGTGGTGGTGTTCGCGTTCCAGCACATCATGGTGGGACTGA
TCCTGCCTGGCATCGTGATTCTGAGCTGCTACTGCATCATCATCAGCAAGCTGAGCCACAGCAAGGGCC
ACCAGAAGCGGAAGGCCCTGAAAACCACCGTGATCCTGATTCTGGCTTTCTTCGCCTGCTGGCTGCCCT
ACTACATCGGCATCAGCATCGACAGCTTCATCCTGCTGGAAATCATCAAGCAGGGCTGCGAGTTCGAGA
ACACCGTGCACAAGTGGATCAGCATTACCGAGGCCCTGGCCTTTTTCCACTGCTGCCTGAACCCTATCC
TGTACGCCTTCCTGGGCGCCAAGTTCAAGACCTCTGCCCAGCACGCCCTGACCAGCGTGTCCAGAGGAA
GCAGCCTGAAGATCCTGAGCAAGGGCAAGAGAGGCGGCCACAGCTCCGTGTCTACAGAGAGCGAGAGCA
GCAGCTTCCACAGCAGCTGAAATCAACCTCTGGATTACAAAATTTGTGAAAGATTGACTGGTATTCTTA
ACTATGTTGCTCCTTTTACGCTATGTGGATACGCTGCTTTAATGCCTTTGTATCATGCTATTGCTTCCC
GTATGGCTTTCATTTTCTCCTCCTTGTATAAATCCTGGTTGCTGTCTCTTTATGAGGAGTTGTGGCCCG
TTGTCAGGCAACGTGGCGTGGTGTGCACTGTGTTTGCTGACGCAACCCCCACTGGTTGGGGCATTGCCA
CCACCTGTCAGCTCCTTTCCGGGACTTTCGCTTTCCCCCTCCCTATTGCCACGGCGGAACTCATCGCCG
CCTGCCTTGCCCGCTGCTGGACAGGGGCTCGGCTGTTGGGCACTGACAATTCCGTGGTGTTGTCGGGGA
AATCATCGTCCTTTCCTTGGCTGCTCGCCTGTGTTGCCACCTGGATTCTGCGCGGGACGTCCTTCTGCT
ACGTCCCTTCGGCCCTCAATCCAGCGGACCTTCCTTCCCGCGGCCTGCTGCCGGCTCTGCGGCCTCTTC
CGCGTCTTCGCCTTCGCCCTCAGACGAGTCGGATCTCCCTTTGGGCCGCCTCCCCGCCTGTTAATTAAA
AAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAA
Exemplary DNA polynucleotide encoding an RNA polynucleotide encoding
CXCR4 D262N
(SEQ ID NO: 52)
TAATACGACTCACTATAAGGACTCACTATTTGTTTTCGCGCCCAGTTGCAAAAAGTGTCGCCACCATGG
AAGGCATCAGCATCTACACCAGCGACAACTACACCGAGGAAATGGGCAGCGGCGACTACGACAGCATGA
AGGAACCCTGCTTCCGGGAAGAGAACGCCAACTTCAACAAGATCTTCCTGCCCACAATCTACAGCATCA
TCTTTCTGACCGGCATCGTGGGCAACGGACTCGTGATCCTCGTGATGGGCTACCAGAAAAAGCTGCGGA
GCATGACCGACAAGTACCGGCTGCACCTGAGCGTGGCCGACCTGCTGTTCGTGATCACCCTGCCTTTCT
GGGCCGTGGACGCCGTGGCCAATTGGTACTTCGGCAACTTCCTGTGCAAGGCCGTGCACGTGATCTACA
CAGTGAACCTGTACAGCAGCGTGCTGATCCTGGCCTTCATCAGCCTGGACAGATACCTGGCCATCGTGC
ACGCCACCAACAGCCAGCGGCCTAGAAAGCTGCTGGCCGAGAAGGTGGTGTACGTGGGCGTGTGGATTC
CCGCCCTGCTGCTGACCATCCCCGACTTCATCTTCGCCAACGTGTCCGAGGCCGACGACCGGTACATCT
GCGACCGGTTCTACCCCAACGACCTGTGGGTGGTGGTGTTCCAGTTCCAGCACATCATGGTGGGACTGA
TCCTGCCTGGCATCGTGATTCTGAGCTGCTACTGCATCATCATCAGCAAGCTGAGCCACAGCAAGGGCC
ACCAGAAGCGGAAGGCCCTGAAAACCACCGTGATCCTGATTCTGGCTTTCTTCGCCTGCTGGCTGCCCT
ACTACATCGGCATCAGCATCAACAGCTTCATCCTGCTGGAAATCATCAAGCAGGGCTGCGAGTTCGAGA
ACACCGTGCACAAGTGGATCAGCATTACCGAGGCCCTGGCCTTTTTCCACTGCTGCCTGAACCCTATCC
TGTACGCCTTCCTGGGCGCCAAGTTCAAGACCTCTGCCCAGCACGCCCTGACCAGCGTGTCCAGAGGAA
GCAGCCTGAAGATCCTGAGCAAGGGCAAGAGAGGCGGCCACAGCTCCGTGTCTACAGAGAGCGAGAGCA
GCAGCTTCCACAGCAGCTGAAATCAACCTCTGGATTACAAAATTTGTGAAAGATTGACTGGTATTCTTA
ACTATGTTGCTCCTTTTACGCTATGTGGATACGCTGCTTTAATGCCTTTGTATCATGCTATTGCTTCCC
GTATGGCTTTCATTTTCTCCTCCTTGTATAAATCCTGGTTGCTGTCTCTTTATGAGGAGTTGTGGCCCG
TTGTCAGGCAACGTGGCGTGGTGTGCACTGTGTTTGCTGACGCAACCCCCACTGGTTGGGGCATTGCCA
CCACCTGTCAGCTCCTTTCCGGGACTTTCGCTTTCCCCCTCCCTATTGCCACGGCGGAACTCATCGCCG
CCTGCCTTGCCCGCTGCTGGACAGGGGCTCGGCTGTTGGGCACTGACAATTCCGTGGTGTTGTCGGGGA
AATCATCGTCCTTTCCTTGGCTGCTCGCCTGTGTTGCCACCTGGATTCTGCGCGGGACGTCCTTCTGCT
ACGTCCCTTCGGCCCTCAATCCAGCGGACCTTCCTTCCCGCGGCCTGCTGCCGGCTCTGCGGCCTCTTC
CGCGTCTTCGCCTTCGCCCTCAGACGAGTCGGATCTCCCTTTGGGCCGCCTCCCCGCCTGTTAATTAAA
AAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAA
Exemplary DNA polynucleotide encoding an RNA polynucleotide encoding
CXCR4 H281A
(SEQ ID NO: 53)
TAATACGACTCACTATAAGGACTCACTATTTGTTTTCGCGCCCAGTTGCAAAAAGTGTCGCCACCATGG
AAGGCATCAGCATCTACACCAGCGACAACTACACCGAGGAAATGGGCAGCGGCGACTACGACAGCATGA
AGGAACCCTGCTTCCGGGAAGAGAACGCCAACTTCAACAAGATCTTCCTGCCCACAATCTACAGCATCA
TCTTTCTGACCGGCATCGTGGGCAACGGACTCGTGATCCTCGTGATGGGCTACCAGAAAAAGCTGCGGA
GCATGACCGACAAGTACCGGCTGCACCTGAGCGTGGCCGACCTGCTGTTCGTGATCACCCTGCCTTTCT
GGGCCGTGGACGCCGTGGCCAATTGGTACTTCGGCAACTTCCTGTGCAAGGCCGTGCACGTGATCTACA
CAGTGAACCTGTACAGCAGCGTGCTGATCCTGGCCTTCATCAGCCTGGACAGATACCTGGCCATCGTGC
ACGCCACCAACAGCCAGCGGCCTAGAAAGCTGCTGGCCGAGAAGGTGGTGTACGTGGGCGTGTGGATTC
CCGCCCTGCTGCTGACCATCCCCGACTTCATCTTCGCCAACGTGTCCGAGGCCGACGACCGGTACATCT
GCGACCGGTTCTACCCCAACGACCTGTGGGTGGTGGTGTTCCAGTTCCAGCACATCATGGTGGGACTGA
TCCTGCCTGGCATCGTGATTCTGAGCTGCTACTGCATCATCATCAGCAAGCTGAGCCACAGCAAGGGCC
ACCAGAAGCGGAAGGCCCTGAAAACCACCGTGATCCTGATTCTGGCTTTCTTCGCCTGCTGGCTGCCCT
ACTACATCGGCATCAGCATCGACAGCTTCATCCTGCTGGAAATCATCAAGCAGGGCTGCGAGTTCGAGA
ACACCGTGGCCAAGTGGATCAGCATTACCGAGGCCCTGGCCTTTTTCCACTGCTGCCTGAACCCTATCC
TGTACGCCTTCCTGGGCGCCAAGTTCAAGACCTCTGCCCAGCACGCCCTGACCAGCGTGTCCAGAGGAA
GCAGCCTGAAGATCCTGAGCAAGGGCAAGAGAGGCGGCCACAGCTCCGTGTCTACAGAGAGCGAGAGCA
GCAGCTTCCACAGCAGCTGAAATCAACCTCTGGATTACAAAATTTGTGAAAGATTGACTGGTATTCTTA
ACTATGTTGCTCCTTTTACGCTATGTGGATACGCTGCTTTAATGCCTTTGTATCATGCTATTGCTTCCC
GTATGGCTTTCATTTTCTCCTCCTTGTATAAATCCTGGTTGCTGTCTCTTTATGAGGAGTTGTGGCCCG
TTGTCAGGCAACGTGGCGTGGTGTGCACTGTGTTTGCTGACGCAACCCCCACTGGTTGGGGCATTGCCA
CCACCTGTCAGCTCCTTTCCGGGACTTTCGCTTTCCCCCTCCCTATTGCCACGGCGGAACTCATCGCCG
CCTGCCTTGCCCGCTGCTGGACAGGGGCTCGGCTGTTGGGCACTGACAATTCCGTGGTGTTGTCGGGGA
AATCATCGTCCTTTCCTTGGCTGCTCGCCTGTGTTGCCACCTGGATTCTGCGCGGGACGTCCTTCTGCT
ACGTCCCTTCGGCCCTCAATCCAGCGGACCTTCCTTCCCGCGGCCTGCTGCCGGCTCTGCGGCCTCTTC
CGCGTCTTCGCCTTCGCCCTCAGACGAGTCGGATCTCCCTTTGGGCCGCCTCCCCGCCTGTTAATTAAA
AAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAA
Exemplary DNA polynucleotide encoding an RNA polynucleotide encoding
ITGA4
(SEQ ID NO: 54)
TAATACGACTCACTATAAGGACTCACTATTTGTTTTCGCGCCCAGTTGCAAAAAGTGTCGCCACCATGG
CTTGGGAGGCTCGGAGAGAACCTGGACCTAGAAGAGCTGCCGTGCGGGAGACTGTCATGCTGCTGCTGT
GCCTGGGGGTGCCCACAGGCAGACCTTACAACGTGGATACCGAGAGCGCCCTGCTGTATCAGGGCCCCC
ACAACACCCTGTTTGGCTACTCTGTGGTGCTGCACAGCCACGGCGCCAACCGCTGGCTGCTGGTGGGCG
CCCCCACCGCCAATTGGCTGGCCAATGCCTCCGTGATCAACCCAGGCGCCATCTACAGATGTCGGATCG
GCAAGAATCCTGGCCAGACATGCGAGCAGCTGCAGCTGGGCTCCCCCAACGGCGAGCCTTGTGGCAAGA
CATGCCTGGAGGAGAGGGACAATCAGTGGCTGGGCGTGACACTGAGCAGACAGCCCGGCGAGAACGGCT
CCATCGTGACATGCGGCCACAGATGGAAGAACATCTTTTACATCAAGAACGAGAATAAGCTGCCAACAG
GCGGCTGCTATGGCGTGCCCCCAGACCTGAGAACAGAGCTGAGCAAGCGGATCGCCCCATGCTACCAGG
ATTATGTGAAGAAGTTTGGCGAGAATTTTGCCTCTTGCCAGGCCGGCATCTCCTCCTTCTACACCAAGG
ATCTGATCGTGATGGGCGCCCCTGGCTCTTCCTATTGGACAGGCTCTCTGTTCGTGTATAATATCACAA
CCAACAAGTACAAGGCCTTCCTGGACAAGCAGAACCAGGTGAAGTTCGGCTCCTATCTGGGCTACAGCG
TGGGCGCCGGCCACTTTCGGTCTCAGCACACCACAGAGGTGGTGGGCGGCGCCCCCCAGCACGAGCAGA
TCGGCAAGGCCTACATCTTCTCCATCGACGAGAAGGAGCTGAATATCCTGCACGAGATGAAGGGCAAGA
AGCTGGGCTCCTACTTTGGCGCCTCCGTGTGCGCCGTGGACCTGAATGCCGACGGCTTTTCCGACCTGC
TGGTGGGCGCCCCAATGCAGTCCACAATCAGAGAGGAGGGCAGAGTGTTCGTGTATATCAATTCCGGCA
GCGGCGCCGTGATGAATGCCATGGAGACCAATCTGGTGGGCTCCGACAAGTATGCCGCCAGATTCGGCG
AGAGCATCGTGAATCTGGGCGACATCGACAACGATGGCTTCGAGGACGTGGCCATCGGCGCCCCACAGG
AGGATGACCTGCAGGGCGCCATCTATATCTATAACGGCCGGGCCGACGGCATCTCTAGCACCTTCTCCC
AGAGAATCGAGGGCCTGCAGATCAGCAAGTCCCTGAGCATGTTCGGCCAGAGCATCTCCGGCCAGATCG
ACGCCGATAATAACGGCTACGTGGATGTGGCCGTGGGCGCCTTTAGAAGCGACTCCGCCGTGCTGCTGA
GAACAAGGCCCGTGGTGATCGTGGATGCCTCCCTGTCTCACCCCGAGTCCGTGAATCGGACAAAGTTTG
ACTGCGTGGAGAATGGCTGGCCAAGCGTGTGCATCGATCTGACACTGTGCTTTTCCTATAAGGGCAAGG
AGGTGCCAGGCTATATCGTGCTGTTTTACAACATGTCTCTGGATGTGAACAGAAAGGCCGAGTCCCCCC
CAAGATTCTACTTTTCCTCTAACGGCACCTCTGATGTGATCACCGGCTCTATCCAGGTGTCCAGCAGGG
AGGCCAATTGCAGAACCCACCAGGCCTTTATGCGGAAGGATGTGCGCGACATCCTGACCCCAATCCAGA
TCGAGGCCGCCTATCACCTGGGCCCCCACGTGATCTCCAAGCGGTCCACCGAGGAGTTCCCTCCACTGC
AGCCAATCCTGCAGCAGAAGAAGGAGAAGGACATCATGAAGAAGACAATCAACTTCGCCAGGTTTTGCG
CCCACGAGAACTGTTCCGCCGACCTGCAGGTGTCTGCCAAGATCGGCTTCCTGAAGCCCCACGAGAACA
AGACATATCTGGCCGTGGGCTCCATGAAGACCCTGATGCTGAACGTGAGCCTGTTTAACGCCGGCGACG
ATGCCTACGAGACAACACTGCACGTGAAGCTGCCAGTGGGCCTGTACTTCATCAAGATCCTGGAGCTGG
AGGAGAAGCAGATCAACTGTGAGGTGACCGATAACTCCGGCGTGGTGCAGCTGGATTGCAGCATCGGCT
ATATCTACGTGGACCACCTGTCCCGCATCGACATCTCTTTTCTGCTGGACGTGTCCAGCCTGTCCCGGG
CCGAGGAGGACCTGTCCATCACAGTGCACGCCACCTGCGAGAATGAGGAGGAGATGGACAACCTGAAGC
ACTCCAGAGTGACAGTGGCCATCCCACTGAAGTACGAGGTGAAGCTGACAGTGCACGGCTTTGTGAATC
CAACCTCCTTCGTGTACGGCTCCAATGACGAGAATGAGCCAGAGACATGTATGGTGGAGAAGATGAACC
TGACATTTCACGTGATCAATACAGGCAATTCTATGGCCCCTAACGTGAGCGTGGAGATCATGGTGCCAA
ATTCTTTCAGCCCACAGACAGACAAGCTGTTTAACATCCTGGACGTGCAGACAACCACAGGCGAGTGTC
ACTTTGAGAACTACCAGAGAGTGTGCGCCCTGGAGCAGCAGAAGTCCGCCATGCAGACACTGAAGGGCA
TCGTGAGATTTCTGAGCAAGACAGATAAGAGGCTGCTGTACTGCATCAAGGCCGATCCCCACTGCCTGA
ATTTTCTGTGCAACTTCGGCAAGATGGAGTCTGGCAAGGAGGCCTCCGTGCACATCCAGCTGGAGGGCA
GACCCTCCATCCTGGAGATGGACGAGACCAGCGCCCTGAAGTTCGAGATCAGAGCCACAGGCTTCCCAG
AGCCCAACCCCCGGGTGATCGAGCTGAACAAGGATGAGAACGTGGCCCACGTGCTGCTGGAGGGCCTGC
ACCACCAGCGGCCCAAGAGATATTTCACCATCGTGATCATCTCCAGCTCTCTGCTGCTGGGCCTGATCG
TGCTGCTGCTGATCTCCTATGTGATGTGGAAGGCCGGCTTCTTTAAGCGGCAGTACAAGTCCATCCTGC
AGGAAGAAAATCGACGCGATTCATGGTCTTACATTAATTCTAAATCAAACGACGACTAAAATCAACCTC
TGGATTACAAAATTTGTGAAAGATTGACTGGTATTCTTAACTATGTTGCTCCTTTTACGCTATGTGGAT
ACGCTGCTTTAATGCCTTTGTATCATGCTATTGCTTCCCGTATGGCTTTCATTTTCTCCTCCTTGTATA
AATCCTGGTTGCTGTCTCTTTATGAGGAGTTGTGGCCCGTTGTCAGGCAACGTGGCGTGGTGTGCACTG
TGTTTGCTGACGCAACCCCCACTGGTTGGGGCATTGCCACCACCTGTCAGCTCCTTTCCGGGACTTTCG
CTTTCCCCCTCCCTATTGCCACGGCGGAACTCATCGCCGCCTGCCTTGCCCGCTGCTGGACAGGGGCTC
GGCTGTTGGGCACTGACAATTCCGTGGTGTTGTCGGGGAAATCATCGTCCTTTCCTTGGCTGCTCGCCT
GTGTTGCCACCTGGATTCTGCGCGGGACGTCCTTCTGCTACGTCCCTTCGGCCCTCAATCCAGCGGACC
TTCCTTCCCGCGGCCTGCTGCCGGCTCTGCGGCCTCTTCCGCGTCTTCGCCTTCGCCCTCAGACGAGTC
GGATCTCCCTTTGGGCCGCCTCCCCGCCTGTTAATTAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAA
Exemplary DNA polynucleotide encoding an RNA polynucleotide encoding
KIT
(SEQ ID NO: 55)
TAATACGACTCACTATAAGGACTCACTATTTGTTTTCGCGCCCAGTTGCAAAAAGTGTCGCCACCATGA
GAGGCGCTCGCGGCGCCTGGGATTTTCTCTGCGTTCTGCTCCTACTGCTTCGCGTCCAGACAGGCTCTT
CTCAACCATCTGTGAGTCCAGGGGAACCGTCTCCACCATCCATCCATCCAGGAAAATCAGACTTAATAG
TCCGCGTGGGCGACGAGATTAGGCTGTTATGCACTGATCCGGGCTTTGTCAAATGGACTTTTGAGATCC
TGGATGAAACGAATGAGAATAAGCAGAATGAATGGATCACGGAAAAGGCAGAAGCCACCAACACCGGCA
AATACACGTGCACCAACAAACACGGCTTAAGCAATTCCATTTATGTGTTTGTTAGAGATCCTGCCAAGC
TTTTCCTTGTTGACCGCTCCTTGTATGGGAAAGAAGACAACGACACGCTGGTCCGCTGTCCTCTCACAG
ACCCAGAAGTGACCAATTATTCCCTCAAGGGGTGCCAGGGGAAGCCTCTTCCCAAGGACTTGAGGTTTA
TTCCTGACCCCAAGGCGGGCATCATGATCAAAAGTGTGAAACGCGCCTACCATCGGCTCTGTCTGCATT
GTTCTGTGGACCAGGAGGGCAAGTCAGTGCTGTCGGAAAAATTCATCCTGAAAGTGAGGCCAGCCTTCA
AAGCTGTGCCTGTTGTGTCTGTGTCCAAAGCAAGCTATCTTCTTAGGGAAGGGGAAGAATTCACAGTGA
CGTGCACAATAAAAGATGTGTCTAGTTCTGTGTACTCAACGTGGAAAAGAGAAAACAGTCAGACTAAAC
TACAGGAGAAATATAATAGCTGGCATCACGGTGACTTCAATTATGAACGTCAGGCAACGTTGACTATCA
GTTCAGCGAGAGTTAATGATTCTGGAGTGTTCATGTGTTATGCCAATAATACTTTTGGATCAGCAAATG
TCACAACAACCTTGGAAGTAGTAGATAAAGGATTCATTAATATCTTCCCCATGATAAACACTACAGTAT
TTGTAAACGATGGAGAAAATGTAGATTTGATTGTTGAATATGAAGCATTCCCCAAACCTGAACACCAGC
AGTGGATCTATATGAACAGAACCTTCACTGATAAATGGGAAGATTATCCCAAGTCTGAGAATGAAAGTA
ATATCAGATACGTAAGTGAACTTCATCTAACGAGATTAAAAGGCACCGAAGGAGGCACTTACACATTCC
TAGTGTCCAATTCTGACGTCAATGCTGCCATAGCATTTAATGTTTATGTGAATACAAAACCAGAAATCC
TGACTTACGACAGGCTCGTGAATGGCATGCTCCAATGTGTGGCAGCAGGATTCCCAGAGCCCACAATAG
ATTGGTATTTTTGTCCAGGAACTGAGCAGAGATGCTCTGCTTCTGTACTGCCAGTGGATGTGCAGACAC
TAAACTCATCTGGGCCACCGTTTGGAAAGCTAGTGGTTCAGAGTTCTATAGATTCTAGTGCATTCAAGC
ACAATGGCACGGTTGAATGTAAGGCTTACAACGATGTGGGCAAGACTTCTGCCTATTTTAACTTTGCAT
TTAAAGGTAACAACAAAGAGCAAATCCATCCCCACACCCTGTTCACTCCTTTGCTGATTGGTTTCGTAA
TCGTAGCTGGCATGATGTGCATTATTGTGATGATTCTGACCTACAAATATTTACAGAAACCCATGTATG
AAGTACAGTGGAAGGTTGTTGAGGAGATAAATGGAAACAATTATGTTTACATAGACCCAACACAACTTC
CTTATGATCACAAATGGGAGTTTCCCAGAAACAGGCTGAGTTTTGGGAAAACCCTGGGTGCTGGAGCTT
TCGGGAAGGTTGTTGAGGCAACTGCTTATGGCTTAATTAAGTCAGATGCGGCCATGACTGTCGCTGTAA
AGATGCTCAAGCCGAGTGCCCATTTGACAGAACGGGAAGCCCTCATGTCTGAACTCAAAGTCCTGAGTT
ACCTTGGTAATCACATGAATATTGTGAATCTACTTGGAGCCTGCACCATTGGAGGGCCCACCCTGGTCA
TTACAGAATATTGTTGCTATGGTGATCTTTTGAATTTTTTGAGAAGAAAACGTGATTCATTTATTTGTT
CAAAGCAGGAAGATCATGCAGAAGCTGCACTTTATAAGAATCTTCTGCATTCAAAGGAGTCTTCCTGCA
GCGATAGTACTAATGAGTACATGGACATGAAACCTGGAGTTTCTTATGTTGTCCCAACCAAGGCCGACA
AAAGGAGATCTGTGAGAATAGGCTCATACATAGAAAGAGATGTGACTCCCGCCATCATGGAGGATGACG
AGTTGGCCCTAGACTTAGAAGACTTGCTGAGCTTTTCTTACCAGGTGGCAAAGGGCATGGCTTTCCTCG
CCTCCAAGAATTGTATTCACAGAGACTTGGCAGCCAGAAATATCCTCCTTACTCATGGTCGGATCACAA
AGATTTGTGATTTTGGTCTAGCCAGAGACATCAAGAATGATTCTAATTATGTGGTTAAAGGAAACGCTC
GACTACCTGTGAAGTGGATGGCACCTGAAAGCATTTTCAACTGTGTATACACGTTTGAAAGTGACGTCT
GGTCCTATGGGATTTTTCTTTGGGAGCTGTTCTCTTTAGGAAGCAGCCCCTATCCTGGAATGCCGGTCG
ATTCTAAGTTCTACAAGATGATCAAGGAAGGCTTCCGGATGCTCAGCCCTGAACACGCACCTGCTGAAA
TGTATGACATAATGAAGACTTGCTGGGATGCAGATCCCCTAAAAAGACCAACATTCAAGCAAATTGTTC
AGCTAATTGAGAAGCAGATTTCAGAGAGCACCAATCATATTTACTCCAACTTAGCAAACTGCAGCCCCA
ACCGACAGAAGCCCGTGGTAGACCATTCTGTGCGGATCAATTCTGTCGGCAGCACCGCTTCCTCCTCCC
AGCCTCTGCTTGTGCACGACGATGTCTGAGCGGCCGCGTCGATCGACAATCAACCTCTGGATTACAAAA
TTTGTGAAAGATTGACTGGTATTCTTAACTATGTTGCTCCTTTTACGCTATGTGGATACGCTGCTTTAA
TGCCTTTGTATCATGCTATTGCTTCCCGTATGGCTTTCATTTTCTCCTCCTTGTATAAATCCTGGTTGC
TGTCTCTTTATGAGGAGTTGTGGCCCGTTGTCAGGCAACGTGGCGTGGTGTGCACTGTGTTTGCTGACG
CAACCCCCACTGGTTGGGGCATTGCCACCACCTGTCAGCTCCTTTCCGGGACTTTCGCTTTCCCCCTCC
CTATTGCCACGGCGGAACTCATCGCCGCCTGCCTTGCCCGCTGCTGGACAGGGGCTCGGCTGTTGGGCA
CTGACAATTCCGTGGTGTTGTCGGGGAAATCATCGTCCTTTCCTTGGCTGCTCGCCTGTGTTGCCACCT
GGATTCTGCGCGGGACGTCCTTCTGCTACGTCCCTTCGGCCCTCAATCCAGCGGACCTTCCTTCCCGCG
GCCTGCTGCCGGCTCTGCGGCCTCTTCCGCGTCTTCGCCTTCGCCCTCAGACGAGTCGGATCTCCCTTT
GGGCCGCCTCCCCGCCTGGAATTCGAGCTCGTAAAAAAAAAAAAAAAAAAAAAAAAAAAAAAAAAAAAA
AAAAAAAAAAAAAAAAAAAAAAAAAAA

### Vector

In one aspect, the present invention provides a vector encoding a RNA polynucleotide according to the present invention.

In one aspect, the present invention provides a vector comprising the DNA polynucleotide according to the present invention.

A vector is a tool that allows or facilitates the transfer of an entity from one environment to another. In accordance with the present invention, and by way of example, some vectors used in recombinant nucleic acid techniques allow entities, such as a segment of nucleic acid (e.g. a heterologous DNA segment, such as a heterologous cDNA segment), to be transferred into a target cell. The vector may serve the purpose of maintaining the heterologous nucleic acid (DNA or RNA) within the cell, facilitating the replication of the vector comprising a segment of nucleic acid, or facilitating the expression of the protein encoded by a segment of nucleic acid.

Vectors may be non-viral or viral. Examples of vectors used in recombinant nucleic acid techniques include, but are not limited to, plasmids, chromosomes, artificial chromosomes and viruses. The vector may be single stranded or double stranded. It may be linear and optionally the vector comprises one or more homology arms. The vector may also be, for example, a naked nucleic acid (e.g. DNA). In its simplest form, the vector may itself be a nucleotide of interest.

The term “vector” includes an expression vector, i.e. a construct capable of in vivo or in vitro/ex vivo expression. Expression may be controlled by a vector sequence, or, for example in the case of insertion at a target site, expression may be controlled by a target sequence. A vector may be integrated or tethered to the cell's DNA.

The vectors used in the invention may be, for example, plasmid or virus vectors and may include a promoter for the expression of a polynucleotide and optionally a regulator of the promoter.

In one embodiment, the vector is a plasmid.

In one embodiment, the vector is a viral vector. Viral delivery systems include but are not limited to adenoviral vectors, adeno-associated viral (AAV) vectors, herpes viral vectors, retroviral vectors, lentiviral vectors and baculoviral vectors. In one embodiment, the vector is a retroviral, adenoviral or adeno-associated viral vector. In one embodiment, the vector is a retroviral vector. In one embodiment, the vector is a lentiviral vector.

In one embodiment, the vector is a Sendai viral vector. Sendai viral vectors may be particularly effective for transient expression of transgenes, such as engraftment enhancers. Furthermore, Sendai viral vectors are typically capable of very efficiently transferring transgenes to HSPCs (e.g. capable of transferring a transgene (e.g. GFP) to cord blood CD34+ cells at an MOI of 3). Sendai viral vectors are typically unable to infect neighbouring cells. In addition, Sendai viral vectors may be temperature sensitive, for example: at a temperature of about 34° C. they may be capable of replication; at a temperature of about 37° C. their replication may be low; and at a temperature of about 38° C. they replication may be prevented. Sendai viral vectors typically do not impact cell viability.

### Cell

In one aspect, the present invention provides a cell comprising the RNA polynucleotide, the DNA polynucleotide, or the vector of the present invention. Suitably, the cell is an isolated cell. Suitably, the cell is a mammalian cell, for example a human cell.

In one aspect, the present invention provides a method for providing a cell or a population of cells comprising the RNA polynucleotide of the present invention.

The cell is not particularly limited and any suitable cell may be used. For example, the cell may be any cell suitable for production of the RNA polynucleotide, DNA polynucleotide, or vector. In some embodiments, the cell is a hematopoietic stem cell (HSC) or a hematopoietic progenitor cell (HPC).

The RNA polynucleotide, DNA polynucleotide, or vector of the present invention may be introduced into cells using a variety of techniques known in the art, such as transformation, transfection and transduction. Several techniques are known in the art, for example transduction with recombinant viral vectors, such as retroviral, lentiviral, adenoviral, adeno-associated viral, baculoviral and herpes simplex viral vectors, Sleeping Beauty vectors; direct injection of nucleic acids and biolistic transformation.

Non-viral delivery systems include but are not limited to transfection methods. Here, transfection includes a process using a non-viral vector to deliver a gene to a target cell. Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs), and combinations thereof.

### Population of Cells

In one aspect, the present invention provides a population or cells comprising the cell of the present invention.

Suitably, the population of cells are mammalian cells, for example human cells. The population of cells may be autologous or allogeneic. Suitably, the population of cells are obtained or obtainable from (mobilized) peripheral blood or cord blood.

Suitably, at least 1%, at least 2%, at least 5%, at least 10%, or at least 20% of the cells in the population of cells are cells of the present invention. Suitably, at least 50%, at least 60%, at least 70%, at least 80%, at least 90%, at least 95%, at least 99%, or 100% of the population of cells are HSPCs. Suitably, the population of cells comprises at least 10×10⁵, at least 50×10⁵, or at least 100×10⁵ cells of the present invention.

### Haematopoietic Stem and Progenitor Cells (HSPCs)

In one aspect, the present invention provides an HSPC comprising the RNA polynucleotide of the present invention. Suitably, the HSPC may be an isolated HSPC.

In one aspect, the present invention provides a population of HSPCs comprising the RNA polynucleotide of the present invention. Suitably, the population may be an isolated population.

In one aspect, the present invention provides a method for providing an HSPC or a population of HSPCs comprising the RNA polynucleotide of the present invention. The RNA polynucleotide may be delivered to an HSPC or a population of HSPCs by transfection.

In some embodiments, the RNA polynucleotide is delivered to the HSPC or the population of HSPCs by electroporation. Electroporation increases cell membrane permeability to nucleic acids using an electrical field and can be used to deliver mRNA to HSPCs (see e.g. Smits, E., et al., 2004. Leukemia, 18(11), pp. 1898-1902).

In some embodiments, the RNA polynucleotide is delivered to the HSPC or the population of HSPCs by an mRNA delivery system. A variety of materials have been developed for mRNA delivery, including lipids, lipid-like materials, polymers and protein derivatives (see e.g. Hou, X., et al., 2021. Lipid nanoparticles for mRNA delivery. Nature Reviews Materials, 6(12), pp. 1078-1094).

In some embodiments, the RNA polynucleotide is delivered to the HSPC or the population of HSPCs by lipid-mediated transfection. Lipid-mediated transfection, also known as “lipofection”, “lipid transfection” or “liposome-based transfection,” uses a lipid complex to deliver polynucleotides to cells. Lipids are amphiphilic molecules that contain three domains: a polar head group, a hydrophobic tail region and a linker between the two domains. Cationic lipids, ionizable lipids and other types of lipid have been explored for mRNA delivery. Lipid nanoparticle-mRNA formulations manufactured by rapid mixing exhibit a stable nanostructure in which mRNA molecules can be encapsulated in the interior core through electrostatic interactions with the lipids (see e.g. Hou, X., et al., 2021. Lipid nanoparticles for mRNA delivery. Nature Reviews Materials, 6(12), pp. 1078-1094).

### Pharmaceutical Composition

In one aspect, the present invention provides a pharmaceutical composition comprising the HSPC or population of HSPCS according to the present invention.

The HSPCs of the invention may be formulated for administration to subjects with a pharmaceutically acceptable carrier, diluent or excipient. Suitable carriers and diluents include isotonic saline solutions, for example phosphate-buffered saline, and potentially contain human serum albumin.

Handling of cell therapy products is preferably performed in compliance with FACT-JACIE International Standards for cellular therapy.

### Kit

In another aspect, the present invention provides a kit comprising one or more RNA polynucleotides, DNA polynucleotides, vectors, cells, cell populations, and/or pharmaceutical compositions of the invention.

The RNA polynucleotides, DNA polynucleotides, vectors, cells, cell populations, and/or pharmaceutical compositions may be provided in suitable containers. The kit may also include instructions for use.

### Kit of Vectors Encoding Engraftment Enhancers

In some embodiments, a kit of the invention comprises: (i) a vector encoding CXCR4 (or a fragment or variant thereof); (ii) a vector encoding CD47 (or a fragment or variant thereof); (iii) a vector encoding ITGA4 (or a fragment or variant thereof); and/or (iv) a vector encoding KIT (or a fragment or variant thereof). The vectors may be any vectors disclosed herein.

In some embodiments, the kit comprises two or more vectors encoding engraftment enhancers. In some embodiments, the kit comprises three or more vectors encoding engraftment enhancers. In some embodiments, the kit comprises four or more vectors encoding engraftment enhancers.

In some embodiments, the kit comprises: (i) a vector encoding CXCR4 (or a fragment or variant thereof); and (ii) a vector encoding CD47 (or a fragment or variant thereof). In some embodiments, the kit comprises: (i) a vector encoding CXCR4 (or a fragment or variant thereof); and (ii) a vector encoding ITGA4 (or a fragment or variant thereof). In some embodiments, the kit comprises: (i) a vector encoding CXCR4 (or a fragment or variant thereof); and (ii) a vector encoding KIT (or a fragment or variant thereof). In some embodiments, the kit comprises: (i) a vector encoding ITGA4 (or a fragment or variant thereof); and (ii) a vector encoding CD47 (or a fragment or variant thereof). In some embodiments, the kit comprises: (i) a vector encoding KIT (or a fragment or variant thereof); and (ii) a vector encoding CD47 (or a fragment or variant thereof). In some embodiments, the kit comprises: (i) a vector encoding ITGA4 (or a fragment or variant thereof); and (ii) a vector encoding KIT (or a fragment or variant thereof).

In some embodiments, the kit comprises: (i) a vector encoding CXCR4 (or a fragment or variant thereof); (ii) a vector encoding ITGA4 (or a fragment or variant thereof); and (iii) a vector encoding KIT (or a fragment or variant thereof). In some embodiments, the kit comprises: (i) a vector encoding CXCR4 (or a fragment or variant thereof); (ii) a vector encoding ITGA4 (or a fragment or variant thereof); and (iii) a vector encoding CD47 (or a fragment or variant thereof). In some embodiments, the kit comprises: (i) a vector encoding CXCR4 (or a fragment or variant thereof); (ii) a vector encoding KIT (or a fragment or variant thereof); and (iii) a vector encoding CD47 (or a fragment or variant thereof).

In some embodiments, the kit comprises: (i) a vector encoding CXCR4 (or a fragment or variant thereof); (ii) a vector encoding CD47 (or a fragment or variant thereof); (iii) a vector encoding ITGA4 (or a fragment or variant thereof); and (iv) a vector encoding KIT (or a fragment or variant thereof).

### Kit of RNA Polynucleotides Encoding Engraftment Enhancers

In some embodiments, a kit of the invention comprises: (i) a RNA polynucleotide encoding CXCR4 (or a fragment or variant thereof); (ii) a RNA polynucleotide encoding CD47 (or a fragment or variant thereof); (iii) a RNA polynucleotide encoding ITGA4 (or a fragment or variant thereof); and/or (iv) a RNA polynucleotide encoding KIT (or a fragment or variant thereof). The RNA polynucleotides may be any RNA polynucleotides disclosed herein.

In some embodiments, the kit comprises two or more RNA polynucleotides encoding engraftment enhancers. In some embodiments, the kit comprises three or more RNA polynucleotides encoding engraftment enhancers. In some embodiments, the kit comprises four or more RNA polynucleotides encoding engraftment enhancers.

In some embodiments, the kit comprises: (i) a RNA polynucleotide encoding CXCR4 (or a fragment or variant thereof); and (ii) a RNA polynucleotide encoding CD47 (or a fragment or variant thereof). In some embodiments, the kit comprises: (i) a RNA polynucleotide encoding CXCR4 (or a fragment or variant thereof); and (ii) a RNA polynucleotide encoding ITGA4 (or a fragment or variant thereof). In some embodiments, the kit comprises: (i) a RNA polynucleotide encoding CXCR4 (or a fragment or variant thereof); and (ii) a RNA polynucleotide encoding KIT (or a fragment or variant thereof). In some embodiments, the kit comprises: (i) a RNA polynucleotide encoding ITGA4 (or a fragment or variant thereof); and (ii) a RNA polynucleotide encoding CD47 (or a fragment or variant thereof). In some embodiments, the kit comprises: (i) a RNA polynucleotide encoding KIT (or a fragment or variant thereof); and (ii) a RNA polynucleotide encoding CD47 (or a fragment or variant thereof). In some embodiments, the kit comprises: (i) a RNA polynucleotide encoding ITGA4 (or a fragment or variant thereof); and (ii) a RNA polynucleotide encoding KIT (or a fragment or variant thereof).

In some embodiments, the kit comprises: (i) a RNA polynucleotide encoding CXCR4 (or a fragment or variant thereof); (ii) a RNA polynucleotide encoding ITGA4 (or a fragment or variant thereof); and (iii) a RNA polynucleotide encoding KIT (or a fragment or variant thereof). In some embodiments, the kit comprises: (i) a RNA polynucleotide encoding CXCR4 (or a fragment or variant thereof); (ii) a RNA polynucleotide encoding ITGA4 (or a fragment or variant thereof); and (iii) a RNA polynucleotide encoding CD47 (or a fragment or variant thereof). In some embodiments, the kit comprises: (i) a RNA polynucleotide encoding CXCR4 (or a fragment or variant thereof); (ii) a RNA polynucleotide encoding KIT (or a fragment or variant thereof); and (iii) a RNA polynucleotide encoding CD47 (or a fragment or variant thereof).

In some embodiments, the kit comprises: (i) a RNA polynucleotide encoding CXCR4 (or a fragment or variant thereof); (ii) a RNA polynucleotide encoding CD47 (or a fragment or variant thereof); (iii) a RNA polynucleotide encoding ITGA4 (or a fragment or variant thereof); and (iv) a RNA polynucleotide encoding KIT (or a fragment or variant thereof).

### Kit of DNA Polynucleotides Encoding RNA Polynucleotides

In some embodiments, a kit of the invention comprises: (i) a DNA polynucleotide encoding a RNA polynucleotide encoding CXCR4 (or a fragment or variant thereof); (ii) a DNA polynucleotide encoding a RNA polynucleotide encoding CD47 (or a fragment or variant thereof); (iii) a DNA polynucleotide encoding a RNA polynucleotide encoding ITGA4 (or a fragment or variant thereof); and/or (iv) a DNA polynucleotide encoding a RNA polynucleotide encoding KIT (or a fragment or variant thereof). The DNA polynucleotides may be any DNA polynucleotides disclosed herein.

In some embodiments, the kit comprises two or more DNA polynucleotides. In some embodiments, the kit comprises three or more DNA polynucleotides. In some embodiments, the kit comprises four or more DNA polynucleotides.

In some embodiments, the kit comprises: (i) a DNA polynucleotide encoding a RNA polynucleotide encoding CXCR4 (or a fragment or variant thereof); and (ii) a DNA polynucleotide encoding a RNA polynucleotide encoding CD47 (or a fragment or variant thereof). In some embodiments, the kit comprises: (i) a DNA polynucleotide encoding a RNA polynucleotide encoding CXCR4 (or a fragment or variant thereof); and (ii) a DNA polynucleotide encoding a RNA polynucleotide encoding ITGA4 (or a fragment or variant thereof). In some embodiments, the kit comprises: (i) a DNA polynucleotide encoding a RNA polynucleotide encoding CXCR4 (or a fragment or variant thereof); and (ii) a DNA polynucleotide encoding a RNA polynucleotide encoding KIT (or a fragment or variant thereof). In some embodiments, the kit comprises: (i) a DNA polynucleotide encoding a RNA polynucleotide encoding ITGA4 (or a fragment or variant thereof); and (ii) a DNA polynucleotide encoding a RNA polynucleotide encoding CD47 (or a fragment or variant thereof). In some embodiments, the kit comprises: (i) a DNA polynucleotide encoding a RNA polynucleotide encoding KIT (or a fragment or variant thereof); and (ii) a DNA polynucleotide encoding a RNA polynucleotide encoding CD47 (or a fragment or variant thereof). In some embodiments, the kit comprises: (i) a DNA polynucleotide encoding a RNA polynucleotide encoding ITGA4 (or a fragment or variant thereof); and (ii) a DNA polynucleotide encoding a RNA polynucleotide encoding KIT (or a fragment or variant thereof).

In some embodiments, the kit comprises: (i) a DNA polynucleotide encoding a RNA polynucleotide encoding CXCR4 (or a fragment or variant thereof); (ii) a DNA polynucleotide encoding a RNA polynucleotide encoding ITGA4 (or a fragment or variant thereof); and (iii) a DNA polynucleotide encoding a RNA polynucleotide encoding KIT (or a fragment or variant thereof). In some embodiments, the kit comprises: (i) a DNA polynucleotide encoding a RNA polynucleotide encoding CXCR4 (or a fragment or variant thereof); (ii) a DNA polynucleotide encoding a RNA polynucleotide encoding ITGA4 (or a fragment or variant thereof); and (iii) a DNA polynucleotide encoding a RNA polynucleotide encoding CD47 (or a fragment or variant thereof). In some embodiments, the kit comprises: (i) a DNA polynucleotide encoding a RNA polynucleotide encoding CXCR4 (or a fragment or variant thereof); (ii) a DNA polynucleotide encoding a RNA polynucleotide encoding KIT (or a fragment or variant thereof); and (iii) a DNA polynucleotide encoding a RNA polynucleotide encoding CD47 (or a fragment or variant thereof).

In some embodiments, the kit comprises: (i) a DNA polynucleotide encoding a RNA polynucleotide encoding CXCR4 (or a fragment or variant thereof); (ii) a DNA polynucleotide encoding a RNA polynucleotide encoding CD47 (or a fragment or variant thereof); (iii) a DNA polynucleotide encoding a RNA polynucleotide encoding ITGA4 (or a fragment or variant thereof); and (iv) a DNA polynucleotide encoding a RNA polynucleotide encoding KIT (or a fragment or variant thereof).

### Method of Treatment

In one aspect, the invention provides a population of haematopoietic stem and/or progenitor cells (HSPCs) for use in a method of therapy, for example gene therapy. The method may comprise an HSPC transplantation, as described herein.

In one aspect, the invention provides a population of haematopoietic stem and/or progenitor cells (HSPCs) for use in a method of treatment. The method may comprise an HSPC transplantation, as described herein.

In one aspect, the present invention provides one or more HSPC mobiliser for use in a method of therapy, for example gene therapy. The method may comprise an HSPC transplantation, as described herein.

In one aspect, the present invention provides an RNA polynucleotide according to the present invention, a DNA polynucleotide according to the present invention, a vector according to the present invention, a cell according to the present invention, a pharmaceutical composition according to the present invention, or a kit according to the present invention, for use in a method of therapy, for example gene therapy.

In one aspect, the present invention provides an HSPC according to the present invention, for use in a method of therapy, for example gene therapy.

In one aspect, the present invention provides a population of HSPCs according to the present invention, for use in a method of therapy, for example gene therapy.

In one aspect, the present invention provides a method of treating a subject in need thereof, comprising the steps:
(a) providing an HSPC according to the present invention or a population of HSPCs according to the present invention; and
(b) administering the HSPC or population of HSPCs to the subject.

It is to be appreciated that all references herein to treatment include curative, palliative and prophylactic treatment. The treatment of mammals, particularly humans, is preferred. Both human and veterinary treatments are within the scope of the invention.

HSPC gene therapy (HSPC-GT) has emerged as an effective treatment modality for a range of diseases (Ferrari, G., et al., 2021. Nature Reviews Genetics, 22(4), pp. 216-234). The invention (e.g. the HSPC gene therapy) may be, for example, useful in the treatment of a disease selected from the group consisting of mucopolysaccharidosis type I (MPS-1), chronic granulomatous disorder (CGD), Fanconi anaemia (FA), sickle cell disease, Pyruvate kinase deficiency (PKD), Leukocyte adhesion deficiency (LAD), metachromatic leukodystrophy (MLD), globoid cell leukodystrophy (GLD), GM₂ gangliosidosis, thalassemia, cancer, a genetic disease and a blood disease. The invention may also be, for example, useful in the treatment of mucopolysaccharidoses disorders and other lysosomal storage disorders.

In addition, or in the alternative, the invention may be useful in the treatment of the disorders listed in WO 1998/005635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the invention may be useful in the treatment of the disorders listed in WO 1998/007859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); anti-inflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the invention may be useful in the treatment of the disorders listed in WO 1998/009985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

Haematopoietic progenitor cells provide short term engraftment. Accordingly, gene therapy by administering haematopoietic progenitor cells (HPCs) may provide a non-permanent effect in the subject. For example, the effect may be limited to about one to six months following administration of the HPCs. An advantage of this approach would be better safety and tolerability, due to the self-limited nature of the therapeutic intervention. Such HPC gene therapy may be suited to treatment of acquired disorders, for example cancer, where time-limited expression of a (potentially toxic) anti-cancer nucleotide of interest may be sufficient to eradicate the disease.

In one aspect, the present invention provides a population of HSPCs, for use in a method of treating or preventing cancer, an immune disorder, a lysosomal storage disorder, a bacterial or viral infection, a genetic disease, or a haemoglobinopathy. The method may comprise an HSPC transplantation, as described herein.

In one aspect, the present invention provides a method of treating or preventing cancer, an immune disorder, a lysosomal storage disorder, a bacterial or viral infection, a genetic disease, or a haemoglobinopathy in a subject in need thereof. The method may comprise an HSPC transplantation, as described herein.

In one aspect, the present invention provides one or more HSPC mobiliser for use in a method of treating or preventing cancer, an immune disorder, a lysosomal storage disorder, a bacterial or viral infection, a genetic disease, or a haemoglobinopathy. The method may comprise an HSPC transplantation, as described herein.

In one aspect, the present invention provides an RNA polynucleotide according to the present invention, a DNA polynucleotide according to the present invention, a vector according to the present invention, a cell according to the present invention, a pharmaceutical composition according to the present invention, or a kit according to the present invention, for use in the treatment or prevention of cancer, an immune disorder, a lysosomal storage disorder, a bacterial or viral infection, a genetic disease, or a haemoglobinopathy.

In one aspect, the present invention provides an HSPC according to the present invention, for use in the treatment or prevention of cancer, an immune disorder, a lysosomal storage disorder, a bacterial or viral infection, a genetic disease, or a haemoglobinopathy.

In one aspect, the present invention provides a population of HSPCs according to the present invention, for use in the treatment or prevention of cancer, an immune disorder, a lysosomal storage disorder, a bacterial or viral infection, a genetic disease, or a haemoglobinopathy.

In one aspect, the present invention provides a method of treating or preventing cancer, an immune disorder, a lysosomal storage disorder, a bacterial or viral infection, a genetic disease, or a haemoglobinopathy in a subject in need thereof, comprising the steps:
(a) providing an HSPC according to the present invention or a population of HSPCs according to the present invention; and
(b) administering the HSPC or population of HSPCs to the subject.

In one aspect, the present invention provides a population of HSPCs, for use in a method of treating or preventing cancer, a primary immunodeficiency, a lysosomal storage disorder, or a haemoglobinopathy. The method may comprise an HSPC transplantation, as described herein.

In one aspect, the present invention provides a method of treating or preventing cancer, a primary immunodeficiency, a lysosomal storage disorder, or a haemoglobinopathy in a subject in need thereof. The method may comprise an HSPC transplantation, as described herein.

In one aspect, the present invention provides one or more HSPC mobiliser for use in a method of treating or preventing cancer, a primary immunodeficiency, a lysosomal storage disorder, or a haemoglobinopathy. The method may comprise an HSPC transplantation, as described herein.

In one aspect, the present invention provides an RNA polynucleotide according to the present invention, a DNA polynucleotide according to the present invention, a vector according to the present invention, a cell according to the present invention, a pharmaceutical composition according to the present invention, or a kit according to the present invention, for use in the treatment or prevention of cancer, a primary immunodeficiency, a lysosomal storage disorder, or a haemoglobinopathy.

In one aspect, the present invention provides an HSPC according to the present invention, for use in the treatment or prevention of cancer, a primary immunodeficiency, a lysosomal storage disorder, or a haemoglobinopathy.

In one aspect, the present invention provides a population of HSPCs according to the present invention, for use in the treatment or prevention of cancer, a primary immunodeficiency, a lysosomal storage disorder, or a haemoglobinopathy.

In one aspect, the present invention provides a method of treating or preventing cancer, a primary immunodeficiency, a lysosomal storage disorder, or a haemoglobinopathy in a subject in need thereof, comprising the steps:
(a) providing an HSPC according to the present invention or a population of HSPCs according to the present invention; and
(b) administering the HSPC or population of HSPCs to the subject.

### Cancer

In one aspect, the present invention provides a population of HSPCs, for use in a method of treating or preventing cancer. The method may comprise an HSPC transplantation, as described herein.

In one aspect, the present invention provides a method of treating or preventing cancer in a subject in need thereof. The method may comprise an HSPC transplantation, as described herein.

In one aspect, the present invention provides one or more HSPC mobiliser for use in a method of treating or preventing cancer. The method may comprise an HSPC transplantation, as described herein.

In one aspect, the present invention provides an RNA polynucleotide according to the present invention, a DNA polynucleotide according to the present invention, a vector according to the present invention, a cell according to the present invention, a pharmaceutical composition according to the present invention, or a kit according to the present invention, for use in the treatment or prevention of cancer.

In one aspect, the present invention provides an HSPC according to the present invention, for use in the treatment or prevention of cancer.

In one aspect, the present invention provides a population of HSPCs according to the present invention, for use in the treatment or prevention of cancer.

In one aspect, the present invention provides a method of treating or preventing cancer in a subject in need thereof, comprising the steps:
(a) providing an HSPC according to the present invention or a population of HSPCs according to the present invention; and
(b) administering the HSPC or population of HSPCs to the subject.

Hematopoietic stem cell transplants (HSCTs) are considered the best treatment option for many hematological malignancies (see e.g. Gratwohl, A., et al., 2003. Leukemia, 17(5), pp. 941-959) and is also a treatment option for patients with solid tumors (see e.g. Gratwohl, A., et al., 2004. Annals of oncology, 15(4), pp. 653-660).

In some embodiments the cancer is a hematological malignancy. Hematological malignancies may include acute myeloid leukemia (AML), acute lymphocytic leukemia (ALL), chronic myeloid leukemia (CML), myelodysplastic syndromes (MDS), lymphoproliferative disorders (LPS) and multiple myeloma (MM).

In some embodiments the cancer is a solid tumor. Solid tumours may include neuroblastoma, glioma, soft tissue sarcoma, germ cell cancer, breast cancer, Ewing's sarcoma, lung cancer, ovarian cancer and other solid tumors.

### Primary Immunodeficiency

In one aspect, the present invention provides a population of HSPCs, for use in a method of treating or preventing a primary immunodeficiency. The method may comprise an HSPC transplantation, as described herein.

In one aspect, the present invention provides a method of treating or preventing a primary immunodeficiency in a subject in need thereof. The method may comprise an HSPC transplantation, as described herein.

In one aspect, the present invention provides one or more HSPC mobiliser for use in a method of treating or preventing a primary immunodeficiency. The method may comprise an HSPC transplantation, as described herein.

In one aspect, the present invention provides an RNA polynucleotide according to the present invention, a DNA polynucleotide according to the present invention, a vector according to the present invention, a cell according to the present invention, a pharmaceutical composition according to the present invention, or a kit according to the present invention, for use in the treatment or prevention of a primary immunodeficiency.

In one aspect, the present invention provides an HSPC according to the present invention, for use in the treatment or prevention of a primary immunodeficiency.

In one aspect, the present invention provides a population of HSPCs according to the present invention, for use in the treatment or prevention of a primary immunodeficiency.

In one aspect, the present invention provides a method of treating or preventing a primary immunodeficiency in a subject in need thereof, comprising the steps:
(a) providing an HSPC according to the present invention or a population of HSPCs according to the present invention; and
(b) administering the HSPC or population of HSPCs to the subject.

Primary immunodeficiencies (PIDs) are a group of heritable disorders that result in an underdeveloped and/or functionally compromised immune system. There are over 430 recognized PIDs as of 2019 (Tangye, S. G., et al., 2020. Journal of clinical immunology, 40(1), pp. 24-64). Patients with severe PIDs experience increase morbidity and mortality and display diverse clinical phenotypes. SCT using HSPCs from an HLA-matched donor can confer a lifelong ‘cure’, with a success rate of more than 90% (see e.g. Ferrari, G., et al., 2021. Nature Reviews Genetics, 22(4), pp. 216-234).

In some embodiments, the primary immunodeficiency is human primary combined immunodeficiency Hyper IgM Syndrome 1 (HIGM-1).

### Lysosomal Storage Disorders

In one aspect, the present invention provides a population of HSPCs, for use in a method of treating or preventing a lysosomal storage disorder. The method may comprise an HSPC transplantation, as described herein.

In one aspect, the present invention provides a method of treating or preventing a lysosomal storage disorder in a subject in need thereof. The method may comprise an HSPC transplantation, as described herein.

In one aspect, the present invention provides one or more HSPC mobiliser for use in a method of treating or preventing a lysosomal storage disorder. The method may comprise an HSPC transplantation, as described herein.

In one aspect, the present invention provides an RNA polynucleotide according to the present invention, a DNA polynucleotide according to the present invention, a vector according to the present invention, a cell according to the present invention, a pharmaceutical composition according to the present invention, or a kit according to the present invention, for use in the treatment or prevention of a lysosomal storage disorder.

In one aspect, the present invention provides an HSPC according to the present invention, for use in the treatment or prevention of a lysosomal storage disorder.

In one aspect, the present invention provides a population of HSPCs according to the present invention, for use in the treatment or prevention of a lysosomal storage disorder.

In one aspect, the present invention provides a method of treating or preventing a lysosomal storage disorder in a subject in need thereof, comprising the steps:
(a) providing an HSPC according to the present invention or a population of HSPCs according to the present invention; and
(b) administering the HSPC or population of HSPCs to the subject.

Lysosomal storage diseases (LSDs) are a group of over 70 inherited metabolic disorders that result from defects in lysosomal function (Platt, F. M., et al., 2018. Nature Reviews Disease Primers, 4(1), pp. 1-25). HSCT is currently the standard of care for infants with Hurler syndrome, and has also been used for other LSDs for which no other treatment was available, the most prominent being metachromatic leukodystrophy and Krabbe disease. In some embodiments, the lysosomal storage disorder is Hurler syndrome.

### Non-Malignant Hematological Disorders

In one aspect, the present invention provides a population of HSPCs, for use in a method of treating or preventing a non-malignant hematological disorder. The method may comprise an HSPC transplantation, as described herein.

In one aspect, the present invention provides a method of treating or preventing a non-malignant hematological disorder in a subject in need thereof. The method may comprise an HSPC transplantation, as described herein.

In one aspect, the present invention provides one or more HSPC mobiliser for use in a method of treating or preventing a non-malignant hematological disorder. The method may comprise an HSPC transplantation, as described herein.

In one aspect, the present invention provides an RNA polynucleotide according to the present invention, a DNA polynucleotide according to the present invention, a vector according to the present invention, a cell according to the present invention, a pharmaceutical composition according to the present invention, or a kit according to the present invention, for use in the treatment or prevention of a non-malignant hematological disorder.

In one aspect, the present invention provides an HSPC according to the present invention, for use in the treatment or prevention of a non-malignant hematological disorder.

In one aspect, the present invention provides a population of HSPCs according to the present invention, for use in the treatment or prevention of a non-malignant hematological disorder.

In one aspect, the present invention provides a method of treating or preventing a non-malignant hematological disorder in a subject in need thereof, comprising the steps:
(a) providing an HSPC according to the present invention or a population of HSPCs according to the present invention; and
(b) administering the HSPC or population of HSPCs to the subject.

Hematopoietic stem cell transplantation (HSCT) is a potentially curative modality for a variety of non-malignant hematological disorders involving bone marrow (BM) failure and thalassemia. It has been successfully used as a replacement therapy for patients with severe aplastic anemia (SAA), B-thalassemia major (BTM), Fanconi anemia (FA), immunodeficiency diseases (ID) and inherited metabolic disorders (IMD) (see e.g. Mahmoud, H. K., et al., 2015. Journal of Advanced Research, 6(3), pp. 449-458).

In some embodiments, the non-malignant hematological disorder is a haemoglobinopathy. Hemoglobinopathy may refer to a group of inherited blood disorders and diseases that primarily affect red blood cells. There are two main groups: abnormal structural hemoglobin variants caused by mutations in the hemoglobin genes, and the thalassemias, which are caused by an underproduction of otherwise normal hemoglobin molecules. The main structural hemoglobin variants are HbS, HbE and HbC. The main types of thalassemia are alpha-thalassemia and beta thalassemia. In some embodiments, the haemoglobinopathy is a thalassemia or sickle cell disease. In some embodiments, the haemoglobinopathy is beta thalassemia or sickle cell disease.

### Variants, Derivatives, Analogues, Homologues and Fragments

In addition to the specific polypeptides and polynucleotides mentioned herein, the invention also encompasses the use of variants, derivatives, analogues, homologues and fragments thereof.

In the context of the invention, a variant of any given sequence is a sequence in which the specific sequence of residues (whether amino acid or nucleic acid residues) has been modified in such a manner that the polypeptide or polynucleotide in question substantially retains at least one of its functions. A variant sequence can be obtained by addition, deletion, substitution, modification, replacement and/or variation of at least one residue present in the naturally-occurring protein.

The term “derivative” as used herein, in relation to proteins or polypeptides of the invention includes any substitution of, variation of, modification of, replacement of, deletion of and/or addition of one (or more) amino acid residues from or to the sequence providing that the resultant protein or polypeptide substantially retains at least one of its functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence substantially retains the required activity or ability. Amino acid substitutions may include the use of non-naturally occurring analogues. Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” as used herein means an entity having a certain homology with the wild type amino acid sequence and the wild type nucleotide sequence. The term “homology” can be equated with “identity”.

A homologous sequence may include an amino acid sequence which may be at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical, preferably at least 95%, at least 96%, at least 97%, at least 98%, or at least 99% identical to the subject sequence. Typically, the homologues will comprise the same active sites etc. as the subject amino acid sequence. Although homology can also be considered in terms of similarity (i.e. amino acid residues having similar chemical properties/functions), in the context of the invention it is preferred to express homology in terms of sequence identity.

A homologous sequence may include a nucleotide sequence which may be at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical, preferably at least 95%, at least 96%, at least 97%, at least 98%, or at least 99% identical to the subject sequence. Although homology can also be considered in terms of similarity, in the context of the invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

The skilled person will understand that they can combine all features of the invention disclosed herein without departing from the scope of the invention as disclosed.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

Preferred features and embodiments of the invention will now be described by way of non-limiting examples.

### Example 1—Chemotherapy-Free Engraftment of Gene Edited Human Hematopoietic Stem Cells Leveraged on Mobilization and mRNA-Based Engineering

### Long-Term Donor Chimerism is Established by Mobilization-Based HSCT

Mobilization regimens, while inducing substantial egress of resident HSPCs from the BM, might also per se avail space for newly transplanted cells. Thus, transplanting HSPCs at the peak of mobilization might enable competition with mobilized recipient cells to repopulate the BM niches, establishing some levels of donor chimerism (FIG. 6A).

We tested two mobilization regimens in mice, one modeling a clinically approved protocol using G-CSF and AMD3100 (G7A), and the other one also comprising BIO5192 (G7AB). C57BL/6J CD45.2 mice were treated with G-CSF using a subcutaneous pump for a week combined with either AMD3100, or the combination of AMD3100 and BIO5192, delivered intraperitoneally at day 6 and 7 (FIG. 1A; FIG. 6B).

We assessed mobilization three hours post-AMD3100 injection and showed a 6-fold increase in White Blood Cells (WBC) counts, an 8-fold increase in progenitors (Lin⁻ SCA1⁺ KIT+; LSK) and a 200-fold increase in the HSC-enriched fraction (LSK CD150⁺ CD48⁻; SLAM HSC) in the circulation (FIG. 1B; FIG. 6C), compared to non-mobilized control mice (Sham). The addition of BIO5192 led to a higher mobilization of WBC, LSK and SLAM HSC, leading to an additional 2-fold increase of SLAM HSC over G7A levels.

To test if these levels of mobilization, and the subsequent BM niche availability, were sufficient to allow engraftment of donor cells, 2×10⁶ CD45.1 lineage negative (Lin⁻) cells, purified from the BM, were transplanted into CD45.2 congenic recipients after the last injection of AMD3100 or AMD3100/BIO5192 (FIG. 1A, C; FIG. 6D).

Donor chimerism reached a median of 20% following the G-CSF/AMD3100 mobilization protocol, and the addition of BIO5192 further increased it to 30%. Chimerism was stable for up to 24 weeks (FIG. 1D), while only a minimal donor chimerism was observed in non-mobilized control mice (<1%).

The myeloid and lymphoid proportion was similar between mobilized and non-mobilized control mice (FIG. 1E, left panel). Donor cells showed a myeloid skewed output in the first weeks after engraftment, likely consistent with early repopulation by short-term progenitors (FIG. 1E, right panel). However, by the end of the experiment, donor cells and recipient cells were similar in terms of lineage composition (FIG. 1E). The chimerism was maintained among differentiated cell subsets, i.e. T cells (CD4 and CD8), B cells (CD19) and myeloid cells (CD11b) in the PB (FIG. 1F), BM (FIG. 1G), including within LSK and SLAM HSC (FIG. 1H), and spleen (FIG. 11), and the lineage distribution was similar for cells of recipient and donor origin in both organs (FIG. 1J-K).

Overall, these data support engraftment of donor long-term multilineage HSPCs. These findings established proof-of-principle of a low-burden HSCT, which entails an exchange of resident with donor HSPCs during mobilization without genotoxic conditioning, hereafter referred to as mobilization-enabled HSCT (M-HSCT).

### M-HSCT Allows Establishing Sufficient Donor Chimerism to Rescue the HIGM-1 Phenotype

To investigate the therapeutic potential of M-HSCT, we took advantage of Cd40Ig^{-/-} mice, which faithfully recapitulate the phenotype of the human primary combined immunodeficiency Hyper IgM Syndrome 1 (HIGM-1; Renshaw, B. R., et al. (1994). J Exp Medicine 180, 1889-1900). Because G-CSF treatment may have a detrimental effect on the BM niche, we investigated the efficiency of mobilization protocols differing for its presence or absence, the G-CSF dose and the duration and combination with other drugs (FIG. 2A).

Removing G-CSF or reducing its dose or treatment time, significantly decreased the mobilization of LSK and SLAM HSC. PB analysis revealed a peak of mobilization of WBC, LSK and SLAM HSC around 3 hours post-AMD3100 or AMD3100/BIO5192 injection (FIG. 2B-D). The addition of AMD3100 to the standard G-CSF treatment increased the mobilized LSK by 3-fold, and BIO5192 further increased it by 5-fold (FIG. 2C). The levels of mobilization observed in Cd40Ig^{-/-} mice were comparable to those obtained in the WT mice (FIG. 7A).

While administration of only AMD3100/BIO5192 had limited impact on blood neutrophils and monocytes counts, they were highly increased after G-CSF treatment (FIG. 7B-D). Moreover, G-CSF treatment increased MMP9, a protease released by myeloid cells, and decreased CXCL12, a chemokine involved in HSPC homing and retention, in the BM stroma, confirming remodeling of extracellular matrix (FIG. 2E-F; FIG. 7E). Intriguingly, when we compared the level of CXCR4 expression on circulating SLAM HSC with or without G-CSF mobilization, we found that it was lower in the former condition, suggesting induced cleavage of the molecule (FIG. 7F). By comparing the number of SLAM HSC in lower limbs BM of untreated and mobilized mice, at the peak of mobilization, we found a 55% decrease in the latter condition (FIG. 2G; left panel). However, only 75% of these cells could be accounted for in the PB (FIG. 2G; right panel), suggesting that some of the egressed cells became trapped in other organs, such as the spleen.

Since current editing protocols are considerably less efficient and more detrimental for mouse cells than for the human counterpart, we transplanted 2×10⁶ WT CD45.1 Lin⁻ cells, purified from the BM, as surrogate of autologous gene corrected cells and transplanted them into Cd40Ig^{-/-} recipients at the peak of mobilization following the G-CSF, AMD3100 and BIO5192 regimen (FIG. 7G). A stable chimerism of about 30% WT cells was reached across all lineages and differentiated cells in PB (FIG. 2H; FIG. 7H), spleen (FIG. 7I) and BM (FIG. 7J-K) of transplanted mice. By the end of the experiment, cellular subsets were comparable in recipient versus donor cells, and between the non-mobilized (Sham) and mobilized group (G7AB) in the PB (FIG. 2I), BM (FIG. 2J) and spleen (FIG. 2K).

Intriguingly, the chimerism experimentally obtained was higher than our estimate of approximate 20% based on data from FIG. 2G and the following postulates: (i) mobilization occurred to the same extent throughout all the mouse BM, (ii) the lower limbs account for 20% of it and (iii) the mobilized and infused cells compete equally. This finding suggests an advantage of the cells harvested from the donor BM and not exposed to the mobilization procedure, possibly conferred by the higher expression of CXCR4 (FIG. 7F) in the context of a limiting amount of CXCL12, highlighting a favorable window of opportunity for engraftment of donor HSPCs right upon mobilization.

To investigate the dynamic of niche repopulation in the context of mobilization, we transplanted 2×10⁶ WT CD45.1 Lin⁻ cells, purified from the BM, following different mobilization protocols. We observed that despite transplanting the same number of cells, the chimerism falls with lower mobilization efficiency. Despite infusing 7 times more donor cells than mobilized ones, the chimerism remained low, indicating that residual non mobilized cells are limiting the exchange, most likely by occupying the niche (FIG. 7L). On the other hand, when varying the numbers of transplanted cells below the saturating dose in mice mobilized with the same protocol, we found a dose dependent engraftment (FIG. 7M).

We then vaccinated the mice with a thymus-dependent antigen (TNP-KLH) and measured whether immunoglobulin (IgG) class switching was restored by the transplantation. Whereas non-mobilized Cd40Ig^{-/-} mice produced nearly undetectable amounts of antigen-specific IgGs, M-HSCT treated mice showed a significant rescue in switched-antibody responses to the primary and recall vaccinations (FIG. 2L). Partial rescue of immune function in transplanted mice was also shown by the presence of splenic germinal B cells, whereas non-mobilized mice failed to engage B cells for germinal center formation (FIG. 2M).

Overall, these findings show the potential of M-HSCT for rescue of the humoral immune response in HIGM-1 mice without the requirement of a conditioning regimen.

M-HSCT Allows Efficient Donor to Recipient Exchange of HSPCs within the Human Niche of Hematochimeric Mice

We next embarked in modeling the feasibility of M-HSCT in humans by using hematochimeric NOD.Cg-Kit^{W41J} Prkdc^{scid} II2rg^{tm1Wjl}/WaskJ (NSGW41) mice, which harbor a mutant Kit receptor decreasing the competition with human HSPCs. We first set up an in vivo model of human HSPC mobilization. NSGW41 mice were transplanted with 3×10⁵ G-CSF mobilized PB (mPB) derived CD34⁺ cells and, once hematopoietic engraftment was established (FIG. 3A), mice were treated with the combination mobilization protocol optimized above (G7AB).

Mobilization led to a substantial increase in WBC counts, murine (LSK) and human (CD34⁺ CD38⁻) progenitors (FIG. 3B). Of note, mobilized human CD34⁺ CD38⁻ cells peaked 3 hours after the last administration of mobilizers, suggesting this time as the best candidate for transplanting human donor cells. In line with PB results, we showed up to 65% decrease in human CD34⁺ CD38⁻ cells in the BM of mobilized mice. This decrease was concurrent with an increase of human CD34⁺ CD38⁻ cells in PB, accounting for 60% of the egressed cells (FIG. 3C).

We then tested CD34⁺ CD38⁻ cells exchange post-mobilization, by transplanting the outgrowth of 1×10⁵ CD34⁺ cells, counted on day 1 post-thawing (FIG. 3D). Prior to infusion, mPB cells from the same donor were transduced with GFP-expressing LV (FIG. 3D-E), to distinguish them from the previously engrafted and now resident cells. This culture step, which models a LV-based gene replacement protocol for therapeutic purposes, led to an increased expression of CXCR4, KIT and ITGA4 (all involved in HSPC homing and retention), counteracting the effect of thawing and prior in vivo G-CSF exposure (FIG. 3F; FIG. 8A-G). Intriguingly, mobilized cells showed increasing CXCR4 MFI upon culture when immunostaining with an N-terminus directed antibody, consistent with the reported N-terminal cleavage of the molecule upon exposure to G-CSF and rescued expression of the full-size molecule in culture.

The size of the human graft continued to increase over time in non-mobilized mice but comprising only a low % of GFP⁺ cells (<1%) even at late time points, showing that in the absence of mobilization there is no engraftment of the newly infused cells (FIG. 3G). On the contrary, when mice received the mobilization treatment, the human graft initially decreased but was then followed by a rescue accompanied by an increasing fraction of GFP⁺ cells, suggesting that these cells were able to effectively compete with the mobilized ones. At the end of the experiment, GFP⁺ cells were a median of 30% of the human graft and found within all human lineages present in the blood of mobilized mice (CD19⁺ B cells, CD13⁺ myeloid cells and CD3⁺ T cells), while being nearly absent in non-mobilized ones (FIG. 3G-1).

Lineage composition was similar between mobilized and non-mobilized mice (FIG. 3J, left panel). Interestingly, GFP⁺ cells showed a myeloid skewed output in the first weeks after transplantation, as observed for newly engrafting progenitors in the C57BL/6J mouse model above (FIG. 3J, right panel). However, GFP⁺ cells were found in all detectable lineages in the following weeks and approached the lineage distribution of the total human graft at the end of the experiment (FIG. 3J). In the BM, spleen and thymus, the human graft levels were similar in mobilized and non-mobilized mice, while the GFP⁺ cells reached up to 30% in the mobilized mice (FIG. 3K-L). Non-mobilized mice had GFP⁺ engraftment below 1%. The lineage distribution between the total human graft and the GFP⁺ cells was similar in all the organs (FIG. 3M-R), with comparable levels of chimerism across all cell subsets, including the most primitive progenitors (HSPCs: CD34⁺ CD38⁻ CD90⁺; FIG. 3S-T), indicating stable and functional engraftment.

These data show that partial chimerism of ex vivo modified HSPCs may potentially be established for human hematopoiesis following M-HSCT and without prior conditioning.

### Transient Overexpression of CXCR4 Increases Chimerism in the Humanized Context, Following M-HSCT

We sought to further improve the engraftment of HSPCs by exploiting transient overexpression of CXCR4.

We optimized a transient in vitro transcription (IVT) platform testing different 5′/3′UTR sequences, poly-adenylation tail lengths and capping (FIG. 9A-D). Our starting construct, pVAX, contained an ARCA capping, followed by a Kozak sequence, a WPRE sequence and a 60 bp polyA tail. The optimized pVAXi construct comprised an AG Cleancap, an Eif4 aptamer at the 5′UTR, a WPRE sequence at the 3′ UTR, followed by a 150 bp polyA tail. Modified uridine (pseudouridine) was incorporated during the mRNA synthesis, followed by HPLC purification, necessary to alleviate interferon response in electroporated cells (FIG. 9E-H).

CXCR4 mRNA electroporation led to an increase in cells expressing CXCR4 and its MFI in the bulk CD34⁺ population (FIG. 4A-B) and in primitive HSPCs (CD34⁺ CD38⁻ CD90⁺; FIG. 4C-D) for up to 3 days. Transmigration assays were used to determine the impact of CXCR4 overexpression on cell migration. HSPCs overexpressing CXCR4, including the more primitive subset, migrated more toward CXCL12, compared to control cells electroporated with GFP mRNA (FIG. 4E-F; FIG. 9I-L). We tested two isoforms of CXCR4, differing from 9 amino acid at the N-terminus (Duquenne, C., et al. (2014). J Immunol 193, 4188-4194; Gupta, S. K., and Pillarisetti, K. (1999). J Immunol Baltim Md 1950 163, 2368-2372). Isoform 1 led to higher response to CXCL12 and was used for the subsequent experiments (FIG. 4G-1).

Impact of CXCR4 overexpression on engraftment was first evaluated in immunodeficient NOD-SCID− IL2Rg^{-/-} (NSG) mice. Cells transiently overexpressing CXCR4 yielded a larger human graft in the PB, reaching a median of 45% human chimerism compared to 35% of cells overexpressing control GFP (FIG. 4J). The lineage distribution was similar in both groups in PB, BM, spleen and thymus (FIG. 4K-L; FIG. 10A-B). Interestingly, BM analysis showed an increased representation of the most primitive fraction of human HSPCs in mice transplanted with CXCR4 overexpressing cells (FIG. 4M).

We next tested the potential advantage contributed by CXCR4 overexpression in niche re-colonization upon M-HSCT. NSGW41 mice stably engrafted with 3×10⁵ CD34⁺ cells were treated for mobilization, then infused with the outgrowth of 2×10⁵ cells, counted at day 1 post thawing, transduced with GFP-LV and transiently overexpressing CXCR4 mRNA from the same donor as the original transplant. In parallel, control mice were transplanted, after mobilization, with HSPCs transduced with GFP-LV and transiently overexpressing GFP mRNA (FIG. 4N).

CXCR4 overexpressing cells efficiently outcompeted the mobilized HSPCs and established a stable chimerism in the human cell graft, while control cells overexpressing GFP engrafted to a lower extent (FIG. 4O-Q). Furthermore, we confirmed that mobilization was indispensable to obtain chimerism with the newly infused cells, whether advantaged or not by CXCR4 overexpression, as observed by the lack of engrafted GFP⁺ cells in non-mobilized mice.

By the end of the experiment, lineage composition was similar in mobilized and non-mobilized mice in PB (FIG. 4R), and the chimerism in each subset remained stable (FIG. 4S). These results were confirmed in the spleen, BM and thymus, where higher levels of GFP were detectable in mice transplanted with cells initially overexpressing CXCR4, without impacting the proportion of progenitors and differentiating cells within the different hematopoietic organs (FIG. 10C-H).

Secondary transplants of matched numbers of human CD34+ cells purified from the BM of primary transplanted mice revealed the presence of human GFP+ grafts in mice transplanted with cells harvested from mobilized groups only, proving successful engraftment of LT-HSC following M-HSCT (FIG. 4T). Moreover, the percentage of GFP+ cells in the secondary recipients was higher in the group transplanted with human cells from primary recipients of CD34+ cell transiently overexpressing CXCR4, versus cell transiently overexpressing control GFP mRNA, emphasizing the advantage of using an engraftment enhancer (FIG. 4U).

M-HSCT Confers a Clear Advantage to Gene Edited Cells when Paired with an Engraftment Enhancer

EX vivo gene correction in autologous HSCT may decrease engraftment efficiency, especially when achieved by gene editing. We first monitored the expression of CXCR4 in cells edited for the site-specific integration of a GFP-expressing cassette into the Adeno-Associated Virus Integration Site 1 (AAVS1) safe harbor locus using a recently optimized protocol. CD34⁺ cells were electroporated with Cas9 RNP assembled with sgRNA targeting AAVS1 and editing enhancers (GSE56/Ad5-E4orf6/7), and immediately transduced with a repair template carrying AAV6-GFP vector (FIG. 11A). CXCR4 expression was decreased in gene-edited cells as compared to electroporated-only cells (FIG. 5A).

To counteract this adverse impact on CXCR4, we co-electroporated cells with CXCR4 mRNA combined with all the gene editing machinery. The control counterparts received GFP mRNA with the same gene editing machinery. While gene edited cells with or without GFP overexpression had decreased migration potential, gene edited HSPCs overexpressing CXCR4 showed higher migration potential in an in vitro migration assay, even when compared to electroporated-only control cells (FIG. 5B).

We next tested whether CXCR4 overexpression could provide an engraftment advantage to edited cells. While the chimerism level of edited cells (stably expressing GFP) only reached a median of 5% for the standard treatment, addition of CXCR4 mRNA allowed a 3-fold enhanced engraftment reaching a median of 15% (FIG. 5C-D). Intriguingly, because HDR efficiency was around 40% as measured in the more primitive subset in vitro (FIG. 11B), the total chimerism reached in vivo by cells edited together with CXCR4 mRNA would correspond to the levels reached by LV-transduced cells and electroporated with CXCR4 mRNA in the previous experiment (40% GFP for cells transduced by LV to 90%). This result supports our previously reported finding that editing with optimized enhancers contains the impact of the procedure on HSPC repopulation properties. Lineage proportion within PB, BM and spleen were similar for edited and non-edited cells, with or without initial overexpression of CXCR4 (FIG. 11C-H).

Overall, these findings show the portability of our strategy across different genetic engineering strategies and the feasibility of establishing a human hematopoietic graft comprising a fraction of edited cells sufficient for providing therapeutic benefits in diseases such as HIGM-1, without resorting to any preconditioning.

Whereas the results of M-HSCT as described here are already promising, multiple strategies could be used to improve its efficacy further. We tested different variants of CXCR4 (lerano, C., et al. (2009). Cell Cycle 8, 1228-1237; McDermott, D. H., et al. (2011). J Cell Mol Med 15, 2071-2081; Rosenkilde, M. M., et al. (2004). J Biol Chem 279, 3033-3041; and Rosenkilde, M. M., et al. (2007). J Biol Chem 282, 27354-27365), which all led to an increased surface over-expression of CXCR4 and were able to endow HSPCs with a further migration advantage (FIG. 5E; FIG. 11I-J). Interestingly, two of these variants were resistant to AMD3100 and even more to AMD3465, while keeping an efficient response to CXCL12, as shown by migration of overexpressing cells in the presence of CXCR4 antagonists (FIG. 5F-G). These variants could be used in the context of M-HSCT to further enhance the competitive advantage of infused cells over the mobilized cells and, conceivably, the extent of chimerism established.

Moreover, other molecules could be exploited beside CXCR4 for endowing the infused cells with a transient competitive advantage. We showed that overexpression of KIT, ITGA4 and CD47 mRNA could all provide an in vivo engraftment advantage (FIG. 5H), similar or even higher than that shown for CXCR4, to infused human cells in the context of M-HSCT, supporting its versatility and broad potential when coupled to mRNA-based genetic engineering of the administered cells.

### Mobilization-based HSCT (H-HSCT)

Here we provide evidence that HSPC mobilization might be successfully exploited in HSCT taking advantage of a window of opportunity opened at the peak of mobilization when donor cells might effectively outcompete those in the circulation for repopulation of the depleted BM niches. Competitive advantage of donor cells results from the rescue during ex vivo culture of a detrimental impact of mobilizers on HSPCs and might be further enhanced by transient over-expression of engraftment effectors. We present proof-of-principle of the therapeutic efficacy of M-HSCT in a mouse model of HIGM-1, although using healthy BM cells as surrogate of autologous genetically corrected ones, and further developed it using human hematochimeric mouse models showing its applicability to human HSPCs and its versatility when coupled to different genetic engineering strategies, such as gene replacement and gene editing. Overall, our findings encourage the eventual disposal of conventional genotoxic conditioning when designing autologous HSPC-GT and should pave the way to its broader and safer use in a relevant number of inherited diseases.

Whereas it is well established that mobilized HSPCs can home and successfully engraft in conditioned recipients, the processes underlying reconstitution to full niche occupancy are less well understood. Whether and to what extent residual HSPCs in the BM and those still in the circulation (in our case) compete with the infused ones in these processes is unknown. Infused HSPCs can be trapped in different non-hematopoietic locations and become phagocytosed, decreasing even more the effective therapeutic dose administered and engraftment. Our findings shed some lights on the dynamics and source of such competition. The short window of time used for M-HSCT suggests that early niche occupancy by the cells in the circulation prevents repopulation from the residual ones in the BM. This hypothesis is also supported by recent reports that describe the possibility of editing HSPCs in vivo, which requires prior cell mobilization and thus suggest that egressed cells from the niche may contribute to long-term haematopoiesis.

Our strategy is built-in the process of ex vivo HSPC engineering. The ex vivo culture allows recovery of surface molecules crucial for homing/engraftment and whose expression has been lowered by the mobilization protocol. On the other hand, when genetic engineering has a detrimental impact on the engraftment or repopulation potential of the treated HSPCs, as reported for gene editing, HSPC fitness can be enhanced by transient overexpression of molecular targets involved in the homing process concomitantly to the genetic modification. Conceivably, this approach would also be compatible with a short process time, in which mobilized cells immediately undergo genetic engineering and exchange. Of note, in the clinical setting where HSPC harvest and M-HSCT were to be performed almost concurrently, mobilized cells in the recipient would have been depleted from the circulation at the time of infusion of the engineered ones thus further reducing competition for engraftment.

Using modified nucleotides and optimizing design of the mRNA increased the time and extent of expression in transfected cells without eliciting innate recognition and detrimental responses. Moreover, the adoption of an “mRNA-only” platform safeguards against the risk of even sporadic integration of the effector transgene into the cellular genome, thus allowing safe capture of powerful gain-of-function effectors to our purpose. These include homing receptors such as CXCR4, adhesion molecules such as ITGA4, and inhibitors of professional phagocytosis such as CD47 but might conceivably be extended to other genes involved in pathways such as self-renewal. Moreover, mRNA-based delivery of engraftment enhancers can be easily incorporated into the ex vivo gene editing process, without increasing its overall time or burden, except for the total amount of transfected mRNA, which must be comprised within the limits of cell tolerability.

Although the M-HSCT protocols described here comprise some reagents not yet approved for clinical use, several new mobilizers are emerging for clinical testing, such as GRO-Beta (CXCL2) that antagonizes CXCR2 (Fukuda, S., et al. (2007). Blood 110, 860-869). As these new reagents become clinically applicable, they may allow even more HSPC mobilization and, thus, more effective exchange, possibly allowing removal of G-CSF treatment. In this case, BM niches may be better preserved allowing faster hematopoietic reconstitution by the gene-corrected cells. It is likely that our strategy might be relatively blind to the nature and mechanisms of action of the mobilizers used, provided that efficient HSPC depletion is achieved, and the newly infused cells are suitably engineered to outcompete the residual ones. Although we have not specifically investigated here the toxicity profile of our strategy, we expect it to be in line with that associated to the clinical use of mobilizers, which have shown an excellent safety record with only minimal adverse effects reported (Mueller, M. M., et al. (2013). Vox Sang 104, 46-54.). Moreover, given the low toxicity associated with the mobilization protocol, serial administration of corrected cells from a stored cell product for multiple administration following mobilization cycles, can be envisioned to further increase engraftment.

Of note, the use of G-CSF and AMD3100 as preparative regimen for patients with severe combined immunodeficiency undergoing HSCT has been tested and reported as inefficient (Dvorak, C. C., et al. (2014). Pediatr Transplant 18, 602-608). However, the percentage of mobilized CD34⁺ cells in the PB were suboptimal highlighting a limited BM vacancy which might explain the low/absent chimerism. Moreover, donor CD34⁺ cells were mobilized but not cultured ex vivo, thus potentially bearing a lower homing and engraftment potential as shown here. Interestingly, increased donor engraftment and event-free survival following addition of G-CSF/AMD3100 to the conditioning regimen of Wiskott-Aldrich Syndrome patients undergoing allogenic HCT has been reported (Balashov, D., et al. (2018). Biol Blood Marrow Tr 24, 1432-1440). Similarly, mixed chimerism after G-CSF/AMD3100 administration in addition to a nonmyeloablative conditioning regimen in patients with acute myelogenous leukemia has been reported (Konopleva, M., et al. (2015). Bone Marrow Transpl 50, 939-946), further supporting the contention that mobilization may enhance engraftment of infused cells.

The strategies developed here could be useful in the context of HSCT-GT, given that autologous cells do not need to overcome immune barriers in the recipient and that a mixed chimerism ranging around 30% might be sufficient for therapeutic benefit in many diseases that are candidates for HSPC-GT (Zimmerman, C., and Shenoy, S. (2020). Front Immunol 11, 1791). These include most primary immunodeficiencies, but might also extend to hemoglobinopathies and some lysosomal storage disorders. Indeed, we provide here proof-of-principle of the phenotypic rescue of HIGM-1 by M-HSCT in the mouse model, albeit using wild-type cells as surrogate of edited cells. Allogeneic HSCT is the only curative treatment currently available for this disease, however matched donors are not always available, and the procedure is associated with high risk of graft rejection, GvHD, infections, liver failure, death; indeed, despite its curative potential, HSCT has little impact on ameliorating survival of HIGM-1 patients. Thus, therapeutic alternatives to treat patients safely and more effectively for whom HSCT is too risky are strongly needed. M-HSCT with autologous cells corrected by gene editing might represent a promising option, where the use of engraftment enhancers might also compensate for the limited efficiency of HDR in primitive HSPCs.

The combination of mobilization and increased engraftment efficiency investigated in our studies might provide a way to entirely bypass the requirement for chemo/radiotherapy in HSPC-GT, conferring long-term therapeutic benefits with considerably less risk and long-term toxicity to patients. Conditioning regimes based on selective immunodepletion might also fit our strategy, as matching engraftment enhancers could be used, such as a mutant KIT not recognized by the anti-KIT antibodies/immunotoxin used for depletion, according to the model demonstrated here for the AMD3100-resistant CXCR4 variant. The combination of our engraftment enhancement strategy with immunotoxin-based strategy or low-dose chemotherapy may further broaden its efficiency and applicability.

### Methods

### Mice

C57Bl/6 Ly45.1, C57Bl/6 Ly45.2 mice were purchased from Charles River Laboratory. Cd40Ig^{-/-} (B6.129S2-Cd401gtm1lmx/J), humanized NSG or NSGW41 mice were purchased from The Jackson Laboratory and maintained in specific-pathogen-free (SPF) conditions. The procedures involving animals were designed and performed with the approval of the Animal Care and Use Committee of the San Raffaele Hospital (IACUC #818 for Cd40Ig^{-/-}, #876 for NSGW41 and NSG) and communicated to the Italian Ministry of Health and local authorities according to Italian law.

### Murine HSPC Transplantation Studies

Donor mice between 6 and 10 weeks of age were euthanized by CO₂, and BM cells were retrieved from femurs, tibias, and humeri. HSPCs were purified by Lin− selection using the mouse Lineage Cell Depletion Kit (Miltenyi Biotec) according to the manufacturer's instructions. Cells were then cultured (for 2 hours or overnight) in serum-free StemSpan medium (StemCell Technologies) containing penicillin, streptomycin, glutamine, and a combination of mouse cytokines (20 ng/ml IL-3, 100 ng/ml SCF, 100 ng/ml Flt-3L, 50 ng/ml TPO all from PeproTech), at a concentration of 2-5×10⁶ cells.ml⁻¹. Purified Lin⁻ cells were transplanted at a total dose of 2×10⁶ cells/mouse into 8 to 12-week-old mobilized mice, three hours after the last injection of AMD3100 and/or Bio5192. Serial collections of blood from the retro-orbital vein were performed to monitor the hematological parameters and donor cell engraftment. At the end of the experiment, BM, thymus and spleen, and lymph nodes were harvested and analyzed.

### ELISA

For ELISA performed on BM matrix, femurs and tibias were repeatedly flushed with 1 mL of cold PBS. Cells were pelleted by centrifugation at 300 g for 10 minutes at 4° C., supernatant was then collected and stored at −80° C. ELISA has been performed following manufacturer instruction after a 1:100 dilution with 1× Assay Diluent for MMP9 (Thermo Scientific) or on undiluted BM matrix for CXCL12 (Sigma-Aldrich).

### In Vivo Immunization and IgG Quantification

Mice were immunized by intraperitoneal injection (i.p.) with 100 μg of TNP-KLH (Lgc Biosearch Technologies) in Imject Alum Adjuvant (1:2) (ThermoFisher Scientific), as described before (Vavassori, V., et al. (2021). Embo Mol Med 13, e13545). Serum was collected at day 0, 7, and 14 after immunization. Mice were boosted as described above on day 21, and serum was collected on day 7 after re-challenge. For IgG quantification, the concentration of antigen-specific IgGs in mouse sera was determined by an enzyme-linked immunosorbent assay (ELISA). Plates were coated with 100 μL/well of 5 μg/ml TNP-KLH in carbonate buffer. Following incubation, plates were washed three times in PBS containing 0.05% Tween20 (Sigma-Aldrich) (Wash Buffer). The plates were then blocked for 1 h using 100 μL/well of PBS containing 1% Bovine Serum Albumine (BSA), followed by a washing step, as described above. Serum samples were serially diluted in wash buffer and 100 μL/well of each diluted sample was added into the plate and incubated for 2 h at room temperature. For determination of the plate background optical density (OD) values, some wells were incubated with wash buffer alone. Following incubation, plates were washed and 100 μL/well of HRP-conjugated goat anti-mouse (Southern Biotech 1:10,000) was added and incubated for 1 h at room temperature. After washing, the plates were incubated for 5 min with 3,3′,5,5′0-tetramethyl benzidine (TMB, Sigma-Aldrich) substrate at room temperature. The reaction was stopped by the addition of 50 μL of 1 M H₂SO₄. The OD values at 450 nm were determined for each well using a Multiskan GO microplate reader (Thermo Fisher Scientific) and normalized to IgG1 standard curves. Results were expressed as mean of duplicate determinations.

### Cell Line and Primary Cells

HEK293T cells were cultured in Iscove's modified Dulbecco's medium (Corning) supplemented with 10% heat-inactivated fetal bovine serum (Euroclone), 100 IU·ml⁻¹ penicillin, 100 μg·ml⁻¹, streptomycin and 2% glutamine.

G-CSF mPB CD34⁺ HSPCs and G-CSF/Mozobil mPB CD34⁺ HSPCs were purchased from Mobilized Leukopak (AllCells) according to TIGET-HPCT protocol approved by the San Raffaele Institute Bioethical Committee and purified with the CliniMACS CD34 Reagent System (Miltenyi Biotec) according to the manufacturer's instructions. HSPCs were seeded at the concentration of 1×10⁶ cells per ml in serum-free StemSpan medium (StemCell Technologies) supplemented with 100 IU·ml⁻¹ penicillin, 100 μg·ml⁻¹ streptomycin, 2% glutamine, 300 ng·ml⁻¹ hSCF, 300 ng·ml⁻¹ hFlt3-L, 100 ng·ml⁻¹ hTPO, 1 μM SR1, 35 nM UM171 and 10 μM PGE2 (except when a subsequent transduction is planned). All cells were cultured in a 5% CO2 humidified atmosphere at 37° C. In vivo, the human HSPC population was defined as CD34⁺ CD38⁻ CD90⁺, and in vitro as CD34⁺ CD133⁺ CD90⁺.

### LV Vector Production and Titration

VSV.G-pseudotyped third-generation self-inactivating SINLV were produced by calcium phosphate transient transfection into 293Tcells. 293T cells were transfected with a solution containing a mix of the LV genome transfer plasmid, bearing the expression cassette for GFP, the packaging plasmids pMDLg/pRRE, pMD2.VSV.G, pKRev(pILVV01) and pAdVantage (Promega). Medium was changed 14-16 hours after transfection and supernatant was collected 30 hours after medium change. LV-containing supernatants were passed through a 0.22 μm filter (Millipore) and transferred into sterile polyallomer tubes (Beckman) and centrifuged at 20,000 g for 120 min at 20° C. (Beckman Optima XL-100KUltracentrifuge). LV pellet was dissolved in the appropriate volume of phosphate buffered saline (PBS) to allow 500× concentration. Concentrated vector stock was aliquoted and stored at −80° C. LV titer was determined by flow cytometry 4-5 days after LV transduction analysis or quantitative PCR, 10-14 days after LV transduction, as described (Milani, M., et al. (2017). Embo Mol Med 9, 1558-1573).

mRNA IVT

GFP, ITGA4, CXCR4, CD47 and KIT DNA coding RNA were synthetized (GeneArt, Thermo Fisher) using Homo Sapiens codon-optimized algorithm. A complementary CXCR4 sequence was produced with reduced uridine content (dU). Coding sequences were subcloned in ‘pVax’ plasmids under the control of the following 5′ aptamer sequence: CapAG—eIF4G aptamer (GACTCACTATTTGTTTTCGCGCCCAGTTGCAAAAAGTGTCG)—Kozak sequence (CCACC)—start codon (ATG). Downstream the codon-optimized sequence follows a woodchuck hepatitis virus posttranscriptional regulatory element and a 120-bp polyA sequence.

For mRNA IVT, pVAX plasmids were linearized with SpeI (New England Biolabs) restriction enzyme and purified by phenol-chloroform extraction. mRNA was in vitro transcribed using the commercial 5×MEGAscript T7 kit (Thermo Fisher) and capped with 5 mM of CleanCapAG (Trilink). Different modified nucleotides were used: 5-Methoxyuridine-5′-Triphosphate (moU), Pseudouridine-5-Triphosphate (pU), 5-Methylcytidine-5′-Triphosphate (mC; Trilink) at a concentration of 7.5 mM. mRNA was purified using RNeasy Plus Mini Kit (Qiagen) followed by high-performance liquid chromatography purification (ADS BIOTEC WAVE System) and Amicon Ultra-15 (30 K) tube (Millipore) concentration. mRNA productions were aliquoted and stored at −80° C. All RNA samples were analyzed by denaturing agarose gel electrophoresis to assess the quality and integrity.

### Transduction

10⁶ CD34⁺ cells/ml were stimulated with 10 μM PGE2 20 hours post-thawing. After 2 hours of pre-stimulation, cells were infected for 14 hours with LV-GFP at multiplicity of infection (MOI) 100. When necessary, cells were electroporated 14 hours post-transduction.

### Electroporation

Cells were electroporated with 5 μg of encoding mRNA, two days post-thawing.

Transfections were performed using the 4D-Nucleofector™ System (Lonza) and following manufacturer's instructions primary cells (P3 Primary Cell 4D-Nucleofector X Kit, program EO-100; Lonza). From 6 h to day 10 after electroporation, target protein expression within HSPC subpopulations was evaluated by flow cytometry.

### Gene Editing of Human HSPCs and Analyses

For AAV6-based gene editing, 1×10⁶ CD34⁺ cells (mobilized with G-CSF) after 3 days of culture in the medium described above were washed with ten volumes of DPBS and electroporated using P3 Primary Cell 4D-Nucleofector X Kit and program EO-100 (Lonza). Cells were electroporated with RNPs at a final concentration of 2.5 μM together with 0.1 nmol of Alt-R Cas9 Electroporation Enhancer (Integrated DNA Technologies), according to the manufacturer's instructions. AAV6 transduction was performed at a dose of 1×10⁴ vg per cell 15 min after electroporation. Additional mRNAs were added in the gene editing mixture as follows: (i) 3.5 μg GSE56/Ad5-E4orf6/7 (Fusion protein with P2A self-cleaving peptide) mRNA, (ii) 3.5 μg GSE56/Ad5-E4orf6/7 mRNA and 3.5 μg GFP mRNA or CXCR4 mRNA. Three and fifteen days after the editing procedure, cells were harvested to measure the percentage of cells expressing the GFP marker by flow cytometry and to extract gDNA for molecular analyses, as described (Ferrari, S., et al. (2021). Nat Protoc 16, 2991-3025).

### In Vitro Migration Assay

Migration was performed using transwells permeable supports (Costar, 5 μm polycarbonate membrane). Briefly, 2×10⁵ mPB CD34⁺ previously electroporated with target mRNA, were seeded in the upper chamber, in StemSpan medium with cytokines. The lower chamber was filled with 600 μl of StemSpan medium with cytokines, supplemented with recombinant CXCL12 (PeproTech, 125 ng/ml). After 3 hours, migrating cells were recovered from the lower chamber and quantitatively evaluated on the BD Accuri™ 06 flow cytometer. For the migration inhibition experiment, Mozobil/AMD3100 and AMD3465 were added at 200 μM.

### Flow Cytometry

Immunophenotypic analyses were performed on fluorescence activated cell sorting FACS Canto II (BD Pharmingen) according to manufacturers' instructions, equipped with DIVA Software and analyzed with the FSC express software (v. 6, 7, De Novo Software). 5×10⁴-2×10⁵ cells (from culture or mouse samples) were harvested, washed with PBS or MACS buffer (PBS pH 7.2, 0.5% BSA, 2 mM EDTA), treated with fragment crystallizable (Fc) Receptor-Block (Miltenyi Biotec), when antibody stained, and then re-suspended in the buffer used for washing. Staining was performed in MACS buffer, incubating cells for 15 minutes at 4° C. in dark with a mix of antibodies listed below, in a final volume of 100 μL. Sphero Rainbow Calibration Particles (Spherotech) beads were used to calibrate the instrument detectors, for consistent MFI measurement, for analysis performed at different times. Single stained and Fluorescence Minus One (FMO)-stained cells were used as controls.

LIVE/DEAD Fixable Dead Cell Stain Kit (Thermo Fisher) or 7-aminoactinomycin (Sigma-Aldrich) was included in the sample preparation for flow cytometry according to the manufacturer's instructions to exclude dead cells from the analysis. Apoptosis analysis was performed on CD34⁺ cells one day after electroporation using Annexin V (Biolegend) and Apoptosis Detection kit with 7-Aminoactinomycin D (7AAD, BD Pharmingen) according to the manufacturers' instructions. Percentages of live (7AAD⁻, AnnexinV⁻), early apoptotic (7AAD⁻, AnnexinV+) and late apoptotic (7AAD⁺, AnnexinV⁺) cells are reported.

For intracellular staining, surface antigens were stained prior to fixation and permeabilization steps, performed using the BD Cytofix/Cytoperm fixation/permeabilization Kit, according to the manufacturer's instructions.

Blood samples were also analysed with the hemocytometer Sysmex KX-21N (Block scientific, Sysmex corporation) to quantify absolute numbers.

### Cd34⁻ HSPC Xenotransplantation Experiments in NSG and NSGW41 Mice

For transplantation into sublethally irradiated (150-180 cGy) NSG mice, 3×10⁵ cord blood CD34⁺ cells, diluted in 200 μL of PBS, were injected intravenously 24 hours after electroporation (performed 48 hours post-thawing).

For transplantation into NOD-B6-SCID II2rγ−/− Kit(W41/W41) mice, 3×10⁵ G-CSF mPB CD34⁺ cells, diluted in 200 μL of PBS, were injected intravenously 48-72 hours post-thawing. Once the human chimerism reached 10%, humanized NSGW41 mice were mobilized and transplanted with 1-3×10⁵ G-CSF mPB CD34⁺ cells, transduced (for stable overexpression of a fluorescent marker) the first day post-thawing and/or electroporated on the third day post-thawing, and transplanted on the fourth day. Mice were randomly distributed to each experimental group.

| FIG. 3 | | |
|---|---|---|
| Gene replacement | 1^{st} transplant | 2^{nd} transplant |
| # G-CSF mPB CD34 | 1 × 10⁵ | 1 × 10⁵ |
| (Counted at day 1) | | (Transplanted at day 3) |

| FIG. 4 | | |
|---|---|---|
| Gene replacement + | | |
| Electroporation | 1^{st} transplant | 2^{nd} transplant |
| # G-CSF mPB CD34 | 1 × 10⁵ | 2 × 10⁵ |
| (Counted at day 1) | | (Transplanted at day 3) |

| FIG. 5 | | |
|---|---|---|
| Gene correction | 1^{st} transplant | 2^{nd} transplant |
| # G-CSF mPB CD34 | 1 × 10⁵ | 3 × 10⁵ |
| (Counted at day 1) | | (Transplanted at day 4) |

Human CD45⁺ cell engraftment, cell lineages and/or GFP⁺ cells were monitored by serial collection of blood from the retro-orbital vein and, at the end of the experiment (>12 weeks after transplantation), BM, thymus and spleen were harvested and analysed by flow cytometry for end-point analyses.

BM was flushed with PBS 2% BSA and 50 μL were stained for surface markers. The remaining cells were mouse cell-depleted and used for additional surface- or intracellular human antigen staining. For mouse cell depletion, BM cells flushed from the femurs and tibia of mice were processed with the Mouse Cell depletion Kit (Miltenyi Biotec) according to the manufacturer's instructions. Thymus was smashed and resuspended in PBS 2% BSA, and spleen was smashed, lysate with ACK Lysing Buffer (ThermoFisher) and resuspended in PBS 2% BSA. After processing, all samples were stained for surface marker and analyzed by flow cytometry. In some experiments, secondary transplantations were performed upon intravenous injection of 10⁶ human CD34+ harvested and purified (CD34 MicroBead Kit—following manufacturer instruction) from the BM of primary engrafted NSGW41 mice to NSG mice (16 weeks).

### Mobilization In Vivo

NSGW41 mice were injected i.v. with CD34⁺ cells as previously described above. After stable engraftment (10 weeks after injection), mice were treated for mobilization. G-CSF (Lenograstim, 250 μg/kg/day) was delivered for 7 days through osmotic pumps positioned subcutaneously (s.c.) (Micro-osmotic pump 1007D, Alzet). At days 6 and 7 after pumps implantation, mice received i.p. injections of AMD3100 (Mozobil, 5 mg/kg/day) and BIO5192 (R&D System, 1 mg/kg/day). 6 hours after last i.p. injections, mice were transplanted i.v. with mobilized-derived CD34⁺ cells, from the same donor, transduced with LV-GFP vector for stable overexpression and electroporated with mRNA for transient overexpression of the indicated gene products.

Different protocols were tested in Cd40Ig^{-/-} mice: (i) s.c. pump delivering 250 μg/kg/day of G-CSF for 7 days (G7); (ii) s.c. pump delivering 250 μg/kg/day of G-CSF for 7 days, with i.p. injections of AMD3100 (5 mg/kg/day) on day 6 and 7 (G7A); (iii) s.c. pump delivering 250 μg/kg/day of G-CSF for 7 days, with i.p. injections of AMD3100 (5 mg/kg/day) and BIO5192 (1 mg/kg/day) on day 6 and 7 (G7AB); (iv) s.c. pump delivering 125 μg/kg/day of G-CSF for 7 days, with i.p. injections of AMD3100 (5 mg/kg/day) and BIO5192 (1 mg/kg/day) on day 6 and 7 (G7AB-H); (v) G-CSF delivered by i.p. injections (125 μg/kg) every 12 hours for four days, with AMD3100 s.c. injections (5 mg/kg) 14 hours after the last dose of G-CSF (G5A); (vi) G-CSF delivered by i.p. injections (125 μg/kg) every 12 hours for four days, with AMD3100 (5 mg/kg) and BIO5192 (1 mg/kg) s.c. injections 14 hours after the last dose of G-CSF (G5AB); (vii) s.c. pump delivering 250 μg/kg/day of G-CSF for 3 days, with i.p. injections of AMD3100 (5 mg/kg/day) and BIO5192 (1 mg/kg/day) on day 6 and 7 (G3AB); (viii) s.c. pump delivering 125 μg/kg/day of G-CSF for 3 days, with i.p. injections of AMD3100 (5 mg/kg/day) and BIO5192 (1 mg/kg/day) on day 6 and 7 (G3AB-H); and (ix) AMD3100 (5 mg/kg/day) and BIO5192 (1 mg/kg/day) i.p. injected for three days.

Bone marrow vacancy and estimated chimerism were calculated based on the following formulas in Cd40Ig^{-/-} mice (summarized in table below):
To determine the total number of SLAM HSC per mouse at steady state (FIG. 2G, left panel), lower limbs (which account for 20% of the total BM) were collected in a defined volume and counted, paralleled with their characterization through FACS. We found 2500 SLAM HSC/lower limbs, therefore reaching a total of 12,500 SLAM HSC/mouse (2500*100/20). This number is in accordance with other published papers (Chen, J., et al. (2008). Experimental hematology, 36(10), 1236-1243; Karpova, D., et al. (2017). Blood, 129(21), 2939-2949; and Singh, P., et al. (2020). Stem cells (Dayton, Ohio), 38(7), 849-859).
To determine the total number of SLAM HSC that egressed from the BM post-G7AB mobilization, we examined the BM of mobilized mice. 1100 SLAM HSC were recovered in the lower limbs, reaching a total of 5500 SLAM HSC in the BM of mobilized mice. Therefore, to determine the number of SLAM HSC egressed, the number of SLAM HSC present post-mobilization was subtracted from the total number of SLAM HSC present in the steady state mouse, leading to an assessment of 7000 SLAM HSC egressing from the BM (12500-5500).
The mobilized SLAM HSC/mL was calculated based on the bleeding performed at the peak of mobilization in mobilized mice (FIG. 2G, right panel). The blood was analyzed through hematocytometer (WBC/mL) and FACS staining (% subpopulation). Based on the WBC and the percentage of each subpopulation, the mobilized SLAM HSC/mL was estimated. To calculate the total number of mobilized SLAM HSC, the concentration of mobilized SLAM HSC/mL was multiplied by the total blood volume estimated to 1.5 mL. Thereby, counts of SLAM HSC in the circulation of mobilized mice (FIG. 2G, right panel) were valued to 3500 SLAM HSC/mL, leading to an estimation of 5250 total SLAM HSC total in the circulation (2500*1.5), corresponding to 75% of the 7000 SLAM HSC egressed from the BM.

| | | Steady | |
|---|---|---|---|
| | | state mice | Mobilized |
| FIG. 2G | | (Sham) | mice (G7AB) |
| Left | # SLAM HSC in lower limbs | 2500 | 1100 |
| panel | # SLAM HSC total BM | 12500 | 5500 |
| | (Lower limbs = 20% total | | |
| | BM) | | |
| | #SLAM HSC egressed | 12500 − 5500 = 7000 | |
| | (# SLAM HSC total BM | | |
| | Sham − # SLAM HSC total | | |
| | BM mobilized) | | |
| Right | # SLAM HSC circulation | 28*1.5 = 43 | 3500*1.5 = 5250 |
| panel | (Total blood volume = | | |
| | 1.5 mL) | | |

| % SLAM HSC in circulation to | 5250*100/7000 = 75% |
|---|---|
| SLAM HSC egressed from the BM | |

Concerning the transplanted Lin− BM Cells:
2×10⁶ Lin⁻ BM cells were used/transplantation. Upon purification of Lin⁻ cells from the BM, we characterized the Lin⁻ population using FACS and determined the percentage of LSK (average 9% Lin−; 9×10⁴ LSK/million of Lin⁻) and SLAM HSC (average 2%; 2000 SLAM HSC/million of Lin⁻) subpopulation. We next calculated the transplanted number of LSK and SLAM HSC from 2×10⁶ Lin⁻ cells, corresponding to 4000 SLAM HSC total.
The ratio recipient to donor was determined by dividing the mobilized recipient SLAM HSC by the transplanted SLAM HSC. As 2×10⁶ Lin⁻ were transplanted each time, except for the dose response experiment (FIG. 7M), the SLAM HSC transplanted always correspond to 4000.

$$BM vacancy ( \% ) = \frac{Total mobilized SLAM HSC * 100}{Total SLAM HSC in the BM}$$

$$Estimated chimerism ( \% ) = \frac{BM vacancy}{( \frac{total SLAM HSC mobilized}{total SLAM HSC infused} ) + 1}$$

Humanized bone marrow vacancy and estimated chimerism were calculated based on the following formulas:

$$BM vacancy ( \% ) = \frac{Total mobilized CD 34 * 100}{Total CD 34 in the BM}$$

$$Estimated chimerism ( \% ) = \frac{BM vacancy}{( \frac{total CD 34 mobilized}{total CD 34 infused} ) + 1}$$

### Molecular Analysis

For HDR digital droplet PCR (ddPCR) analysis, 5-50 ng of gDNA were analyzed using the QX200 Droplet Digital PCR System (Bio-Rad) according to the manufacturer's instructions. HDR ddPCR primers and probes were designed on the junction between the vector sequence and the targeted locus. Human TTC5 (Bio-Rad) was used for normalization. DNA was extracted using Qiamp DNA micro kit (Qiagen) or Qiamp DNA mini kit according to starting number of cells (as suggested by manufacturers). DNA was subsequently quantified and checked for purity. Vector copies per diploid genome (vector copy number, VCN) were quantified by ddPCR starting from 5-50 ng of template gDNA using the following primers (HIV sense: 5′-TACTGACGCTCTCGCACC-3′; HIV antisense: 5′-TCTCGACGCAGGACTCG-3′) and probe (FAM-ATCTCTCTCCTTCTAGCCTC-MGBNFQ) against the primer binding site region of LVs. Endogenous DNA amount was quantified by a primer/probe set against the human telomerase gene (Telo sense: 5′-GGCACACGTGGCTTTTCG-3′; Telo antisense: 5′-GGTGAACCTCGTAAGTTTATGCAA-3′; Telo probe: VIC 5′-TCAGGACGTCGAGTGGACACGGTG-3′ TAMRA). Copies per genome were calculated by the formula=(ng LV/ng endogenous DNA)×(no of LV integrations in the standard curve). All reactions were carried out in duplicate. Each ddPCR run carries an internal control in the form of a CEMA301 cell line stably carrying a single vector integrant previously validated by Southern blot analysis.

For gene expression analyses, total RNA was extracted using RNeasy Plus Micro Kit (QIAGEN), according to the manufacturer's instructions and DNAse treatment was performed using RNase-free DNAse Set (QIAGEN). Complementary DNA was synthesized with SuperScript VILO IV cDNA Synthesis Kit (Thermo Fisher) with EzDNAse treatment. cDNA was then used for quantitative PCR (qPCR) in a Viia7 Real-time PCR thermal cycler using TaqMan Gene Expression Assays (Applied Biosystems) mapping to human IRF7, OAS1, ISG15 and RIG-1 genes. Data were analyzed with QuantStudio Real-Time PCR software v.1.1 (Applied Biosystem). Relative expression of each target gene was first normalized to HPRT and then represented as fold changes (2-AACt) relative to the untreated cells.

### Statistical Analysis

Here, the n indicates the number of biologically independent samples, animals or experiments. For some experiments, different HSPC donors were pooled. Data were summarized as mean±SEM. Inferential techniques were carried out whenever they were necessary for the interpretation of the data, otherwise descriptive statistics are reported. The Mann-Whitney test was performed to compare two independent groups, while in presence of more than two independent groups the Kruskal-Wallis test followed by post hoc analysis using Dunn's test was used. In presence of dependent observations and longitudinal comparisons the mixed-effects model (REML) were performed, followed by post hoc analysis with Sidak's test (when groups=2) or Tukey's test (when groups >2) and/or by post hoc analysis with Dunnett's test (within group). In all the analyses, P-values less than 0.05 were considered significant (*P<0.05, **P<0.01, ***P<0.001, ****P<0.0001. “ns” means non-significance). All statistical analyses were performed using R 3.5.0 (http://www.R-project.org/) or GraphPad Prism v8.

### Example 2

### Results

### AMD3100-Resistant CXCR4 Variant Increases Exchange Efficiency In Vivo

To investigate the potential of CXCR4 variants resistant to the AMD3100 antagonist used for HSPC mobilization, we employed the same mRNA-based transient overexpression approach described before to express these variants in human mobilized peripheral blood CD34+ cells. Our results showed that transplantation of HSPCs overexpressing the drug-resistant CXCR4 variant into hematochimeric mice resulted in enhanced exchange with the mobilized recipient cells, providing an advantage compared to cells overexpressing wild-type CXCR4, in particular when transplanting cells at the time of AMD3100 and BIO5192 injection, as shown in FIG. 12b. These findings indicate that the use of CXCR4 variants resistant to AMD3100 can further increase the advantage of donor cells.

In addition to their enhanced resistance to the AMD3100, we also evaluated the effect of CXCR4 variants on mobilization efficiency using the AMD3100 analogue AMD3465. Our results showed that the mobilization efficiency was not affected by the use of AMD3465 compared to AMD3100 in the standard protocol with G-CSF, and BIO5192 (FIG. 12c). Furthermore, we found that the exchange efficiency remained unchanged regardless of the use of AMD3100 or AMD3465 when transplanting donor cells at the peak of mobilization (FIG. 12d).

Altogether, these findings indicate that the use of CXCR4 variants resistant to AMD3100 does not compromise the efficiency of mobilization and exchange, which is an important factor for the clinical translation of this strategy. Our strategy leverages on HSPC mobilization and transient overexpression of a drug-resistant CXCR4, which provides a broader and safer clinical application of HSPC gene transfer and gene editing. This is particularly promising as may minimize the toxicity associated with current conditioning procedures.

### Materials and Methods

### Mobilization-Based Transplant

When performing the mobilization experiment we used the protocol previously described. In the experiments described in FIG. 12b, HSPC transplant was performed on the last day of treatment, right after AMD3100 and BIO5192 injections.

### EMBODIMENTS

Various preferred features and embodiments of the present invention will now be described with reference to the following numbered paragraphs (paras).

1. A population of haematopoietic stem and/or progenitor cell (HSPCs) for use in a method of therapy, the method comprising the steps of:
(a) administering one or more HSPC mobiliser to a subject to mobilise endogenous HSPCs from the subject's bone marrow; and
(b) administering the population of HSPCs to the subject.

2. A method for haematopoietic stem and/or progenitor cell (HSPC) transplantation in a subject in need thereof, comprising the steps:
(a) administering one or more HSPC mobiliser to the subject to mobilise the subject's endogenous HSPCs; and
(b) administering a population of HSPCs to the subject.

3, The population of HSPCs for use according to para 1, or the method according to para 2, wherein the population of HSPCs is administered at or after the peak of mobilisation, optionally wherein the population of HSPCs is administered at the peak of mobilisation.

4. The population of HSPCs for use according to para 1 or 3, or the method according to para 2 or 3, wherein the population of HSPCs is administered within about 9 hours, within about 6 hours, or within about 3 hours after step (a), optionally wherein the population of HSPCs is administered about 2-4 hours or about 3 hours after step (a).

5. The population of HSPCs for use according to para 1 or 3, or the method according to para 2 or 3, wherein the population of HSPCs is administered concurrently with step (a).

6. The population of HSPCs for use according to any of paras 1 or 3-5, or the method according to any of paras 2-5, wherein the one or more HPSC mobiliser is selected from a granulocyte colony-stimulating factor (G-CSF), a CXCR4 antagonist and a VLA-4 antagonist, or any combination thereof.

7. The population of HSPCs for use according to any of paras 1 or 3-6, or the method according to any of paras 2-6, wherein:
(i) the subject is administered a G-CSF for at least about 5 days before the population of HSPCs is administered;
(ii) the subject is administered a CXCR4 antagonist for at least about 1 day before the population of HSPCs is administered; and/or
(iii) the subject is administered a VLA-4 antagonist for at least about 1 day before the population of HSPCs is administered.

8. The population of HSPCs for use according to any of paras 1 or 3-7, or the method according to any of paras 2-7, wherein:
(i) the subject is administered a G-CSF for about 7 days before the population of HSPCs is administered;
(ii) the subject is administered a CXCR4 antagonist for about 2 days before the population of HSPCs is administered; and
(iii) optionally, the subject is administered a VLA-4 antagonist for about 2 days before the population of HSPCs is administered.

9. The population of HSPCs for use according to any of paras 1 or 3-8, or the method according to any of paras 2-8, wherein the population of HSPCs are autologous HSPCs or allogenic HSPCs, preferably wherein the population of HSPCs are autologous HSPCs.

10. The population of HSPCs for use according to any of paras 1 or 3-9, or the method according to any of paras 2-9, wherein the method further comprises a step of harvesting the mobilized endogenous HSPCs prior to administering the population of HSPCs.

11. The population of HSPCs for use according to any of paras 1 or 3-10, or the method according to any of paras 2-10, wherein the population of HSPCs is cultured ex vivo prior to administration, optionally wherein the method further comprises a step of genetically engineering the population of HSPCs prior to administering the population of HSPCs, and/or wherein the population of HSPCs are genetically engineered to express a transgene, gene-edited, and/or gene-corrected.

12. The population of HSPCs for use according to any of paras 1 or 3-11, or the method according to any of paras 2-11, wherein the population of HSPCs are genetically engineered to express one or more engraftment enhancer, preferably wherein the one or more engraftment enhancer is expressed transiently.

13. The population of HSPCs for use according to para 12, or the method according to para 12, wherein the one or more engraftment enhancer are each expressed from an RNA polynucleotide comprising a protein-coding sequence encoding an engraftment enhancer, optionally wherein the RNA is delivered to the population of HSPCs by electroporation or by lipid-mediated transfection.

14. The population of HSPCs for use according to para 13, or the method according to para 13, wherein the protein-coding sequence is operably linked to a translation non-blocking eIF4G aptamer.

15. The population of HSPCs for use according to para 13 or 14, or the method according to para 13 or 14, wherein the protein-coding sequence is operably linked to a woodchuck hepatitis virus post-transcriptional regulatory element (WPRE).

16. The population of HSPCs for use according to any of paras 13-15, or the method according to any of paras 13-15, wherein the protein-coding sequence is operably linked to a polyA tail, wherein the polyA tail is at least about 100, at least about 110, at least about 120, at least about 130, at least about 140, or at least about 150 nucleotides in length.

17. The population of HSPCs for use according to any of paras 13-16, or the method according to any of paras 13-16, wherein the RNA polynucleotide is 5′ capped mRNA, wherein the 5′ cap is m7G(5′)ppp(5′)(2′OMeA)pG.

18. The population of HSPCs for use according to any of paras 13-17, or the method according to any of paras 13-17, wherein the RNA polynucleotide comprises modified uridine, preferably pseudouridine.

19. The population of HSPCs for use according to any of paras 12-18, or the method according to any of paras 12-18, wherein the one or more engraftment enhancer is selected from C-X-C chemokine receptor type 4 (CXCR4) or a fragment or variant thereof, CD47 or a fragment or variant thereof, integrin alpha-4 (ITGA4) or a fragment or variant thereof, and tyrosine-protein kinase KIT (KIT) or a fragment or variant thereof, or any combination thereof.

20. The population of HSPCs for use according to any of paras 12-19, or the method according to any of paras 12-19, wherein the one or more engraftment enhancer comprises two or more, three or more, or four or more engraftment enhancers selected from: CXCR4 or a fragment or variant thereof, CD47 or a fragment or variant thereof, ITGA4 or a fragment or variant thereof, and KIT or a fragment or variant thereof.

21. The population of HSPCs for use according to any of paras 1 or 2-20, or the method according to any of paras 2-20, wherein the population of HSPCs are genetically engineered to transiently express CXCR4 or a fragment or variant thereof.

22. The population of HSPCs for use according to para 21, or the method according to para 21, wherein the CXCR4 or a fragment or variant thereof comprises or consists of an amino acid sequence having at least 70% identity to any of SEQ ID NOs: 1-9, preferably wherein the CXCR4 or a fragment or variant thereof comprises or consists of the amino acid sequence of SEQ ID NO: 2.

23. The population of HSPCs for use according to para 21 or 22, or the method according to para 21 or 22, wherein the CXCR4 or a fragment or variant thereof comprises or consists of the amino acid sequence of any of SEQ ID NOs: 3-9, preferably wherein the CXCR4 or a fragment or variant thereof comprises or consists of the amino acid sequence of any of SEQ ID NOs: 6-9.

24. The population of HSPCs for use according to any of paras 21-23, or the method according to any of paras 21-23, wherein the CXCR4 variant has increased resistance to a CXCR4 antagonist and/or has maintained or increased response to SDF-1.

25. The population of HSPCs for use according to any of paras 1 or 3-24, or the method according to any of paras 2-24, wherein the population of HSPCs are genetically engineered to transiently express CD47 or a fragment or variant thereof.

26. The population of HSPCs for use according to para 25, or the method according to para 25, wherein the CD47 or a fragment or variant thereof comprises or consists of an amino acid sequence having at least 70% identity to any of SEQ ID NOs: 23-26.

27. The population of HSPCs for use according to any of paras 1 or 3-26, or the method according to any of paras 2-26, wherein the population of HSPCs are genetically engineered to transiently express ITGA4 or a fragment or variant thereof.

28. The population of HSPCs for use according to para 27, or the method according to para 27, wherein the ITGA4 or a fragment or variant thereof comprises or consists of an amino acid sequence having at least 70% identity to SEQ ID NO: 29.

29. The population of HSPCs for use according to para 27 or 28, or the method according to para 27 or 28, wherein the ITGA4 variant has increased resistance to a VLA-4 antagonist

30. The population of HSPCs for use according to any of paras 1 or 3-29, or the method according to any of paras 2-29, wherein the population of HSPCs are genetically engineered to transiently express KIT or a fragment or variant thereof.

31. The population of HSPCs for use according to para 30, or the method according to para 30, wherein the KIT or a fragment or variant thereof comprises or consists of an amino acid sequence having at least 70% identity to SEQ ID NO: 31.

32. The population of HSPCs for use according to para 30 or 31, or the method according to para 30 or 31, wherein the KIT variant has increased resistance to a KIT-directed antibody or immunotoxin and/or has maintained or increased response to SCF.

33. The population of HSPCs for use according to any of paras 1 or 3-32, or the method according to any of paras 2-32, wherein the subject does not undergo chemotherapy or radiotherapy conditioning prior to administration of the HSPCs.

34. The population of HSPCs for use according to any of paras 1 or 3-33, or the method according to any of paras 2-33, wherein the subject has a primary immunodeficiency, a lysosomal storage disorder, a haemoglobinopathy, or cancer.

35. The population of HSPCs for use according to any of paras 1 or 3-34, or the method according to any of paras 2-34, wherein the subject has a primary immunodeficiency, such as human primary combined immunodeficiency Hyper IgM Syndrome 1 (HIGM-1).

36. The population of HSPCs for use according to any of paras 1 or 3-35, or the method according to any of paras 2-35, wherein the chimerism level of the population of HSPCs in the subject's bone marrow reaches a level of at least 10%, at least 15%, at least 20%, at least 25%, at least 30%, at least 35%, or at least 40%.

37. The population of HSPCs for use according to para 36, or the method according to para 32, wherein the chimerism level of the population of HSPCs in the subject's bone marrow is stable for at least 24 weeks.

38. The population of HSPCs for use according to any of paras 1 or 3-37, or the method according to any of paras 2-37, wherein steps (a) and (b) are repeated one or more times, two or more times, or three or more times.

39. Use of a C-X-C chemokine receptor type 4 (CXCR4) variant, integrin alpha-4 (ITGA4), and/or tyrosine-protein kinase KIT (KIT), for increasing engraftment by haematopoietic stem and/or progenitor cells (HSPCs), wherein the CXCR4 variant comprises one or more amino acid substitution selected from: V160L, A175F, Q200A, D262N, and H281A.

40. The use of para 39, wherein the HSPCs are genetically engineered to express the CXCR4 variant, ITGA4, and/or KIT, preferably wherein the HSPCs are genetically engineered to transiently express the CXCR4 variant, ITGA4, and/or KIT.

41. The use of para 39 or 40, wherein the HSPCs are transduced or transfected with one or more vectors encoding the CXCR4 variant, ITGA4, and/or KIT, preferably wherein the one or more vectors are RNA vectors.

42. The use of any of paras 39-41, wherein the HSPCs are genetically engineered to express two or more, three or more, or four or more of: the CXCR4 variant, ITGA4, KIT, CXCR4, and CD47.

43. A method for increasing engraftment by haematopoietic stem and/or progenitor cells (HSPCs), wherein the method comprises the step of genetically engineering the HSPCs to express a CXCR4 variant, ITGA4, and/or KIT, wherein the CXCR4 variant comprises one or more amino acid substitution selected from: V160L, A175F, Q200A, D262N, and H281A.

44. The use of any of paras 39-42 or the method of para 43, wherein the CXCR4 variant, ITGA4, and/or KIT are expressed transiently or stably by the HSPCs, preferably transiently.

45. The use of any of paras 39-42 or 44, or the method of para 43 or 44, wherein the CXCR4 variant comprises one or more amino acid substitution selected from: A175F, Q200A, D262N, and H281A.

46. The use of any of paras 39-42 or 44-45, or the method of any of paras 43-45, wherein the CXCR4 variant comprises or consists of the amino acid sequence of any of SEQ ID NOs: 3-9, preferably wherein the CXCR4 variant comprises or consists of the amino acid sequence of any of SEQ ID NOs: 6-9.

47. A population of genetically engineered haematopoietic stem and/or progenitor cells (HSPCs) obtainable by the method of any of paras 43 to 46.

48. A population of genetically engineered haematopoietic stem and/or progenitor cells (HSPCs), wherein the HSPCs are genetically engineered to express a CXCR4 variant, ITGA4, and/or KIT, preferably wherein the HSPCs are genetically engineered to transiently express a CXCR4 variant, ITGA4, and/or KIT, wherein the CXCR4 variant comprises one or more amino acid substitution selected from: V160L, A175F, Q200A, D262N, and H281A, preferably wherein the CXCR4 variant comprises one or more amino acid substitution selected from: A175F, Q200A, D262N, and H281A.

49. The population of genetically engineered HSPCs according to para 48, wherein the HSPCs are genetically engineered to express two or more, three or more, or four or more of: the CXCR4 variant, ITGA4, KIT, CXCR4, and CD47, preferably wherein the HSPCs are genetically engineered to transiently express two or more, three or more, or four or more of: the CXCR4 variant, ITGA4, KIT, CXCR4, and CD47.

50. A pharmaceutical composition comprising the population of genetically engineered haematopoietic stem and/or progenitor cells (HSPCs) of any of paras 47-49 and a pharmaceutically acceptable carrier, diluent or excipient.

51. A population of genetically engineered haematopoietic stem and/or progenitor cells (HSPCs) according to any of paras 47-50 for use in therapy.

52. A population of genetically engineered haematopoietic stem and/or progenitor cells (HSPCs) according to any of paras 47-50 for use in the treatment or prevention of cancer, an immune disorder, a lysosomal storage disorder, a bacterial or viral infection, a genetic disease, or a hemoglobinopathy.

53. A method for haematopoietic stem and/or progenitor cell (HSPC) transplantation, comprising the steps:
(a) providing a population of HSPCs which are genetically engineered to express a CXCR4 variant, ITGA4, and/or KIT, wherein the CXCR4 variant comprises one or more amino acid substitution selected from: V160L, A175F, Q200A, D262N, and H281A, preferably wherein the CXCR4 variant comprises one or more amino acid substitution selected from: A175F, Q200A, D262N, and H281A; and
(b) administering the HSPCs to a subject.

54. A method of treating or preventing cancer, an immune disorder, a lysosomal storage disorder, a bacterial or viral infection, a genetic disease, or a hemoglobinopathy, comprising the steps:
(a) providing a population of haematopoietic stem and/or progenitor cells (HSPCs) which are genetically engineered to express a CXCR4 variant, ITGA4, and/or KIT, wherein the CXCR4 variant comprises one or more amino acid substitution selected from: V160L, A175F, Q200A, D262N, and H281A, preferably wherein the CXCR4 variant comprises one or more amino acid substitution selected from: A175F, Q200A, D262N, and H281A; and
(b) administering the HSPCs to a subject.

55. The population of genetically engineered HSPCs for use according to para 51 or 52, or the method of para 53 or 54, wherein the subject is subjected to a mild myeloablative, reduced intensity or non-myeloablative conditioning regimen before administration of the HSPCs.

56. The population of genetically engineered HSPCs for use according to any of paras 51, 52 or 55, or the method of any of paras 53-55, wherein the subject:
(a) is subjected to a regimen for mobilisation of endogenous HSPCs; or
(b) is subjected to conditioning with one or more HSPC-specific immunotoxins.

57. The population of genetically engineered HSPCs for use according to any one of paras 51, 52, 55 or 56, or the method of any of paras 53-56, wherein the subject is subjected to a regimen for mobilisation of endogenous HSPCs.

58. The population of genetically engineered HSPCs for use according to any of paras 51, 52 or 55-57, or the method of any of paras 53-57, wherein the population of HSPCs is administered at or after the peak of mobilisation, optionally wherein the population of HSPCs is administered at the peak of mobilisation.

59. The population of genetically engineered HSPCs for use according to any of paras 51, 52 or 55-58, or the method of any of paras 53-58, wherein the population of HSPCs is administered within about 9 hours, within about 6 hours, or within about 3 hours after the regimen for mobilisation of endogenous HSPCs is completed, optionally wherein the population of HSPCs is administered about 2-4 hours or about 3 hours after the regimen for mobilisation of endogenous HSPCs is completed.

60. The population of genetically engineered HSPCs for use according to any of paras 51, 52 or 55-59, or the method of any of paras 53-59, wherein the population of HSPCs is administered concurrently with the regimen for mobilisation of endogenous HSPCs.

61. The population of genetically engineered HSPCs for use according to any of paras 51, 52 or 55-60, or the method of any of paras 53-60, wherein the regimen for mobilisation of endogenous HSPCs comprises administering one or more HPSC mobiliser selected from a granulocyte colony-stimulating factor (G-CSF), a CXCR4 antagonist and a VLA-4 antagonist, or any combination thereof.

62. The population of genetically engineered HSPCs for use according to any of paras 51, 52 or 55-61, or the method of any of paras 53-61, wherein the regimen for mobilisation of endogenous HSPCs comprises:
(i) administering a G-CSF for at least about 5 days;
(ii) administering a CXCR4 antagonist for at least about 1 day; and/or
(iii) administering a VLA-4 antagonist for at least about 1 day.

63. The population of HSPCs for use according to any of paras 51, 52 or 55-62, or the method according of any of paras 53-62, wherein:
(i) administering a G-CSF for about 7 days;
(ii) administering a CXCR4 antagonist for about 2 days; and
(iii) administering a VLA-4 antagonist for about 2 days.

64. The population of genetically engineered HSPCs for use according to any of paras 51, 52 or 55-63, or the method of any of paras 53-63, wherein the subject does not undergo chemotherapy or radiotherapy conditioning before administration of the HSPCs.

65. The population of genetically engineered HSPCs for use according to any of paras 51, 52 or 55-64, or the method of any of paras 53-64, wherein the CXCR4 variant comprises or consists of the amino acid sequence of any of SEQ ID NOs: 3-9, preferably wherein the CXCR4 variant comprises or consists of the amino acid sequence of any of SEQ ID NOs: 6-9.

66. An RNA polynucleotide comprising a protein-coding sequence.

67. The RNA polynucleotide according to para 66, wherein the protein-coding sequence is operably linked to a Kozak sequence.

68. The RNA polynucleotide according to para 66 or 67, wherein the protein-coding sequence is operably linked to a translation non-blocking eIF4F aptamer.

69. The RNA polynucleotide according to para 68, wherein the translation non-blocking eIF4F aptamer is a translation non-blocking eIF4G aptamer.

70. The RNA polynucleotide according to para 68 or 69, wherein the translation non-blocking eIF4F aptamer comprises or consists of a nucleotide sequence having at least 90% identity to any of SEQ ID NOs: 33-36.

71. The RNA polynucleotide according to any of paras 68-70, wherein the translation non-blocking eIF4F aptamer comprises or consists of a nucleotide sequence having at least 90% identity to SEQ ID NO: 34.

72. The RNA polynucleotide according to any of paras 66-71, wherein the protein-coding sequence is operably linked to a woodchuck hepatitis virus post-transcriptional regulatory element (WPRE).

73. The RNA polynucleotide according to para 72, wherein the WPRE comprises or consists of a nucleotide sequence having at least 70% identity to SEQ ID NO: 37.

74. The RNA polynucleotide according to any of paras 66-73, wherein the protein-coding sequence is operably linked to a polyA tail, wherein the polyA tail is at least about 100, at least about 110, at least about 120, at least about 130, at least about 140, or at least about 150 nucleotides in length.

75. The RNA polynucleotide according to any of paras 66-74, wherein the RNA polynucleotide is a 5′ capped mRNA, wherein the 5′ cap is m7G(5′)ppp(5′)(2′OMeA)pG.

76. The RNA polynucleotide according to any of paras 66-75, wherein the RNA polynucleotide comprises modified uridine, preferably pseudouridine.

77. The RNA polynucleotide according to any of paras 66-76, wherein the RNA polynucleotide comprises from 5′ to 3′: a m7G(5′)ppp(5′)(2′OMeA)pG cap; a translation non-blocking eIF4F aptamer; a Kozak sequence; a protein-coding sequence; a WPRE; and a polyA tail comprising at least about 100 nucleotides.

78. The RNA polynucleotide according to any of paras 66-77, wherein the transgene is an engraftment enhancer.

79. The RNA polynucleotide according to para 78, wherein the engraftment enhancer is selected from CXCR4 or a fragment or variant thereof, CD47 or a fragment or variant thereof, ITGA4 or a fragment or variant thereof, and KIT or a fragment or variant thereof.

80. The RNA polynucleotide according to para 78 or 79, wherein the engraftment enhancer is CXCR4 or a fragment or variant thereof.

81. The RNA polynucleotide according to para 80, wherein the CXCR4 or a fragment or variant thereof comprises or consists of an amino acid sequence having at least 70% identity to any of SEQ ID NOs: 1-9, preferably wherein the CXCR4 or a fragment or variant thereof comprises or consists of the amino acid sequence of any of SEQ ID NOs: 3-9, more preferably wherein the CXCR4 or a fragment or variant thereof comprises or consists of the amino acid sequence of any of SEQ ID NOs: 6-9.

82. The RNA polynucleotide according to para 80 or 81, wherein the protein-coding sequence comprises or consists of a nucleotide sequence having at least 70% identity to any of SEQ ID NOs: 10-22, preferably wherein the protein-coding sequence comprises or consists of the nucleotide sequence of any of SEQ ID NOs: 19-22.

83. The RNA polynucleotide according to para 78 or 79, wherein the engraftment enhancer is CD47 or a fragment or variant thereof.

84. The RNA polynucleotide according to para 83, wherein the CD47 or a fragment or variant thereof comprises or consists of an amino acid sequence having at least 70% identity to any of SEQ ID NOs: 23-26.

85. The RNA polynucleotide according to para 83 or 84, wherein the protein-coding sequence comprises or consists of a nucleotide sequence having at least 70% identity to SEQ ID NO: 27 or 28.

86. The RNA polynucleotide according to para 78 or 79, wherein the engraftment enhancer is ITGA4 or a fragment or variant thereof.

87. The RNA polynucleotide according to para 86, wherein the ITGA4 or a fragment or variant thereof comprises or consists of an amino acid sequence having at least 70% identity to SEQ ID NO: 29.

88. The RNA polynucleotide according to para 86 or 87, wherein the protein-coding sequence comprises or consists of a nucleotide sequence having at least 70% identity to SEQ ID NO: 30.

89. The RNA polynucleotide according to para 78 or 79, wherein the engraftment enhancer is KIT or a fragment or variant thereof.

90. The RNA polynucleotide according to para 89, wherein the KIT or a fragment or variant thereof comprises or consists of an amino acid sequence having at least 70% identity to SEQ ID NO: 31.

91. The RNA polynucleotide according to para 89 or 90, wherein the protein-coding sequence comprises or consists of a nucleotide sequence having at least 70% identity to SEQ ID NO: 32.

92. A DNA polynucleotide encoding the RNA polynucleotide according to any of paras 66-91.

93. A vector comprising the DNA polynucleotide according to para 92.

94. An isolated cell comprising the RNA polynucleotide according to any of paras 66-91, the DNA polynucleotide according to para 92, or the vector according to para 93.

95. A method for the production of the RNA polynucleotide according to any of paras 66-91, comprising the step of in vitro transcribing the DNA polynucleotide according to para 92.

96. The method according to para 95, wherein the method comprises transcribing the DNA with modified uridine, preferably pseudouridine.

97. The method according to para 95 or 96, wherein the method comprises capping the RNA polynucleotide with m7G(5′)ppp(5′)(2′OMeA)pG.

98. The method according to any of paras 95-97, wherein the method comprise purifying the RNA polynucleotide.

99. A method for delivering the RNA polynucleotide according to any of paras 66-91, wherein the RNA polynucleotide is delivered to a population of HSPCs by electroporation or by lipid-mediated transfection.

100. Use of the RNA polynucleotide according to any of paras 78-91 for increasing engraftment by haematopoietic stem and/or progenitor cells (HSPCs).

101. A method for increasing engraftment by haematopoietic stem and/or progenitor cells (HSPCs), wherein the method comprises the step of transfecting the RNA polynucleotide according to any of paras 78-91 into the HSPCs.

102. An isolated haematopoietic stem and/or progenitor cell (HSPC) comprising the RNA polynucleotide according to any of paras 78-91.

103. A population of isolated haematopoietic stem and/or progenitor cells (HSPCs) according to para 102.

104. A pharmaceutical composition comprising the isolated HSPC of para 102, or a population of HSPCs of para 103, and a pharmaceutically acceptable carrier, diluent or excipient.

105. An isolated HSPC according to para 102, or a population of HSPCs according to para 103, for use in therapy.

106. An isolated HSPC according to para 102, or a population of HSPCs according to para 103, for use in the treatment or prevention of cancer, an immune disorder, a lysosomal storage disorder, a bacterial or viral infection, a genetic disease, or a hemoglobinopathy.

107. A method for haematopoietic stem and/or progenitor cell (HSPC) transplantation, comprising the steps:
(a) providing a population of HSPCs comprising the RNA polynucleotide according to any of paras 78-91; and
(b) administering the HSPCs to a subject.

108. A method of treating or preventing cancer, an immune disorder, a lysosomal storage disorder, a bacterial or viral infection, a genetic disease, or a hemoglobinopathy, comprising the steps:
(a) providing a population of HSPCs comprising the RNA polynucleotide according to any of paras 78-91; and
(b) administering the HSPCs to a subject.

All publications mentioned in the above specification are herein incorporated by reference. Various modifications and variations of the disclosed methods and materials of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A population of haematopoietic stem and/or progenitor cell (HSPCs) for use in a method of therapy, the method comprising the steps of:
(a) administering one or more HSPC mobiliser to a subject to mobilise endogenous HSPCs from the subject's bone marrow; and
(b) administering the population of HSPCs to the subject.

2. The population of HSPCs for use according to claim 1, wherein the population of HSPCs is administered at or after the peak of mobilisation.

3. The population of HSPCs for use according to claim 1 or 2, wherein the one or more HPSC mobiliser is selected from a granulocyte colony-stimulating factor (G-CSF), a CXCR4 antagonist and a VLA-4 antagonist, or any combination thereof.

4. The population of HSPCs for use according to any of claims 1-3, wherein the population of HSPCs are autologous HSPCs.

5. The population of HSPCs for use according to any of claims 1-4, wherein the population of HSPCs is cultured ex vivo prior to administration.

6. The population of HSPCs for use according to any of claims 1-5, wherein the population of HSPCs are genetically engineered to express a transgene, gene-edited, and/or gene-corrected.

7. The population of HSPCs for use according to any of claims 1-6, wherein the population of HSPCs are genetically engineered to express one or more engraftment enhancer.

8. The population of HSPCs for use according to claim 7, wherein the one or more engraftment enhancer is selected from C-X-C chemokine receptor type 4 (CXCR4) or a fragment or variant thereof, CD47 or a fragment or variant thereof, integrin alpha-4 (ITGA4) or a fragment or variant thereof, and tyrosine-protein kinase KIT (KIT) or a fragment or variant thereof, or any combination thereof.

9. A population of genetically engineered haematopoietic stem and/or progenitor cells (HSPCs), wherein the HSPCs are genetically engineered to express a CXCR4 variant, ITGA4, and/or KIT, wherein the CXCR4 variant comprises one or more amino acid substitution selected from: V160L, A175F, Q200A, D262N, and H281A.

10. A method for haematopoietic stem and/or progenitor cell (HSPC) transplantation, comprising the steps:
(a) providing a population of haematopoietic stem and/or progenitor cells (HSPCs) which are genetically engineered to express a CXCR4 variant, ITGA4, and/or KIT, wherein the CXCR4 variant comprises one or more amino acid substitution selected from: V160L, A175F, Q200A, D262N, and H281A; and
(b) administering the HSPCs to a subject.


SEQUENCE LISTING (62 sequences)

SEQ ID NO: 1 - protein, 356 aa, synthetic construct
  REGION 1..356: note = CXCR4 isoform II
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 2 - protein, 352 aa, synthetic construct
  REGION 1..352: note = CXCR4 isoform I
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 3 - protein, 337 aa, synthetic construct
  REGION 1..337: note = CXCR4 Whim isoform II
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGK

SEQ ID NO: 4 - protein, 333 aa, synthetic construct
  REGION 1..333: note = CXCR4 Whim isoform I
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGK

SEQ ID NO: 5 - protein, 352 aa, synthetic construct
  REGION 1..352: note = CXCR4 V160L
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGLWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 6 - protein, 352 aa, synthetic construct
  REGION 1..352: note = CXCR4 A175F
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFFNVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 7 - protein, 352 aa, synthetic construct
  REGION 1..352: note = CXCR4 Q200A
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFAFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 8 - protein, 352 aa, synthetic construct
  REGION 1..352: note = CXCR4 D262N
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISINSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 9 - protein, 352 aa, synthetic construct
  REGION 1..352: note = CXCR4 H281A
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVAKWISITEALAFFHCCLNPILYAFLGAKFKTSAQHALTSVSRGSSLKILSKGKRGGHSSVSTESESSSFHSS

SEQ ID NO: 10 - DNA, 1068 bp, synthetic construct
  misc_feature 1..1068: note = CXCR4 isoform II
  source 1..1068: mol_type = other DNA
atgtctattcctctgcccctgctgcagatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagc

SEQ ID NO: 11 - DNA, 1068 bp, synthetic construct
  misc_feature 1..1068: note = CXCR4 isoform II
  source 1..1068: mol_type = other DNA
atgtccattcctttgcctcttttgcagatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagc

SEQ ID NO: 12 - DNA, 1056 bp, synthetic construct
  misc_feature 1..1056: note = CXCR4 isoform I
  source 1..1056: mol_type = other DNA
atggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagc

SEQ ID NO: 13 - DNA, 1056 bp, synthetic construct
  misc_feature 1..1056: note = CXCR4 isoform I
  source 1..1056: mol_type = other DNA
atggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagc

SEQ ID NO: 14 - DNA, 1068 bp, synthetic construct
  misc_feature 1..1068: note = CXCR4 Whim isoform II
  source 1..1068: mol_type = other DNA
atgtccattcctttgcctcttttgcagatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagtgaggtggacattcatctgtttccactgagtctgagtcttcaagttttcactccagc

SEQ ID NO: 15 - DNA, 1068 bp, synthetic construct
  misc_feature 1..1068: note = CXCR4 Whim isoform II
  source 1..1068: mol_type = other DNA
atgtctattcctctgcccctgctgcagatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagtgaggcggccacagctccgtgtctacagagagcgagagcagcagcttccacagcagc

SEQ ID NO: 16 - DNA, 1056 bp, synthetic construct
  misc_feature 1..1056: note = CXCR4 Whim isoform I
  source 1..1056: mol_type = other DNA
atggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagtgaggtggacattcatctgtttccactgagtctgagtcttcaagttttcactccagc

SEQ ID NO: 17 - DNA, 1056 bp, synthetic construct
  misc_feature 1..1056: note = CXCR4 Whim isoform I
  source 1..1056: mol_type = other DNA
atggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagtgaggcggccacagctccgtgtctacagagagcgagagcagcagcttccacagcagc

SEQ ID NO: 18 - DNA, 1056 bp, synthetic construct
  misc_feature 1..1056: note = CXCR4 V160L
  source 1..1056: mol_type = other DNA
atggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcctgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagc

SEQ ID NO: 19 - DNA, 1056 bp, synthetic construct
  misc_feature 1..1056: note = CXCR4 A175F
  source 1..1056: mol_type = other DNA
atggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcttcaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagc

SEQ ID NO: 20 - DNA, 1056 bp, synthetic construct
  misc_feature 1..1056: note = CXCR4 Q200A
  source 1..1056: mol_type = other DNA
atggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttcgcgttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagc

SEQ ID NO: 21 - DNA, 1056 bp, synthetic construct
  misc_feature 1..1056: note = CXCR4 D262N
  source 1..1056: mol_type = other DNA
atggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcaacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagc

SEQ ID NO: 22 - DNA, 1056 bp, synthetic construct
  misc_feature 1..1056: note = CXCR4 H281A
  source 1..1056: mol_type = other DNA
atggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtggccaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagc

SEQ ID NO: 23 - protein, 323 aa, synthetic construct
  REGION 1..323: note = CD47
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 24 - protein, 292 aa, synthetic construct
  REGION 1..292: note = CD47
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 25 - protein, 305 aa, synthetic construct
  REGION 1..305: note = CD47
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 26 - protein, 311 aa, synthetic construct
  REGION 1..311: note = CD47
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLN

SEQ ID NO: 27 - DNA, 969 bp, synthetic construct
  misc_feature 1..969: note = CD47
  source 1..969: mol_type = other DNA
atgtggcctctcgtggccgctctgctgctcgggagcgcttgttgcggcagcgcccagctgctgttcaacaaaaccaagtccgtcgagttcaccttctgcaacgacacagtggtgatcccctgcttcgtcaccaacatggaggctcagaataccaccgaggtctacgtcaagtggaaattcaagggcagagacatctacaccttcgacggagccctcaacaagagcacagtgcctaccgacttttccagcgccaagattgaggtgagccaactcctgaagggagacgccagcctgaagatggacaagagcgatgccgtcagccacacaggaaactacacctgcgaggtgacagagctcaccagagagggcgagaccatcatcgagctcaaatacagagtggtgtcctggttctcccccaacgagaacatcctcatcgtgatcttccccatcttcgccatcctgctgttctggggccagttcggcatcaaaaccctgaagtatagatccggcggcatggacgagaaaacaatcgccctgctggtggccggcctcgtgattaccgtgatcgtcatcgtgggcgccatcctcttcgtgcccggagagtacagcctcaagaacgccaccggcctgggcctgattgtgacctccacaggcattctgatcctgctgcactactacgtgttcagcacagccattggcctcacaagcttcgtgatcgccatcctggtcatccaggtgatcgcctacatcctcgccgtggtcggactcagcctctgtattgccgcttgcatccccatgcacggacccctcctgatctccggcctcagcattctggctctcgctcagctgctcggcctggtgtacatgaagttcgtcgccagcaaccagaagaccatccaaccccccagaaaggccgtcgaagagcctctgaacgcctttaaggagagcaagggcatgatgaacgacgag

SEQ ID NO: 28 - DNA, 969 bp, synthetic construct
  misc_feature 1..969: note = CD47
  source 1..969: mol_type = other DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaa

SEQ ID NO: 29 - protein, 1032 aa, synthetic construct [sequence omitted]

SEQ ID NO: 30 - DNA, 3096 bp, synthetic construct
  misc_feature 1..3096: note = ITGA4
  source 1..3096: mol_type = other DNA
atggcttgggaggctcggagagaacctggacctagaagagctgccgtgcgggagactgtcatgctgctgctgtgcctgggggtgcccacaggcagaccttacaacgtggataccgagagcgccctgctgtatcagggcccccacaacaccctgtttggctactctgtggtgctgcacagccacggcgccaaccgctggctgctggtgggcgcccccaccgccaattggctggccaatgcctccgtgatcaacccaggcgccatctacagatgtcggatcggcaagaatcctggccagacatgcgagcagctgcagctgggctcccccaacggcgagccttgtggcaagacatgcctggaggagagggacaatcagtggctgggcgtgacactgagcagacagcccggcgagaacggctccatcgtgacatgcggccacagatggaagaacatcttttacatcaagaacgagaataagctgccaacaggcggctgctatggcgtgcccccagacctgagaacagagctgagcaagcggatcgccccatgctaccaggattatgtgaagaagtttggcgagaattttgcctcttgccaggccggcatctcctccttctacaccaaggatctgatcgtgatgggcgcccctggctcttcctattggacaggctctctgttcgtgtataatatcacaaccaacaagtacaaggccttcctggacaagcagaaccaggtgaagttcggctcctatctgggctacagcgtgggcgccggccactttcggtctcagcacaccacagaggtggtgggcggcgccccccagcacgagcagatcggcaaggcctacatcttctccatcgacgagaaggagctgaatatcctgcacgagatgaagggcaagaagctgggctcctactttggcgcctccgtgtgcgccgtggacctgaatgccgacggcttttccgacctgctggtgggcgccccaatgcagtccacaatcagagaggagggcagagtgttcgtgtatatcaattccggcagcggcgccgtgatgaatgccatggagaccaatctggtgggctccgacaagtatgccgccagattcggcgagagcatcgtgaatctgggcgacatcgacaacgatggcttcgaggacgtggccatcggcgccccacaggaggatgacctgcagggcgccatctatatctataacggccgggccgacggcatctctagcaccttctcccagagaatcgagggcctgcagatcagcaagtccctgagcatgttcggccagagcatctccggccagatcgacgccgataataacggctacgtggatgtggccgtgggcgcctttagaagcgactccgccgtgctgctgagaacaaggcccgtggtgatcgtggatgcctccctgtctcaccccgagtccgtgaatcggacaaagtttgactgcgtggagaatggctggccaagcgtgtgcatcgatctgacactgtgcttttcctataagggcaaggaggtgccaggctatatcgtgctgttttacaacatgtctctggatgtgaacagaaaggccgagtcccccccaagattctacttttcctctaacggcacctctgatgtgatcaccggctctatccaggtgtccagcagggaggccaattgcagaacccaccaggcctttatgcggaaggatgtgcgcgacatcctgaccccaatccagatcgaggccgcctatcacctgggcccccacgtgatctccaagcggtccaccgaggagttccctccactgcagccaatcctgcagcagaagaaggagaaggacatcatgaagaagacaatcaacttcgccaggttttgcgcccacgagaactgttccgccgacctgcaggtgtctgccaagatcggcttcctgaagccccacgagaacaagacatatctggccgtgggctccatgaagaccctgatgctgaacgtgagcctgtttaacgccggcgacgatgcctacgagacaacactgcacgtgaagctgccagtgggcctgtacttcatcaagatcctggagctggaggagaagcagatcaactgtgaggtgaccgataactccggcgtggtgcagctggattgcagcatcggctatatctacgtggaccacctgtcccgcatcgacatctcttttctgctggacgtgtccagcctgtcccgggccgaggaggacctgtccatcacagtgcacgccacctgcgagaatgaggaggagatggacaacctgaagcactccagagtgacagtggccatcccactgaagtacgaggtgaagctgacagtgcacggctttgtgaatccaacctccttcgtgtacggctccaatgacgagaatgagccagagacatgtatggtggagaagatgaacctgacatttcacgtgatcaatacaggcaattctatggcccctaacgtgagcgtggagatcatggtgccaaattctttcagcccacagacagacaagctgtttaacatcctggacgtgcagacaaccacaggcgagtgtcactttgagaactaccagagagtgtgcgccctggagcagcagaagtccgccatgcagacactgaagggcatcgtgagatttctgagcaagacagataagaggctgctgtactgcatcaaggccgatccccactgcctgaattttctgtgcaacttcggcaagatggagtctggcaaggaggcctccgtgcacatccagctggagggcagaccctccatcctggagatggacgagaccagcgccctgaagttcgagatcagagccacaggcttcccagagcccaacccccgggtgatcgagctgaacaaggatgagaacgtggcccacgtgctgctggagggcctgcaccaccagcggcccaagagatatttcaccatcgtgatcatctccagctctctgctgctgggcctgatcgtgctgctgctgatctcctatgtgatgtggaaggccggcttctttaagcggcagtacaagtccatcctgcaggaagaaaatcgacgcgattcatggtcttacattaattctaaatcaaacgacgac

SEQ ID NO: 31 - protein, 976 aa, synthetic construct [sequence omitted]

SEQ ID NO: 32 - DNA, 2928 bp, synthetic construct
  misc_feature 1..2928: note = KIT
  source 1..2928: mol_type = other DNA
atgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtc

SEQ ID NO: 33 - RNA, 40 nt, synthetic construct
  misc_feature 1..40: note = translation non-blocking eIF4G aptamer
  source 1..40: mol_type = other RNA
actcactatttgttttcgcgcccagttgcaaaaagtgtcg

SEQ ID NO: 34 - RNA, 41 nt, synthetic construct
  misc_feature 1..41: note = translation non-blocking eIF4G aptamer
  source 1..41: mol_type = other RNA
gactcactatttgttttcgcgcccagttgcaaaaagtgtcg

SEQ ID NO: 35 - RNA, 40 nt, synthetic construct
  misc_feature 1..40: note = translation non-blocking eIF4G aptamer
  source 1..40: mol_type = other RNA
tccgcggcgccatctcatgtttagttgtcctatgtcgagc

SEQ ID NO: 36 - RNA, 41 nt, synthetic construct
  misc_feature 1..41: note = translation non-blocking eIF4G aptamer
  source 1..41: mol_type = other RNA
tccgtagaaacgcgttaaggtgaaagtttgagggctcctca

SEQ ID NO: 37 - RNA, 592 nt, synthetic construct
  misc_feature 1..592: note = WPRE
  source 1..592: mol_type = other RNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 38 - skipped

SEQ ID NO: 39 - RNA, 1858 nt, synthetic construct
  misc_feature 1..1858: note = CXCR4 WT
  source 1..1858: mol_type = other RNA
taatacgactcactataaggactcactatttgttttcgcgcccagttgcaaaaagtgtcgccaccatggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagctgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 40 - RNA, 1858 nt, synthetic construct
  misc_feature 1..1858: note = CXCR4 V160L
  source 1..1858: mol_type = other RNA
taatacgactcactataaggactcactatttgttttcgcgcccagttgcaaaaagtgtcgccaccatggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcctgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagctgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 41 - RNA, 1858 nt, synthetic construct
  misc_feature 1..1858: note = CXCR4 A175F
  source 1..1858: mol_type = other RNA
taatacgactcactataaggactcactatttgttttcgcgcccagttgcaaaaagtgtcgccaccatggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcttcaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagctgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 42 - RNA, 1858 nt, synthetic construct
  misc_feature 1..1858: note = CXCR4 Q200A
  source 1..1858: mol_type = other RNA
taatacgactcactataaggactcactatttgttttcgcgcccagttgcaaaaagtgtcgccaccatggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttcgcgttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagctgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 43 - RNA, 1858 nt, synthetic construct
  misc_feature 1..1858: note = CXCR4 D262N
  source 1..1858: mol_type = other RNA
taatacgactcactataaggactcactatttgttttcgcgcccagttgcaaaaagtgtcgccaccatggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcaacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagctgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 44 - RNA, 1858 nt, synthetic construct
  misc_feature 1..1858: note = CXCR4 H281A
  source 1..1858: mol_type = other RNA
taatacgactcactataaggactcactatttgttttcgcgcccagttgcaaaaagtgtcgccaccatggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtggccaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagctgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 45 - RNA, 3898 nt, synthetic construct
  misc_feature 1..3898: note = ITGA4
  source 1..3898: mol_type = other RNA
taatacgactcactataaggactcactatttgttttcgcgcccagttgcaaaaagtgtcgccaccatggcttgggaggctcggagagaacctggacctagaagagctgccgtgcgggagactgtcatgctgctgctgtgcctgggggtgcccacaggcagaccttacaacgtggataccgagagcgccctgctgtatcagggcccccacaacaccctgtttggctactctgtggtgctgcacagccacggcgccaaccgctggctgctggtgggcgcccccaccgccaattggctggccaatgcctccgtgatcaacccaggcgccatctacagatgtcggatcggcaagaatcctggccagacatgcgagcagctgcagctgggctcccccaacggcgagccttgtggcaagacatgcctggaggagagggacaatcagtggctgggcgtgacactgagcagacagcccggcgagaacggctccatcgtgacatgcggccacagatggaagaacatcttttacatcaagaacgagaataagctgccaacaggcggctgctatggcgtgcccccagacctgagaacagagctgagcaagcggatcgccccatgctaccaggattatgtgaagaagtttggcgagaattttgcctcttgccaggccggcatctcctccttctacaccaaggatctgatcgtgatgggcgcccctggctcttcctattggacaggctctctgttcgtgtataatatcacaaccaacaagtacaaggccttcctggacaagcagaaccaggtgaagttcggctcctatctgggctacagcgtgggcgccggccactttcggtctcagcacaccacagaggtggtgggcggcgccccccagcacgagcagatcggcaaggcctacatcttctccatcgacgagaaggagctgaatatcctgcacgagatgaagggcaagaagctgggctcctactttggcgcctccgtgtgcgccgtggacctgaatgccgacggcttttccgacctgctggtgggcgccccaatgcagtccacaatcagagaggagggcagagtgttcgtgtatatcaattccggcagcggcgccgtgatgaatgccatggagaccaatctggtgggctccgacaagtatgccgccagattcggcgagagcatcgtgaatctgggcgacatcgacaacgatggcttcgaggacgtggccatcggcgccccacaggaggatgacctgcagggcgccatctatatctataacggccgggccgacggcatctctagcaccttctcccagagaatcgagggcctgcagatcagcaagtccctgagcatgttcggccagagcatctccggccagatcgacgccgataataacggctacgtggatgtggccgtgggcgcctttagaagcgactccgccgtgctgctgagaacaaggcccgtggtgatcgtggatgcctccctgtctcaccccgagtccgtgaatcggacaaagtttgactgcgtggagaatggctggccaagcgtgtgcatcgatctgacactgtgcttttcctataagggcaaggaggtgccaggctatatcgtgctgttttacaacatgtctctggatgtgaacagaaaggccgagtcccccccaagattctacttttcctctaacggcacctctgatgtgatcaccggctctatccaggtgtccagcagggaggccaattgcagaacccaccaggcctttatgcggaaggatgtgcgcgacatcctgaccccaatccagatcgaggccgcctatcacctgggcccccacgtgatctccaagcggtccaccgaggagttccctccactgcagccaatcctgcagcagaagaaggagaaggacatcatgaagaagacaatcaacttcgccaggttttgcgcccacgagaactgttccgccgacctgcaggtgtctgccaagatcggcttcctgaagccccacgagaacaagacatatctggccgtgggctccatgaagaccctgatgctgaacgtgagcctgtttaacgccggcgacgatgcctacgagacaacactgcacgtgaagctgccagtgggcctgtacttcatcaagatcctggagctggaggagaagcagatcaactgtgaggtgaccgataactccggcgtggtgcagctggattgcagcatcggctatatctacgtggaccacctgtcccgcatcgacatctcttttctgctggacgtgtccagcctgtcccgggccgaggaggacctgtccatcacagtgcacgccacctgcgagaatgaggaggagatggacaacctgaagcactccagagtgacagtggccatcccactgaagtacgaggtgaagctgacagtgcacggctttgtgaatccaacctccttcgtgtacggctccaatgacgagaatgagccagagacatgtatggtggagaagatgaacctgacatttcacgtgatcaatacaggcaattctatggcccctaacgtgagcgtggagatcatggtgccaaattctttcagcccacagacagacaagctgtttaacatcctggacgtgcagacaaccacaggcgagtgtcactttgagaactaccagagagtgtgcgccctggagcagcagaagtccgccatgcagacactgaagggcatcgtgagatttctgagcaagacagataagaggctgctgtactgcatcaaggccgatccccactgcctgaattttctgtgcaacttcggcaagatggagtctggcaaggaggcctccgtgcacatccagctggagggcagaccctccatcctggagatggacgagaccagcgccctgaagttcgagatcagagccacaggcttcccagagcccaacccccgggtgatcgagctgaacaaggatgagaacgtggcccacgtgctgctggagggcctgcaccaccagcggcccaagagatatttcaccatcgtgatcatctccagctctctgctgctgggcctgatcgtgctgctgctgatctcctatgtgatgtggaaggccggcttctttaagcggcagtacaagtccatcctgcaggaagaaaatcgacgcgattcatggtcttacattaattctaaatcaaacgacgactaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 46 - RNA, 3684 nt, synthetic construct
  misc_feature 1..3684: note = KIT
  source 1..3684: mol_type = other RNA
taatacgactcactataaggactcactatttgttttcgcgcccagttgcaaaaagtgtcgccaccatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcggccgcgtcgatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcgtaaaaaaaaaaaaaaaaaaaaaaaaaaaaaaaaaaaaaaaaaaaaaaaaaaaaaaaaaaaaaaaa

SEQ ID NO: 47 - protein, 972 aa, synthetic construct
  REGION 1..972: note = KIT
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 48 - DNA, 1858 bp, synthetic construct
  misc_feature 1..1858: note = CXCR4 WT
  source 1..1858: mol_type = other DNA
taatacgactcactataaggactcactatttgttttcgcgcccagttgcaaaaagtgtcgccaccatggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagctgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 49 - DNA, 1858 bp, synthetic construct
  misc_feature 1..1858: note = CXCR4 V160L
  source 1..1858: mol_type = other DNA
taatacgactcactataaggactcactatttgttttcgcgcccagttgcaaaaagtgtcgccaccatggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcctgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagctgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 50 - DNA, 1858 bp, synthetic construct
  misc_feature 1..1858: note = CXCR4 A175F
  source 1..1858: mol_type = other DNA
taatacgactcactataaggactcactatttgttttcgcgcccagttgcaaaaagtgtcgccaccatggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcttcaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagctgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 51 - DNA, 1858 bp, synthetic construct
  misc_feature 1..1858: note = CXCR4 Q200A
  source 1..1858: mol_type = other DNA
taatacgactcactataaggactcactatttgttttcgcgcccagttgcaaaaagtgtcgccaccatggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttcgcgttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagctgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 52 - DNA, 1858 bp, synthetic construct
  misc_feature 1..1858: note = CXCR4 D262N
  source 1..1858: mol_type = other DNA
taatacgactcactataaggactcactatttgttttcgcgcccagttgcaaaaagtgtcgccaccatggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcaacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagctgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 53 - DNA, 1858 bp, synthetic construct
  misc_feature 1..1858: note = CXCR4 H281A
  source 1..1858: mol_type = other DNA
taatacgactcactataaggactcactatttgttttcgcgcccagttgcaaaaagtgtcgccaccatggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtggccaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagctgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 54 - DNA, 3898 bp, synthetic construct
  misc_feature 1..3898: note = ITGA4
  source 1..3898: mol_type = other DNA
taatacgactcactataaggactcactatttgttttcgcgcccagttgcaaaaagtgtcgccaccatggcttgggaggctcggagagaacctggacctagaagagctgccgtgcgggagactgtcatgctgctgctgtgcctgggggtgcccacaggcagaccttacaacgtggataccgagagcgccctgctgtatcagggcccccacaacaccctgtttggctactctgtggtgctgcacagccacggcgccaaccgctggctgctggtgggcgcccccaccgccaattggctggccaatgcctccgtgatcaacccaggcgccatctacagatgtcggatcggcaagaatcctggccagacatgcgagcagctgcagctgggctcccccaacggcgagccttgtggcaagacatgcctggaggagagggacaatcagtggctgggcgtgacactgagcagacagcccggcgagaacggctccatcgtgacatgcggccacagatggaagaacatcttttacatcaagaacgagaataagctgccaacaggcggctgctatggcgtgcccccagacctgagaacagagctgagcaagcggatcgccccatgctaccaggattatgtgaagaagtttggcgagaattttgcctcttgccaggccggcatctcctccttctacaccaaggatctgatcgtgatgggcgcccctggctcttcctattggacaggctctctgttcgtgtataatatcacaaccaacaagtacaaggccttcctggacaagcagaaccaggtgaagttcggctcctatctgggctacagcgtgggcgccggccactttcggtctcagcacaccacagaggtggtgggcggcgccccccagcacgagcagatcggcaaggcctacatcttctccatcgacgagaaggagctgaatatcctgcacgagatgaagggcaagaagctgggctcctactttggcgcctccgtgtgcgccgtggacctgaatgccgacggcttttccgacctgctggtgggcgccccaatgcagtccacaatcagagaggagggcagagtgttcgtgtatatcaattccggcagcggcgccgtgatgaatgccatggagaccaatctggtgggctccgacaagtatgccgccagattcggcgagagcatcgtgaatctgggcgacatcgacaacgatggcttcgaggacgtggccatcggcgccccacaggaggatgacctgcagggcgccatctatatctataacggccgggccgacggcatctctagcaccttctcccagagaatcgagggcctgcagatcagcaagtccctgagcatgttcggccagagcatctccggccagatcgacgccgataataacggctacgtggatgtggccgtgggcgcctttagaagcgactccgccgtgctgctgagaacaaggcccgtggtgatcgtggatgcctccctgtctcaccccgagtccgtgaatcggacaaagtttgactgcgtggagaatggctggccaagcgtgtgcatcgatctgacactgtgcttttcctataagggcaaggaggtgccaggctatatcgtgctgttttacaacatgtctctggatgtgaacagaaaggccgagtcccccccaagattctacttttcctctaacggcacctctgatgtgatcaccggctctatccaggtgtccagcagggaggccaattgcagaacccaccaggcctttatgcggaaggatgtgcgcgacatcctgaccccaatccagatcgaggccgcctatcacctgggcccccacgtgatctccaagcggtccaccgaggagttccctccactgcagccaatcctgcagcagaagaaggagaaggacatcatgaagaagacaatcaacttcgccaggttttgcgcccacgagaactgttccgccgacctgcaggtgtctgccaagatcggcttcctgaagccccacgagaacaagacatatctggccgtgggctccatgaagaccctgatgctgaacgtgagcctgtttaacgccggcgacgatgcctacgagacaacactgcacgtgaagctgccagtgggcctgtacttcatcaagatcctggagctggaggagaagcagatcaactgtgaggtgaccgataactccggcgtggtgcagctggattgcagcatcggctatatctacgtggaccacctgtcccgcatcgacatctcttttctgctggacgtgtccagcctgtcccgggccgaggaggacctgtccatcacagtgcacgccacctgcgagaatgaggaggagatggacaacctgaagcactccagagtgacagtggccatcccactgaagtacgaggtgaagctgacagtgcacggctttgtgaatccaacctccttcgtgtacggctccaatgacgagaatgagccagagacatgtatggtggagaagatgaacctgacatttcacgtgatcaatacaggcaattctatggcccctaacgtgagcgtggagatcatggtgccaaattctttcagcccacagacagacaagctgtttaacatcctggacgtgcagacaaccacaggcgagtgtcactttgagaactaccagagagtgtgcgccctggagcagcagaagtccgccatgcagacactgaagggcatcgtgagatttctgagcaagacagataagaggctgctgtactgcatcaaggccgatccccactgcctgaattttctgtgcaacttcggcaagatggagtctggcaaggaggcctccgtgcacatccagctggagggcagaccctccatcctggagatggacgagaccagcgccctgaagttcgagatcagagccacaggcttcccagagcccaacccccgggtgatcgagctgaacaaggatgagaacgtggcccacgtgctgctggagggcctgcaccaccagcggcccaagagatatttcaccatcgtgatcatctccagctctctgctgctgggcctgatcgtgctgctgctgatctcctatgtgatgtggaaggccggcttctttaagcggcagtacaagtccatcctgcaggaagaaaatcgacgcgattcatggtcttacattaattctaaatcaaacgacgactaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgttaattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 55 - DNA, 3684 bp, synthetic construct
  misc_feature 1..3684: note = KIT
  source 1..3684: mol_type = other DNA
taatacgactcactataaggactcactatttgttttcgcgcccagttgcaaaaagtgtcgccaccatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcggccgcgtcgatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcgtaaaaaaaaaaaaaaaaaaaaaaaaaaaaaaaaaaaaaaaaaaaaaaaaaaaaaaaaaaaaaaaa

SEQ ID NO: 56 - DNA, 41 bp, synthetic construct
  misc_feature 1..41: note = aptamer
  source 1..41: mol_type = other DNA
gactcactatttgttttcgcgcccagttgcaaaaagtgtcg

SEQ ID NO: 57 - DNA, 18 bp, synthetic construct
  misc_feature 1..18: note = HIV sense
  source 1..18: mol_type = other DNA
tactgacgctctcgcacc

SEQ ID NO: 58 - DNA, 17 bp, synthetic construct
  misc_feature 1..17: note = HIV antisense
  source 1..17: mol_type = other DNA
tctcgacgcaggactcg

SEQ ID NO: 59 - DNA, 20 bp, synthetic construct
  misc_feature 1..20: note = Probe
  source 1..20: mol_type = other DNA
atctctctccttctagcctc

SEQ ID NO: 60 - DNA, 18 bp, synthetic construct
  misc_feature 1..18: note = Telo sense
  source 1..18: mol_type = other DNA
ggcacacgtggcttttcg

SEQ ID NO: 61 - DNA, 24 bp, synthetic construct
  misc_feature 1..24: note = Telo antisense
  source 1..24: mol_type = other DNA
ggtgaacctcgtaagtttatgcaa

SEQ ID NO: 62 - DNA, 24 bp, synthetic construct
  misc_feature 1..24: note = Telo probe
  source 1..24: mol_type = other DNA
tcaggacgtcgagtggacacggtg